(19) United States
(12) Patent Application Publication
(10) Pub. No.: US 2024/0318176 A1
(43) Pub. Date: Sep. 26, 2024

(54) MUSCLE TARGETING COMPLEXES AND USES THEREOF FOR TREATING DYSTROPHINOPATHIES

(71) Applicant: Dyne Therapeutics, Inc., Waltham, MA (US)

(72) Inventors: Cody A. Desjardins, Waltham, MA (US); Kim Tang, Waltham, MA (US); James McSwiggen, Arvada, CO (US); Romesh R. Subramanian, Framingham, MA (US); Timothy Weeden, Waltham, MA (US); Mohammed T. Qatanani, Waltham, MA (US); Brendan Quinn, Waltham, MA (US); John Najim, Waltham, MA (US)

(73) Assignee: Dyne Therapeutics, Inc., Waltham, MA (US)

(21) Appl. No.: 18/577,374

(22) PCT Filed: Jul. 8, 2022
(86) PCT No.: PCT/US2022/073527

§ 371 Date: Jan. 8, 2024

Related U.S. Application Data
(60) Provisional application No. 63/219,999, filed on Jul. 9, 2021.

(51) Int. Cl. C12N 15/113; A61K 39/00; A61K 47/68; C07K 16/28

(52) U.S. Cl. CPC C12N 15/113; A61K 47/6807; A61K 47/6849; C07K 16/2881; A61K 2039/505; C07K 2317/55; C12N 2310/11; C12N 2310/3233; C12N 2310/3513; C12N 2320/32; C12N 2320/33

(57) ABSTRACT

Aspects of the disclosure relate to complexes comprising a muscle-targeting agent covalently linked to a molecular payload. In some embodiments, the muscle-targeting agent specifically binds to an internalizing cell surface receptor on muscle cells. In some embodiments, the molecular payload promotes the expression or activity of a functional dystrophin protein. In some embodiments, the molecular payload is an oligonucleotide, such as an antisense oligonucleotide, e.g., an oligonucleotide that causes exon skipping in a mRNA expressed from a mutant DMD allele.

### RELATED APPLICATIONS

This application claims priority under 35 U.S.C. § 119(e) to U.S. Provisional Application Ser. No. 63/219,999, entitled “MUSCLE TARGETING COMPLEXES AND USES THEREOF FOR TREATING DYSTROPHINOPATHIES”, filed on Jul. 9, 2021, the contents of which are incorporated herein by reference in their entirety.

### FIELD OF THE INVENTION

The present application relates to targeting complexes for delivering molecular payloads (e.g., oligonucleotides) to cells and uses thereof, particularly uses relating to treatment of disease.

### REFERENCE TO AN ELECTRONIC SEQUENCE LISTING

The contents of the electronic sequence listing (D082470065WO00-SEQ-COB.xml; Size: 2,801,833 bytes; and Date of Creation: Jul. 7, 2022) is herein incorporated by reference in its entirety.

### BACKGROUND OF INVENTION

Dystrophinopathies are a group of distinct neuromuscular diseases that result from mutations in the gene encoding dystrophin. Dystrophinopathies include Duchenne muscular dystrophy, Becker muscular dystrophy, and X-linked dilated cardiomyopathy. The DMD gene (“DMD”), which encodes dystrophin, is a large gene, containing 79 exons and about 2.6 million total base pairs. Numerous mutations in DMD, including exonic frameshift, deletion, substitution, and duplicative mutations, are able to diminish the expression of functional dystrophin, leading to dystrophinopathies. Several agents that target exons of human DMD have been approved by the U.S. Food and Drug Administration (FDA), including casimersen, viltolarsen, golodirsen, and eteplirsen.

### SUMMARY OF INVENTION

According to some aspects, the disclosure provides complexes that target muscle cells for purposes of delivering molecular payloads to those cells, as well as molecular payloads that can be used therein. In some embodiments, complexes provided herein are particularly useful for delivering molecular payloads that increase or restore expression or activity of functional dystrophin protein. In some embodiments, complexes comprise oligonucleotide based molecular payloads that promote expression of functional dystrophin protein through an in-frame exon skipping mechanism or suppression of stop codons, such as by facilitating skipping of DMD exon 55. In some embodiments, molecular payloads provided herein are useful for facilitating exon skipping in a DMD sequence, such as skipping of DMD exon 55. Accordingly, in some embodiments, complexes provided herein comprise muscle-targeting agents (e.g., muscle targeting antibodies) that specifically bind to receptors on the surface of muscle cells for purposes of delivering molecular payloads to the muscle cells. In some embodiments, the complexes are taken up into the cells via a receptor mediated internalization, following which the molecular payload may be released to perform a function inside the cells. For example, complexes engineered to deliver oligonucleotides may release the oligonucleotides such that the oligonucleotides can promote expression of functional dystrophin protein (e.g., through an exon skipping mechanism, such as by facilitating skipping of DMD exon 55) in the muscle cells. In some embodiments, the oligonucleotides are released by endosomal cleavage of covalent linkers connecting oligonucleotides and muscle-targeting agents of the complexes. Complexes and molecular payloads provided herein can be used for treating subjects having a mutated DMD gene, such as a mutated DMD gene that is amenable to exon 55 skipping.

According to some aspects, complexes comprising an anti-transferrin receptor 1 (TfR1) antibody covalently linked to an oligonucleotide configured for inducing skipping of exon 55 in a DMD pre-mRNA are provided herein, wherein the oligonucleotide comprises a region of complementarity that is complementary with at least 8 consecutive nucleotides of any one of SEQ ID NOs: 160-779.

In some embodiments, the anti-TfR1 antibody comprises:
(i) a heavy chain complementarity determining region 1 (CDR-H1) of SEQ ID NO: 33, a heavy chain complementarity determining region 2 (CDR-H2) of SEQ ID NO: 34, a heavy chain complementarity determining region 3 (CDR-H3) of SEQ ID NO: 35, a light chain complementarity determining region 1 (CDR-L1) of SEQ ID NO: 36, a light chain complementarity determining region 2 (CDR-L2) of SEQ ID NO: 37, and a light chain complementarity determining region 3 (CDR-L3) of SEQ ID NO: 32;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6;
(iii) a CDR-H1 of SEQ ID NO: 7, a CDR-H2 of SEQ ID NO: 20, a CDR-H3 of SEQ ID NO: 9, a CDR-L1 of SEQ ID NO: 10, a CDR-L2 of SEQ ID NO: 11, and a CDR-L3 of SEQ ID NO: 6;
(iv) a CDR-H1 of SEQ ID NO: 7, a CDR-H2 of SEQ ID NO: 24, a CDR-H3 of SEQ ID NO: 9, a CDR-L1 of SEQ ID NO: 10, a CDR-L2 of SEQ ID NO: 11, and a CDR-L3 of SEQ ID NO: 6;
(v) a CDR-H1 of SEQ ID NO: 51, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50;
(vi) a CDR-H1 of SEQ ID NO: 64, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50; or
(vii) a CDR-H1 of SEQ ID NO: 67, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50.

In some embodiments, the anti-TfR1 antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a VH comprising an amino acid sequence at least 85% identical to SEQ ID NO: 69; and/or a VL comprising an amino acid sequence at least 85% identical to SEQ ID NO: 70;
(iii) a VH comprising an amino acid sequence at least 85% identical to SEQ ID NO: 71; and/or a VL comprising an amino acid sequence at least 85% identical to SEQ ID NO: 70;
(iv) a VH comprising an amino acid sequence at least 85% identical to SEQ ID NO: 72; and/or a VL comprising an amino acid sequence at least 85% identical to SEQ ID NO: 70;
(v) a VH comprising an amino acid sequence at least 85% identical to SEQ ID NO: 73; and/or a VL comprising an amino acid sequence at least 85% identical to SEQ ID NO: 74;
(vi) a VH comprising an amino acid sequence at least 85% identical to SEQ ID NO: 73; and/or a VL comprising an amino acid sequence at least 85% identical to SEQ ID NO: 75;
(vii) a VH comprising an amino acid sequence at least 85% identical to SEQ ID NO: 76; and/or a VL comprising an amino acid sequence at least 85% identical to SEQ ID NO: 74;
(viii) a VH comprising an amino acid sequence at least 85% identical to SEQ ID NO: 77; and/or a VL comprising an amino acid sequence at least 85% identical to SEQ ID NO: 78;
(ix) a VH comprising an amino acid sequence at least 85% identical to SEQ ID NO: 79; and/or a VL comprising an amino acid sequence at least 85% identical to SEQ ID NO: 80; or
(x) a VH comprising an amino acid sequence at least 85% identical to SEQ ID NO: 77; and/or a VL comprising an amino acid sequence at least 85% identical to SEQ ID NO: 80.

In some embodiments, the anti-TfR1 antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 69 and a VL comprising the amino acid sequence of SEQ ID NO: 70;
(iii) a VH comprising the amino acid sequence of SEQ ID NO: 71 and a VL comprising the amino acid sequence of SEQ ID NO: 70;
(iv) a VH comprising the amino acid sequence of SEQ ID NO: 72 and a VL comprising the amino acid sequence of SEQ ID NO: 70;
(v) a VH comprising the amino acid sequence of SEQ ID NO: 73 and a VL comprising the amino acid sequence of SEQ ID NO: 74;
(vi) a VH comprising the amino acid sequence of SEQ ID NO: 73 and a VL comprising the amino acid sequence of SEQ ID NO: 75;
(vii) a VH comprising the amino acid sequence of SEQ ID NO: 76 and a VL comprising the amino acid sequence of SEQ ID NO: 74;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.

In some embodiments, the anti-TfR1 antibody is a Fab fragment, a Fab′ fragment, a F(ab′)2 fragment, an scFv, an Fv, or a full-length IgG.

In some embodiments, the anti-TfR1 antibody is a Fab fragment.

In some embodiments, the anti-TfR1 antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 97; and/or a light chain comprising an amino acid sequence at least 85% identical to SEQ ID NO: 85;
(iii) a heavy chain comprising an amino acid sequence at least 85% identical to SEQ ID NO: 98; and/or a light chain comprising an amino acid sequence at least 85% identical to SEQ ID NO: 85;
(iv) a heavy chain comprising an amino acid sequence at least 85% identical to SEQ ID NO: 99; and/or a light chain comprising an amino acid sequence at least 85% identical to SEQ ID NO: 85;
(v) a heavy chain comprising an amino acid sequence at least 85% identical to SEQ ID NO: 100; and/or a light chain comprising an amino acid sequence at least 85% identical to SEQ ID NO: 89;
(vi) a heavy chain comprising an amino acid sequence at least 85% identical to SEQ ID NO: 100; and/or a light chain comprising an amino acid sequence at least 85% identical to SEQ ID NO: 90;
(vii) a heavy chain comprising an amino acid sequence at least 85% identical to SEQ ID NO: 101; and/or a light chain comprising an amino acid sequence at least 85% identical to SEQ ID NO: 89;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.

In some embodiments, the anti-TfR1 antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 97; and a light chain comprising the amino acid sequence of SEQ ID NO: 85;
(iii) a heavy chain comprising the amino acid sequence of SEQ ID NO: 98; and a light chain comprising the amino acid sequence of SEQ ID NO: 85;
(iv) a heavy chain comprising the amino acid sequence of SEQ ID NO: 99; and a light chain comprising the amino acid sequence of SEQ ID NO: 85;
(v) a heavy chain comprising the amino acid sequence of SEQ ID NO: 100; and a light chain comprising the amino acid sequence of SEQ ID NO: 89;
(vi) a heavy chain comprising the amino acid sequence of SEQ ID NO: 100; and a light chain comprising the amino acid sequence of SEQ ID NO: 90;
(vii) a heavy chain comprising the amino acid sequence of SEQ ID NO: 101; and a light chain comprising the amino acid sequence of SEQ ID NO: 89;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.

In some embodiments, the anti-TfR1 antibody does not specifically bind to the transferrin binding site of the transferrin receptor 1 and/or the anti-TfR1 antibody does not inhibit binding of transferrin to the transferrin receptor 1.

In some embodiments, the oligonucleotide comprises a region of complementarity to at least 4 consecutive nucleotides of a splicing feature of the DMD pre-mRNA.

In some embodiments, the splicing feature is an exonic splicing enhancer (ESE) in exon 55 of the DMD pre-mRNA, optionally wherein the ESE comprises a sequence of any one of SEQ ID NOs: 2031-2061.

In some embodiments, the splicing feature is a branch point, a splice donor site, or a splice acceptor site, optionally wherein the splicing feature is across the junction of exon 54 and intron 54, in intron 54, across the junction of intron 54 and exon 55, across the junction of exon 55 and intron 55, in intron 55, or across the junction of intron 55 and exon 56 of the DMD pre-mRNA, and further optionally wherein the splicing feature comprises a sequence of any one of SEQ ID NOs: 2028-2030, 2062, and 2063.

In some embodiments, the oligonucleotide comprises a sequence complementary to any one of SEQ ID NOs: 160-779 or comprises a sequence of any one of SEQ ID NOs: 780-2019, wherein each thymine base (T) may independently and optionally be replaced with a uracil base (U), and each U may independently and optionally be replaced with a T.

In some embodiments, the oligonucleotide comprises a sequence of any one of SEQ ID NOs: 1400, 1402-1406, 1408, 1409, 1413, 1418-1420, 1483-1491, 1493, 1495, 1496, 1502-1506, 1508, 1510-1512, 1514, 1522-1524, 1529-1531, 1534, 1535, 1559, 1583, 1587, 1591, 1596, 1597, 1598, 1604, 1606, 1607, 1638, 1641, 1693-1695, 1702, 1703, 1766, 1813, 1988, and 1995, wherein each thymine base (T) may independently and optionally be replaced with a uracil base (U), and each U may independently and optionally be replaced with a T.

In some embodiments, the oligonucleotide comprises one or more phosphorodiamidate morpholinos, optionally wherein the oligonucleotide is a phosphorodiamidate morpholino oligomer (PMO).

In some embodiments, the anti-TfR1 antibody is covalently linked to the oligonucleotide via a cleavable linker, optionally wherein the cleavable linker comprises a valine-citrulline sequence.

In some embodiments, the anti-TfR1 antibody is covalently linked to the oligonucleotide via conjugation to a lysine residue or a cysteine residue of the antibody.

According to some aspects, oligonucleotides that target DMD are provided herein, wherein the oligonucleotide comprises a region of complementarity to any one of SEQ ID NOs: 160-779, optionally wherein the region of complementarity comprises at least 15 consecutive nucleosides complementary to any one of SEQ ID NOs: 160-779.

In some embodiments, the oligonucleotide comprises at least 15 consecutive nucleosides of any one of SEQ ID NOs: 780-2019, optionally wherein the oligonucleotide comprises a sequence of any one of SEQ ID NOs: 780-2019, wherein each thymine base (T) may independently and optionally be replaced with a uracil base (U), and each U may independently and optionally be replaced with a T.

According to some aspects, methods of delivering an oligonucleotide to a cell are provided herein, the method comprising contacting the cell with a complex disclosed herein or with an oligonucleotide disclosed herein.

According to some aspects, methods of promoting the expression or activity of a dystrophin protein in a cell are provided herein, the method comprising contacting the cell with a complex disclosed herein or with an oligonucleotide disclosed herein in an amount effective for promoting internalization of the oligonucleotide to the cell, optionally wherein the cell is a muscle cell.

In some embodiments, the cell comprises a DMD gene that is amenable to skipping of exon 55.

In some embodiments, the dystrophin protein is a truncated dystrophin protein.

### BRIEF DESCRIPTION OF THE DRAWINGS

FIG. 1 shows data illustrating that conjugates containing anti-TfR1 Fab (3M12 VH4/Vκ3) conjugated to a DMD exon-skipping oligonucleotide resulted in enhanced exon skipping compared to the naked DMD exon skipping oligo in Duchenne muscular dystrophy patient myotubes.

### DETAILED DESCRIPTION OF INVENTION

Aspects of the disclosure relate to a recognition that while certain molecular payloads (e.g., oligonucleotides, peptides, small molecules) can have beneficial effects in muscle cells, it has proven challenging to effectively target such cells. Accordingly, as described herein, the present disclosure provides complexes comprising muscle-targeting agents covalently linked to molecular payloads in order to overcome such challenges. In some embodiments, the complexes are particularly useful for delivering molecular payloads that modulate (e.g., promote) the expression or activity of dystrophin protein (e.g., a truncated dystrophin protein) or DMD (e.g., a mutated DMD allele). In some embodiments, complexes provided herein may comprise oligonucleotides that promote expression and activity of dystrophin protein or DMD, such as by facilitating in-frame exon skipping and/or suppression of premature stop codons. For example, complexes may comprise oligonucleotides that induce skipping of exon(s) of DMD RNA (e.g., pre-mRNA), such as oligonucleotides that induce skipping of exon 55. In some embodiments, synthetic nucleic acid payloads (e.g., DNA or RNA payloads) may be used that express one or more proteins that promote normal expression and activity of dystrophin protein or DMD.

Duchenne muscular dystrophy is an X-linked muscular disorder caused by one or more mutations in the DMD gene located on Xp21. Dystrophin protein typically forms the dystrophin-associated glycoprotein complex (DGC) at the sarcolemma, which links the muscle sarcomeric structure to the extracellular matrix and protects the sarcolemma from contraction-induced injury. In patients with Duchenne muscular dystrophy, the dystrophin protein is generally absent and muscle fibers typically become damaged due to mechanical overextension. Mutations in the DMD gene are associated with two types of muscular dystrophy, Duchenne muscular dystrophy and Becker muscular dystrophy, depending on whether the translational reading frame is lost or maintained. Becker muscular dystrophy is a clinically milder form of Duchenne muscular dystrophy, and is characterized by features similar to Duchenne muscular dystrophy. In some embodiments, exon skipping induced by oligonucleotides (e.g., delivered using complexes provided herein) can be used to restore the reading frame of a mutated DMD allele resulting in production of a truncated dystrophin protein that is sufficiently functional to improve muscle function. In some embodiments, such exon skipping converts a Duchenne muscular dystrophy phenotype into a milder Becker muscular dystrophy phenotype.

Further aspects of the disclosure, including a description of defined terms, are provided below.

### I. Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Branch point: As used herein, the term “branch point” or “branch site” refers to a nucleic acid sequence motif within an intron of a gene or pre-mRNA that is involved in splicing of pre-mRNA into mRNA (i.e., removing introns from the pre-mRNA), and can be referred to as a splicing feature. A branch point is typically located 18 to 40 nucleotides from the 3′ end of an intron, and contains an adenine but is otherwise relatively unrestricted in sequence. Common sequence motifs for branch points are YNYYRAY, YTRAC, and YNYTRAY, where Y is a pyrimidine, N is any nucleotide, R is any purine, and A is adenine. During splicing, the pre-mRNA is cleaved at the 5′ end of the intron, which then attaches to the branch point region downstream through transesterification bonding between guanines and adenines from the 5′ end and the branch point, respectively, to form a looped lariat structure.

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

CDR-grafted antibody: The term “CDR-grafted antibody” refers to antibodies which comprise heavy and light chain variable region sequences from one species but in which the sequences of one or more of the CDR regions of VH and/or (e.g., and) VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleosides or two sets of nucleosides. In particular, complementary is a term that characterizes an extent of hydrogen bond pairing that brings about binding between two nucleosides or two sets of nucleosides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

DMD: As used herein, the term “DMD” refers to a gene that encodes dystrophin protein, a key component of the dystrophin-glycoprotein complex, which bridges the inner cytoskeleton and the extracellular matrix in muscle cells, particularly muscle fibers. Deletions, duplications, and point mutations in DMD may cause dystrophinopathies, such as Duchenne muscular dystrophy, Becker muscular dystrophy, or cardiomyopathy. Alternative promoter usage and alternative splicing result in numerous distinct transcript variants and protein isoforms for this gene. In some embodiments, a dystrophin gene (DMD or DMD gene) may be a human (Gene ID: 1756), non-human primate (e.g., Gene ID: 465559), or rodent gene (e.g., Gene ID: 13405; Gene ID: 24907). In addition, multiple human transcript variants (e.g., as annotated under GenBank RefSeq Accession Numbers: NM_000109.3, NM_004006.2, NM_004009.3, NM_004010.3 and NM_004011.3) have been characterized that encode different protein isoforms.

DMD allele: As used herein, the term “DMD allele” refers to any one of alternative forms (e.g., wild-type or mutant forms) of a DMD gene. In some embodiments, a DMD allele may encode for dystrophin that retains its normal and typical functions. In some embodiments, a DMD allele may comprise one or more mutations that results in muscular dystrophy. Common mutations that lead to Duchenne muscular dystrophy involve frameshift, deletion, substitution, and duplicative mutations of one or more of 79 exons present in a dystrophin allele, e.g., exon 8, exon 23, exon 41, exon 44, exon 45, exon 50, exon 51, exon 52, exon 53, or exon 55. Further examples of DMD mutations are disclosed, for example, in Flanigan K M, et al., Mutational spectrum of DMD mutations in dystrophinopathy patients: application of modern diagnostic techniques to a large cohort. Hum Mutat. 2009 December; 30 (12):1657-66, the contents of which are incorporated herein by reference in its entirety.

Dystrophinopathy: As used herein, the term “dystrophinopathy” refers to a muscle disease results from one or more mutated DMD alleles. Dystrophinopathies include a spectrum of conditions (ranging from mild to severe) that includes Duchenne muscular dystrophy, Becker muscular dystrophy, and DMD-associated dilated cardiomyopathy (DCM). In some embodiments, at one end of the spectrum, dystrophinopathy is phenotypically associated with an asymptomatic increase in serum concentration of creatine phosphokinase (CK) and/or (e.g., and) muscle cramps with myoglobinuria. In some embodiments, at the other end of the spectrum, dystrophinopathy is phenotypically associated with progressive muscle diseases that are generally classified as Duchenne or Becker muscular dystrophy when skeletal muscle is primarily affected and as DMD-associated dilated cardiomyopathy (DCM) when the heart is primarily affected. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200. Becker muscular dystrophy is associated with OMIM Entry #300376. Dilated cardiomyopathy is associated with OMIM Entry X #302045.

Exonic splicing enhancer (ESE): As used herein, the term “exonic splicing enhancer” or “ESE” refers to a nucleic acid sequence motif within an exon of a gene, pre-mRNA, or mRNA that directs or enhances splicing of pre-mRNA into mRNA, e.g., as described in Blencowe et al., Trends Biochem Sci 25, 106-10. (2000), incorporated herein by reference. ESEs can be referred to as splicing features. ESEs may direct or enhance splicing, for example, to remove one or more introns and/or one or more exons from a gene transcript. ESE motifs are typically 6-8 nucleobases in length. SR proteins (e.g., proteins encoded by the gene SRSF1, SRSF2, SRSF3, SRSF4, SRSF5, SRSF6, SRSF7, SRSF8, SRSF9, SRSF10, SRSF11, SRSF12, TRA2A or TRA2B) bind to ESEs through their RNA recognition motif region to facilitate splicing. ESE motifs can be identified through a number of methods, including those described in Cartegni et al., Nucleic Acids Research, 2003, Vol. 31, No. 13, 3568-3571, incorporated herein by reference.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding non-human CDR sequences. In one embodiment, humanized anti-TfR1 antibodies and antigen binding portions are provided. Such antibodies may be generated by obtaining murine anti-TfR1 monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or (e.g., and) an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or (e.g., and) chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad. Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

Muscle-targeting agent: As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload.

Muscle-targeting antibody: As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkages. In some embodiments, an oligonucleotide may comprise one or more phosphorothioate linkages, which may be in the Rp or Sp stereochemical conformation.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Splice acceptor site: As used herein, the term “splice acceptor site” or “splice acceptor” refers to a nucleic acid sequence motif at the 3′ end of an intron or across an intron/exon junction of a gene or pre-mRNA that is involved in splicing of pre-mRNA into mRNA (i.e., removing introns from the pre-mRNA), and can be referred to as a splicing feature. A splice acceptor site includes a terminal AG sequence at the 3′ end of an intron, which is typically preceded (5′-ward) by a region high in pyrimidines (C/U). Upstream from the splice acceptor site is the branch point. Formation of a lariat loop intermediate structure by a transesterification reaction between the branch point and the splice donor site releases a 3′-OH of the 5′ exon, which subsequently reacts with the first nucleotide of the 3′ exon, thereby joining the exons and releasing the intron lariat. The AG sequence at the 3′ end of the intron in the splice acceptor site is known to be critical for proper splicing, as changing one of these nucleotides results in inhibition of splicing. Rarely, alternative splice acceptor sites have an AC at the 3′ end of the intron, instead of the more common AG. A common splice acceptor site motif has a sequence of or similar to [Y-rich region]-NCAGG or YₓNYAGG, in which Y represents a pyrimidine, N represents any nucleotide, and x is a number from 4 to 20. The cut site follows the AG, which represent the 3′-terminal nucleotides of the excised intron.

Splice donor site: As used herein, the term “splice donor site” or “splice donor” refers to a nucleic acid sequence motif at the 5′ end of an intron or across an exon/intron junction of a gene or pre-mRNA that is involved in splicing of pre-mRNA into mRNA (i.e., removing introns from the pre-mRNA), and can be referred to as a splicing feature. A splice donor site includes a terminal GU sequence at the 5′ end of the intron, within a larger and fairly unconstrained sequence. During splicing, the 2′-OH of a nucleotide within the branch point initiates a transesterification reaction via a nucleophilic attack on the 5′ G of the intron within the splice donor site. The G is thereby cleaved from the pre-mRNA and bonds instead to the branch point nucleotide, forming a loop lariat structure. The 3′ nucleotide of the upstream exon subsequently binds the splice acceptor site, joining the exons and excising the intron. A typical splice donor site has a sequence of or similar to GGGURAGU or AGGURNG, in which R represents a purine and N represents any nucleotide. The cut site precedes the first GU (i.e., GG/GURAGU or AG/GURNG), which represent the 5′-terminal nucleotides of the excised intron.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a mutated DMD gene sequence, e.g., a mutation in an exon of a DMD gene sequence. In some embodiments, a subject has a dystrophinopathy, e.g., Duchenne muscular dystrophy. In some embodiments, a subject is a patient that has a mutation of the DMD gene that is amenable to exon 55 skipping.

Transferrin receptor: As used herein, the term, “transferrin receptor” (also known as TFRC, CD71, p90, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-0-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleosides and oligonucleotides comprising the 2′-modified nucleosides have increased affinity to a target sequences, relative to an unmodified oligonucleotide. Examples of structures of 2′-modified nucleosides are provided below:

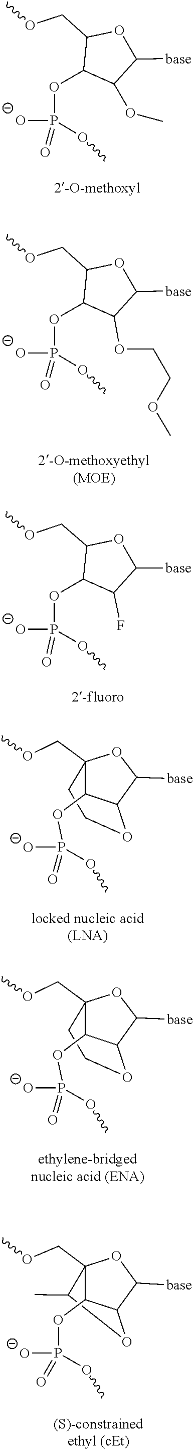

These examples are shown with phosphate groups, but any internucleoside linkages are contemplated between 2′-modified nucleosides.

### II. Complexes

Provided herein are complexes that comprise a targeting agent, e.g. an antibody, covalently linked to a molecular payload. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens.

A complex may be used to modulate the activity or function of at least one gene, protein, and/or (e.g., and) nucleic acid. In some embodiments, the molecular payload present within a complex is responsible for the modulation of a gene, protein, and/or (e.g., and) nucleic acids. A molecular payload may be a small molecule, protein, nucleic acid, oligonucleotide, or any molecular entity capable of modulating the activity or function of a gene, protein, and/or (e.g., and) nucleic acid in a cell.

In some embodiments, a complex comprises a muscle-targeting agent, e.g., an anti-transferrin receptor antibody, covalently linked to a molecular payload, e.g., an antisense oligonucleotide that targets DMD to promote exon skipping, e.g., in a transcript encoded from a mutated DMD allele. In some embodiments, the complex targets a DMD pre-mRNA to promote skipping of exon 55 in the DMD pre-mRNA.

### A. Muscle-Targeting Agents

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. It should be appreciated that various types of muscle-targeting agents may be used in accordance with the disclosure, and that any muscle targets (e.g., muscle surface proteins) can be targeted by any type of muscle-targeting agent described herein. For example, the muscle-targeting agent may comprise, or consist of, a small molecule, a nucleic acid (e.g., DNA or RNA), a peptide (e.g., an antibody), a lipid (e.g., a microvesicle), or a sugar moiety (e.g., a polysaccharide). Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or (e.g., and) a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload into muscle tissue. Binding to muscle surface recognition elements followed by endocytosis can allow even large molecules such as antibodies to enter muscle cells. As another example molecular payloads conjugated to transferrin or anti-TfR1 antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or (e.g., and) cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 March, 39(13):78309; the entire contents of each of which are incorporated herein by reference.

a. Anti-Transferrin Receptor (TfR) Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload, into a muscle cell. As used herein, an antibody that binds to a transferrin receptor may be referred to interchangeably as an, transferrin receptor antibody, an anti-transferrin receptor antibody, or an anti-TfR1 antibody. Antibodies that bind, e.g. specifically bind, to a transferrin receptor may be internalized into the cell, e.g. through receptor-mediated endocytosis, upon binding to a transferrin receptor.

It should be appreciated that anti-TfR1 antibodies may be produced, synthesized, and/or (e.g., and) derivatized using several known methodologies, e.g. library design using phage display. Exemplary methodologies have been characterized in the art and are incorporated by reference (Diez, P. et al. “High-throughput phage-display screening in array format”, Enzyme and microbial technology, 2015, 79, 34-41.; Christoph M. H. and Stanley, J. R. “Antibody Phage Display: Technique and Applications” J Invest Dermatol. 2014, 134:2.; Engleman, Edgar (Ed.) “Human Hybridomas and Monoclonal Antibodies.” 1985, Springer.). In other embodiments, an anti-TfR1 antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522.; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052.).

In some embodiments, the anti-TfR1 antibody described herein binds to transferrin receptor with high specificity and affinity. In some embodiments, the anti-TfR1 antibody described herein specifically binds to any extracellular epitope of a transferrin receptor or an epitope that becomes exposed to an antibody. In some embodiments, anti-TfR1 antibodies provided herein bind specifically to transferrin receptor from human, non-human primates, mouse, rat, etc. In some embodiments, anti-TfR1 antibodies provided herein bind to human transferrin receptor. In some embodiments, the anti-TfR1 antibody described herein binds to an amino acid segment of a human or non-human primate transferrin receptor, as provided in SEQ ID NOs: 105-108. In some embodiments, the anti-TfR1 antibody described herein binds to an amino acid segment corresponding to amino acids 90-96 of a human transferrin receptor as set forth in SEQ ID NO: 105, which is not in the apical domain of the transferrin receptor.

In some embodiments, the anti-TfR1 antibodies described herein (e.g., Anti-TfR clone 8 in Table 2 below) bind an epitope in TfR1, wherein the epitope comprises residues in amino acids 214-241 and/or amino acids 354-381 of SEQ ID NO: 105. In some embodiments, the anti-TfR1 antibodies described herein bind an epitope comprising residues in amino acids 214-241 and amino acids 354-381 of SEQ ID NO: 105. In some embodiments, the anti-TfR1 antibodies described herein bind an epitope comprising one or more of residues Y222, T227, K231, H234, T367, S368, S370, T376, and S378 of human TfR1 as set forth in SEQ ID NO: 105. In some embodiments, the anti-TfR1 antibodies described herein bind an epitope comprising residues Y222, T227, K231, H234, T367, S368, S370, T376, and S378 of human TfR1 as set forth in SEQ ID NO: 105.

In some embodiments, the anti-TfR1 antibody described herein (e.g., 3M12 in Table 2 below and its variants) bind an epitope in TfR1, wherein the epitope comprises residues in amino acids 258-291 and/or amino acids 358-381 of SEQ ID NO: 105. In some embodiments, the anti-TfR1 antibodies (e.g., 3M12 in Table 2 below and its variants) described herein bind an epitope comprising residues in amino acids amino acids 258-291 and amino acids 358-381 of SEQ ID NO: 105. In some embodiments, the anti-TfR1 antibodies described herein (e.g., 3M12 in Table 2 below and its variants) bind an epitope comprising one or more of residues K261, S273, Y282, T362, S368, S370, and K371 of human TfR1 as set forth in SEQ ID NO: 105. In some embodiments, the anti-TfR1 antibodies described herein (e.g., 3M12 in Table 2 below and its variants) bind an epitope comprising residues K261, S273, Y282, T362, S368, S370, and K371 of human TfR1 as set forth in SEQ ID NO: 105.

An example human transferrin receptor amino acid sequence, corresponding to NCBI sequence NP_003225.2 (transferrin receptor protein 1 isoform 1, Homo sapiens) is as follows:

(SEQ ID NO: 105)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 106)
MMDQARSAFSNLFGGEPLSYTRFSLARQVDGDNSHVEMKLGVDEEENTD
NNTKPNGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 107)
MMDQARSAFSNLFGGEPLSYTRFSLARQVDGDNSHVEMKLGVDEEENTDN
NTKANGTKPKRCGGNICYGTIAVIIFFLIGFMIGYLGYCKGVEPKTECER
LAGTESPAREEPEEDFPAAPRLYWDDLKRKLSEKLDTTDFTSTIKLLNEN
LYVPREAGSQKDENLALYIENQFREFKLSKVWRDQHFVKIQVKDSAQNSV
IIVDKNGGLVYLVENPGGYVAYSKAATVTGKLVHANFGTKKDFEDLDSPV
NGSIVIVRAGKITFAEKVANAESLNAIGVLIYMDQTKFPIVKADLSFFGH
AHLGTGDPYTPGFPSFNHTQFPPSQSSGLPNIPVQTISRAAAEKLFGNME
GDCPSDWKTDSTCKMVTSENKSVKLTVSNVLKETKILNIFGVIKGFVEPD
HYVVVGAQRDAWGPGAAKSSVGTALLLKLAQMFSDMVLKDGFQPSRSIIF
ASWSAGDFGSVGATEWLEGYLSSLHLKAFTYINLDKAVLGTSNFKVSASP
LLYTLIEKTMQDVKHPVTGRSLYQDSNWASKVEKLTLDNAAFPFLAYSGI
PAVSFCFCEDTDYPYLGTTMDTYKELVERIPELNKVARAAAEVAGQFVIK
LTHDTELNLDYERYNSQLLLFLRDLNQYRADVKEMGLSLQWLYSARGDFF
RATSRLTTDFRNAEKRDKFVMKKLNDRVMRVEYYFLSPYVSPKESPFRHV
FWGSGSHTLSALLESLKLRRQNNSAFNETLFRNQLALATWTIQGAANALS
GDVWDIDNEF.

An example mouse transferrin receptor amino acid sequence, corresponding to NCBI sequence NP_001344227.1 (transferrin receptor protein 1, Mus musculus) is as follows:

(SEQ ID NO: 108)
MMDQARSAFSNLFGGEPLSYTRFSLARQVDGDNSHVEMKLAADEEENADN
NMKASVRKPKRFNGRLCFAAIALVIFFLIGFMSGYLGYCKRVEQKEECVK
LAETEETDKSETMETEDVPTSSRLYWADLKTLLSEKLNSIEFADTIKQLS
QNTYTPREAGSQKDESLAYYIENQFHEFKFSKVWRDEHYVKIQVKSSIGQ
NMVTIVQSNGNLDPVESPEGYVAFSKPTEVSGKLVHANFGTKKDFEELSY
SVNGSLVIVRAGEITFAEKVANAQSFNAIGVLIYMDKNKFPVVEADLALF
GHAHLGTGDPYTPGFPSFNHTQFPPSQSSGLPNIPVQTISRAAAEKLFGK
MEGSCPARWNIDSSCKLELSQNQNVKLIVKNVLKERRILNIFGVIKGYEE
PDRYVVVGAQRDALGAGVAAKSSVGTGLLLKLAQVFSDMISKDGFRPSRS
IIFASWTAGDFGAVGATEWLEGYLSSLHLKAFTYINLDKVVLGTSNFKVS
ASPLLYTLMGKIMQDVKHPVDGKSLYRDSNWISKVEKLSFDNAAYPFLAY
SGIPAVSFCFCEDADYPYLGTRLDTYEALTQKVPQLNQMVRTAAEVAGQL
IIKLTHDVELNLDYEMYNSKLLSFMKDLNQFKTDIRDMGLSLQWLYSARG
DYFRATSRLTTDFHNAEKTNRFVMREINDRIMKVEYHFLSPYVSPRESPF
RHIFWGSGSHTLSALVENLKLRQKNITAFNETLFRNQLALATWTIQGVAN
ALSGDIWNIDNEF

In some embodiments, an anti-TfR1 antibody binds to an amino acid segment of the receptor as follows: FVKIQVKDSAQNSVIIVDKNGRLVYLVENPGGYVAYSKAATVTGKLVHANFGTKKDFE DLYTPVNGSIVIVRAGKITFAEKVANAESLNAIGVLIYMDQTKFPIVNAELSFFGHAHLG TGDPYTPGFPSFNHTQFPPSRSSGLPNIPVQTISRAAAEKLFGNMEGDCPSDWKTDSTCR MVTSESKNVKLTVSNVLKE (SEQ ID NO: 109) and does not inhibit the binding interactions between transferrin receptors and transferrin and/or (e.g., and) human hemochromatosis protein (also known as HFE). In some embodiments, the anti-TfR1 antibody described herein does not bind an epitope in SEQ ID NO: 109.

Appropriate methodologies may be used to obtain and/or (e.g., and) produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g. ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988.).

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, the anti-TfR1 antibody of the present disclosure comprises a VL domain and/or (e.g., and) a VH domain of any one of the anti-TfR1 antibodies selected from any one of Tables 2-7, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, agents binding to transferrin receptor, e.g., anti-TfR1 antibodies, are capable of targeting muscle cell and/or (e.g., and) mediate the transportation of an agent across the blood brain barrier. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Antibodies that bind, e.g. specifically bind, to a transferrin receptor may be internalized into the cell, e.g. through receptor-mediated endocytosis, upon binding to a transferrin receptor.

Provided herein, in some aspects, are humanized antibodies that bind to transferrin receptor with high specificity and affinity. In some embodiments, the humanized anti-TfR1 antibody described herein specifically binds to any extracellular epitope of a transferrin receptor or an epitope that becomes exposed to an antibody. In some embodiments, the humanized anti-TfR1 antibodies provided herein bind specifically to transferrin receptor from human, non-human primates, mouse, rat, etc. In some embodiments, the humanized anti-TfR1 antibodies provided herein bind to human transferrin receptor. In some embodiments, the humanized anti-TfR1 antibody described herein binds to an amino acid segment of a human or non-human primate transferrin receptor, as provided in SEQ ID NOs: 105-108. In some embodiments, the humanized anti-TfR1 antibody described herein binds to an amino acid segment corresponding to amino acids 90-96 of a human transferrin receptor as set forth in SEQ ID NO: 105, which is not in the apical domain of the transferrin receptor. In some embodiments, the humanized anti-TfR1 antibodies described herein binds to TfR1 but does not bind to TfR2.

In some embodiments, an anti-TFR1 antibody specifically binds a TfR1 (e.g., a human or non-human primate TfR1) with binding affinity (e.g., as indicated by Kd) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, the anti-TfR1 antibodies described herein bind to TfR1 with a KD of sub-nanomolar range. In some embodiments, the anti-TfR1 antibodies described herein selectively bind to transferrin receptor 1 (TfR1) but do not bind to transferrin receptor 2 (TfR2). In some embodiments, the anti-TfR1 antibodies described herein bind to human TfR1 and cyno TfR1 (e.g., with a Kd of 10⁻⁷ M, 10⁻⁸ M, 10⁻⁹ M, 10⁻¹⁰ M, 10⁻¹¹ M, 10⁻¹² M, 10⁻¹³ M, or less), but do not bind to a mouse TfR1. The affinity and binding kinetics of the anti-TfR1 antibody can be tested using any suitable method including but not limited to biosensor technology (e.g., OCTET or BIACORE). In some embodiments, binding of any one of the anti-TfR1 antibodies described herein does not complete with or inhibit transferrin binding to the TfR1. In some embodiments, binding of any one of the anti-TfR1 antibodies described herein does not complete with or inhibit HFE-beta-2-microglobulin binding to the TfR1.

Non-limiting examples of anti-TfR1 antibodies are provided in Table 2.

TABLE 2
Examples of Anti-TfR1 Antibodies

| | No. | | | |
|---|---|---|---|---|
| Ab | system | IMGT | Kabat | Chothia |
| 3-A4 | CDR- | GFNIKDDY (SEQ ID NO: | DDYMY (SEQ ID NO: 7) | GFNIKDD (SEQ ID NO: 12) |
| | H1 | 1) | | |
| | CDR- | IDPENGDT (SEQ ID NO: | WIDPENGDTEYASKFQD | ENG (SEQ ID NO: 13) |
| | H2 | 2) | (SEQ ID NO: 8) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS (SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPENGDT | | |
| | | EYASKFQDKATVTADTSSNTAYLQLSSLTSEDTAVYYCTLWLRRGLDYWGQGTSVTVS | | |
| | | S (SEQ ID NO: 17) | | |
| | VL | DIVMTQAAPSVPVTPGESVSISCRSSKSLLHSNGYTYLFWFLQRPGQSPQLLIYRMSNLA | | |
| | | SGVPDRFSGSGSGTAFTLRISRVEAEDVGVYYCMQHLEYPFTFGGGTKLEIK (SEQ ID | | |
| | | NO: 18) | | |
| 3-A4 | CDR- | GFNIKDDY (SEQ ID NO: | DDYMY (SEQ ID NO: 7) | GFNIKDD (SEQ ID NO: 12) |
| N54T* | H1 | 1) | | |
| | CDR- | IDPETGDT (SEQ ID NO: | WIDPETGDTEYASKFQD | ETG (SEQ ID NO: 21) |
| | H2 | 19) | (SEQ ID NO: 20) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS(SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPETGDT | | |
| | | EYASKFQDKATVTADTSSNTAYLQLSSLTSEDTAVYYCTLWLRRGLDYWGQGTSVTVS | | |
| | | S (SEQ ID NO: 22) | | |
| | VL | DIVMTQAAPSVPVTPGESVSISCRSSKSLLHSNGYTYLFWFLQRPGQSPQLLIYRMSNLA | | |
| | | SGVPDRFSGSGSGTAFTLRISRVEAEDVGVYYCMQHLEYPFTFGGGTKLEIK (SEQ ID | | |
| | | NO: 18) | | |
| 3-A4 | CDR- | GFNIKDDY (SEQ ID NO: | DDYMY (SEQ ID NO: 7) | GFNIKDD (SEQ ID NO: 12) |
| N54S* | H1 | 1) | | |
| | CDR- | IDPESGDT (SEQ ID NO: | WIDPESGDTEYASKFQD | ESG (SEQ ID NO: 25) |
| | H2 | 23) | (SEQ ID NO: 24) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS (SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPESGDT | | |
| | | EYASKFQDKATVTADTSSNTAYLQLSSLTSEDTAVYYCTLWLRRGLDYWGQGTSVTVS | | |
| | | S (SEQ ID NO: 26) | | |
| | VL | DIVMTQAAPSVPVTPGESVSISCRSSKSLLHSNGYTYLFWFLQRPGQSPQLLIYRMSNLA | | |
| | | SGVPDRFSGSGSGTAFTLRISRVEAEDVGVYYCMQHLEYPFTFGGGTKLEIK (SEQ ID | | |
| | | NO: 18) | | |
| 3-M12 | CDR- | GYSITSGYY (SEQ ID | SGYYWN (SEQ ID NO: 33) | GYSITSGY (SEQ ID NO: |
| | H1 | NO: 27) | | 38) |
| | CDR- | ITFDGAN (SEQ ID NO: | YITFDGANNYNPSLKN (SEQ | FDG (SEQ ID NO: 39) |
| | H2 | 28) | ID NO: 34) | |
| | CDR- | TRSSYDYDVLDY (SEQ | SSYDYDVLDY (SEQ ID NO: | SYDYDVLD (SEQ ID NO: |
| | H3 | ID NO: 29) | 35) | 40) |
| | CDR- | QDISNF (SEQ ID NO: 30) | RASQDISNFLN (SEQ ID NO: | SQDISNF (SEQ ID NO: 41) |
| | L1 | | 36) | |
| | CDR- | YTS (SEQ ID NO: 31) | YTSRLHS (SEQ ID NO: 37) | YTS (SEQ ID NO: 31) |
| | L2 | | | |
| | CDR- | QQGHTLPYT (SEQ ID | QQGHTLPYT (SEQ ID NO: 32) | GHTLPY (SEQ ID NO: 42) |
| | L3 | NO: 32) | | |
| | VH | DVQLQESGPGLVKPSQSLSLTCSVTGYSITSGYYWNWIRQFPGNKLEWMGYITFDGAN | | |
| | | NYNPSLKNRISITRDTSKNQFFLKLTSVTTEDTATYYCTRSSYDYDVLDYWGQGTTLTV | | |
| | | SS (SEQ ID NO: 43) | | |
| | VL | DIQMTQTTSSLSASLGDRVTISCRASQDISNFLNWYQQRPDGTVKLLIYYTSRLHSGVPS | | |
| | | RFSGSGSGTDFSLTVSNLEQEDIATYFCQQGHTLPYTFGGGTKLEIK (SEQ ID NO: 44) | | |
| 5-H12 | CDR- | GYSFTDYC (SEQ ID NO: | DYCIN (SEQ ID NO: 51) | GYSFTDY (SEQ ID NO: 56) |
| | H1 | 45) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYCINWVNQRPGQGLEWIGWIYPGSGNTR | | |
| | | YSERFKGKATLTVDTSSNTAYMQLSSLTSEDSAVYFCAREDYYPYHGMDYWGQGTSV | | |
| | | TVSS (SEQ ID NO: 61) | | |
| | VL | DIVLTQSPTSLAVSLGQRATISCRASESVDGYDNSFMHWYQQKPGQPPKLLIFRASNLES | | |
| | | GIPARFSGSGSRTDFTLTINPVEAADVATYYCQQSSEDPWTFGGGTKLEIK (SEQ ID NO: | | |
| | | 62) | | |
| 5-H12 | CDR- | GYSFTDYY (SEQ ID | DYYIN (SEQ ID NO: 64) | GYSFTDY (SEQ ID NO: 56) |
| C33Y* | H1 | NO: 63) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYYINWVNQRPGQGLEWIGWIYPGSGNTR | | |
| | | YSERFKGKATLTVDTSSNTAYMQLSSLTSEDSAVYFCAREDYYPYHGMDYWGQGTSV | | |
| | | TVSS (SEQ ID NO: 65) | | |
| | VL | DIVLTQSPTSLAVSLGQRATISCRASESVDGYDNSFMHWYQQKPGQPPKLLIFRASNLES | | |
| | | GIPARFSGSGSRTDFTLTINPVEAADVATYYCQQSSEDPWTFGGGTKLEIK (SEQ ID NO: | | |
| | | 62) | | |
| 5-H12 | CDR- | GYSFTDYD (SEQ ID | DYDIN (SEQ ID NO: 67) | GYSFTDY (SEQ ID NO: 56) |
| C33D* | H1 | NO: 66) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYDINWVNQRPGQGLEWIGWIYPGSGNTRY | | |
| | | SERFKGKATLTVDTSSNTAYMQLSSLTSEDSAVYFCAREDYYPYHGMDYWGQGTSVTV | | |
| | | SS (SEQ ID NO: 68) | | |
| | VL | DIVLTQSPTSLAVSLGQRATISCRASESVDGYDNSFMHWYQQKPGQPPKLLIFRASNLES | | |
| | | GIPARFSGSGSRTDFTLTINPVEAADVATYYCQQSSEDPWTFGGGTKLEIK (SEQ ID NO: | | |
| | | 62) | | |
| Anti- | CDR- | GYSFTSYW (SEQ ID | SYWIG (SEQ ID NO: 144) | GYSFTSY (SEQ ID NO: |
| TfR | H1 | NO: 138) | | 149) |
| clone 8 | CDR- | IYPGDSDT (SEQ ID NO: | IIYPGDSDTRYSPSFQGQ | GDS (SEQ ID NO: 150) |
| | H2 | 139) | (SEQ ID NO: 145) | |
| | CDR- | ARFPYDSSGYYSFDY | FPYDSSGYYSFDY (SEQ ID | PYDSSGYYSFD (SEQ ID |
| | H3 | (SEQ ID NO: 140) | NO: 146) | NO: 151) |
| | CDR- | QSISSY (SEQ ID NO: | RASQSISSYLN (SEQ ID NO: | SQSISSY (SEQ ID NO: 152) |
| | L1 | 141) | 147) | |
| | CDR- | AAS (SEQ ID NO: 142) | AASSLQS (SEQ ID NO: 148) | AAS (SEQ ID NO: 142) |
| | L2 | | | |
| | CDR- | QQSYSTPLT (SEQ ID | QQSYSTPLT (SEQ ID NO: | SYSTPL (SEQ ID NO: 153) |
| | L3 | NO: 143) | 143) | |

*mutation positions are according to Kabat numbering of the respective VH sequences containing the mutations

In some embodiments, the anti-TfR1 antibody of the present disclosure is a humanized variant of any one of the anti-TfR1 antibodies provided in Table 2. In some embodiments, the anti-TfR1 antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 in any one of the anti-TfR1 antibodies provided in Table 2, and comprises a humanized heavy chain variable region and/or (e.g., and) a humanized light chain variable region.

Examples of amino acid sequences of anti-TfR1 antibodies described herein are provided in Table 3.

TABLE 3
Variable Regions of Anti-TfR1 Antibodies

| Antibody | Variable Region Amino Acid Sequence** |
|---|---|
| 3A4 | V_{H}: |
| VH3 (N54T*)/Vκ4 | EVQLVQSGSELKKPGASVKVSCTASGFNIKDDYMYWVRQPPGKGLEWIGWIDP |
| | ETGDTEYASKFQDRVTVTADTSTNTAYMELSSLRSEDTAVYYCTLWLRRGLD |
| | YWGQGTLVTVSS (SEQ ID NO: 69) |
| | V_{L}: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGTK |
| | VEIK (SEQ ID NO: 70) |
| 3A4 | V_{H}: |
| VH3 (N54S*)/Vκ4 | EVQLVQSGSELKKPGASVKVSCTASGFNIKDDYMYWVRQPPGKGLEWIGWIDP |
| | ESGDTEYASKFQDRVTVTADTSTNTAYMELSSLRSEDTAVYYCTLWLRRGLD |
| | YWGQGTLVTVSS (SEQ ID NO: 71) |
| | V_{L}: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGTK |
| | VEIK (SEQ ID NO: 70) |
| 3A4 | VH: |
| VH3 /Vκ4 | EVQLVQSGSELKKPGASVKVSCTASGFNIKDDYMYWVRQPPGKGLEWIGWIDP |
| | ENGDTEYASKFQDRVTVTADTSTNTAYMELSSLRSEDTAVYYCTLWLRRGLD |
| | YWGQGTLVTVSS (SEQ ID NO: 72) |
| | VL: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGTK |
| | VEIK (SEQ ID NO: 70) |
| 3M12 | VH: |
| VH3/Vκ2 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSS (SEQ ID NO: 73) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIK (SEQ |
| | ID NO: 74) |
| 3M12 | VH: |
| VH3/Vκ3 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSS (SEQ ID NO: 73) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIK (SEQ |
| | ID NO: 75) |
| 3M12 | VH: |
| VH4/Vκ2 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSS (SEQ ID NO: 76) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIK (SEQ |
| | ID NO: 74) |
| 3M12 | VH: |
| VH4/Vκ3 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSS (SEQ ID NO: 76) |
| | VL: |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIK (SEQ |
| | ID NO: 75) |
| 5H12 | VH: |
| VH5 (C33Y*)/Vκ3 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSS (SEQ ID NO: 77) |
| | VL: |
| | DIVLTQSPDSLAVSLGERATINCRASESVDGYDNSFMHWYQQKPGQPPKLLIFR |
| | ASNLESGVPDRFSGSGSRTDFTLTISSLQAEDVAVYYCQQSSEDPWTFGQGTKL |
| | EIK (SEQ ID NO: 78) |
| 5H12 | VH: |
| VH5 (C33D*)/Vκ4 | QVQLVQSGAEVKKPGASVKVSCKASGYSFTDYDINWVRQAPGQGLEWMGWIY |
| | PGSGNTRYSERFKGRVTITRDTSASTAYMELSSLRSEDTAVYYCAREDYYPYH |
| | GMDYWGQGTLVTVSS (SEQ ID NO: 79) |
| | VL: |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKL |
| | EIK (SEQ ID NO: 80) |
| 5H12 | VH: |
| VH5 (C33Y*)/Vκ4 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSS (SEQ ID NO: 77) |
| | VL: |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKL |
| | EIK (SEQ ID NO: 80) |
| Anti-TfR clone 8 | VH: |
| | QVQLVQSGAEVKKPGESLKISCKGSGYSFTSYWIGWVRQMPGKGLEWMGIIYP |
| | GDSDTRYSPSFQGQVTISADKSISTAYLQWSSLKASDTAMYYCARFPYDSSGYY |
| | SFDYWGQGTLVTVSS (SEQ ID NO: 154) |
| | VL: |
| | DIQMTQSPSSLSASVGDRVTITCRASQSISSYLNWYQQKPGKAPKLLIYAASSLQ |
| | SGVPSRFSGSGSGTDFTLTISSLQPEDFATYYCQQSYSTPLTFGGGTKVEIK (SEQ |
| | ID NO: 155) |

*mutation positions are according to Kabat numbering of the respective VH sequences containing the mutations
**CDRs according to the Kabat numbering system are bolded

In some embodiments, the anti-TfR1 antibody of the present disclosure comprises a VH comprising the CDR-H1, CDR-H2, and CDR-H3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the CDR-H1, CDR-H2, and CDR-H3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the amino acid sequence of SEQ ID NO: 69 and a VL comprising the amino acid sequence of SEQ ID NO: 70.

In some embodiments, the anti-TfR1 antibody of the present disclosure comprises a VH comprising the amino acid sequence of SEQ ID NO: 71 and a VL comprising the amino acid sequence of SEQ ID NO: 70.

In some embodiments, the anti-TfR1 antibody of the present disclosure comprises a VH comprising the amino acid sequence of SEQ ID NO: 72 and a VL comprising the amino acid sequence of SEQ ID NO: 70.

In some embodiments, the anti-TfR1 antibody of the present disclosure comprises a VH comprising the amino acid sequence of SEQ ID NO: 73 and a VL comprising the amino acid sequence of SEQ ID NO: 74.

In some embodiments, the anti-TfR1 antibody of the present disclosure comprises a VH comprising the amino acid sequence of SEQ ID NO: 73 and a VL comprising the amino acid sequence of SEQ ID NO: 75.

In some embodiments, the anti-TfR1 antibody of the present disclosure comprises a VH comprising the amino acid sequence of SEQ ID NO: 76 and a VL comprising the amino acid sequence of SEQ ID NO: 74.

In some embodiments, the anti-TfR1 antibody of the present disclosure comprises a VH comprising the amino acid sequence of SEQ ID NO: 76 and a VL comprising the amino acid sequence of SEQ ID NO: 75.

In some embodiments, the anti-TfR1 antibody of the present disclosure comprises a VH comprising the amino acid sequence of SEQ ID NO: 77 and a VL comprising the amino acid sequence of SEQ ID NO: 78.

In some embodiments, the anti-TfR1 antibody of the present disclosure comprises a VH comprising the amino acid sequence of SEQ ID NO: 79 and a VL comprising the amino acid sequence of SEQ ID NO: 80.

In some embodiments, the anti-TfR1 antibody of the present disclosure comprises a VH comprising the amino acid sequence of SEQ ID NO: 77 and a VL comprising the amino acid sequence of SEQ ID NO: 80.

In some embodiments, the anti-TfR1 antibody of the present disclosure comprises a VH comprising the amino acid sequence of SEQ ID NO: 154 and a VL comprising the amino acid sequence of SEQ ID NO: 155.

In some embodiments, the anti-TfR1 antibody described herein is a full-length IgG, which can include a heavy constant region and a light constant region from a human antibody. In some embodiments, the heavy chain of any of the anti-TfR1 antibodies as described herein may comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 81)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the heavy chain of any of the anti-TfR1 antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcγ receptor binding (Bruhns, P., et al. (2009) and Xu, D. et al. (2000)). The mutant human IgG1 constant region is provided below (mutations bonded and underlined):

(SEQ ID NO: 82)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Other antibody heavy and light chain constant regions are well known in the art, e.g., those provided in the IMGT database (www.imgt.org) or at www.vbase2.org/vbstat.php, both of which are incorporated by reference herein.

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region as set forth in SEQ ID NO: 81. In some embodiments, the anti-TfR1 antibody described herein comprises heavy chain comprising any one of the VH as listed in Table 3 or any variants thereof and a heavy chain constant region as set forth in SEQ ID NO: 82.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of IgG heavy chain and light chain amino acid sequences of the anti-TfR1 antibodies described are provided in Table 4 below.

TABLE 4
Heavy chain and light chain sequences of examples of anti-TfR1 IgGs

| Antibody | IgG Heavy Chain/Light Chain Sequences** |
|---|---|
| 3A4 | Heavy Chain (with wild type human IgG1 constant region) |
| VH3 (N54T*)/Vκ4 | EVQLVQSGSELKKPGASVKVSCTASGFNIKDDYMYWVRQPPGKGLEWIGWIDPE |
| | TGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 84) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with wild type human IgG1 constant region) |
| VH3 (N54S*)/Vκ4 | EVQLVQSGSELKKPGASVKVSCTASGFNIKDDYMYWVRQPPGKGLEWIGWIDPE |
| | SGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 86) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with wild type human IgG1 constant region) |
| VH3 /Vκ4 | EVQLVQSGSELKKPGASVKVSCTASGFNIKDDYMYWVRQPPGKGLEWIGWIDPE |
| | NGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 87) |
| | Light Chain (with kappa light chain constant region) |
| | DIVMTQSPLSLPVTPGEPASISCRSSKSLLHSNGYTYLFWFQORPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3M12 | Heavy Chain (with wild type human IgG1 constant region) |
| VH3/Vκ2 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with wild type human IgG1 constant region) |
| VH3/Vκ3 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 3M12 | Heavy Chain (with wild type human IgG1 constant region) |
| VH4/Vκ2 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK (SEQ ID NO: 91) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with wild type human IgG1 constant region) |
| VH4/Vκ3 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK (SEQ ID NO: 91) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 5H12 | Heavy Chain (with wild type human IgG1 constant region) |
| VH5 (C33Y*)/Vκ3 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 92) |
| | Light Chain (with kappa light chain constant region) |
| | DIVLTQSPDSLAVSLGERATINCRASESVDGYDNSFMHWYQQKPGQPPKLLIFRAS |
| | NLESGVPDRESGSGSRTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 93) |
| 5H12 | Heavy Chain (with wild type human IgG1 constant region) |
| VH5 (C33D*)/Vκ4 | QVQLVQSGAEVKKPGASVKVSCKASGYSFTDYDINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 94) |
| | Light Chain (with kappa light chain constant region) |
| | DIVMTQSPDSLAVSLGERATINCRASESVDGYDNSFMHWYQQKPGQPPKLLIFRA |
| | SNLESGVPDRESGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| 5H12 | Heavy Chain (with wild type human IgG1 constant region) |
| VH5 (C33Y*)/Vκ4 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 92) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| Anti-TfR clone 8 | VH: |
| | QVQLVQSGAEVKKPGESLKISCKGSGYSFTSYWIGWVRQMPGKGLEWMGIIYPG |
| | DSDTRYSPSFQGQVTISADKSISTAYLQWSSLKASDTAMYYCARFPYDSSGYYSF |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 156) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: |
| | 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 84, 86, 87, 88, 91, 92, 94, and 156. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 84, 86, 87, 88, 91, 92, 94, and 156. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 84, 86, 87, 88, 91, 92, 94, and 156. Alternatively or in addition (e.g., in addition), the anti-TfR1 antibody described herein comprises a light chain comprising the amino acid sequence of any one of SEQ ID NOs: 85, 89, 90, 93, 95 and 157.

In some embodiments, the anti-TfR1 antibody of the present disclosure comprises a heavy chain comprising the amino acid sequence of SEQ ID NO: 84 and a light chain comprising the amino acid sequence of SEQ ID NO: 85.

In some embodiments, the anti-TfR1 antibody of the present disclosure comprises a heavy chain comprising the amino acid sequence of SEQ ID NO: 86 and a light chain comprising the amino acid sequence of SEQ ID NO: 85.

In some embodiments, the anti-TfR1 antibody of the present disclosure comprises a heavy chain comprising the amino acid sequence of SEQ ID NO: 87 and a light chain comprising the amino acid sequence of SEQ ID NO: 85.

In some embodiments, the anti-TfR1 antibody of the present disclosure comprises a heavy chain comprising the amino acid sequence of SEQ ID NO: 88 and a light chain comprising the amino acid sequence of SEQ ID NO: 89.

In some embodiments, the anti-TfR1 antibody of the present disclosure comprises a heavy chain comprising the amino acid sequence of SEQ ID NO: 88 and a light chain comprising the amino acid sequence of SEQ ID NO: 90.

In some embodiments, the anti-TfR1 antibody of the present disclosure comprises a heavy chain comprising the amino acid sequence of SEQ ID NO: 91 and a light chain comprising the amino acid sequence of SEQ ID NO: 89.

In some embodiments, the anti-TfR1 antibody of the present disclosure comprises a heavy chain comprising the amino acid sequence of SEQ ID NO: 91 and a light chain comprising the amino acid sequence of SEQ ID NO: 90.

In some embodiments, the anti-TfR1 antibody of the present disclosure comprises a heavy chain comprising the amino acid sequence of SEQ ID NO: 92 and a light chain comprising the amino acid sequence of SEQ ID NO: 93.

In some embodiments, the anti-TfR1 antibody of the present disclosure comprises a heavy chain comprising the amino acid sequence of SEQ ID NO: 94 and a light chain comprising the amino acid sequence of SEQ ID NO: 95.

In some embodiments, the anti-TfR1 antibody of the present disclosure comprises a heavy chain comprising the amino acid sequence of SEQ ID NO: 92 and a light chain comprising the amino acid sequence of SEQ ID NO: 95.

In some embodiments, the anti-TfR1 antibody of the present disclosure comprises a heavy chain comprising the amino acid sequence of SEQ ID NO: 156 and a light chain comprising the amino acid sequence of SEQ ID NO: 157.

In some embodiments, the anti-TfR1 antibody is a Fab fragment, Fab′ fragment, or F(ab′)2 fragment of an intact antibody (full-length antibody). Antigen binding fragment of an intact antibody (full-length antibody) can be prepared via routine methods (e.g., recombinantly or by digesting the heavy chain constant region of a full-length IgG using an enzyme such as papain). For example, F(ab′)2 fragments can be produced by pepsin or papain digestion of an antibody molecule, and Fab fragments that can be generated by reducing the disulfide bridges of F(ab′)2 fragments. In some embodiments, a heavy chain constant region in a Fab fragment of the anti-TfR1 antibody described herein comprises the amino acid sequence of:

(SEQ ID NO: 96)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHT

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 96. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 96. In some embodiments, the anti-TfR1 antibody described herein comprises a heavy chain comprising any one of the VH as listed in Table 3 or any variants thereof and a heavy chain constant region as set forth in SEQ ID NO: 96.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of Fab heavy chain and light chain amino acid sequences of the anti-TfR1 antibodies described are provided in Table 5 below.

TABLE 5
Heavy chain and light chain sequences of examples of anti-TfR1 Fabs

| Antibody | Fab Heavy Chain/Light Chain Sequences** |
|---|---|
| 3A4 | Heavy Chain (with partial human IgG1 constant region) |
| VH3 (N54T*)/Vκ4 | EVQLVQSGSELKKPGASVKVSCTASGFNIKDDYMYWVRQPPGKGLEWIGWIDPE |
| | TGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHT (SEQ ID NO: 97) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with partial human IgG1 constant region) |
| VH3 (N54S*)/Vκ4 | EVQLVQSGSELKKPGASVKVSCTASGFNIKDDYMYWVRQPPGKGLEWIGWIDPE |
| | SGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHT (SEQ ID NO: 98) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with partial human IgG1 constant region) |
| VH3 /Vκ4 | EVQLVQSGSELKKPGASVKVSCTASGFNIKDDYMYWVRQPPGKGLEWIGWIDPE |
| | NGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHT (SEQ ID NO: 99) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3M12 | Heavy Chain (with partial human IgG1 constant region) |
| VH3/Vκ2 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHT (SEQ ID NO: 100) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with partial human IgG1 constant region) |
| VH3/Vκ3 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHT (SEQ ID NO: 100) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 3M12 | Heavy Chain (with partial human IgG1 constant region) |
| VH4/Vκ2 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHT (SEQ ID NO: 101) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with partial human IgG1 constant region) |
| VH4/Vκ3 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHT (SEQ ID NO: 101) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 5H12 | Heavy Chain (with partial human IgG1 constant region) |
| VH5 (C33Y*)/Vκ3 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 102) |
| | Light Chain (with kappa light chain constant region) |
| | DIVLTQSPDSLAVSLGERATINCRASESVDGYDNSFMHWYQQKPGQPPKLLIFRAS |
| | NLESGVPDRFSGSGSRTDFTLTISSLQAEDVAVYYCOQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 93) |
| 5H12 | Heavy Chain (with partial human IgG1 constant region) |
| VH5 (C33D*)/Vκ4 | QVQLVQSGAEVKKPGASVKVSCKASGYSFTDYDINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 103) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQOSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| 5H12 | Heavy Chain (with partial human IgG1 constant region) |
| VH5 (C33Y*)/Vκ4 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 102) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| Anti-TfR clone 8 | VH: |
| Version 1 | QVQLVQSGAEVKKPGESLKISCKGSGYSFTSYWIGWVRQMPGKGLEWMGIIYPG |
| | DSDTRYSPSFQGQVTISADKSISTAYLQWSSLKASDTAMYYCARFPYDSSGYYSF |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCP (SEQ ID NO: 158) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: |
| | 157) |
| Anti-TfR clone 8 | VH: |
| Version 2 | QVQLVQSGAEVKKPGESLKISCKGSGYSFTSYWIGWVRQMPGKGLEWMGIIYPG |
| | DSDTRYSPSFQGQVTISADKSISTAYLQWSSLKASDTAMYYCARFPYDSSGYYSF |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 159) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: |
| | 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 97-103, 158 and 159. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 97-103, 158 and 159. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 97-103, 158 and 159. Alternatively or in addition (e.g., in addition), the anti-TfR1 antibody described herein comprises a light chain comprising the amino acid sequence of any one of SEQ ID NOs: 85, 89, 90, 93, 95, and 157.

In some embodiments, the anti-TfR1 antibody of the present disclosure comprises a heavy chain comprising the amino acid sequence of SEQ ID NO: 97 and a light chain comprising the amino acid sequence of SEQ ID NO: 85.

In some embodiments, the anti-TfR1 antibody of the present disclosure comprises a heavy chain comprising the amino acid sequence of SEQ ID NO: 98 and a light chain comprising the amino acid sequence of SEQ ID NO: 85.

In some embodiments, the anti-TfR1 antibody of the present disclosure comprises a heavy chain comprising the amino acid sequence of SEQ ID NO: 99 and a light chain comprising the amino acid sequence of SEQ ID NO: 85.

In some embodiments, the anti-TfR1 antibody of the present disclosure comprises a heavy chain comprising the amino acid sequence of SEQ ID NO: 100 and a light chain comprising the amino acid sequence of SEQ ID NO: 89.

In some embodiments, the anti-TfR1 antibody of the present disclosure comprises a heavy chain comprising the amino acid sequence of SEQ ID NO: 100 and a light chain comprising the amino acid sequence of SEQ ID NO: 90.

In some embodiments, the anti-TfR1 antibody of the present disclosure comprises a heavy chain comprising the amino acid sequence of SEQ ID NO: 101 and a light chain comprising the amino acid sequence of SEQ ID NO: 89.

In some embodiments, the anti-TfR1 antibody of the present disclosure comprises a heavy chain comprising the amino acid sequence of SEQ ID NO: 101 and a light chain comprising the amino acid sequence of SEQ ID NO: 90.

In some embodiments, the anti-TfR1 antibody of the present disclosure comprises a heavy chain comprising the amino acid sequence of SEQ ID NO: 102 and a light chain comprising the amino acid sequence of SEQ ID NO: 93.

In some embodiments, the anti-TfR1 antibody of the present disclosure comprises a heavy chain comprising the amino acid sequence of SEQ ID NO: 103 and a light chain comprising the amino acid sequence of SEQ ID NO: 95.

In some embodiments, the anti-TfR1 antibody of the present disclosure comprises a heavy chain comprising the amino acid sequence of SEQ ID NO: 102 and a light chain comprising the amino acid sequence of SEQ ID NO: 95.

In some embodiments, the anti-TfR1 antibody of the present disclosure comprises a heavy chain comprising the amino acid sequence of SEQ ID NO: 158 and a light chain comprising the amino acid sequence of SEQ ID NO: 157.

In some embodiments, the anti-TfR1 antibody of the present disclosure comprises a heavy chain comprising the amino acid sequence of SEQ ID NO: 159 and a light chain comprising the amino acid sequence of SEQ ID NO: 157.

### Other Known Anti-TfR1 Antibodies

Any other appropriate anti-TfR1 antibodies known in the art may be used as the muscle-targeting agent in the complexes disclosed herein. Examples of known anti-TfR1 antibodies, including associated references and binding epitopes, are listed in Table 6. In some embodiments, the anti-TfR1 antibody comprises the complementarity determining regions (CDR-H1, CDR-H2, CDR-H3, CDR-L1, CDR-L2, and CDR-L3) of any of the anti-TfR1 antibodies provided herein, e.g., anti-TfR1 antibodies listed in Table 6.

TABLE 6
List of anti-TfR1 antibody clones, including associated
references and binding epitope information.

| Antibody Clone | | |
|---|---|---|
| Name | Reference(s) | Epitope/Notes |
| OKT9 | U.S. Pat. No. 4,364,934, filed Dec. 4, 1979, | Apical domain of TfR1 |
| | entitled “MONOCLONAL ANTIBODY TO | (residues 305-366 of |
| | A HUMAN EARLY THYMOCYTE | human TfR1 sequence |
| | ANTIGEN AND METHODS FOR | XM_052730.3, available |
| | PREPARING SAME” | in GenBank) |
| | Schneider C. et al. “Structural features of the | |
| | cell surface receptor for transferrin that is | |
| | recognized by the monoclonal antibody | |
| | OKT9.” J Biol Chem. 1982, 257: 14, 8516- | |
| | 8522. | |
| (From JCR) | WO 2015/098989, filed Dec. 24, 2014, | Apical domain (residues |
| Clone M11 | “Novel anti-Transferrin receptor antibody | 230-244 and 326-347 of |
| Clone M23 | that passes through blood-brain barrier” | TfR1) and protease-like |
| Clone M27 | U.S. Pat. No. 9,994,641, filed | domain (residues 461- |
| Clone B84 | Dec. 24, 2014, “Novel anti-Transferrin | 473) |
| | receptor antibody that passes through | |
| | blood-brain barrier” | |
| (From | WO 2016/081643, filed May 26, 2016, | Apical domain and non- |
| Genentech) | entitled “ANTI-TRANSFERRIN | apical regions |
| 7A4, 8A2, 15D2, | RECEPTOR ANTIBODIES AND | |
| 10D11, 7B10, | METHODS OF USE” | |
| 15G11, 16G5, | U.S. Pat. No. 9,708,406, filed | |
| 13C3, 16G4, | May 20, 2014, “Anti-transferrin receptor | |
| 16F6, 7G7, 4C2, | antibodies and methods of use” | |
| 1B12, and 13D4 | | |
| (From Armagen) | Lee et al. “Targeting Rat Anti-Mouse | |
| 8D3 | Transferrin Receptor Monoclonal Antibodies | |
| | through Blood-Brain Barrier in Mouse” | |
| | 2000, J Pharmacol. Exp. Ther., 292: 1048- | |
| | 1052. | |
| | US Patent App. 2010/077498, filed | |
| | Sep. 11, 2008, entitled “COMPOSITIONS AND | |
| | METHODS FOR BLOOD-BRAIN | |
| | BARRIER DELIVERY IN THE MOUSE” | |
| OX26 | Haobam, B. et al. 2014. Rab17- | |
| | mediated recycling endosomes contribute to | |
| | autophagosome formation in response to | |
| | Group A Streptococcus invasion. Cellular | |
| | microbiology. 16: 1806-21. | |
| DF1513 | Ortiz-Zapater E et al. Trafficking of | |
| | the human transferrin receptor in plant cells: | |
| | effects of tyrphostin A23 and brefeldin A. | |
| | Plant J 48: 757-70 (2006). | |
| 1A1B2, 66IG10, | Commercially available anti- | Novus Biologicals |
| MEM-189, | transferrin receptor antibodies. | 8100 Southpark Way, A- |
| JF0956, 29806, | | 8 Littleton CO 80120 |
| 1A1B2, | | |
| TFRC/1818, | | |
| 1E6, 66Ig10, | | |
| TFRC/1059, | | |
| Q1/71, 23D10, | | |
| 13E4, | | |
| TFRC/1149, ER- | | |
| MP21, | | |
| YTA74.4, BU54, | | |
| 2B6, RI7 217 | | |
| (From INSERM) | US Patent App. 2011/0311544A1, | Does not compete with |
| BA120g | filed Jun. 15, 2005, entitled “ANTI-CD71 | OKT9 |
| | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 epitope” |
| | Jun. 7, 2004, entitled “TRANSFERRIN | |
| | RECEPTOR ANTIBODIES” | |
| (Salk Institute) | Trowbridge, I. S. et al. “Anti-transferrin | |
| B3/25 | receptor monoclonal antibody and toxin- | |
| T58/30 | antibody conjugates affect growth of | |
| | human tumour cells.” Nature, 1981, | |
| | volume 294, pages 171-173 | |
| R17 217.1.3, | Commercially available anti- | BioXcell |
| 5E9C11, | transferrin receptor antibodies. | 10 Technology Dr., Suite |
| OKT9 (BE0023 | | 2B |
| clone) | | West Lebanon, NH |
| | | 03784-1671 USA |
| BK19.9, B3/25, | Gatter, K. C. et al. “Transferrin receptors | |
| T56/14 and | in human tissues: their distribution and | |
| T58/1 | possible clinical relevance.” J Clin | |
| | Pathol. 1983 May; 36(5): 539-45. | |

| | Additional Anti-TfR1 antibody SEQ ID NOs | | | | |
|---|---|---|---|---|---|
| Anto-TfR1 antibody | | VH/VL | CDR1 | CDR2 | CDR3 |
| CDRH1 (SEQ ID NO: 2179) | VH1 | 2194 | 2187 | 2188 | 2181 |
| CDRH2 (SEQ ID NO: 2180) | VH2 | 2195 | 2187 | 2189 | 2181 |
| CDRH3 (SEQ ID NO: 2181) | VH3 | 2196 | 2187 | 2190 | 2181 |
| CDRL1 (SEQ ID NO: 2182) | VH4 | 2197 | 2187 | 2189 | 2181 |
| CDRL2 (SEQ ID NO: 2183) | VL1 | 2198 | 2182 | 2183 | 115 |
| CDRL3 (SEQ ID NO: 2184) | VL2 | 2199 | 2182 | 2183 | 115 |
| VH (SEQ ID NO: 2185) | VL3 | 2200 | 2182 | 2191 | 2184 |
| VL (SEQ ID NO: 2186) | VL4 | 2201 | 2182 | 2193 | 2184 |

In some embodiments, anti-TfR1 antibodies of the present disclosure include one or more of the CDR-H (e.g., CDR-H1, CDR-H-2, and CDR-H3) amino acid sequences from any one of the anti-TfR1 antibodies selected from Table 6. In some embodiments, anti-TfR1 antibodies include the CDR-L1, CDR-L2, and CDR-L3 as provided for any one of the anti-TfR1 antibodies selected from Table 6. In some embodiments, anti-TfR1 antibodies include the CDR-H1, CDR-H2, CDR-H-3, CDR-L1, CDR-L2, and CDR-L3 as provided for any one of the anti-TfR1 antibodies selected from Table 6.

In some embodiments, anti-TfR1 antibodies of the disclosure include any antibody that includes a heavy chain variable domain and/or (e.g., and) a light chain variable domain of any anti-TfR1 antibody, such as any one of the anti-TfR1 antibodies selected from Table 6. In some embodiments, anti-TfR1 antibodies of the disclosure include any antibody that includes the heavy chain variable and light chain variable pairs of any anti-TfR1 antibody, such as any one of the anti-TfR1 antibodies selected from Table 6.

Aspects of the disclosure provide anti-TfR1 antibodies having a heavy chain variable (VH) and/or (e.g., and) a light chain variable (VL) domain amino acid sequence homologous to any of those described herein. In some embodiments, the anti-TfR1 antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-TfR1 antibody, such as any one of the anti-TfR1 antibodies selected from Table 6. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-TfR1 antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR1 antibody, such as any one of the anti-TfR1 antibodies selected from Table 6.

An example of a transferrin receptor antibody that may be used in accordance with the present disclosure is described in International Application Publication WO 2016/081643, incorporated herein by reference. The amino acid sequences of this antibody are provided in Table 7.

TABLE 7
Heavy chain and light chain CDRs of an example of a known anti-TfR1 antibody

| Sequence Type | Kabat | Chothia | Contact |
|---|---|---|---|
| CDR-H1 | SYWMH (SEQ ID | GYTFTSY (SEQ ID NO: 116) | TSYWMH (SEQ ID NO: 118) |
| | NO: 110) | | |
| CDR-H2 | EINPTNGRTNYIE | NPTNGR (SEQ ID NO: 117) | WIGEINPTNGRTN (SEQ ID |
| | KFKS (SEQ ID | | NO: 119) |
| | NO: 111) | | |
| CDR-H3 | GTRAYHY (SEQ | GTRAYHY (SEQ ID NO: | ARGTRA (SEQ ID NO: 120) |
| | ID NO: 112) | 112) | |
| CDR-L1 | RASDNLYSNLA | RASDNLYSNLA (SEQ ID | YSNLAWY (SEQ ID NO: 121) |
| | (SEQ ID NO: 113) | NO: 113) | |
| CDR-L2 | DATNLAD (SEQ | DATNLAD (SEQ ID NO: | LLVYDATNLA (SEQ ID NO: |
| | ID NO: 114) | 114) | 122) |
| CDR-L3 | QHFWGTPLT | QHFWGTPLT (SEQ ID NO: | QHFWGTPL (SEQ ID NO: |
| | (SEQ ID NO: 115) | 115) | 123) |

| | |
|---|---|
| Murine VH | QVQLQQPGAELVKPGASVKLSCKASGYTFTSYWMHWVKQRPGQGLEWIGEINP |
| | TNGRTNYIEKFKSKATLTVDKSSSTAYMQLSSLTSEDSAVYYCARGTRAYHYW |
| | GQGTSVTVSS (SEQ ID NO: 124) |
| Murine VL | DIQMTQSPASLSVSVGETVTITCRASDNLYSNLAWYQQKQGKSPQLLVYDATNL |
| | ADGVPSRFSGSGSGTQYSLKINSLQSEDFGTYYCQHFWGTPLTFGAGTKLELK |
| | (SEQ ID NO: 125) |
| Humanized VH | EVQLVQSGAEVKKPGASVKVSCKASGYTFTSYWMHWVRQAPGQRLEWIGEIN |
| | PTNGRTNYIEKFKSRATLTVDKSASTAYMELSSLRSEDTAVYYCARGTRAYHY |
| | WGQGTMVTVSS (SEQ ID NO: 128) |
| Humanized VL | DIQMTQSPSSLSASVGDRVTITCRASDNLYSNLAWYQQKPGKSPKLLVYDATNL |
| | ADGVPSRFSGSGSGTDYTLTISSLQPEDFATYYCQHFWGTPLTFGQGTKVEIK |
| | (SEQ ID NO: 129) |
| HC of chimeric | QVQLQQPGAELVKPGASVKLSCKASGYTFTSYWMHWVKQRPGQGLEWIGEINP |
| full-length IgG1 | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEAL |
| | HNHYTQKSLSLSPGK (SEQ ID NO: 132) |
| LC of chimeric | DIQMTQSPASLSVSVGETVTITCRASDNLYSNLAWYQQKQGKSPQLLVYDATNL |
| full-length IgG1 | ADGVPSRFSGSGSGTQYSLKINSLQSEDFGTYYCQHFWGTPLTFGAGTKLELKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 133) |
| HC of fully human | EVQLVQSGAEVKKPGASVKVSCKASGYTFTSYWMHWVRQAPGQRLEWIGEIN |
| full-length IgG1 | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEA |
| | LHNHYTQKSLSLSPGK (SEQ ID NO: 134) |
| LC of fully human | DIQMTQSPSSLSASVGDRVTITCRASDNLYSNLAWYQQKPGKSPKLLVYDATNL |
| full-length IgG1 | ADGVPSRFSGSGSGTDYTLTISSLQPEDFATYYCQHFWGTPLTFGQGTKVEIKRT |
| | VAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| | (SEQ ID NO: 135) |
| HC of chimeric | QVQLQQPGAELVKPGASVKLSCKASGYTFTSYWMHWVKQRPGQGLEWIGEINP |
| Fab | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCP (SEQ ID NO: 136) |
| HC of fully human | EVQLVQSGAEVKKPGASVKVSCKASGYTFTSYWMHWVRQAPGQRLEWIGEIN |
| Fab | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCP (SEQ ID NO: 137) |

In some embodiments, the anti-TfR1 antibody of the present disclosure comprises a CDR-H1, a CDR-H2, and a CDR-H3 that are the same as the CDR-H1, CDR-H2, and CDR-H3 shown in Table 7. Alternatively or in addition (e.g., in addition), the anti-TfR1 antibody of the present disclosure comprises a CDR-L1, a CDR-L2, and a CDR-L3 that are the same as the CDR-L1, CDR-L2, and CDR-L3 shown in Table 7.

In some embodiments, the anti-TfR1 antibody of the present disclosure comprises a CDR-L3, which contains no more than 3 amino acid variations (e.g., no more than 3, 2, or 1 amino acid variation) as compared with the CDR-L3 as shown in Table 7. In some embodiments, the anti-TfR1 antibody of the present disclosure comprises a CDR-L3 containing one amino acid variation as compared with the CDR-L3 as shown in Table 7. In some embodiments, the anti-TfR1 antibody of the present disclosure comprises a CDR-L3 of QHFAGTPLT (SEQ ID NO: 126) (according to the Kabat and Chothia definition system) or QHFAGTPL (SEQ ID NO: 127) (according to the Contact definition system). In some embodiments, the anti-TfR1 antibody of the present disclosure comprises a CDR-H1, a CDR-H2, a CDR-H3, a CDR-L1 and a CDR-L2 that are the same as the CDR-H1, CDR-H2, and CDR-H3 shown in Table 7, and comprises a CDR-L3 of QHFAGTPLT (SEQ ID NO: 126) (according to the Kabat and Chothia definition system) or QHFAGTPL (SEQ ID NO: 127) (according to the Contact definition system).

In some embodiments, the anti-TfR1 antibody of the present disclosure comprises heavy chain CDRs that collectively are at least 80% (e.g., 80%, 85%, 90%, 95%, or 98%) identical to the heavy chain CDRs as shown in Table 7. Alternatively or in addition (e.g., in addition), the anti-TfR1 antibody of the present disclosure comprises light chain CDRs that collectively are at least 80% (e.g., 80%, 85%, 90%, 95%, or 98%) identical to the light chain CDRs as shown in Table 7.

In some embodiments, the anti-TfR1 antibody of the present disclosure comprises a VH comprising the amino acid sequence of SEQ ID NO: 124. Alternatively or in addition (e.g., in addition), the anti-TfR1 antibody of the present disclosure comprises a VL comprising the amino acid sequence of SEQ ID NO: 125.

In some embodiments, the anti-TfR1 antibody of the present disclosure comprises a VH comprising the amino acid sequence of SEQ ID NO: 128. Alternatively or in addition (e.g., in addition), the anti-TfR1 antibody of the present disclosure comprises a VL comprising the amino acid sequence of SEQ ID NO: 129.

In some embodiments, the anti-TfR1 antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 128. Alternatively or in addition (e.g., in addition), the anti-TfR1 antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 129.

In some embodiments, the anti-TfR1 antibody of the present disclosure is a full-length IgG1 antibody, which can include a heavy constant region and a light constant region from a human antibody. In some embodiments, the heavy chain of any of the anti-TfR1 antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of human IgG1 constant region is given below:

(SEQ ID NO: 81)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, the anti-TfR1 antibody described herein is a chimeric antibody that comprises a heavy chain comprising the amino acid sequence of SEQ ID NO: 132. Alternatively or in addition (e.g., in addition), the anti-TfR1 antibody described herein comprises a light chain comprising the amino acid sequence of SEQ ID NO: 133.

In some embodiments, the anti-TfR1 antibody described herein is a fully human antibody that comprises a heavy chain comprising the amino acid sequence of SEQ ID NO: 134. Alternatively or in addition (e.g., in addition), the anti-TfR1 antibody described herein comprises a light chain comprising the amino acid sequence of SEQ ID NO: 135.

In some embodiments, the anti-TfR1 antibody is an antigen binding fragment (Fab) of an intact antibody (full-length antibody). In some embodiments, the anti-TfR1 Fab described herein comprises a heavy chain comprising the amino acid sequence of SEQ ID NO: 136. Alternatively or in addition (e.g., in addition), the anti-TfR1 Fab described herein comprises a light chain comprising the amino acid sequence of SEQ ID NO: 133. In some embodiments, the anti-TfR1 Fab described herein comprises a heavy chain comprising the amino acid sequence of SEQ ID NO: 137. Alternatively or in addition (e.g., in addition), the anti-TfR1 Fab described herein comprises a light chain comprising the amino acid sequence of SEQ ID NO: 135.

The anti-TfR1 antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, the anti-TfR1 antibody described herein is an scFv. In some embodiments, the anti-TfR1 antibody described herein is an scFv-Fab (e.g., scFv fused to a portion of a constant region). In some embodiments, the anti-TfR1 antibody described herein is an scFv fused to a constant region (e.g., human IgG1 constant region as set forth in SEQ ID NO: 81).

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of an anti-TfR1 antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-TfR1 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall′Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-TfR1 antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-TfR1 antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide). In some embodiments, the signal peptide comprises the amino acid sequence of MGWSCIILFLVATATGVHS (SEQ ID NO: 104).

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

b. Other Muscle-Targeting Antibodies

In some embodiments, the muscle-targeting antibody is an antibody that specifically binds hemojuvelin, caveolin-3, Duchenne muscular dystrophy peptide, myosin IIb or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall′Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like Cκ or Cλ. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g. a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 2170) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 2170). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for Duchenne muscular dystrophy. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 2171) was identified and this muscle-targeting peptide showed improved binding to C2C12 cells relative to the ASSLNIA (SEQ ID NO: 2170) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 2172) appeared most frequently. Accordingly, in some embodiments, the muscle-targeting agent comprises the amino acid sequence TARGEHKEEELI (SEQ ID NO: 2172).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or (e.g., and) derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7.; Zhang, L. “Molecular profiling of heart endothelial cells.” Circulation, 2005, 112:11, 1601-11.; McGuire, M. J. et al. “In vitro selection of a peptide with high selectivity for cardiomyocytes in vivo.” J Mol Biol. 2004, 342:1, 171-82.). Exemplary muscle-targeting peptides comprise an amino acid sequence of the following group: CQAQGQLVC (SEQ ID NO: 2173), CSERSMNFC (SEQ ID NO: 2174), CPKTRRVPC (SEQ ID NO: 2175), WLSEAGPVVTVRALRGTGSW (SEQ ID NO: 2176), ASSLNIA (SEQ ID NO: 2170), CMQHSMRVC (SEQ ID NO: 2177), and DDTRHWG (SEQ ID NO: 2178). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147.).

iii. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Muscle-Targeting Aptamers

A muscle-targeting agent may be an aptamer, e.g. an RNA aptamer, which preferentially targets muscle cells relative to other cell types. In some embodiments, a muscle-targeting aptamer has not been previously characterized or disclosed. These aptamers may be conceived of, produced, synthesized, and/or (e.g., and) derivatized using any of several methodologies, e.g. Systematic Evolution of Ligands by Exponential Enrichment. Exemplary methodologies have been characterized in the art and are incorporated by reference (Yan, A. C. and Levy, M. “Aptamers and aptamer targeted delivery” RNA biology, 2009, 6:3, 316-20.; Germer, K. et al. “RNA aptamers and their therapeutic and diagnostic applications.” Int. J. Biochem. Mol. Biol. 2013; 4: 27-40.). In some embodiments, a muscle-targeting aptamer has been previously disclosed (see, e.g. Phillippou, S. et al. “Selection and Identification of Skeletal-Muscle-Targeted RNA Aptamers.” Mol Ther Nucleic Acids. 2018, 10:199-214.; Thiel, W. H. et al. “Smooth Muscle Cell-targeted RNA Aptamer Inhibits Neointimal Formation.” Mol Ther. 2016, 24:4, 779-87.). Exemplary muscle-targeting aptamers include the A01B RNA aptamer and RNA Apt 14. In some embodiments, an aptamer is a nucleic acid-based aptamer, an oligonucleotide aptamer or a peptide aptamer. In some embodiments, an aptamer may be about 5-15 kDa, about 5-10 kDa, about 10⁻¹⁵ kDa, about 1-5 Da, about 1-3 kDa, or smaller.

v. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is any muscle targeting agent described herein (e.g., antibodies, nucleic acids, small molecules, peptides, aptamers, lipids, sugar moieties) that target SLC superfamily of transporters. In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or (e.g., and) lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

### B. Molecular Payloads

Some aspects of the disclosure provide molecular payloads, e.g., for modulating a biological outcome, e.g., the transcription of a DNA sequence, the splicing and processing of an RNA sequence, the expression of a protein, or the activity of a protein. In some embodiments, a molecular payload is linked to, or otherwise associated with a muscle-targeting agent. In some embodiments, such molecular payloads are capable of targeting to a muscle cell, e.g., via specifically binding to a nucleic acid or protein in the muscle cell following delivery to the muscle cell by an associated muscle-targeting agent. It should be appreciated that various types of molecular payloads may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, the molecular payload is an oligonucleotide that comprises a strand having a region of complementarity to a mutated DMD allele. Exemplary molecular payloads are described in further detail herein, however, it should be appreciated that the exemplary molecular payloads provided herein are not meant to be limiting.

i. Oligonucleotides

Aspects of the disclosure relate to oligonucleotides configured to modulate (e.g., increase) expression of dystrophin, e.g., from a DMD allele. In some embodiments, oligonucleotides provided herein are configured to alter splicing of DMD pre-mRNA to promote expression of dystrophin protein (e.g., a functional truncated dystrophin protein). In some embodiments, oligonucleotides provided herein are configured to promote skipping of one or more exons in DMD, e.g., in a mutated DMD allele, in order to restore the reading frame. In some embodiments, the oligonucleotides allow for functional dystrophin protein expression (e.g., as described in Watanabe N, Nagata T, Satou Y, et al. NS-065/NCNP-01: an antisense oligonucleotide for potential treatment of exon 53 skipping in Duchenne muscular dystrophy. Mol Ther Nucleic Acids. 2018; 13:442-449). In some embodiments, oligonucleotides provided are configured to promote skipping of exon 55 to produce a shorter but functional version of dystrophin (e.g., containing an in-frame deletion). In some embodiments, oligonucleotides are provided that promote exon 55 skipping (e.g., which may be relevant in a substantial number of patients, including, for example, patients amenable to exon 55 skipping, such as those having deletions in DMD exons 3-54, 4-54, 5-54, 6-54, 9-54, 10-54, 11-54, 13-54, 14-54, 15-54, 16-54, 17-54, 19-54, 21-54, 23-54, 24-54, 25-54, 26-54, 27-54, 28-54, 29-54, 30-54, 31-54, 32-54, 33-54, 34-54, 35-54, 36-54, 37-54, 38-54, 39-54, 40-54, 41-54, 42-54, 43-54, 45-54, 47-54, 48-54, 49-54, 50-54, 52-54, 54, 56, 56-62, 56-65, 56-68, 56-70, 56-71, 56-72, 56-73, or 56-74).

Table 8 provides non-limiting examples of sequences of oligonucleotides that are useful for targeting DMD, e.g., for exon skipping, and for target sequences within DMD. In some embodiments, an oligonucleotide may comprise any antisense sequence provided in Table 8 or a sequence complementary to a target sequence provided in Table 8.

TABLE 8
Oligonucleotide sequences for targeting DMD.

| SEQ | | SEQ | Antisense | SEQ | Antisense | |
|---|---|---|---|---|---|---|
| ID | Target sequence† | ID | Sequence† | ID | Sequence† | |
| NO | (5′ to 3′) | NO | (5′ to 3′) | NO | (5′ to 3′) | Target Site |
| 160 | GGAAGAAACUCAU | 780 | UGCAGUAAUCUAU | 1400 | TGCAGTAATCTAT | Exon 55 |
| | AGAUUACUGCA | | GAGUUUCUUCC | | GAGTTTCTTCC | |
| 161 | GAAACAACUGCCA | 781 | GUAGGACAUUGGC | 1401 | GTAGGACATTGGC | Exon 55 |
| | AUGUCCUAC | | AGUUGUUUC | | AGTTGTTTC | |
| 162 | GAAACAACUGCCA | 782 | UGUAGGACAUUGG | 1402 | TGTAGGACATTGG | Exon 55 |
| | AUGUCCUACA | | CAGUUGUUUC | | CAGTTGTTTC | |
| 163 | GAAACAACUGCCA | 783 | CUGUAGGACAUUG | 1403 | CTGTAGGACATTG | Exon 55 |
| | AUGUCCUACAG | | GCAGUUGUUUC | | GCAGTTGTTTC | |
| 164 | GAAACAACUGCCA | 784 | CCUGUAGGACAUU | 1404 | CCTGTAGGACATT | Exon 55 |
| | AUGUCCUACAGG | | GGCAGUUGUUUC | | GGCAGTTGTTTC | |
| 165 | AAACAACUGCCAA | 785 | CCUGUAGGACAUU | 1405 | CCTGTAGGACATT | Exon 55 |
| | UGUCCUACAGG | | GGCAGUUGUUU | | GGCAGTTGTTT | |
| 166 | AAACAACUGCCAA | 786 | UCCUGUAGGACAU | 1406 | TCCTGTAGGACAT | Exon 55 |
| | UGUCCUACAGGA | | UGGCAGUUGUUU | | TGGCAGTTGTTT | |
| 167 | AACAACUGCCAAU | 787 | CUGUAGGACAUUG | 1407 | CTGTAGGACATTG | Exon 55 |
| | GUCCUACAG | | GCAGUUGUU | | GCAGTTGTT | |
| 168 | AACAACUGCCAAU | 788 | CCUGUAGGACAUU | 1408 | CCTGTAGGACATT | Exon 55 |
| | GUCCUACAGG | | GGCAGUUGUU | | GGCAGTTGTT | |
| 169 | AACAACUGCCAAU | 789 | UCCUGUAGGACAU | 1409 | TCCTGTAGGACAT | Exon 55 |
| | GUCCUACAGGA | | UGGCAGUUGUU | | TGGCAGTTGTT | |
| 170 | ACAACUGCCAAUG | 790 | UGUAGGACAUUGG | 1410 | TGTAGGACATTGG | Exon 55 |
| | UCCUACA | | CAGUUGU | | CAGTTGT | |
| 171 | ACAACUGCCAAUG | 791 | CUGUAGGACAUUG | 1411 | CTGTAGGACATTG | Exon 55 |
| | UCCUACAG | | GCAGUUGU | | GCAGTTGT | |
| 172 | ACAACUGCCAAUG | 792 | CCUGUAGGACAUU | 1412 | CCTGTAGGACATT | Exon 55 |
| | UCCUACAGG | | GGCAGUUGU | | GGCAGTTGT | |
| 173 | ACAACUGCCAAUG | 793 | UCCUGUAGGACAU | 1413 | TCCTGTAGGACAT | Exon 55 |
| | UCCUACAGGA | | UGGCAGUUGU | | TGGCAGTTGT | |
| 174 | CAACUGCCAAUGU | 794 | CCUGUAGGACAUU | 1414 | CCTGTAGGACATT | Exon 55 |
| | CCUACAGG | | GGCAGUUG | | GGCAGTTG | |
| 175 | CAACUGCCAAUGU | 795 | UCCUGUAGGACAU | 1415 | TCCTGTAGGACAT | Exon 55 |
| | CCUACAGGA | | UGGCAGUUG | | TGGCAGTTG | |
| 176 | AACUGCCAAUGUC | 796 | UCCUGUAGGACAU | 1416 | TCCTGTAGGACAT | Exon 55 |
| | CUACAGGA | | UGGCAGUU | | TGGCAGTT | |
| 177 | ACUGCCAAUGUCC | 797 | UCCUGUAGGACAU | 1417 | TCCTGTAGGACAT | Exon 55 |
| | UACAGGA | | UGGCAGU | | TGGCAGT | |
| 178 | AGAAACUCAUAGA | 798 | UGUUGCAGUAAUC | 1418 | TGTTGCAGTAATC | Exon 55 |
| | UUACUGCAACA | | UAUGAGUUUCU | | TATGAGTTTCT | |
| 179 | AGAAACUCAUAGA | 799 | CUGUUGCAGUAAU | 1419 | CTGTTGCAGTAAT | Exon 55 |
| | UUACUGCAACAG | | CUAUGAGUUUCU | | CTATGAGTTTCT | |
| 180 | GAAACUCAUAGAU | 800 | CUGUUGCAGUAAU | 1420 | CTGTTGCAGTAAT | Exon 55 |
| | UACUGCAACAG | | CUAUGAGUUUC | | CTATGAGTTTC | |
| 181 | GAUGAUACCAGAA | 801 | AUGUGGACUUUUC | 1421 | ATGTGGACTTTTC | Exon 54 |
| | AAGUCCACAU | | UGGUAUCAUC | | TGGTATCATC | |
| 182 | GAUGAUACCAGAA | 802 | UCAUGUGGACUUU | 1422 | TCATGTGGACTTT | Exon 54 |
| | AAGUCCACAUGA | | UCUGGUAUCAUC | | TCTGGTATCATC | |
| 183 | AUGAUACCAGAAA | 803 | UCAUGUGGACUUU | 1423 | TCATGTGGACTTT | Exon 54 |
| | AGUCCACAUGA | | UCUGGUAUCAU | | TCTGGTATCAT | |
| 184 | AUGAUACCAGAAA | 804 | AUCAUGUGGACUU | 1424 | ATCATGTGGACTT | Exon 54 |
| | AGUCCACAUGAU | | UUCUGGUAUCAU | | TTCTGGTATCAT | |
| 185 | UGAUACCAGAAAA | 805 | UCAUGUGGACUUU | 1425 | TCATGTGGACTTT | Exon 54 |
| | GUCCACAUGA | | UCUGGUAUCA | | TCTGGTATCA | |
| 186 | UGAUACCAGAAAA | 806 | AUCAUGUGGACUU | 1426 | ATCATGTGGACTT | Exon 54 |
| | GUCCACAUGAU | | UUCUGGUAUCA | | TTCTGGTATCA | |
| 187 | GAUACCAGAAAAG | 807 | UCAUGUGGACUUU | 1427 | TCATGTGGACTTT | Exon 54 |
| | UCCACAUGA | | UCUGGUAUC | | TCTGGTATC | |
| 188 | GAUACCAGAAAAG | 808 | AUCAUGUGGACUU | 1428 | ATCATGTGGACTT | Exon 54 |
| | UCCACAUGAU | | UUCUGGUAUC | | TTCTGGTATC | |
| 189 | GAUACCAGAAAAG | 809 | UUAUCAUGUGGAC | 1429 | TTATCATGTGGAC | Exon 54 |
| | UCCACAUGAUAA | | UUUUCUGGUAUC | | TTTTCTGGTATC | |
| 190 | AUACCAGAAAAGU | 810 | UCAUGUGGACUUU | 1430 | TCATGTGGACTTT | Exon 54 |
| | CCACAUGA | | UCUGGUAU | | TCTGGTAT | |
| 191 | AUACCAGAAAAGU | 811 | AUCAUGUGGACUU | 1431 | ATCATGTGGACTT | Exon 54 |
| | CCACAUGAU | | UUCUGGUAU | | TTCTGGTAT | |
| 192 | AUACCAGAAAAGU | 812 | GUUAUCAUGUGGA | 1432 | GTTATCATGTGGA | Exon 54 |
| | CCACAUGAUAAC | | CUUUUCUGGUAU | | CTTTTCTGGTAT | |
| 193 | UACCAGAAAAGUC | 813 | UCAUGUGGACUUU | 1433 | TCATGTGGACTTT | Exon 54 |
| | CACAUGA | | UCUGGUA | | TCTGGTA | |
| 194 | UACCAGAAAAGUC | 814 | AUCAUGUGGACUU | 1434 | ATCATGTGGACTT | Exon 54 |
| | CACAUGAU | | UUCUGGUA | | TTCTGGTA | |
| 195 | UACCAGAAAAGUC | 815 | UUAUCAUGUGGAC | 1435 | TTATCATGTGGAC | Exon 54 |
| | CACAUGAUAA | | UUUUCUGGUA | | TTTTCTGGTA | |
| 196 | UACCAGAAAAGUC | 816 | GUUAUCAUGUGGA | 1436 | GTTATCATGTGGA | Exon 54 |
| | CACAUGAUAAC | | CUUUUCUGGUA | | CTTTTCTGGTA | |
| 197 | UACCAGAAAAGUC | 817 | UGUUAUCAUGUGG | 1437 | TGTTATCATGTGG | Exon 54 |
| | CACAUGAUAACA | | ACUUUUCUGGUA | | ACTTTTCTGGTA | |
| 198 | ACCAGAAAAGUCC | 818 | AUCAUGUGGACUU | 1438 | ATCATGTGGACTT | Exon 54 |
| | ACAUGAU | | UUCUGGU | | TTCTGGT | |
| 199 | ACCAGAAAAGUCC | 819 | UUAUCAUGUGGAC | 1439 | TTATCATGTGGAC | Exon 54 |
| | ACAUGAUAA | | UUUUCUGGU | | TTTTCTGGT | |
| 200 | ACCAGAAAAGUCC | 820 | GUUAUCAUGUGGA | 1440 | GTTATCATGTGGA | Exon 54 |
| | ACAUGAUAAC | | CUUUUCUGGU | | CTTTTCTGGT | |
| 201 | ACCAGAAAAGUCC | 821 | UGUUAUCAUGUGG | 1441 | TGTTATCATGTGG | Exon 54 |
| | ACAUGAUAACA | | ACUUUUCUGGU | | ACTTTTCTGGT | |
| 202 | ACCAGAAAAGUCC | 822 | CUGUUAUCAUGUG | 1442 | CTGTTATCATGTG | Exon 54 |
| | ACAUGAUAACAG | | GACUUUUCUGGU | | GACTTTTCTGGT | |
| 203 | CCAGAAAAGUCCA | 823 | UUAUCAUGUGGAC | 1443 | TTATCATGTGGAC | Exon 54 |
| | CAUGAUAA | | UUUUCUGG | | TTTTCTGG | |
| 204 | CCAGAAAAGUCCA | 824 | GUUAUCAUGUGGA | 1444 | GTTATCATGTGGA | Exon 54 |
| | CAUGAUAAC | | CUUUUCUGG | | CTTTTCTGG | |
| 205 | CCAGAAAAGUCCA | 825 | UGUUAUCAUGUGG | 1445 | TGTTATCATGTGG | Exon 54 |
| | CAUGAUAACA | | ACUUUUCUGG | | ACTTTTCTGG | |
| 206 | CCAGAAAAGUCCA | 826 | CUGUUAUCAUGUG | 1446 | CTGTTATCATGTG | Exon 54 |
| | CAUGAUAACAG | | GACUUUUCUGG | | GACTTTTCTGG | |
| 207 | CCAGAAAAGUCCA | 827 | UCUGUUAUCAUGU | 1447 | TCTGTTATCATGT | Exon 54 |
| | CAUGAUAACAGA | | GGACUUUUCUGG | | GGACTTTTCTGG | |
| 208 | CAGAAAAGUCCAC | 828 | GUUAUCAUGUGGA | 1448 | GTTATCATGTGGA | Exon 54 |
| | AUGAUAAC | | CUUUUCUG | | CTTTTCTG | |
| 209 | CAGAAAAGUCCAC | 829 | UGUUAUCAUGUGG | 1449 | TGTTATCATGTGG | Exon 54 |
| | AUGAUAACA | | ACUUUUCUG | | ACTTTTCTG | |
| 210 | CAGAAAAGUCCAC | 830 | CUGUUAUCAUGUG | 1450 | CTGTTATCATGTG | Exon 54 |
| | AUGAUAACAG | | GACUUUUCUG | | GACTTTTCTG | |
| 211 | CAGAAAAGUCCAC | 831 | UCUGUUAUCAUGU | 1451 | TCTGTTATCATGT | Exon 54 |
| | AUGAUAACAGA | | GGACUUUUCUG | | GGACTTTTCTG | |
| 212 | CAGAAAAGUCCAC | 832 | CUCUGUUAUCAUG | 1452 | CTCTGTTATCATG | Exon 54 |
| | AUGAUAACAGAG | | UGGACUUUUCUG | | TGGACTTTTCTG | |
| 213 | AGAAAAGUCCACA | 833 | UCUGUUAUCAUGU | 1453 | TCTGTTATCATGT | Exon 54 |
| | UGAUAACAGA | | GGACUUUUCU | | GGACTTTTCT | |
| 214 | AGAAAAGUCCACA | 834 | CUCUGUUAUCAUG | 1454 | CTCTGTTATCATG | Exon 54 |
| | UGAUAACAGAG | | UGGACUUUUCU | | TGGACTTTTCT | |
| 215 | AGAAAAGUCCACA | 835 | UCUCUGUUAUCAU | 1455 | TCTCTGTTATCAT | Exon 54 |
| | UGAUAACAGAGA | | GUGGACUUUUCU | | GTGGACTTTTCT | |
| 216 | GAAAAGUCCACAU | 836 | CUGUUAUCAUGUG | 1456 | CTGTTATCATGTG | Exon 54 |
| | GAUAACAG | | GACUUUUC | | GACTTTTC | |
| 217 | GAAAAGUCCACAU | 837 | UCUGUUAUCAUGU | 1457 | TCTGTTATCATGT | Exon 54 |
| | GAUAACAGA | | GGACUUUUC | | GGACTTTTC | |
| 218 | GAAAAGUCCACAU | 838 | CUCUGUUAUCAUG | 1458 | CTCTGTTATCATG | Exon 54 |
| | GAUAACAGAG | | UGGACUUUUC | | TGGACTTTTC | |
| 219 | GAAAAGUCCACAU | 839 | UCUCUGUUAUCAU | 1459 | TCTCTGTTATCAT | Exon 54 |
| | GAUAACAGAGA | | GUGGACUUUUC | | GTGGACTTTTC | |
| 220 | GAAAAGUCCACAU | 840 | UUCUCUGUUAUCA | 1460 | TTCTCTGTTATCA | Exon 54 |
| | GAUAACAGAGAA | | UGUGGACUUUUC | | TGTGGACTTTTC | |
| 221 | AAAAGUCCACAUG | 841 | CUCUGUUAUCAUG | 1461 | CTCTGTTATCATG | Exon 54 |
| | AUAACAGAG | | UGGACUUUU | | TGGACTTTT | |
| 222 | AAAAGUCCACAUG | 842 | UCUCUGUUAUCAU | 1462 | TCTCTGTTATCAT | Exon 54 |
| | AUAACAGAGA | | GUGGACUUUU | | GTGGACTTTT | |
| 223 | AAAAGUCCACAUG | 843 | UUCUCUGUUAUCA | 1463 | TTCTCTGTTATCA | Exon 54 |
| | AUAACAGAGAA | | UGUGGACUUUU | | TGTGGACTTTT | |
| 224 | AAAAGUCCACAUG | 844 | AUUCUCUGUUAUC | 1464 | ATTCTCTGTTATC | Exon 54 |
| | AUAACAGAGAAU | | AUGUGGACUUUU | | ATGTGGACTTTT | |
| 225 | AAAGUCCACAUGA | 845 | CUCUGUUAUCAUG | 1465 | CTCTGTTATCATG | Exon 54 |
| | UAACAGAG | | UGGACUUU | | TGGACTTT | |
| 226 | AAAGUCCACAUGA | 846 | UCUCUGUUAUCAU | 1466 | TCTCTGTTATCAT | Exon 54 |
| | UAACAGAGA | | GUGGACUUU | | GTGGACTTT | |
| 227 | AAAGUCCACAUGA | 847 | UUCUCUGUUAUCA | 1467 | TTCTCTGTTATCA | Exon 54 |
| | UAACAGAGAA | | UGUGGACUUU | | TGTGGACTTT | |
| 228 | AAAGUCCACAUGA | 848 | AUUCUCUGUUAUC | 1468 | ATTCTCTGTTATC | Exon 54 |
| | UAACAGAGAAU | | AUGUGGACUUU | | ATGTGGACTTT | |
| 229 | AAAGUCCACAUGA | 849 | UAUUCUCUGUUAU | 1469 | TATTCTCTGTTAT | Exon 54 |
| | UAACAGAGAAUA | | CAUGUGGACUUU | | CATGTGGACTTT | |
| 230 | AAGUCCACAUGAU | 850 | CUCUGUUAUCAUG | 1470 | CTCTGTTATCATG | Exon 54 |
| | AACAGAG | | UGGACUU | | TGGACTT | |
| 231 | AAGUCCACAUGAU | 851 | UCUCUGUUAUCAU | 1471 | TCTCTGTTATCAT | Exon 54 |
| | AACAGAGA | | GUGGACUU | | GTGGACTT | |
| 232 | AAGUCCACAUGAU | 852 | UUCUCUGUUAUCA | 1472 | TTCTCTGTTATCA | Exon 54 |
| | AACAGAGAA | | UGUGGACUU | | TGTGGACTT | |
| 233 | AAGUCCACAUGAU | 853 | AUUCUCUGUUAUC | 1473 | ATTCTCTGTTATC | Exon 54 |
| | AACAGAGAAU | | AUGUGGACUU | | ATGTGGACTT | |
| 234 | AAGUCCACAUGAU | 854 | UAUUCUCUGUUAU | 1474 | TATTCTCTGTTAT | Exon 54 |
| | AACAGAGAAUA | | CAUGUGGACUU | | CATGTGGACTT | |
| 235 | AAGUCCACAUGAU | 855 | AUAUUCUCUGUUA | 1475 | ATATTCTCTGTTA | Exon 54 |
| | AACAGAGAAUAU | | UCAUGUGGACUU | | TCATGTGGACTT | |
| 236 | AGUCCACAUGAUA | 856 | UUCUCUGUUAUCA | 1476 | TTCTCTGTTATCA | Exon 54 |
| | ACAGAGAA | | UGUGGACU | | TGTGGACT | |
| 237 | AGUCCACAUGAUA | 857 | AUUCUCUGUUAUC | 1477 | ATTCTCTGTTATC | Exon 54 |
| | ACAGAGAAU | | AUGUGGACU | | ATGTGGACT | |
| 238 | AGUCCACAUGAUA | 858 | UAUUCUCUGUUAU | 1478 | TATTCTCTGTTAT | Exon 54 |
| | ACAGAGAAUA | | CAUGUGGACU | | CATGTGGACT | |
| 239 | AGUCCACAUGAUA | 859 | AUAUUCUCUGUUA | 1479 | ATATTCTCTGTTA | Exon 54 |
| | ACAGAGAAUAU | | UCAUGUGGACU | | TCATGTGGACT | |
| 240 | AGUCCACAUGAUA | 860 | GAUAUUCUCUGUU | 1480 | GATATTCTCTGTT | Exon 54 |
| | ACAGAGAAUAUC | | AUCAUGUGGACU | | ATCATGTGGACT | |
| 241 | GUCCACAUGAUAA | 861 | GAUAUUCUCUGUU | 1481 | GATATTCTCTGTT | Exon 54 |
| | CAGAGAAUAUC | | AUCAUGUGGAC | | ATCATGTGGAC | |
| 242 | GUCCACAUGAUAA | 862 | UGAUAUUCUCUGU | 1482 | TGATATTCTCTGT | Exon 54 |
| | CAGAGAAUAUCA | | UAUCAUGUGGAC | | TATCATGTGGAC | |
| 243 | GGAAGAAACUCAU | 863 | UUGCAGUAAUCUA | 1483 | TTGCAGTAATCTA | Exon 55 |
| | AGAUUACUGCAA | | UGAGUUUCUUCC | | TGAGTTTCTTCC | |
| 244 | GCUGAAACAACUG | 864 | UAGGACAUUGGCA | 1484 | TAGGACATTGGCA | Exon 55 |
| | CCAAUGUCCUA | | GUUGUUUCAGC | | GTTGTTTCAGC | |
| 245 | GCUGAAACAACUG | 865 | GUAGGACAUUGGC | 1485 | GTAGGACATTGGC | Exon 55 |
| | CCAAUGUCCUAC | | AGUUGUUUCAGC | | AGTTGTTTCAGC | |
| 246 | UGAAACAACUGCC | 866 | GUAGGACAUUGGC | 1486 | GTAGGACATTGGC | Exon 55 |
| | AAUGUCCUAC | | AGUUGUUUCA | | AGTTGTTTCA | |
| 247 | UGAAACAACUGCC | 867 | UGUAGGACAUUGG | 1487 | TGTAGGACATTGG | Exon 55 |
| | AAUGUCCUACA | | CAGUUGUUUCA | | CAGTTGTTTCA | |
| 248 | UGAAACAACUGCC | 868 | CUGUAGGACAUUG | 1488 | CTGTAGGACATTG | Exon 55 |
| | AAUGUCCUACAG | | GCAGUUGUUUCA | | GCAGTTGTTTCA | |
| 249 | AACAACUGCCAAU | 869 | AUCCUGUAGGACA | 1489 | ATCCTGTAGGACA | Exon 55 |
| | GUCCUACAGGAU | | UUGGCAGUUGUU | | TTGGCAGTTGTT | |
| 250 | ACAACUGCCAAUG | 870 | AUCCUGUAGGACA | 1490 | ATCCTGTAGGACA | Exon 55 |
| | UCCUACAGGAU | | UUGGCAGUUGU | | TTGGCAGTTGT | |
| 251 | CAACUGCCAAUGU | 871 | AUCCUGUAGGACA | 1491 | ATCCTGTAGGACA | Exon 55 |
| | CCUACAGGAU | | UUGGCAGUUG | | TTGGCAGTTG | |
| 252 | AACUGCCAAUGUC | 872 | AUCCUGUAGGACA | 1492 | ATCCTGTAGGACA | Exon 55 |
| | CUACAGGAU | | UUGGCAGUU | | TTGGCAGTT | |
| 253 | AACUGCCAAUGUC | 873 | AGCAUCCUGUAGG | 1493 | AGCATCCTGTAGG | Exon 55 |
| | CUACAGGAUGCU | | ACAUUGGCAGUU | | ACATTGGCAGTT | |
| 254 | ACUGCCAAUGUCC | 874 | AUCCUGUAGGACA | 1494 | ATCCTGTAGGACA | Exon 55 |
| | UACAGGAU | | UUGGCAGU | | TTGGCAGT | |
| 255 | ACUGCCAAUGUCC | 875 | AGCAUCCUGUAGG | 1495 | AGCATCCTGTAGG | Exon 55 |
| | UACAGGAUGCU | | ACAUUGGCAGU | | ACATTGGCAGT | |
| 256 | CUGCCAAUGUCCU | 876 | AGCAUCCUGUAGG | 1496 | AGCATCCTGTAGG | Exon 55 |
| | ACAGGAUGCU | | ACAUUGGCAG | | ACATTGGCAG | |
| 257 | UGCCAAUGUCCUA | 877 | AGCAUCCUGUAGG | 1497 | AGCATCCTGTAGG | Exon 55 |
| | CAGGAUGCU | | ACAUUGGCA | | ACATTGGCA | |
| 258 | GCCAAUGUCCUAC | 878 | AGCAUCCUGUAGG | 1498 | AGCATCCTGTAGG | Exon 55 |
| | AGGAUGCU | | ACAUUGGC | | ACATTGGC | |
| 259 | AGAUGAUACCAGA | 879 | GGACUUUUCUGGU | 1499 | GGACTTTTCTGGT | Exon 54 |
| | AAAGUCC | | AUCAUCU | | ATCATCT | |
| 260 | AGAUGAUACCAGA | 880 | UGUGGACUUUUCU | 1500 | TGTGGACTTTTCT | Exon 54 |
| | AAAGUCCACA | | GGUAUCAUCU | | GGTATCATCT | |
| 261 | AGAUGAUACCAGA | 881 | AUGUGGACUUUUC | 1501 | ATGTGGACTTTTC | Exon 54 |
| | AAAGUCCACAU | | UGGUAUCAUCU | | TGGTATCATCT | |
| 262 | CUGAAACAACUGC | 882 | GUAGGACAUUGGC | 1502 | GTAGGACATTGGC | Exon 55 |
| | CAAUGUCCUAC | | AGUUGUUUCAG | | AGTTGTTTCAG | |
| 263 | CUGAAACAACUGC | 883 | UGUAGGACAUUGG | 1503 | TGTAGGACATTGG | Exon 55 |
| | CAAUGUCCUACA | | CAGUUGUUUCAG | | CAGTTGTTTCAG | |
| 264 | ACAACUGCCAAUG | 884 | CAUCCUGUAGGAC | 1504 | CATCCTGTAGGAC | Exon 55 |
| | UCCUACAGGAUG | | AUUGGCAGUUGU | | ATTGGCAGTTGT | |
| 265 | CAACUGCCAAUGU | 885 | CAUCCUGUAGGAC | 1505 | CATCCTGTAGGAC | Exon 55 |
| | CCUACAGGAUG | | AUUGGCAGUUG | | ATTGGCAGTTG | |
| 266 | AACUGCCAAUGUC | 886 | CAUCCUGUAGGAC | 1506 | CATCCTGTAGGAC | Exon 55 |
| | CUACAGGAUG | | AUUGGCAGUU | | ATTGGCAGTT | |
| 267 | ACUGCCAAUGUCC | 887 | CAUCCUGUAGGAC | 1507 | CATCCTGTAGGAC | Exon 55 |
| | UACAGGAUG | | AUUGGCAGU | | ATTGGCAGT | |
| 268 | ACUGCCAAUGUCC | 888 | UAGCAUCCUGUAG | 1508 | TAGCATCCTGTAG | Exon 55 |
| | UACAGGAUGCUA | | GACAUUGGCAGU | | GACATTGGCAGT | |
| 269 | CUGCCAAUGUCCU | 889 | CAUCCUGUAGGAC | 1509 | CATCCTGTAGGAC | Exon 55 |
| | ACAGGAUG | | AUUGGCAG | | ATTGGCAG | |
| 270 | CUGCCAAUGUCCU | 890 | UAGCAUCCUGUAG | 1510 | TAGCATCCTGTAG | Exon 55 |
| | ACAGGAUGCUA | | GACAUUGGCAG | | GACATTGGCAG | |
| 271 | UGCCAAUGUCCUA | 891 | UAGCAUCCUGUAG | 1511 | TAGCATCCTGTAG | Exon 55 |
| | CAGGAUGCUA | | GACAUUGGCA | | GACATTGGCA | |
| 272 | UGCCAAUGUCCUA | 892 | GGUAGCAUCCUGU | 1512 | GGTAGCATCCTGT | Exon 55 |
| | CAGGAUGCUACC | | AGGACAUUGGCA | | AGGACATTGGCA | |
| 273 | GCCAAUGUCCUAC | 893 | UAGCAUCCUGUAG | 1513 | TAGCATCCTGTAG | Exon 55 |
| | AGGAUGCUA | | GACAUUGGC | | GACATTGGC | |
| 274 | GCCAAUGUCCUAC | 894 | GGUAGCAUCCUGU | 1514 | GGTAGCATCCTGT | Exon 55 |
| | AGGAUGCUACC | | AGGACAUUGGC | | AGGACATTGGC | |
| 275 | CCAAGGGAGUAAA | 895 | UCAGCUCUUUUAC | 1515 | TCAGCTCTTTTAC | Exon 55 |
| | AGAGCUGA | | UCCCUUGG | | TCCCTTGG | |
| 276 | CCAAGGGAGUAAA | 896 | AUCAGCUCUUUUA | 1516 | ATCAGCTCTTTTA | Exon 55 |
| | AGAGCUGAU | | CUCCCUUGG | | CTCCCTTGG | |
| 277 | CCAAGGGAGUAAA | 897 | CAUCAGCUCUUUU | 1517 | CATCAGCTCTTTT | Exon 55 |
| | AGAGCUGAUG | | ACUCCCUUGG | | ACTCCCTTGG | |
| 278 | CCAAGGGAGUAAA | 898 | UCAUCAGCUCUUU | 1518 | TCATCAGCTCTTT | Exon 55 |
| | AGAGCUGAUGA | | UACUCCCUUGG | | TACTCCCTTGG | |
| 279 | CAAGGGAGUAAAA | 899 | UCAUCAGCUCUUU | 1519 | TCATCAGCTCTTT | Exon 55 |
| | GAGCUGAUGA | | UACUCCCUUG | | TACTCCCTTG | |
| 280 | CAAGGGAGUAAAA | 900 | UUCAUCAGCUCUU | 1520 | TTCATCAGCTCTT | Exon 55 |
| | GAGCUGAUGAA | | UUACUCCCUUG | | TTACTCCCTTG | |
| 281 | AGGGAGUAAAAGA | 901 | UUUCAUCAGCUCU | 1521 | TTTCATCAGCTCT | Exon 55 |
| | GCUGAUGAAA | | UUUACUCCCU | | TTTACTCCCT | |
| 282 | CUGAUGAAACAAU | 902 | GACUUACUUGCCA | 1522 | GACTTACTTGCCA | Exon 55/intron 55 |
| | GGCAAGUAAGUC | | UUGUUUCAUCAG | | TTGTTTCATCAG | junction |
| 283 | UGAUGAAACAAUG | 903 | GACUUACUUGCCA | 1523 | GACTTACTTGCCA | Exon 55/intron 55 |
| | GCAAGUAAGUC | | UUGUUUCAUCA | | TTGTTTCATCA | junction |
| 284 | GAUGAAACAAUGG | 904 | GACUUACUUGCCA | 1524 | GACTTACTTGCCA | Exon 55/intron 55 |
| | CAAGUAAGUC | | UUGUUUCAUC | | TTGTTTCATC | junction |
| 285 | CCUGGAAGGUUCC | 905 | GCAUCAUCGGAAC | 1525 | GCATCATCGGAAC | Exon 56 |
| | GAUGAUGC | | CUUCCAGG | | CTTCCAGG | |
| 286 | CCUGGAAGGUUCC | 906 | UGCAUCAUCGGAA | 1526 | TGCATCATCGGAA | Exon 56 |
| | GAUGAUGCA | | CCUUCCAGG | | CCTTCCAGG | |
| 287 | CAGAUGAUACCAG | 907 | UGUGGACUUUUCU | 1527 | TGTGGACTTTTCT | Exon 54 |
| | AAAAGUCCACA | | GGUAUCAUCUG | | GGTATCATCTG | |
| 288 | CAGAUGAUACCAG | 908 | AUGUGGACUUUUC | 1528 | ATGTGGACTTTTC | Exon 54 |
| | AAAAGUCCACAU | | UGGUAUCAUCUG | | TGGTATCATCTG | |
| 289 | CUGCCAAUGUCCU | 909 | GUAGCAUCCUGUA | 1529 | GTAGCATCCTGTA | Exon 55 |
| | ACAGGAUGCUAC | | GGACAUUGGCAG | | GGACATTGGCAG | |
| 290 | UGCCAAUGUCCUA | 910 | GUAGCAUCCUGUA | 1530 | GTAGCATCCTGTA | Exon 55 |
| | CAGGAUGCUAC | | GGACAUUGGCA | | GGACATTGGCA | |
| 291 | GCCAAUGUCCUAC | 911 | GUAGCAUCCUGUA | 1531 | GTAGCATCCTGTA | Exon 55 |
| | AGGAUGCUAC | | GGACAUUGGC | | GGACATTGGC | |
| 292 | CCAAUGUCCUACA | 912 | GUAGCAUCCUGUA | 1532 | GTAGCATCCTGTA | Exon 55 |
| | GGAUGCUAC | | GGACAUUGG | | GGACATTGG | |
| 293 | AGGGAGUAAAAGA | 913 | GUUUCAUCAGCUC | 1533 | GTTTCATCAGCTC | Exon 55 |
| | GCUGAUGAAAC | | UUUUACUCCCU | | TTTTACTCCCT | |
| 294 | UGAUGAAACAAUG | 914 | UGACUUACUUGCC | 1534 | TGACTTACTTGCC | Exon 55/intron 55 |
| | GCAAGUAAGUCA | | AUUGUUUCAUCA | | ATTGTTTCATCA | junction |
| 295 | GAUGAAACAAUGG | 915 | UGACUUACUUGCC | 1535 | TGACTTACTTGCC | Exon 55/intron 55 |
| | CAAGUAAGUCA | | AUUGUUUCAUC | | ATTGTTTCATC | junction |
| 296 | CCUGGAAGGUUCC | 916 | CUGCAUCAUCGGA | 1536 | CTGCATCATCGGA | Exon 56 |
| | GAUGAUGCAG | | ACCUUCCAGG | | ACCTTCCAGG | |
| 297 | GGAAGGUUCCGAU | 917 | CUGCAUCAUCGGA | 1537 | CTGCATCATCGGA | Exon 56 |
| | GAUGCAG | | ACCUUCC | | ACCTTCC | |
| 298 | GAUCCAAUUGAAC | 918 | UGCUGAGAAUUGU | 1538 | TGCTGAGAATTGT | Intron 55 |
| | AAUUCUCAGCA | | UCAAUUGGAUC | | TCAATTGGATC | |
| 299 | AGGUUCCGAUGAU | 919 | ACAGGACUGCAUC | 1539 | ACAGGACTGCATC | Exon 56 |
| | GCAGUCCUGU | | AUCGGAACCU | | ATCGGAACCT | |
| 300 | GGUUCCGAUGAUG | 920 | ACAGGACUGCAUC | 1540 | ACAGGACTGCATC | Exon 56 |
| | CAGUCCUGU | | AUCGGAACC | | ATCGGAACC | |
| 301 | CCUGGAAGGUUCC | 921 | ACUGCAUCAUCGG | 1541 | ACTGCATCATCGG | Exon 56 |
| | GAUGAUGCAGU | | AACCUUCCAGG | | AACCTTCCAGG | |
| 302 | CUGGAAGGUUCCG | 922 | ACUGCAUCAUCGG | 1542 | ACTGCATCATCGG | Exon 56 |
| | AUGAUGCAGU | | AACCUUCCAG | | AACCTTCCAG | |
| 303 | GGAAGGUUCCGAU | 923 | ACUGCAUCAUCGG | 1543 | ACTGCATCATCGG | Exon 56 |
| | GAUGCAGU | | AACCUUCC | | AACCTTCC | |
| 304 | GAAGGUUCCGAUG | 924 | ACAGGACUGCAUC | 1544 | ACAGGACTGCATC | Exon 56 |
| | AUGCAGUCCUGU | | AUCGGAACCUUC | | ATCGGAACCTTC | |
| 305 | GGUUCCGAUGAUG | 925 | GUAACAGGACUGC | 1545 | GTAACAGGACTGC | Exon 56 |
| | CAGUCCUGUUAC | | AUCAUCGGAACC | | ATCATCGGAACC | |
| 306 | GUUCCGAUGAUGC | 926 | GUAACAGGACUGC | 1546 | GTAACAGGACTGC | Exon 56 |
| | AGUCCUGUUAC | | AUCAUCGGAAC | | ATCATCGGAAC | |
| 307 | GUGGAUCCAAUUG | 927 | GAGAAUUGUUCAA | 1547 | GAGAATTGTTCAA | Intron 55 |
| | AACAAUUCUC | | UUGGAUCCAC | | TTGGATCCAC | |
| 308 | GGAUCCAAUUGAA | 928 | UGCUGAGAAUUGU | 1548 | TGCTGAGAATTGT | Intron 55 |
| | CAAUUCUCAGCA | | UCAAUUGGAUCC | | TCAATTGGATCC | |
| 309 | GAUCCAAUUGAAC | 929 | AUGCUGAGAAUUG | 1549 | ATGCTGAGAATTG | Intron 55 |
| | AAUUCUCAGCAU | | UUCAAUUGGAUC | | TTCAATTGGATC | |
| 310 | AAGGUUCCGAUGA | 930 | ACAGGACUGCAUC | 1550 | ACAGGACTGCATC | Exon 56 |
| | UGCAGUCCUGU | | AUCGGAACCUU | | ATCGGAACCTT | |
| 311 | AGGUUCCGAUGAU | 931 | GGACUGCAUCAUC | 1551 | GGACTGCATCATC | Exon 56 |
| | GCAGUCC | | GGAACCU | | GGAACCT | |
| 312 | GUGGAUCCAAUUG | 932 | UGAGAAUUGUUCA | 1552 | TGAGAATTGTTCA | Intron 55 |
| | AACAAUUCUCA | | AUUGGAUCCAC | | ATTGGATCCAC | |
| 313 | AGGUUCCGAUGAU | 933 | UAACAGGACUGCA | 1553 | TAACAGGACTGCA | Exon 56 |
| | GCAGUCCUGUUA | | UCAUCGGAACCU | | TCATCGGAACCT | |
| 314 | GGUUCCGAUGAUG | 934 | UAACAGGACUGCA | 1554 | TAACAGGACTGCA | Exon 56 |
| | CAGUCCUGUUA | | UCAUCGGAACC | | TCATCGGAACC | |
| 315 | CUGGAAGGUUCCG | 935 | GGACUGCAUCAUC | 1555 | GGACTGCATCATC | Exon 56 |
| | AUGAUGCAGUCC | | GGAACCUUCCAG | | GGAACCTTCCAG | |
| 316 | UGGAAGGUUCCGA | 936 | GGACUGCAUCAUC | 1556 | GGACTGCATCATC | Exon 56 |
| | UGAUGCAGUCC | | GGAACCUUCCA | | GGAACCTTCCA | |
| 317 | GGAAGGUUCCGAU | 937 | GGACUGCAUCAUC | 1557 | GGACTGCATCATC | Exon 56 |
| | GAUGCAGUCC | | GGAACCUUCC | | GGAACCTTCC | |
| 318 | UGUGGAUCCAAUU | 938 | GAGAAUUGUUCAA | 1558 | GAGAATTGTTCAA | Intron 55 |
| | GAACAAUUCUC | | UUGGAUCCACA | | TTGGATCCACA | |
| 319 | UUGUGGAUCCAAU | 939 | GAGAAUUGUUCAA | 1559 | GAGAATTGTTCAA | Intron 55 |
| | UGAACAAUUCUC | | UUGGAUCCACAA | | TTGGATCCACAA | |
| 320 | UGUGGAUCCAAUU | 940 | UGAGAAUUGUUCA | 1560 | TGAGAATTGTTCA | Intron 55 |
| | GAACAAUUCUCA | | AUUGGAUCCACA | | ATTGGATCCACA | |
| 321 | GUUCCGAUGAUGC | 941 | ACAGGACUGCAUC | 1561 | ACAGGACTGCATC | Exon 56 |
| | AGUCCUGU | | AUCGGAAC | | ATCGGAAC | |
| 322 | UCCGAUGAUGCAG | 942 | UAACAGGACUGCA | 1562 | TAACAGGACTGCA | Exon 56 |
| | UCCUGUUA | | UCAUCGGA | | TCATCGGA | |
| 323 | UCCGAUGAUGCAG | 943 | GUAACAGGACUGC | 1563 | GTAACAGGACTGC | Exon 56 |
| | UCCUGUUAC | | AUCAUCGGA | | ATCATCGGA | |
| 324 | GUUCCGAUGAUGC | 944 | UAACAGGACUGCA | 1564 | TAACAGGACTGCA | Exon 56 |
| | AGUCCUGUUA | | UCAUCGGAAC | | TCATCGGAAC | |
| 325 | UUCCGAUGAUGCA | 945 | GUAACAGGACUGC | 1565 | GTAACAGGACTGC | Exon 56 |
| | GUCCUGUUAC | | AUCAUCGGAA | | ATCATCGGAA | |
| 326 | CUCCAAAUUCACA | 946 | CAAGCGAUGAAUG | 1566 | CAAGCGATGAATG | Intron 55 |
| | UUCAUCGCUUG | | UGAAUUUGGAG | | TGAATTTGGAG | |
| 327 | GAAGGUUCCGAUG | 947 | GGACUGCAUCAUC | 1567 | GGACTGCATCATC | Exon 56 |
| | AUGCAGUCC | | GGAACCUUC | | GGAACCTTC | |
| 328 | AAGGUUCCGAUGA | 948 | GGACUGCAUCAUC | 1568 | GGACTGCATCATC | Exon 56 |
| | UGCAGUCC | | GGAACCUU | | GGAACCTT | |
| 329 | GGAGCUUGGGAGG | 949 | UCGUCUUGAACCC | 1569 | TCGTCTTGAACCC | Intron 54 |
| | GUUCAAGACGA | | UCCCAAGCUCC | | TCCCAAGCTCC | |
| 330 | GGAGCUUGGGAGG | 950 | AUCGUCUUGAACC | 1570 | ATCGTCTTGAACC | Intron 54 |
| | GUUCAAGACGAU | | CUCCCAAGCUCC | | CTCCCAAGCTCC | |
| 331 | UGGCUGUAAUAAU | 951 | CACCACCCCAUUA | 1571 | CACCACCCCATTA | Intron 54 |
| | GGGGUGGUG | | UUACAGCCA | | TTACAGCCA | |
| 332 | UGGCUGUAAUAAU | 952 | UCACCACCCCAUU | 1572 | TCACCACCCCATT | Intron 54 |
| | GGGGUGGUGA | | AUUACAGCCA | | ATTACAGCCA | |
| 333 | GGCUGUAAUAAUG | 953 | UCACCACCCCAUU | 1573 | TCACCACCCCATT | Intron 54 |
| | GGGUGGUGA | | AUUACAGCC | | ATTACAGCC | |
| 334 | GGGGUGGUGAAAC | 954 | CCAUCCAGUUUCA | 1574 | CCATCCAGTTTCA | Intron 54 |
| | UGGAUGG | | CCACCCC | | CCACCCC | |
| 335 | UUGGCUGUAAUAA | 955 | UCACCACCCCAUU | 1575 | TCACCACCCCATT | Intron 54 |
| | UGGGGUGGUGA | | AUUACAGCCAA | | ATTACAGCCAA | |
| 336 | GGGGUGGUGAAAC | 956 | UCCAUCCAGUUUC | 1576 | TCCATCCAGTTTC | Intron 54 |
| | UGGAUGGA | | ACCACCCC | | ACCACCCC | |
| 337 | GCUGUAAUAAUGG | 957 | UCACCACCCCAUU | 1577 | TCACCACCCCATT | Intron 54 |
| | GGUGGUGA | | AUUACAGC | | ATTACAGC | |
| 338 | UGGGGUGGUGAAA | 958 | CCAUCCAGUUUCA | 1578 | CCATCCAGTTTCA | Intron 54 |
| | CUGGAUGG | | CCACCCCA | | CCACCCCA | |
| 339 | UGGCUGUAAUAAU | 959 | UUUCACCACCCCA | 1579 | TTTCACCACCCCA | Intron 54 |
| | GGGGUGGUGAAA | | UUAUUACAGCCA | | TTATTACAGCCA | |
| 340 | GGCUGUAAUAAUG | 960 | UUUCACCACCCCA | 1580 | TTTCACCACCCCA | Intron 54 |
| | GGGUGGUGAAA | | UUAUUACAGCC | | TTATTACAGCC | |
| 341 | UGGGGUGGUGAAA | 961 | CAUCCAGUUUCAC | 1581 | CATCCAGTTTCAC | Intron 54 |
| | CUGGAUG | | CACCCCA | | CACCCCA | |
| 342 | UGGGGUGGUGAAA | 962 | UCCAUCCAGUUUC | 1582 | TCCATCCAGTTTC | Intron 54 |
| | CUGGAUGGA | | ACCACCCCA | | ACCACCCCA | |
| 343 | AUGGCAAGUAAGU | 963 | GGAAAUGCCUGAC | 1583 | GGAAATGCCTGAC | Exon 55/intron 55 |
| | CAGGCAUUUCC | | UUACUUGCCAU | | TTACTTGCCAT | junction |
| 344 | GCUGUAAUAAUGG | 964 | AGUUUCACCACCC | 1584 | AGTTTCACCACCC | Intron 54 |
| | GGUGGUGAAACU | | CAUUAUUACAGC | | CATTATTACAGC | |
| 345 | AUGGGGUGGUGAA | 965 | CAUCCAGUUUCAC | 1585 | CATCCAGTTTCAC | Intron 54 |
| | ACUGGAUG | | CACCCCAU | | CACCCCAT | |
| 346 | AUGGGGUGGUGAA | 966 | CCAUCCAGUUUCA | 1586 | CCATCCAGTTTCA | Intron 54 |
| | ACUGGAUGG | | CCACCCCAU | | CCACCCCAT | |
| 347 | GCAAGUAAGUCAG | 967 | GCGGAAAUGCCUG | 1587 | GCGGAAATGCCTG | Exon 55/intron 55 |
| | GCAUUUCCGC | | ACUUACUUGC | | ACTTACTTGC | junction |
| 348 | GGCUGUAAUAAUG | 968 | GUUUCACCACCCC | 1588 | GTTTCACCACCCC | Intron 54 |
| | GGGUGGUGAAAC | | AUUAUUACAGCC | | ATTATTACAGCC | |
| 349 | AAUGGGGUGGUGA | 969 | CAUCCAGUUUCAC | 1589 | CATCCAGTTTCAC | Intron 54 |
| | AACUGGAUG | | CACCCCAUU | | CACCCCATT | |
| 350 | AUGGGGUGGUGAA | 970 | UCCAUCCAGUUUC | 1590 | TCCATCCAGTTTC | Intron 54 |
| | ACUGGAUGGA | | ACCACCCCAU | | ACCACCCCAT | |
| 351 | UGGCAAGUAAGUC | 971 | GCGGAAAUGCCUG | 591 | GCGGAAATGCCTG | Exon 55/intron 55 |
| | AGGCAUUUCCGC | | ACUUACUUGCCA | | ACTTACTTGCCA | junction |
| 352 | GCUGUAAUAAUGG | 972 | UUUCACCACCCCA | 1592 | TTTCACCACCCCA | Intron 54 |
| | GGUGGUGAAA | | UUAUUACAGC | | TTATTACAGC | |
| 353 | UAAUGGGGUGGUG | 973 | CAUCCAGUUUCAC | 1593 | CATCCAGTTTCAC | Intron 54 |
| | AAACUGGAUG | | CACCCCAUUA | | CACCCCATTA | |
| 354 | UAAUGGGGUGGUG | 974 | CCAUCCAGUUUCA | 1594 | CCATCCAGTTTCA | Intron 54 |
| | AAACUGGAUGG | | CCACCCCAUUA | | CCACCCCATTA | |
| 355 | AAUGGGGUGGUGA | 975 | CCAUCCAGUUUCA | 1595 | CCATCCAGTTTCA | Intron 54 |
| | AACUGGAUGG | | CCACCCCAUU | | CCACCCCATT | |
| 356 | AUGGCAAGUAAGU | 976 | GAAAUGCCUGACU | 1596 | GAAATGCCTGACT | Exon 55/intron 55 |
| | CAGGCAUUUC | | UACUUGCCAU | | TACTTGCCAT | junction |
| 357 | GGCAAGUAAGUCA | 977 | GCGGAAAUGCCUG | 1597 | GCGGAAATGCCTG | Exon 55/intron 55 |
| | GGCAUUUCCGC | | ACUUACUUGCC | | ACTTACTTGCC | junction |
| 358 | GGCAAGUAAGUCA | 978 | AGCGGAAAUGCCU | 1598 | AGCGGAAATGCCT | Exon 55/intron 55 |
| | GGCAUUUCCGCU | | GACUUACUUGCC | | GACTTACTTGCC | junction |
| 359 | AAUGGGGUGGUGA | 979 | UCCAUCCAGUUUC | 1599 | TCCATCCAGTTTC | Intron 54 |
| | AACUGGAUGGA | | ACCACCCCAUU | | ACCACCCCATT | |
| 360 | GGGUGGUGAAACU | 980 | UCCAUCCAGUUUC | 1600 | TCCATCCAGTTTC | Intron 54 |
| | GGAUGGA | | ACCACCC | | ACCACCC | |
| 361 | AUAAUGGGGUGGU | 981 | CAUCCAGUUUCAC | 1601 | CATCCAGTTTCAC | Intron 54 |
| | GAAACUGGAUG | | CACCCCAUUAU | | CACCCCATTAT | |
| 362 | AUAAUGGGGUGGU | 982 | CCAUCCAGUUUCA | 1602 | CCATCCAGTTTCA | Intron 54 |
| | GAAACUGGAUGG | | CCACCCCAUUAU | | CCACCCCATTAT | |
| 363 | UAAUGGGGUGGUG | 983 | UCCAUCCAGUUUC | 1603 | TCCATCCAGTTTC | Intron 54 |
| | AAACUGGAUGGA | | ACCACCCCAUUA | | ACCACCCCATTA | |
| 364 | AAUGGCAAGUAAG | 984 | GAAAUGCCUGACU | 1604 | GAAATGCCTGACT | Exon 55/intron 55 |
| | UCAGGCAUUUC | | UACUUGCCAUU | | TACTTGCCATT | junction |
| 365 | AAUAAUGGGGUGG | 985 | CAUCCAGUUUCAC | 1605 | CATCCAGTTTCAC | Intron 54 |
| | UGAAACUGGAUG | | CACCCCAUUAUU | | CACCCCATTATT | |
| 366 | AAUGGCAAGUAAG | 986 | GGAAAUGCCUGAC | 1606 | GGAAATGCCTGAC | Exon 55/intron 55 |
| | UCAGGCAUUUCC | | UUACUUGCCAUU | | TTACTTGCCATT | junction |
| 367 | UGGCAAGUAAGUC | 987 | GGAAAUGCCUGAC | 1607 | GGAAATGCCTGAC | Exon 55/intron 55 |
| | AGGCAUUUCC | | UUACUUGCCA | | TTACTTGCCA | junction |
| 368 | CCGAUGAUGCAGU | 988 | GUAACAGGACUGC | 1608 | GTAACAGGACTGC | Exon 56 |
| | CCUGUUAC | | AUCAUCGG | | ATCATCGG | |
| 369 | UCCAAAUUCACAU | 989 | ACAAGCGAUGAAU | 1609 | ACAAGCGATGAAT | Intron 55 |
| | UCAUCGCUUGU | | GUGAAUUUGGA | | GTGAATTTGGA | |
| 370 | GUAAUAAUGGGGU | 990 | GUUUCACCACCCC | 1610 | GTTTCACCACCCC | Intron 54 |
| | GGUGAAAC | | AUUAUUAC | | ATTATTAC | |
| 371 | GCUGUAAUAAUGG | 991 | GUUUCACCACCCC | 1611 | GTTTCACCACCCC | Intron 54 |
| | GGUGGUGAAAC | | AUUAUUACAGC | | ATTATTACAGC | |
| 372 | GCUUUGGAAGAAA | 992 | GUAAUCUAUGAGU | 1612 | GTAATCTATGAGT | Exon 55 |
| | CUCAUAGAUUAC | | UUCUUCCAAAGC | | TTCTTCCAAAGC | |
| 373 | UUGGAAGAAACUC | 993 | GCAGUAAUCUAUG | 1613 | GCAGTAATCTATG | Exon 55 |
| | AUAGAUUACUGC | | AGUUUCUUCCAA | | AGTTTCTTCCAA | |
| 374 | UGGAAGAAACUCA | 994 | GCAGUAAUCUAUG | 1614 | GCAGTAATCTATG | Exon 55 |
| | UAGAUUACUGC | | AGUUUCUUCCA | | AGTTTCTTCCA | |
| 375 | UGGAAGAAACUCA | 995 | UGCAGUAAUCUAU | 1615 | TGCAGTAATCTAT | Exon 55 |
| | UAGAUUACUGCA | | GAGUUUCUUCCA | | GAGTTTCTTCCA | |
| 376 | GGAAGAAACUCAU | 996 | GCAGUAAUCUAUG | 1616 | GCAGTAATCTATG | Exon 55 |
| | AGAUUACUGC | | AGUUUCUUCC | | AGTTTCTTCC | |
| 377 | GAAGAAACUCAUA | 997 | UGCAGUAAUCUAU | 1617 | TGCAGTAATCTAT | Exon 55 |
| | GAUUACUGCA | | GAGUUUCUUC | | GAGTTTCTTC | |
| 378 | AAACAACUGCCAA | 998 | UGUAGGACAUUGG | 1618 | TGTAGGACATTGG | Exon 55 |
| | UGUCCUACA | | CAGUUGUUU | | CAGTTGTTT | |
| 379 | AAACAACUGCCAA | 999 | CUGUAGGACAUUG | 1619 | CTGTAGGACATTG | Exon 55 |
| | UGUCCUACAG | | GCAGUUGUUU | | GCAGTTGTTT | |
| 380 | AACAACUGCCAAU | 1000 | GUAGGACAUUGGC | 1620 | GTAGGACATTGGC | Exon 55 |
| | GUCCUAC | | AGUUGUU | | AGTTGTT | |
| 381 | AACAACUGCCAAU | 1001 | UGUAGGACAUUGG | 1621 | TGTAGGACATTGG | Exon 55 |
| | GUCCUACA | | CAGUUGUU | | CAGTTGTT | |
| 382 | CAACUGCCAAUGU | 1002 | CUGUAGGACAUUG | 1622 | CTGTAGGACATTG | Exon 55 |
| | CCUACAG | | GCAGUUG | | GCAGTTG | |
| 383 | AACUGCCAAUGUC | 1003 | CCUGUAGGACAUU | 1623 | CCTGTAGGACATT | Exon 55 |
| | CUACAGG | | GGCAGUU | | GGCAGTT | |
| 384 | GAUGAAAACAGCC | 1004 | GGAUUUUUUGGCU | 1624 | GGATTTTTTGGCT | Exon 56 |
| | AAAAAAUCC | | GUUUUCAUC | | GTTTTCATC | |
| 385 | GAUGAAAACAGCC | 1005 | AGGAUUUUUUGGC | 1625 | AGGATTTTTTGGC | Exon 56 |
| | AAAAAAUCCU | | UGUUUUCAUC | | TGTTTTCATC | |
| 386 | GAUGAAAACAGCC | 1006 | CAGGAUUUUUUGG | 1626 | CAGGATTTTTTGG | Exon 56 |
| | AAAAAAUCCUG | | CUGUUUUCAUC | | CTGTTTTCATC | |
| 387 | GAUGAAAACAGCC | 1007 | UCAGGAUUUUUUG | 1627 | TCAGGATTTTTTG | Exon 56 |
| | AAAAAAUCCUGA | | GCUGUUUUCAUC | | GCTGTTTTCATC | |
| 388 | AUGAAAACAGCCA | 1008 | CUCAGGAUUUUUU | 1628 | CTCAGGATTTTTT | Exon 56 |
| | AAAAAUCCUGAG | | GGCUGUUUUCAU | | GGCTGTTTTCAT | |
| 389 | UGAAAACAGCCAA | 1009 | CUCAGGAUUUUUU | 1629 | CTCAGGATTTTTT | Exon 56 |
| | AAAAUCCUGAG | | GGCUGUUUUCA | | GGCTGTTTTCA | |
| 390 | UGAAAACAGCCAA | 1010 | UCUCAGGAUUUUU | 1630 | TCTCAGGATTTTT | Exon 56 |
| | AAAAUCCUGAGA | | UGGCUGUUUUCA | | TGGCTGTTTTCA | |
| 391 | GAAAACAGCCAAA | 1011 | UCUCAGGAUUUUU | 1631 | TCTCAGGATTTTT | Exon 56 |
| | AAAUCCUGAGA | | UGGCUGUUUUC | | TGGCTGTTTTC | |
| 392 | GAAAACAGCCAAA | 1012 | AUCUCAGGAUUUU | 1632 | ATCTCAGGATTTT | Exon 56 |
| | AAAUCCUGAGAU | | UUGGCUGUUUUC | | TTGGCTGTTTTC | |
| 393 | AAACAGCCAAAAA | 1013 | GAUCUCAGGAUUU | 1633 | GATCTCAGGATTT | Exon 56 |
| | AUCCUGAGAUC | | UUUGGCUGUUU | | TTTGGCTGTTT | |
| 394 | AAACAGCCAAAAA | 1014 | GGAUCUCAGGAUU | 1634 | GGATCTCAGGATT | Exon 56 |
| | AUCCUGAGAUCC | | UUUUGGCUGUUU | | TTTTGGCTGTTT | |
| 395 | AACAGCCAAAAAA | 1015 | GGAUCUCAGGAUU | 1635 | GGATCTCAGGATT | Exon 56 |
| | UCCUGAGAUCC | | UUUUGGCUGUU | | TTTTGGCTGTT | |
| 396 | AACAGCCAAAAAA | 1016 | GGGAUCUCAGGAU | 1636 | GGGATCTCAGGAT | Exon 56 |
| | UCCUGAGAUCCC | | UUUUUGGCUGUU | | TTTTTGGCTGTT | |
| 397 | CCUGAGAUCCCUG | 1017 | GAACCUUCCAGGG | 1637 | GAACCTTCCAGGG | Exon 56 |
| | GAAGGUUC | | AUCUCAGG | | ATCTCAGG | |
| 398 | GAAGAAACUCAUA | 1018 | GUUGCAGUAAUCU | 1638 | GTTGCAGTAATCT | Exon 55 |
| | GAUUACUGCAAC | | AUGAGUUUCUUC | | ATGAGTTTCTTC | |
| 399 | AAGAAACUCAUAG | 1019 | GUUGCAGUAAUCU | 1639 | GTTGCAGTAATCT | Exon 55 |
| | AUUACUGCAAC | | AUGAGUUUCUU | | ATGAGTTTCTT | |
| 400 | AAGAAACUCAUAG | 1020 | UGUUGCAGUAAUC | 1640 | TGTTGCAGTAATC | Exon 55 |
| | AUUACUGCAACA | | UAUGAGUUUCUU | | TATGAGTTTCTT | |
| 401 | AGAAACUCAUAGA | 1021 | GUUGCAGUAAUCU | 1641 | GTTGCAGTAATCT | Exon 55 |
| | UUACUGCAAC | | AUGAGUUUCU | | ATGAGTTTCT | |
| 402 | GAAACUCAUAGAU | 1022 | GUUGCAGUAAUCU | 1642 | GTTGCAGTAATCT | Exon 55 |
| | UACUGCAAC | | AUGAGUUUC | | ATGAGTTTC | |
| 403 | GAAACUCAUAGAU | 1023 | UGUUGCAGUAAUC | 1643 | TGTTGCAGTAATC | Exon 55 |
| | UACUGCAACA | | UAUGAGUUUC | | TATGAGTTTC | |
| 404 | AAACUCAUAGAUU | 1024 | CUGUUGCAGUAAU | 1644 | CTGTTGCAGTAAT | Exon 55 |
| | ACUGCAACAG | | CUAUGAGUUU | | CTATGAGTTT | |
| 405 | AACUCAUAGAUUA | 1025 | GUUGCAGUAAUCU | 1645 | GTTGCAGTAATCT | Exon 55 |
| | CUGCAAC | | AUGAGUU | | ATGAGTT | |
| 406 | AACUCAUAGAUUA | 1026 | UGUUGCAGUAAUC | 1646 | TGTTGCAGTAATC | Exon 55 |
| | CUGCAACA | | UAUGAGUU | | TATGAGTT | |
| 407 | AACUCAUAGAUUA | 1027 | CUGUUGCAGUAAU | 1647 | CTGTTGCAGTAAT | Exon 55 |
| | CUGCAACAG | | CUAUGAGUU | | CTATGAGTT | |
| 408 | ACUCAUAGAUUAC | 1028 | UGUUGCAGUAAUC | 1648 | TGTTGCAGTAATC | Exon 55 |
| | UGCAACA | | UAUGAGU | | TATGAGT | |
| 409 | ACUCAUAGAUUAC | 1029 | CUGUUGCAGUAAU | 1649 | CTGTTGCAGTAAT | Exon 55 |
| | UGCAACAG | | CUAUGAGU | | CTATGAGT | |
| 410 | GAUGAUACCAGAA | 1030 | UGGACUUUUCUGG | 1650 | TGGACTTTTCTGG | Exon 54 |
| | AAGUCCA | | UAUCAUC | | TATCATC | |
| 411 | GAUGAUACCAGAA | 1031 | GUGGACUUUUCUG | 1651 | GTGGACTTTTCTG | Exon 54 |
| | AAGUCCAC | | GUAUCAUC | | GTATCATC | |
| 412 | GAUGAUACCAGAA | 1032 | UGUGGACUUUUCU | 1652 | TGTGGACTTTTCT | Exon 54 |
| | AAGUCCACA | | GGUAUCAUC | | GGTATCATC | |
| 413 | GAUGAUACCAGAA | 1033 | CAUGUGGACUUUU | 1653 | CATGTGGACTTTT | Exon 54 |
| | AAGUCCACAUG | | CUGGUAUCAUC | | CTGGTATCATC | |
| 414 | AUGAUACCAGAAA | 1034 | AUGUGGACUUUUC | 1654 | ATGTGGACTTTTC | Exon 54 |
| | AGUCCACAU | | UGGUAUCAU | | TGGTATCAT | |
| 415 | AUGAUACCAGAAA | 1035 | CAUGUGGACUUUU | 1655 | CATGTGGACTTTT | Exon 54 |
| | AGUCCACAUG | | CUGGUAUCAU | | CTGGTATCAT | |
| 416 | UGAUACCAGAAAA | 1036 | UGUGGACUUUUCU | 1656 | TGTGGACTTTTCT | Exon 54 |
| | GUCCACA | | GGUAUCA | | GGTATCA | |
| 417 | UGAUACCAGAAAA | 1037 | AUGUGGACUUUUC | 1657 | ATGTGGACTTTTC | Exon 54 |
| | GUCCACAU | | UGGUAUCA | | TGGTATCA | |
| 418 | UGAUACCAGAAAA | 1038 | CAUGUGGACUUUU | 1658 | CATGTGGACTTTT | Exon 54 |
| | GUCCACAUG | | CUGGUAUCA | | CTGGTATCA | |
| 419 | UGAUACCAGAAAA | 1039 | UAUCAUGUGGACU | 1659 | TATCATGTGGACT | Exon 54 |
| | GUCCACAUGAUA | | UUUCUGGUAUCA | | TTTCTGGTATCA | |
| 420 | GAUACCAGAAAAG | 1040 | AUGUGGACUUUUC | 1660 | ATGTGGACTTTTC | Exon 54 |
| | UCCACAU | | UGGUAUC | | TGGTATC | |
| 421 | GAUACCAGAAAAG | 1041 | CAUGUGGACUUUU | 1661 | CATGTGGACTTTT | Exon 54 |
| | UCCACAUG | | CUGGUAUC | | CTGGTATC | |
| 422 | GAUACCAGAAAAG | 1042 | UAUCAUGUGGACU | 1662 | TATCATGTGGACT | Exon 54 |
| | UCCACAUGAUA | | UUUCUGGUAUC | | TTTCTGGTATC | |
| 423 | AUACCAGAAAAGU | 1043 | CAUGUGGACUUUU | 1663 | CATGTGGACTTTT | Exon 54 |
| | CCACAUG | | CUGGUAU | | CTGGTAT | |
| 424 | AUACCAGAAAAGU | 1044 | UAUCAUGUGGACU | 1664 | TATCATGTGGACT | Exon 54 |
| | CCACAUGAUA | | UUUCUGGUAU | | TTTCTGGTAT | |
| 425 | AUACCAGAAAAGU | 1045 | UUAUCAUGUGGAC | 1665 | TTATCATGTGGAC | Exon 54 |
| | CCACAUGAUAA | | UUUUCUGGUAU | | TTTTCTGGTAT | |
| 426 | UACCAGAAAAGUC | 1046 | UAUCAUGUGGACU | 1666 | TATCATGTGGACT | Exon 54 |
| | CACAUGAUA | | UUUCUGGUA | | TTTCTGGTA | |
| 427 | ACCAGAAAAGUCC | 1047 | UAUCAUGUGGACU | 1667 | TATCATGTGGACT | Exon 54 |
| | ACAUGAUA | | UUUCUGGU | | TTTCTGGT | |
| 428 | CCAGAAAAGUCCA | 1048 | UAUCAUGUGGACU | 1668 | TATCATGTGGACT | Exon 54 |
| | CAUGAUA | | UUUCUGG | | TTTCTGG | |
| 429 | CAGAAAAGUCCAC | 1049 | UUAUCAUGUGGAC | 1669 | TTATCATGTGGAC | Exon 54 |
| | AUGAUAA | | UUUUCUG | | TTTTCTG | |
| 430 | AGAAAAGUCCACA | 1050 | GUUAUCAUGUGGA | 1670 | GTTATCATGTGGA | Exon 54 |
| | UGAUAAC | | CUUUUCU | | CTTTTCT | |
| 431 | AGAAAAGUCCACA | 1051 | UGUUAUCAUGUGG | 1671 | TGTTATCATGTGG | Exon 54 |
| | UGAUAACA | | ACUUUUCU | | ACTTTTCT | |
| 432 | AGAAAAGUCCACA | 1052 | CUGUUAUCAUGUG | 1672 | CTGTTATCATGTG | Exon 54 |
| | UGAUAACAG | | GACUUUUCU | | GACTTTTCT | |
| 433 | GAAAAGUCCACAU | 1053 | UGUUAUCAUGUGG | 1673 | TGTTATCATGTGG | Exon 54 |
| | GAUAACA | | ACUUUUC | | ACTTTTC | |
| 434 | AAAAGUCCACAUG | 1054 | UCUGUUAUCAUGU | 1674 | TCTGTTATCATGT | Exon 54 |
| | AUAACAGA | | GGACUUUU | | GGACTTTT | |
| 435 | AAAGUCCACAUGA | 1055 | UCUGUUAUCAUGU | 1675 | TCTGTTATCATGT | Exon 54 |
| | UAACAGA | | GGACUUU | | GGACTTT | |
| 436 | AGUCCACAUGAUA | 1056 | UCUCUGUUAUCAU | 1676 | TCTCTGTTATCAT | Exon 54 |
| | ACAGAGA | | GUGGACU | | GTGGACT | |
| 437 | GUCCACAUGAUAA | 1057 | UUCUCUGUUAUCA | 1677 | TTCTCTGTTATCA | Exon 54 |
| | CAGAGAA | | UGUGGAC | | TGTGGAC | |
| 438 | GUCCACAUGAUAA | 1058 | AUUCUCUGUUAUC | 1678 | ATTCTCTGTTATC | Exon 54 |
| | CAGAGAAU | | AUGUGGAC | | ATGTGGAC | |
| 439 | GUCCACAUGAUAA | 1059 | UAUUCUCUGUUAU | 1679 | TATTCTCTGTTAT | Exon 54 |
| | CAGAGAAUA | | CAUGUGGAC | | CATGTGGAC | |
| 440 | GUCCACAUGAUAA | 1060 | AUAUUCUCUGUUA | 1680 | ATATTCTCTGTTA | Exon 54 |
| | CAGAGAAUAU | | UCAUGUGGAC | | TCATGTGGAC | |
| 441 | CCACAUGAUAACA | 1061 | UAUUCUCUGUUAU | 1681 | TATTCTCTGTTAT | Exon 54 |
| | GAGAAUA | | CAUGUGG | | CATGTGG | |
| 442 | GAAGAAACUCAUA | 1062 | UUGCAGUAAUCUA | 1682 | TTGCAGTAATCTA | Exon 55 |
| | GAUUACUGCAA | | UGAGUUUCUUC | | TGAGTTTCTTC | |
| 443 | GAAGCUGAAACAA | 1063 | GGACAUUGGCAGU | 1683 | GGACATTGGCAGT | Exon 55 |
| | CUGCCAAUGUCC | | UGUUUCAGCUUC | | TGTTTCAGCTTC | |
| 444 | AAGCUGAAACAAC | 1064 | GGACAUUGGCAGU | 1684 | GGACATTGGCAGT | Exon 55 |
| | UGCCAAUGUCC | | UGUUUCAGCUU | | TGTTTCAGCTT | |
| 445 | AAGCUGAAACAAC | 1065 | AGGACAUUGGCAG | 1685 | AGGACATTGGCAG | Exon 55 |
| | UGCCAAUGUCCU | | UUGUUUCAGCUU | | TTGTTTCAGCTT | |
| 446 | AGCUGAAACAACU | 1066 | GACAUUGGCAGUU | 1686 | GACATTGGCAGTT | Exon 55 |
| | GCCAAUGUC | | GUUUCAGCU | | GTTTCAGCT | |
| 447 | AGCUGAAACAACU | 1067 | GGACAUUGGCAGU | 1687 | GGACATTGGCAGT | Exon 55 |
| | GCCAAUGUCC | | UGUUUCAGCU | | TGTTTCAGCT | |
| 448 | AGCUGAAACAACU | 1068 | AGGACAUUGGCAG | 1688 | AGGACATTGGCAG | Exon 55 |
| | GCCAAUGUCCU | | UUGUUUCAGCU | | TTGTTTCAGCT | |
| 449 | AGCUGAAACAACU | 1069 | UAGGACAUUGGCA | 1689 | TAGGACATTGGCA | Exon 55 |
| | GCCAAUGUCCUA | | GUUGUUUCAGCU | | GTTGTTTCAGCT | |
| 450 | GCUGAAACAACUG | 1070 | GACAUUGGCAGUU | 1690 | GACATTGGCAGTT | Exon 55 |
| | CCAAUGUC | | GUUUCAGC | | GTTTCAGC | |
| 451 | GCUGAAACAACUG | 1071 | GGACAUUGGCAGU | 1691 | GGACATTGGCAGT | Exon 55 |
| | CCAAUGUCC | | UGUUUCAGC | | TGTTTCAGC | |
| 452 | GCUGAAACAACUG | 1072 | AGGACAUUGGCAG | 1692 | AGGACATTGGCAG | Exon 55 |
| | CCAAUGUCCU | | UUGUUUCAGC | | TTGTTTCAGC | |
| 453 | CAACUGCCAAUGU | 1073 | GCAUCCUGUAGGA | 1693 | GCATCCTGTAGGA | Exon 55 |
| | CCUACAGGAUGC | | CAUUGGCAGUUG | | CATTGGCAGTTG | |
| 454 | AACUGCCAAUGUC | 1074 | GCAUCCUGUAGGA | 1694 | GCATCCTGTAGGA | Exon 55 |
| | CUACAGGAUGC | | CAUUGGCAGUU | | CATTGGCAGTT | |
| 455 | ACUGCCAAUGUCC | 1075 | GCAUCCUGUAGGA | 1695 | GCATCCTGTAGGA | Exon 55 |
| | UACAGGAUGC | | CAUUGGCAGU | | CATTGGCAGT | |
| 456 | CUGCCAAUGUCCU | 1076 | AUCCUGUAGGACA | 1696 | ATCCTGTAGGACA | Exon 55 |
| | ACAGGAU | | UUGGCAG | | TTGGCAG | |
| 457 | CUGCCAAUGUCCU | 1077 | GCAUCCUGUAGGA | 1697 | GCATCCTGTAGGA | Exon 55 |
| | ACAGGAUGC | | CAUUGGCAG | | CATTGGCAG | |
| 458 | UGCCAAUGUCCUA | 1078 | GCAUCCUGUAGGA | 1698 | GCATCCTGTAGGA | Exon 55 |
| | CAGGAUGC | | CAUUGGCA | | CATTGGCA | |
| 459 | GCCAAUGUCCUAC | 1079 | GCAUCCUGUAGGA | 1699 | GCATCCTGTAGGA | Exon 55 |
| | AGGAUGC | | CAUUGGC | | CATTGGC | |
| 460 | CCAAUGUCCUACA | 1080 | AGCAUCCUGUAGG | 1700 | AGCATCCTGTAGG | Exon 55 |
| | GGAUGCU | | ACAUUGG | | ACATTGG | |
| 461 | AGAGCUGAUGAAA | 1081 | CUUGCCAUUGUUU | 1701 | CTTGCCATTGTTT | Exon 55/intron 55 |
| | CAAUGGCAAG | | CAUCAGCUCU | | CATCAGCTCT | junction |
| 462 | AGAGCUGAUGAAA | 1082 | ACUUGCCAUUGUU | 1702 | ACTTGCCATTGTT | Exon 55/intron 55 |
| | CAAUGGCAAGU | | UCAUCAGCUCU | | TCATCAGCTCT | junction |
| 463 | AGAGCUGAUGAAA | 1083 | UACUUGCCAUUGU | 1703 | TACTTGCCATTGT | Exon 55/intron 55 |
| | CAAUGGCAAGUA | | UUCAUCAGCUCU | | TTCATCAGCTCT | junction |
| 464 | GAGCUGAUGAAAC | 1084 | ACUUGCCAUUGUU | 1704 | ACTTGCCATTGTT | Exon 55/intron 55 |
| | AAUGGCAAGU | | UCAUCAGCUC | | TCATCAGCTC | junction |
| 465 | GAGCUGAUGAAAC | 1085 | UACUUGCCAUUGU | 1705 | TACTTGCCATTGT | Exon 55/intron 55 |
| | AAUGGCAAGUA | | UUCAUCAGCUC | | TTCATCAGCTC | junction |
| 466 | GAGCUGAUGAAAC | 1086 | UUACUUGCCAUUG | 1706 | TTACTTGCCATTG | Exon 55/intron 55 |
| | AAUGGCAAGUAA | | UUUCAUCAGCUC | | TTTCATCAGCTC | junction |
| 467 | AGCUGAUGAAACA | 1087 | CUUACUUGCCAUU | 1707 | CTTACTTGCCATT | Exon 55/intron 55 |
| | AUGGCAAGUAAG | | GUUUCAUCAGCU | | GTTTCATCAGCT | junction |
| 468 | GCUGAUGAAACAA | 1088 | CUUACUUGCCAUU | 1708 | CTTACTTGCCATT | Exon 55/intron 55 |
| | UGGCAAGUAAG | | GUUUCAUCAGC | | GTTTCATCAGC | junction |
| 469 | GCUGAUGAAACAA | 1089 | ACUUACUUGCCAU | 1709 | ACTTACTTGCCAT | Exon 55/intron 55 |
| | UGGCAAGUAAGU | | UGUUUCAUCAGC | | TGTTTCATCAGC | junction |
| 470 | CUGAUGAAACAAU | 1090 | ACUUACUUGCCAU | 1710 | ACTTACTTGCCAT | Exon 55/intron 55 |
| | GGCAAGUAAGU | | UGUUUCAUCAG | | TGTTTCATCAG | junction |
| 471 | UAUCACAACCUGG | 1091 | GGCUGUUUUCAUC | 1711 | GGCTGTTTTCATC | Exon 56 |
| | AUGAAAACAGCC | | CAGGUUGUGAUA | | CAGGTTGTGATA | |
| 472 | AUCACAACCUGGA | 1092 | GGCUGUUUUCAUC | 1712 | GGCTGTTTTCATC | Exon 56 |
| | UGAAAACAGCC | | CAGGUUGUGAU | | CAGGTTGTGAT | |
| 473 | AUCACAACCUGGA | 1093 | UGGCUGUUUUCAU | 1713 | TGGCTGTTTTCAT | Exon 56 |
| | UGAAAACAGCCA | | CCAGGUUGUGAU | | CCAGGTTGTGAT | |
| 474 | UCACAACCUGGAU | 1094 | GGCUGUUUUCAUC | 1714 | GGCTGTTTTCATC | Exon 56 |
| | GAAAACAGCC | | CAGGUUGUGA | | CAGGTTGTGA | |
| 475 | UCACAACCUGGAU | 1095 | UGGCUGUUUUCAU | 1715 | TGGCTGTTTTCAT | Exon 56 |
| | GAAAACAGCCA | | CCAGGUUGUGA | | CCAGGTTGTGA | |
| 476 | UCACAACCUGGAU | 1096 | UUGGCUGUUUUCA | 1716 | TTGGCTGTTTTCA | Exon 56 |
| | GAAAACAGCCAA | | UCCAGGUUGUGA | | TCCAGGTTGTGA | |
| 477 | CACAACCUGGAUG | 1097 | UUUGGCUGUUUUC | 1717 | TTTGGCTGTTTTC | Exon 56 |
| | AAAACAGCCAAA | | AUCCAGGUUGUG | | ATCCAGGTTGTG | |
| 478 | ACAACCUGGAUGA | 1098 | UUUUGGCUGUUUU | 1718 | TTTTGGCTGTTTT | Exon 56 |
| | AAACAGCCAAAA | | CAUCCAGGUUGU | | CATCCAGGTTGT | |
| 479 | GGAUGAAAACAGC | 1099 | GAUUUUUUGGCUG | 1719 | GATTTTTTGGCTG | Exon 56 |
| | CAAAAAAUC | | UUUUCAUCC | | TTTTCATCC | |
| 480 | GGAUGAAAACAGC | 1100 | GGAUUUUUUGGCU | 1720 | GGATTTTTTGGCT | Exon 56 |
| | CAAAAAAUCC | | GUUUUCAUCC | | GTTTTCATCC | |
| 481 | GGAUGAAAACAGC | 1101 | AGGAUUUUUUGGC | 1721 | AGGATTTTTTGGC | Exon 56 |
| | CAAAAAAUCCU | | UGUUUUCAUCC | | TGTTTTCATCC | |
| 482 | GGAUGAAAACAGC | 1102 | CAGGAUUUUUUGG | 1722 | CAGGATTTTTTGG | Exon 56 |
| | CAAAAAAUCCUG | | CUGUUUUCAUCC | | CTGTTTTCATCC | |
| 483 | ACAGCCAAAAAAU | 1103 | GGAUCUCAGGAUU | 1723 | GGATCTCAGGATT | Exon 56 |
| | CCUGAGAUCC | | UUUUGGCUGU | | TTTTGGCTGT | |
| 484 | ACAGCCAAAAAAU | 1104 | GGGAUCUCAGGAU | 1724 | GGGATCTCAGGAT | Exon 56 |
| | CCUGAGAUCCC | | UUUUUGGCUGU | | TTTTTGGCTGT | |
| 485 | CCUGAGAUCCCUG | 1105 | UCGGAACCUUCCA | 1725 | TCGGAACCTTCCA | Exon 56 |
| | GAAGGUUCCGA | | GGGAUCUCAGG | | GGGATCTCAGG | |
| 486 | CCUGAGAUCCCUG | 1106 | AUCGGAACCUUCC | 1726 | ATCGGAACCTTCC | Exon 56 |
| | GAAGGUUCCGAU | | AGGGAUCUCAGG | | AGGGATCTCAGG | |
| 487 | CUGAGAUCCCUGG | 1107 | UCGGAACCUUCCA | 1727 | TCGGAACCTTCCA | Exon 56 |
| | AAGGUUCCGA | | GGGAUCUCAG | | GGGATCTCAG | |
| 488 | CUGAGAUCCCUGG | 1108 | AUCGGAACCUUCC | 1728 | ATCGGAACCTTCC | Exon 56 |
| | AAGGUUCCGAU | | AGGGAUCUCAG | | AGGGATCTCAG | |
| 489 | CUGAGAUCCCUGG | 1109 | CAUCGGAACCUUC | 1729 | CATCGGAACCTTC | Exon 56 |
| | AAGGUUCCGAUG | | CAGGGAUCUCAG | | CAGGGATCTCAG | |
| 490 | UGAGAUCCCUGGA | 1110 | UCGGAACCUUCCA | 1730 | TCGGAACCTTCCA | Exon 56 |
| | AGGUUCCGA | | GGGAUCUCA | | GGGATCTCA | |
| 491 | UGAGAUCCCUGGA | 1111 | AUCGGAACCUUCC | 1731 | ATCGGAACCTTCC | Exon 56 |
| | AGGUUCCGAU | | AGGGAUCUCA | | AGGGATCTCA | |
| 492 | UGAGAUCCCUGGA | 1112 | CAUCGGAACCUUC | 1732 | CATCGGAACCTTC | Exon 56 |
| | AGGUUCCGAUG | | CAGGGAUCUCA | | CAGGGATCTCA | |
| 493 | UGAGAUCCCUGGA | 1113 | UCAUCGGAACCUU | 1733 | TCATCGGAACCTT | Exon 56 |
| | AGGUUCCGAUGA | | CCAGGGAUCUCA | | CCAGGGATCTCA | |
| 494 | GAGAUCCCUGGAA | 1114 | UCGGAACCUUCCA | 1734 | TCGGAACCTTCCA | Exon 56 |
| | GGUUCCGA | | GGGAUCUC | | GGGATCTC | |
| 495 | GAGAUCCCUGGAA | 1115 | AUCGGAACCUUCC | 1735 | ATCGGAACCTTCC | Exon 56 |
| | GGUUCCGAU | | AGGGAUCUC | | AGGGATCTC | |
| 496 | GAGAUCCCUGGAA | 1116 | CAUCGGAACCUUC | 1736 | CATCGGAACCTTC | Exon 56 |
| | GGUUCCGAUG | | CAGGGAUCUC | | CAGGGATCTC | |
| 497 | GAGAUCCCUGGAA | 1117 | UCAUCGGAACCUU | 1737 | TCATCGGAACCTT | Exon 56 |
| | GGUUCCGAUGA | | CCAGGGAUCUC | | CCAGGGATCTC | |
| 498 | AGAUCCCUGGAAG | 1118 | CAUCGGAACCUUC | 1738 | CATCGGAACCTTC | Exon 56 |
| | GUUCCGAUG | | CAGGGAUCU | | CAGGGATCT | |
| 499 | AGAUCCCUGGAAG | 1119 | UCAUCGGAACCUU | 1739 | TCATCGGAACCTT | Exon 56 |
| | GUUCCGAUGA | | CCAGGGAUCU | | CCAGGGATCT | |
| 500 | GAUCCCUGGAAGG | 1120 | AUCGGAACCUUCC | 1740 | ATCGGAACCTTCC | Exon 56 |
| | UUCCGAU | | AGGGAUC | | AGGGATC | |
| 501 | GAUCCCUGGAAGG | 1121 | CAUCGGAACCUUC | 1741 | CATCGGAACCTTC | Exon 56 |
| | UUCCGAUG | | CAGGGAUC | | CAGGGATC | |
| 502 | GAUCCCUGGAAGG | 1122 | UCAUCGGAACCUU | 1742 | TCATCGGAACCTT | Exon 56 |
| | UUCCGAUGA | | CCAGGGAUC | | CCAGGGATC | |
| 503 | AUCCCUGGAAGGU | 1123 | UCAUCGGAACCUU | 1743 | TCATCGGAACCTT | Exon 56 |
| | UCCGAUGA | | CCAGGGAU | | CCAGGGAT | |
| 504 | UCCCUGGAAGGUU | 1124 | UCAUCGGAACCUU | 1744 | TCATCGGAACCTT | Exon 56 |
| | CCGAUGA | | CCAGGGA | | CCAGGGA | |
| 505 | AGAUGAUACCAGA | 1125 | UGGACUUUUCUGG | 1745 | TGGACTTTTCTGG | Exon 54 |
| | AAAGUCCA | | UAUCAUCU | | TATCATCT | |
| 506 | AGAUGAUACCAGA | 1126 | GUGGACUUUUCUG | 1746 | GTGGACTTTTCTG | Exon 54 |
| | AAAGUCCAC | | GUAUCAUCU | | GTATCATCT | |
| 507 | AGAUGAUACCAGA | 1127 | CAUGUGGACUUUU | 1747 | CATGTGGACTTTT | Exon 54 |
| | AAAGUCCACAUG | | CUGGUAUCAUCU | | CTGGTATCATCT | |
| 508 | UCCACAUGAUAAC | 1128 | GAUAUUCUCUGUU | 1748 | GATATTCTCTGTT | Exon 54 |
| | AGAGAAUAUC | | AUCAUGUGGA | | ATCATGTGGA | |
| 509 | CUGAAACAACUGC | 1129 | GGACAUUGGCAGU | 1749 | GGACATTGGCAGT | Exon 55 |
| | CAAUGUCC | | UGUUUCAG | | TGTTTCAG | |
| 510 | CUGAAACAACUGC | 1130 | AGGACAUUGGCAG | 1750 | AGGACATTGGCAG | Exon 55 |
| | CAAUGUCCU | | UUGUUUCAG | | TTGTTTCAG | |
| 511 | CUGAAACAACUGC | 1131 | UAGGACAUUGGCA | 1751 | TAGGACATTGGCA | Exon 55 |
| | CAAUGUCCUA | | GUUGUUUCAG | | GTTGTTTCAG | |
| 512 | UGCCAAUGUCCUA | 1132 | CAUCCUGUAGGAC | 1752 | CATCCTGTAGGAC | Exon 55 |
| | CAGGAUG | | AUUGGCA | | ATTGGCA | |
| 513 | CCAAUGUCCUACA | 1133 | UAGCAUCCUGUAG | 1753 | TAGCATCCTGTAG | Exon 55 |
| | GGAUGCUA | | GACAUUGG | | GACATTGG | |
| 514 | CCAAUGUCCUACA | 1134 | GGUAGCAUCCUGU | 1754 | GGTAGCATCCTGT | Exon 55 |
| | GGAUGCUACC | | AGGACAUUGG | | AGGACATTGG | |
| 515 | CUCCAAGGGAGUA | 1135 | AUCAGCUCUUUUA | 1755 | ATCAGCTCTTTTA | Exon 55 |
| | AAAGAGCUGAU | | CUCCCUUGGAG | | CTCCCTTGGAG | |
| 516 | UCCAAGGGAGUAA | 1136 | UCAGCUCUUUUAC | 1756 | TCAGCTCTTTTAC | Exon 55 |
| | AAGAGCUGA | | UCCCUUGGA | | TCCCTTGGA | |
| 517 | UCCAAGGGAGUAA | 1137 | AUCAGCUCUUUUA | 1757 | ATCAGCTCTTTTA | Exon 55 |
| | AAGAGCUGAU | | CUCCCUUGGA | | CTCCCTTGGA | |
| 518 | CCAAGGGAGUAAA | 1138 | CAGCUCUUUUACU | 1758 | CAGCTCTTTTACT | Exon 55 |
| | AGAGCUG | | CCCUUGG | | CCCTTGG | |
| 519 | CCAAGGGAGUAAA | 1139 | UUCAUCAGCUCUU | 1759 | TTCATCAGCTCTT | Exon 55 |
| | AGAGCUGAUGAA | | UUACUCCCUUGG | | TTACTCCCTTGG | |
| 520 | CAAGGGAGUAAAA | 1140 | CAUCAGCUCUUUU | 1760 | CATCAGCTCTTTT | Exon 55 |
| | GAGCUGAUG | | ACUCCCUUG | | ACTCCCTTG | |
| 521 | CAAGGGAGUAAAA | 1141 | UUUCAUCAGCUCU | 1761 | TTTCATCAGCTCT | Exon 55 |
| | GAGCUGAUGAAA | | UUUACUCCCUUG | | TTTACTCCCTTG | |
| 522 | AGGGAGUAAAAGA | 1142 | UCAUCAGCUCUUU | 1762 | TCATCAGCTCTTT | Exon 55 |
| | GCUGAUGA | | UACUCCCU | | TACTCCCT | |
| 523 | AGGGAGUAAAAGA | 1143 | UUCAUCAGCUCUU | 1763 | TTCATCAGCTCTT | Exon 55 |
| | GCUGAUGAA | | UUACUCCCU | | TTACTCCCT | |
| 524 | AAGAGCUGAUGAA | 1144 | UUGCCAUUGUUUC | 1764 | TTGCCATTGTTTC | Exon 55 |
| | ACAAUGGCAA | | AUCAGCUCUU | | ATCAGCTCTT | |
| 525 | AAGAGCUGAUGAA | 1145 | CUUGCCAUUGUUU | 1765 | CTTGCCATTGTTT | Exon 55/intron 55 |
| | ACAAUGGCAAG | | CAUCAGCUCUU | | CATCAGCTCTT | junction |
| 526 | AAGAGCUGAUGAA | 1146 | ACUUGCCAUUGUU | 1766 | ACTTGCCATTGTT | Exon 55/intron 55 |
| | ACAAUGGCAAGU | | UCAUCAGCUCUU | | TCATCAGCTCTT | junction |
| 527 | AUGAAACAAUGGC | 1147 | GACUUACUUGCCA | 1767 | GACTTACTTGCCA | Exon 55/intron 55 |
| | AAGUAAGUC | | UUGUUUCAU | | TTGTTTCAT | junction |
| 528 | UCCAAGGUGAAAU | 1148 | GUGUGAGCUUCAA | 1768 | GTGTGAGCTTCAA | Exon 56 |
| | UGAAGCUCACAC | | UUUCACCUUGGA | | TTTCACCTTGGA | |
| 529 | CCAAGGUGAAAUU | 1149 | GUGUGAGCUUCAA | 1769 | GTGTGAGCTTCAA | Exon 56 |
| | GAAGCUCACAC | | UUUCACCUUGG | | TTTCACCTTGG | |
| 530 | CCAAGGUGAAAUU | 1150 | UGUGUGAGCUUCA | 1770 | TGTGTGAGCTTCA | Exon 56 |
| | GAAGCUCACACA | | AUUUCACCUUGG | | ATTTCACCTTGG | |
| 531 | CAAGGUGAAAUUG | 1151 | CUGUGUGAGCUUC | 1771 | CTGTGTGAGCTTC | Exon 56 |
| | AAGCUCACACAG | | AAUUUCACCUUG | | AATTTCACCTTG | |
| 532 | AAGGUGAAAUUGA | 1152 | UCUGUGUGAGCUU | 1772 | TCTGTGTGAGCTT | Exon 56 |
| | AGCUCACACAGA | | CAAUUUCACCUU | | CAATTTCACCTT | |
| 533 | AGGUGAAAUUGAA | 1153 | UCUGUGUGAGCUU | 1773 | TCTGTGTGAGCTT | Exon 56 |
| | GCUCACACAGA | | CAAUUUCACCU | | CAATTTCACCT | |
| 534 | AGGUGAAAUUGAA | 1154 | AUCUGUGUGAGCU | 1774 | ATCTGTGTGAGCT | Exon 56 |
| | GCUCACACAGAU | | UCAAUUUCACCU | | TCAATTTCACCT | |
| 535 | UUAUCACAACCUG | 1155 | GCUGUUUUCAUCC | 1775 | GCTGTTTTCATCC | Exon 56 |
| | GAUGAAAACAGC | | AGGUUGUGAUAA | | AGGTTGTGATAA | |
| 536 | UGGAUGAAAACAG | 1156 | GGAUUUUUUGGCU | 1776 | GGATTTTTTGGCT | Exon 56 |
| | CCAAAAAAUCC | | GUUUUCAUCCA | | GTTTTCATCCA | |
| 537 | UGGAUGAAAACAG | 1157 | AGGAUUUUUUGGC | 1777 | AGGATTTTTTGGC | Exon 56 |
| | CCAAAAAAUCCU | | UGUUUUCAUCCA | | TGTTTTCATCCA | |
| 538 | CCUGAGAUCCCUG | 1158 | CGGAACCUUCCAG | 1778 | CGGAACCTTCCAG | Exon 56 |
| | GAAGGUUCCG | | GGAUCUCAGG | | GGATCTCAGG | |
| 539 | CUGAGAUCCCUGG | 1159 | CGGAACCUUCCAG | 1779 | CGGAACCTTCCAG | Exon 56 |
| | AAGGUUCCG | | GGAUCUCAG | | GGATCTCAG | |
| 540 | GAGAUCCCUGGAA | 1160 | AUCAUCGGAACCU | 1780 | ATCATCGGAACCT | Exon 56 |
| | GGUUCCGAUGAU | | UCCAGGGAUCUC | | TCCAGGGATCTC | |
| 541 | AGAUCCCUGGAAG | 1161 | AUCAUCGGAACCU | 1781 | ATCATCGGAACCT | Exon 56 |
| | GUUCCGAUGAU | | UCCAGGGAUCU | | TCCAGGGATCT | |
| 542 | GAUCCCUGGAAGG | 1162 | AUCAUCGGAACCU | 1782 | ATCATCGGAACCT | Exon 56 |
| | UUCCGAUGAU | | UCCAGGGAUC | | TCCAGGGATC | |
| 543 | GAUCCCUGGAAGG | 1163 | GCAUCAUCGGAAC | 1783 | GCATCATCGGAAC | Exon 56 |
| | UUCCGAUGAUGC | | CUUCCAGGGAUC | | CTTCCAGGGATC | |
| 544 | AUCCCUGGAAGGU | 1164 | AUCAUCGGAACCU | 1784 | ATCATCGGAACCT | Exon 56 |
| | UCCGAUGAU | | UCCAGGGAU | | TCCAGGGAT | |
| 545 | AUCCCUGGAAGGU | 1165 | GCAUCAUCGGAAC | 1785 | GCATCATCGGAAC | Exon 56 |
| | UCCGAUGAUGC | | CUUCCAGGGAU | | CTTCCAGGGAT | |
| 546 | AUCCCUGGAAGGU | 1166 | UGCAUCAUCGGAA | 1786 | TGCATCATCGGAA | Exon 56 |
| | UCCGAUGAUGCA | | CCUUCCAGGGAU | | CCTTCCAGGGAT | |
| 547 | UCCCUGGAAGGUU | 1167 | AUCAUCGGAACCU | 1787 | ATCATCGGAACCT | Exon 56 |
| | CCGAUGAU | | UCCAGGGA | | TCCAGGGA | |
| 548 | UCCCUGGAAGGUU | 1168 | GCAUCAUCGGAAC | 1788 | GCATCATCGGAAC | Exon 56 |
| | CCGAUGAUGC | | CUUCCAGGGA | | CTTCCAGGGA | |
| 549 | UCCCUGGAAGGUU | 1169 | UGCAUCAUCGGAA | 1789 | TGCATCATCGGAA | Exon 56 |
| | CCGAUGAUGCA | | CCUUCCAGGGA | | CCTTCCAGGGA | |
| 550 | CCCUGGAAGGUUC | 1170 | GCAUCAUCGGAAC | 1790 | GCATCATCGGAAC | Exon 56 |
| | CGAUGAUGC | | CUUCCAGGG | | CTTCCAGGG | |
| 551 | CCCUGGAAGGUUC | 1171 | UGCAUCAUCGGAA | 1791 | TGCATCATCGGAA | Exon 56 |
| | CGAUGAUGCA | | CCUUCCAGGG | | CCTTCCAGGG | |
| 552 | CUGGAAGGUUCCG | 1172 | GCAUCAUCGGAAC | 1792 | GCATCATCGGAAC | Exon 56 |
| | AUGAUGC | | CUUCCAG | | CTTCCAG | |
| 553 | CUGGAAGGUUCCG | 1173 | UGCAUCAUCGGAA | 1793 | TGCATCATCGGAA | Exon 56 |
| | AUGAUGCA | | CCUUCCAG | | CCTTCCAG | |
| 554 | UGGAAGGUUCCGA | 1174 | UGCAUCAUCGGAA | 1794 | TGCATCATCGGAA | Exon 56 |
| | UGAUGCA | | CCUUCCA | | CCTTCCA | |
| 555 | GGCUUACAGAAGC | 1175 | GCAGUUGUUUCAG | 1795 | GCAGTTGTTTCAG | Exon 55 |
| | UGAAACAACUGC | | CUUCUGUAAGCC | | CTTCTGTAAGCC | |
| 556 | GGGAGUAAAAGAG | 1176 | UUUCAUCAGCUCU | 1796 | TTTCATCAGCTCT | Exon 55 |
| | CUGAUGAAA | | UUUACUCCC | | TTTACTCCC | |
| 557 | GGGAGUAAAAGAG | 1177 | GUUUCAUCAGCUC | 1797 | GTTTCATCAGCTC | Exon 55 |
| | CUGAUGAAAC | | UUUUACUCCC | | TTTTACTCCC | |
| 558 | AAAAGAGCUGAUG | 1178 | UUGCCAUUGUUUC | 1798 | TTGCCATTGTTTC | Exon 55 |
| | AAACAAUGGCAA | | AUCAGCUCUUUU | | ATCAGCTCTTTT | |
| 559 | AGGUUCCGAUGAU | 1179 | AGGACUGCAUCAU | 1799 | AGGACTGCATCAT | Exon 56 |
| | GCAGUCCU | | CGGAACCU | | CGGAACCT | |
| 560 | AGGUUCCGAUGAU | 1180 | CAGGACUGCAUCA | 1800 | CAGGACTGCATCA | Exon 56 |
| | GCAGUCCUG | | UCGGAACCU | | TCGGAACCT | |
| 561 | GGUUCCGAUGAUG | 1181 | AGGACUGCAUCAU | 1801 | AGGACTGCATCAT | Exon 56 |
| | CAGUCCU | | CGGAACC | | CGGAACC | |
| 562 | GGUUCCGAUGAUG | 1182 | CAGGACUGCAUCA | 1802 | CAGGACTGCATCA | Exon 56 |
| | CAGUCCUG | | UCGGAACC | | TCGGAACC | |
| 563 | CAGAUGAUACCAG | 1183 | GGACUUUUCUGGU | 1803 | GGACTTTTCTGGT | Exon 54 |
| | AAAAGUCC | | AUCAUCUG | | ATCATCTG | |
| 564 | CAGAUGAUACCAG | 1184 | UGGACUUUUCUGG | 1804 | TGGACTTTTCTGG | Exon 54 |
| | AAAAGUCCA | | UAUCAUCUG | | TATCATCTG | |
| 565 | CAGAUGAUACCAG | 1185 | GUGGACUUUUCUG | 1805 | GTGGACTTTTCTG | Exon 54 |
| | AAAAGUCCAC | | GUAUCAUCUG | | GTATCATCTG | |
| 566 | CAAUGUCCUACAG | 1186 | GUAGCAUCCUGUA | 1806 | GTAGCATCCTGTA | Exon 55 |
| | GAUGCUAC | | GGACAUUG | | GGACATTG | |
| 567 | AAGGGAGUAAAAG | 1187 | UCAUCAGCUCUUU | 1807 | TCATCAGCTCTTT | Exon 55 |
| | AGCUGAUGA | | UACUCCCUU | | TACTCCCTT | |
| 568 | AAGGGAGUAAAAG | 1188 | UUCAUCAGCUCUU | 1808 | TTCATCAGCTCTT | Exon 55 |
| | AGCUGAUGAA | | UUACUCCCUU | | TTACTCCCTT | |
| 569 | AAGGGAGUAAAAG | 1189 | UUUCAUCAGCUCU | 1809 | TTTCATCAGCTCT | Exon 55 |
| | AGCUGAUGAAA | | UUUACUCCCUU | | TTTACTCCCTT | |
| 570 | AAGGGAGUAAAAG | 1190 | GUUUCAUCAGCUC | 1810 | GTTTCATCAGCTC | Exon 55 |
| | AGCUGAUGAAAC | | UUUUACUCCCUU | | TTTTACTCCCTT | |
| 571 | AAAGAGCUGAUGA | 1191 | UGCCAUUGUUUCA | 1811 | TGCCATTGTTTCA | Exon 55 |
| | AACAAUGGCA | | UCAGCUCUUU | | TCAGCTCTTT | |
| 572 | AAAGAGCUGAUGA | 1192 | UUGCCAUUGUUUC | 1812 | TTGCCATTGTTTC | Exon 55 |
| | AACAAUGGCAA | | AUCAGCUCUUU | | ATCAGCTCTTT | |
| 573 | AAAGAGCUGAUGA | 1193 | CUUGCCAUUGUUU | 1813 | CTTGCCATTGTTT | Exon 55/intron 55 |
| | AACAAUGGCAAG | | CAUCAGCUCUUU | | CATCAGCTCTTT | junction |
| 574 | AUGAAACAAUGGC | 1194 | UGACUUACUUGCC | 1814 | TGACTTACTTGCC | Exon 55/intron 55 |
| | AAGUAAGUCA | | AUUGUUUCAU | | ATTGTTTCAT | junction |
| 575 | CCAGGGACAAAAC | 1195 | GCAACUAUUUUGU | 1815 | GCAACTATTTTGT | Intron 55 |
| | AAAAUAGUUGC | | UUUGUCCCUGG | | TTTGTCCCTGG | |
| 576 | GCAAUUCUCCAAA | 1196 | GAAUGUGAAUUUG | 1816 | GAATGTGAATTTG | Intron 55 |
| | UUCACAUUC | | GAGAAUUGC | | GAGAATTGC | |
| 577 | CAAUUCUCCAAAU | 1197 | UGAAUGUGAAUUU | 1817 | TGAATGTGAATTT | Intron 55 |
| | UCACAUUCA | | GGAGAAUUG | | GGAGAATTG | |
| 578 | AUUCUCCAAAUUC | 1198 | GAUGAAUGUGAAU | 1818 | GATGAATGTGAAT | Intron 55 |
| | ACAUUCAUC | | UUGGAGAAU | | TTGGAGAAT | |
| 579 | GGUGAAAUUGAAG | 1199 | CAUCUGUGUGAGC | 1819 | CATCTGTGTGAGC | Exon 56 |
| | CUCACACAGAUG | | UUCAAUUUCACC | | TTCAATTTCACC | |
| 580 | CUCACACAGAUGU | 1200 | AGGUUGUGAUAAA | 1820 | AGGTTGTGATAAA | Exon 56 |
| | UUAUCACAACCU | | CAUCUGUGUGAG | | CATCTGTGTGAG | |
| 581 | ACAACCUGGAUGA | 1201 | GGCUGUUUUCAUC | 1821 | GGCTGTTTTCATC | Exon 56 |
| | AAACAGCC | | CAGGUUGU | | CAGGTTGT | |
| 582 | ACAACCUGGAUGA | 1202 | UGGCUGUUUUCAU | 1822 | TGGCTGTTTTCAT | Exon 56 |
| | AAACAGCCA | | CCAGGUUGU | | CCAGGTTGT | |
| 583 | ACAACCUGGAUGA | 1203 | UUGGCUGUUUUCA | 1823 | TTGGCTGTTTTCA | Exon 56 |
| | AAACAGCCAA | | UCCAGGUUGU | | TCCAGGTTGT | |
| 584 | CUGGAUGAAAACA | 1204 | GGAUUUUUUGGCU | 1824 | GGATTTTTTGGCT | Exon 56 |
| | GCCAAAAAAUCC | | GUUUUCAUCCAG | | GTTTTCATCCAG | |
| 585 | AGAUCCCUGGAAG | 1205 | CAUCAUCGGAACC | 1825 | CATCATCGGAACC | Exon 56 |
| | GUUCCGAUGAUG | | UUCCAGGGAUCU | | TTCCAGGGATCT | |
| 586 | GAUCCCUGGAAGG | 1206 | CAUCAUCGGAACC | 1826 | CATCATCGGAACC | Exon 56 |
| | UUCCGAUGAUG | | UUCCAGGGAUC | | TTCCAGGGATC | |
| 587 | AUCCCUGGAAGGU | 1207 | CAUCAUCGGAACC | 1827 | CATCATCGGAACC | Exon 56 |
| | UCCGAUGAUG | | UUCCAGGGAU | | TTCCAGGGAT | |
| 588 | UCCCUGGAAGGUU | 1208 | CAUCAUCGGAACC | 1828 | CATCATCGGAACC | Exon 56 |
| | CCGAUGAUG | | UUCCAGGGA | | TTCCAGGGA | |
| 589 | UCCCUGGAAGGUU | 1209 | CUGCAUCAUCGGA | 1829 | CTGCATCATCGGA | Exon 56 |
| | CCGAUGAUGCAG | | ACCUUCCAGGGA | | ACCTTCCAGGGA | |
| 590 | CCCUGGAAGGUUC | 1210 | CAUCAUCGGAACC | 1830 | CATCATCGGAACC | Exon 56 |
| | CGAUGAUG | | UUCCAGGG | | TTCCAGGG | |
| 591 | CCCUGGAAGGUUC | 1211 | CUGCAUCAUCGGA | 1831 | CTGCATCATCGGA | Exon 56 |
| | CGAUGAUGCAG | | ACCUUCCAGGG | | ACCTTCCAGGG | |
| 592 | CUGGAAGGUUCCG | 1212 | CUGCAUCAUCGGA | 1832 | CTGCATCATCGGA | Exon 56 |
| | AUGAUGCAG | | ACCUUCCAG | | ACCTTCCAG | |
| 593 | UGGAAGGUUCCGA | 1213 | CUGCAUCAUCGGA | 1833 | CTGCATCATCGGA | Exon 56 |
| | UGAUGCAG | | ACCUUCCA | | ACCTTCCA | |
| 594 | GAUGAUGCAGUCC | 1214 | GUCUUUGUAACAG | 1834 | GTCTTTGTAACAG | Exon 56 |
| | UGUUACAAAGAC | | GACUGCAUCAUC | | GACTGCATCATC | |
| 595 | GCUUACAGAAGCU | 1215 | GGCAGUUGUUUCA | 1835 | GGCAGTTGTTTCA | Exon 55 |
| | GAAACAACUGCC | | GCUUCUGUAAGC | | GCTTCTGTAAGC | |
| 596 | GGGAGUAAAAGAG | 1216 | UGUUUCAUCAGCU | 1836 | TGTTTCATCAGCT | Exon 55 |
| | CUGAUGAAACA | | CUUUUACUCCC | | CTTTTACTCCC | |
| 597 | GAGUAAAAGAGCU | 1217 | CAUUGUUUCAUCA | 1837 | CATTGTTTCATCA | Exon 55 |
| | GAUGAAACAAUG | | GCUCUUUUACUC | | GCTCTTTTACTC | |
| 598 | UAAAAGAGCUGAU | 1218 | GCCAUUGUUUCAU | 1838 | GCCATTGTTTCAT | Exon 55 |
| | GAAACAAUGGC | | CAGCUCUUUUA | | CAGCTCTTTTA | |
| 599 | GAUCCAAUUGAAC | 1219 | GCUGAGAAUUGUU | 1839 | GCTGAGAATTGTT | Intron 55 |
| | AAUUCUCAGC | | CAAUUGGAUC | | CAATTGGATC | |
| 600 | AAGGUUCCGAUGA | 1220 | CAGGACUGCAUCA | 1840 | CAGGACTGCATCA | Exon 56 |
| | UGCAGUCCUG | | UCGGAACCUU | | TCGGAACCTT | |
| 601 | CAAGGGAGUAAAA | 1221 | UCAGCUCUUUUAC | 1841 | TCAGCTCTTTTAC | Exon 55 |
| | GAGCUGA | | UCCCUUG | | TCCCTTG | |
| 602 | AGGGAGUAAAAGA | 1222 | UGUUUCAUCAGCU | 1842 | TGTTTCATCAGCT | Exon 55 |
| | GCUGAUGAAACA | | CUUUUACUCCCU | | CTTTTACTCCCT | |
| 603 | GCCAGGGACAAAA | 1223 | GCAACUAUUUUGU | 1843 | GCAACTATTTTGT | Intron 55 |
| | CAAAAUAGUUGC | | UUUGUCCCUGGC | | TTTGTCCCTGGC | |
| 604 | UGCAAUUCUCCAA | 1224 | GAAUGUGAAUUUG | 1844 | GAATGTGAATTTG | Intron 55 |
| | AUUCACAUUC | | GAGAAUUGCA | | GAGAATTGCA | |
| 605 | GCAAUUCUCCAAA | 1225 | UGAAUGUGAAUUU | 1843 | TGAATGTGAATTT | Intron 55 |
| | UUCACAUUCA | | GGAGAAUUGC | | GGAGAATTGC | |
| 606 | AAUUCUCCAAAUU | 1226 | GAUGAAUGUGAAU | 1846 | GATGAATGTGAAT | Intron 55 |
| | CACAUUCAUC | | UUGGAGAAUU | | TTGGAGAATT | |
| 607 | AUUCUCCAAAUUC | 1227 | CGAUGAAUGUGAA | 1847 | CGATGAATGTGAA | Intron 55 |
| | ACAUUCAUCG | | UUUGGAGAAU | | TTTGGAGAAT | |
| 608 | UUCUCCAAAUUCA | 1228 | CGAUGAAUGUGAA | 1848 | CGATGAATGTGAA | Intron 55 |
| | CAUUCAUCG | | UUUGGAGAA | | TTTGGAGAA | |
| 609 | UCUCCAAAUUCAC | 1229 | CGAUGAAUGUGAA | 1849 | CGATGAATGTGAA | Intron 55 |
| | AUUCAUCG | | UUUGGAGA | | TTTGGAGA | |
| 610 | GGUAAUUCUGCAC | 1230 | GAAGAAGAAUAUG | 1850 | GAAGAAGAATATG | Intron 55 |
| | AUAUUCUUCUUC | | UGCAGAAUUACC | | TGCAGAATTACC | |
| 611 | GCUCACACAGAUG | 1231 | GGUUGUGAUAAAC | 1851 | GGTTGTGATAAAC | Exon 56 |
| | UUUAUCACAACC | | AUCUGUGUGAGC | | ATCTGTGTGAGC | |
| 612 | UCACAACCUGGAU | 1232 | CUGUUUUCAUCCA | 1852 | CTGTTTTCATCCA | Exon 56 |
| | GAAAACAG | | GGUUGUGA | | GGTTGTGA | |
| 613 | CACAACCUGGAUG | 1233 | GGCUGUUUUCAUC | 1853 | GGCTGTTTTCATC | Exon 56 |
| | AAAACAGCC | | CAGGUUGUG | | CAGGTTGTG | |
| 614 | CACAACCUGGAUG | 1234 | UGGCUGUUUUCAU | 1854 | TGGCTGTTTTCAT | Exon 56 |
| | AAAACAGCCA | | CCAGGUUGUG | | CCAGGTTGTG | |
| 615 | CACAACCUGGAUG | 1235 | UUGGCUGUUUUCA | 1855 | TTGGCTGTTTTCA | Exon 56 |
| | AAAACAGCCAA | | UCCAGGUUGUG | | TCCAGGTTGTG | |
| 616 | ACAACCUGGAUGA | 1236 | UUUGGCUGUUUUC | 1856 | TTTGGCTGTTTTC | Exon 56 |
| | AAACAGCCAAA | | AUCCAGGUUGU | | ATCCAGGTTGT | |
| 617 | CCUGGAUGAAAAC | 1237 | GAUUUUUUGGCUG | 1857 | GATTTTTTGGCTG | Exon 56 |
| | AGCCAAAAAAUC | | UUUUCAUCCAGG | | TTTTCATCCAGG | |
| 618 | CCCUGGAAGGUUC | 1238 | ACUGCAUCAUCGG | 1858 | ACTGCATCATCGG | Exon 56 |
| | CGAUGAUGCAGU | | AACCUUCCAGGG | | AACCTTCCAGGG | |
| 619 | UGGAAGGUUCCGA | 1239 | ACUGCAUCAUCGG | 1859 | ACTGCATCATCGG | Exon 56 |
| | UGAUGCAGU | | AACCUUCCA | | AACCTTCCA | |
| 620 | UGGAAGGUUCCGA | 1240 | AGGACUGCAUCAU | 1860 | AGGACTGCATCAT | Exon 56 |
| | UGAUGCAGUCCU | | CGGAACCUUCCA | | CGGAACCTTCCA | |
| 621 | GGAAGGUUCCGAU | 1241 | AGGACUGCAUCAU | 1861 | AGGACTGCATCAT | Exon 56 |
| | GAUGCAGUCCU | | CGGAACCUUCC | | CGGAACCTTCC | |
| 622 | GGAAGGUUCCGAU | 1242 | CAGGACUGCAUCA | 1862 | CAGGACTGCATCA | Exon 56 |
| | GAUGCAGUCCUG | | UCGGAACCUUCC | | TCGGAACCTTCC | |
| 623 | GAAGGUUCCGAUG | 1243 | ACUGCAUCAUCGG | 1863 | ACTGCATCATCGG | Exon 56 |
| | AUGCAGU | | AACCUUC | | AACCTTC | |
| 624 | GUUCCGAUGAUGC | 1244 | UGUAACAGGACUG | 1864 | TGTAACAGGACTG | Exon 56 |
| | AGUCCUGUUACA | | CAUCAUCGGAAC | | CATCATCGGAAC | |
| 625 | GGGAGUAAAAGAG | 1245 | UUGUUUCAUCAGC | 1865 | TTGTTTCATCAGC | Exon 55 |
| | CUGAUGAAACAA | | UCUUUUACUCCC | | TCTTTTACTCCC | |
| 626 | GGAUCCAAUUGAA | 1246 | GCUGAGAAUUGUU | 1866 | GCTGAGAATTGTT | Intron 55 |
| | CAAUUCUCAGC | | CAAUUGGAUCC | | CAATTGGATCC | |
| 627 | GAAGGUUCCGAUG | 1247 | CAGGACUGCAUCA | 1867 | CAGGACTGCATCA | Exon 56 |
| | AUGCAGUCCUG | | UCGGAACCUUC | | TCGGAACCTTC | |
| 628 | AGGUUCCGAUGAU | 1248 | AACAGGACUGCAU | 1868 | AACAGGACTGCAT | Exon 56 |
| | GCAGUCCUGUU | | CAUCGGAACCU | | CATCGGAACCT | |
| 629 | GGUUCCGAUGAUG | 1249 | AACAGGACUGCAU | 1869 | AACAGGACTGCAT | Exon 56 |
| | CAGUCCUGUU | | CAUCGGAACC | | CATCGGAACC | |
| 630 | CAAGGGAGUAAAA | 1250 | AUCAGCUCUUUUA | 1870 | ATCAGCTCTTTTA | Exon 55 |
| | GAGCUGAU | | CUCCCUUG | | CTCCCTTG | |
| 631 | AGCACUCUUGUGG | 1251 | GUUCAAUUGGAUC | 1871 | GTTCAATTGGATC | Intron 55 |
| | AUCCAAUUGAAC | | CACAAGAGUGCU | | CACAAGAGTGCT | |
| 632 | GCCAGGGACAAAA | 1252 | CUAUUUUGUUUUG | 1872 | CTATTTTGTTTTG | Intron 55 |
| | CAAAAUAG | | UCCCUGGC | | TCCCTGGC | |
| 633 | UUGCAAUUCUCCA | 1253 | GAAUGUGAAUUUG | 1873 | GAATGTGAATTTG | Intron 55 |
| | AAUUCACAUUC | | GAGAAUUGCAA | | GAGAATTGCAA | |
| 634 | UGCAAUUCUCCAA | 1254 | UGAAUGUGAAUUU | 1874 | TGAATGTGAATTT | Intron 55 |
| | AUUCACAUUCA | | GGAGAAUUGCA | | GGAGAATTGCA | |
| 635 | GCAAUUCUCCAAA | 1255 | AUGAAUGUGAAUU | 1875 | ATGAATGTGAATT | Intron 55 |
| | UUCACAUUCAU | | UGGAGAAUUGC | | TGGAGAATTGC | |
| 636 | CAAUUCUCCAAAU | 1256 | GAUGAAUGUGAAU | 1876 | GATGAATGTGAAT | Intron 55 |
| | UCACAUUCAUC | | UUGGAGAAUUG | | TTGGAGAATTG | |
| 637 | AAUUCUCCAAAUU | 1257 | CGAUGAAUGUGAA | 1877 | CGATGAATGTGAA | Intron 55 |
| | CACAUUCAUCG | | UUUGGAGAAUU | | TTTGGAGAATT | |
| 638 | AUUCUCCAAAUUC | 1258 | GCGAUGAAUGUGA | 1878 | GCGATGAATGTGA | Intron 55 |
| | ACAUUCAUCGC | | AUUUGGAGAAU | | ATTTGGAGAAT | |
| 639 | UUCUCCAAAUUCA | 1259 | GCGAUGAAUGUGA | 1879 | GCGATGAATGTGA | Intron 55 |
| | CAUUCAUCGC | | AUUUGGAGAA | | ATTTGGAGAA | |
| 640 | UCUCCAAAUUCAC | 1260 | GCGAUGAAUGUGA | 1880 | GCGATGAATGTGA | Intron 55 |
| | AUUCAUCGC | | AUUUGGAGA | | ATTTGGAGA | |
| 641 | GUGAAAUUGAAGC | 1261 | ACAUCUGUGUGAG | 1881 | ACATCTGTGTGAG | Exon 56 |
| | UCACACAGAUGU | | CUUCAAUUUCAC | | CTTCAATTTCAC | |
| 642 | UAUCACAACCUGG | 1262 | CUGUUUUCAUCCA | 1882 | CTGTTTTCATCCA | Exon 56 |
| | AUGAAAACAG | | GGUUGUGAUA | | GGTTGTGATA | |
| 643 | AUCACAACCUGGA | 1263 | CUGUUUUCAUCCA | 1883 | CTGTTTTCATCCA | Exon 56 |
| | UGAAAACAG | | GGUUGUGAU | | GGTTGTGAT | |
| 644 | CCUGGAAGGUUCC | 1264 | GACUGCAUCAUCG | 1884 | GACTGCATCATCG | Exon 56 |
| | GAUGAUGCAGUC | | GAACCUUCCAGG | | GAACCTTCCAGG | |
| 645 | CUGGAAGGUUCCG | 1265 | GACUGCAUCAUCG | 1885 | GACTGCATCATCG | Exon 56 |
| | AUGAUGCAGUC | | GAACCUUCCAG | | GAACCTTCCAG | |
| 646 | UGGAAGGUUCCGA | 1266 | GACUGCAUCAUCG | 1886 | GACTGCATCATCG | Exon 56 |
| | UGAUGCAGUC | | GAACCUUCCA | | GAACCTTCCA | |
| 647 | GGAAGGUUCCGAU | 1267 | GACUGCAUCAUCG | 1887 | GACTGCATCATCG | Exon 56 |
| | GAUGCAGUC | | GAACCUUCC | | GAACCTTCC | |
| 648 | GGAGUAAAAGAGC | 1268 | AUUGUUUCAUCAG | 1888 | ATTGTTTCATCAG | Exon 55 |
| | UGAUGAAACAAU | | CUCUUUUACUCC | | CTCTTTTACTCC | |
| 649 | UGUGGAUCCAAUU | 1269 | GAAUUGUUCAAUU | 1889 | GAATTGTTCAATT | Intron 55 |
| | GAACAAUUC | | GGAUCCACA | | GGATCCACA | |
| 650 | AAGGUUCCGAUGA | 1270 | AACAGGACUGCAU | 1890 | AACAGGACTGCAT | Exon 56 |
| | UGCAGUCCUGUU | | CAUCGGAACCUU | | CATCGGAACCTT | |
| 651 | AUAAUGGGGUGGU | 1271 | CAGUUUCACCACC | 1891 | CAGTTTCACCACC | Intron 54 |
| | GAAACUG | | CCAUUAU | | CCATTAT | |
| 652 | GCACUCUUGUGGA | 1272 | UGUUCAAUUGGAU | 1892 | TGTTCAATTGGAT | Intron 55 |
| | UCCAAUUGAACA | | CCACAAGAGUGC | | CCACAAGAGTGC | |
| 653 | GGAUCCAAUUGAA | 1273 | CUGAGAAUUGUUC | 1893 | CTGAGAATTGTTC | Intron 55 |
| | CAAUUCUCAG | | AAUUGGAUCC | | AATTGGATCC | |
| 654 | GCCAGGGACAAAA | 1274 | ACUAUUUUGUUUU | 1894 | ACTATTTTGTTTT | Intron 55 |
| | CAAAAUAGU | | GUCCCUGGC | | GTCCCTGGC | |
| 655 | UUUGCAAUUCUCC | 1275 | GAAUGUGAAUUUG | 1895 | GAATGTGAATTTG | Intron 55 |
| | AAAUUCACAUUC | | GAGAAUUGCAAA | | GAGAATTGCAAA | |
| 656 | UUGCAAUUCUCCA | 1276 | UGAAUGUGAAUUU | 1896 | TGAATGTGAATTT | Intron 55 |
| | AAUUCACAUUCA | | GGAGAAUUGCAA | | GGAGAATTGCAA | |
| 657 | UGCAAUUCUCCAA | 1277 | AUGAAUGUGAAUU | 1897 | ATGAATGTGAATT | Intron 55 |
| | AUUCACAUUCAU | | UGGAGAAUUGCA | | TGGAGAATTGCA | |
| 658 | GCAAUUCUCCAAA | 1278 | GAUGAAUGUGAAU | 1898 | GATGAATGTGAAT | Intron 55 |
| | UUCACAUUCAUC | | UUGGAGAAUUGC | | TTGGAGAATTGC | |
| 659 | CAAUUCUCCAAAU | 1279 | CGAUGAAUGUGAA | 1899 | CGATGAATGTGAA | Intron 55 |
| | UCACAUUCAUCG | | UUUGGAGAAUUG | | TTTGGAGAATTG | |
| 660 | AAUUCUCCAAAUU | 1280 | GCGAUGAAUGUGA | 1900 | GCGATGAATGTGA | Intron 55 |
| | CACAUUCAUCGC | | AUUUGGAGAAUU | | ATTTGGAGAATT | |
| 661 | AUUCUCCAAAUUC | 1281 | AGCGAUGAAUGUG | 1901 | AGCGATGAATGTG | Intron 55 |
| | ACAUUCAUCGCU | | AAUUUGGAGAAU | | AATTTGGAGAAT | |
| 662 | UCUCCAAAUUCAC | 1282 | AGCGAUGAAUGUG | 1902 | AGCGATGAATGTG | Intron 55 |
| | AUUCAUCGCU | | AAUUUGGAGA | | AATTTGGAGA | |
| 663 | UCCAAAUUCACAU | 1283 | GCGAUGAAUGUGA | 1903 | GCGATGAATGTGA | Intron 55 |
| | UCAUCGC | | AUUUGGA | | ATTTGGA | |
| 664 | UUAUCACAACCUG | 1284 | CUGUUUUCAUCCA | 1904 | CTGTTTTCATCCA | Exon 56 |
| | GAUGAAAACAG | | GGUUGUGAUAA | | GGTTGTGATAA | |
| 665 | UAUCACAACCUGG | 1285 | GCUGUUUUCAUCC | 1905 | GCTGTTTTCATCC | Exon 56 |
| | AUGAAAACAGC | | AGGUUGUGAUA | | AGGTTGTGATA | |
| 666 | UGUGGAUCCAAUU | 1286 | AGAAUUGUUCAAU | 1906 | AGAATTGTTCAAT | Intron 55 |
| | GAACAAUUCU | | UGGAUCCACA | | TGGATCCACA | |
| 667 | GUGGAUCCAAUUG | 1287 | GAAUUGUUCAAUU | 1907 | GAATTGTTCAATT | Intron 55 |
| | AACAAUUC | | GGAUCCAC | | GGATCCAC | |
| 668 | UCCAAUUGAACAA | 1288 | AUGCUGAGAAUUG | 1908 | ATGCTGAGAATTG | Intron 55 |
| | UUCUCAGCAU | | UUCAAUUGGA | | TTCAATTGGA | |
| 669 | CCAGGGACAAAAC | 1289 | ACUAUUUUGUUUU | 1909 | ACTATTTTGTTTT | Intron 55 |
| | AAAAUAGU | | GUCCCUGG | | GTCCCTGG | |
| 670 | AAUAAUGGGGUGG | 1290 | CAGUUUCACCACC | 1910 | CAGTTTCACCACC | Intron 54 |
| | UGAAACUG | | CCAUUAUU | | CCATTATT | |
| 671 | UGGAUCCAAUUGA | 1291 | CUGAGAAUUGUUC | 1911 | CTGAGAATTGTTC | Intron 55 |
| | ACAAUUCUCAG | | AAUUGGAUCCA | | AATTGGATCCA | |
| 672 | AGCCAGGGACAAA | 1292 | ACUAUUUUGUUUU | 1912 | ACTATTTTGTTTT | Intron 55 |
| | ACAAAAUAGU | | GUCCCUGGCU | | GTCCCTGGCT | |
| 673 | GCCAGGGACAAAA | 1293 | AACUAUUUUGUUU | 1913 | AACTATTTTGTTT | Intron 55 |
| | CAAAAUAGUU | | UGUCCCUGGC | | TGTCCCTGGC | |
| 674 | UUCUCCAAAUUCA | 1294 | AGCGAUGAAUGUG | 1914 | AGCGATGAATGTG | Intron 55 |
| | CAUUCAUCGCU | | AAUUUGGAGAA | | AATTTGGAGAA | |
| 675 | UCUCCAAAUUCAC | 1295 | AAGCGAUGAAUGU | 1915 | AAGCGATGAATGT | Intron 55 |
| | AUUCAUCGCUU | | GAAUUUGGAGA | | GAATTTGGAGA | |
| 676 | CUCCAAAUUCACA | 1296 | GCGAUGAAUGUGA | 1916 | GCGATGAATGTGA | Intron 55 |
| | UUCAUCGC | | AUUUGGAG | | ATTTGGAG | |
| 677 | UCCAAAUUCACAU | 1297 | AGCGAUGAAUGUG | 1917 | AGCGATGAATGTG | Intron 55 |
| | UCAUCGCU | | AAUUUGGA | | AATTTGGA | |
| 678 | GUAAUUCUGCACA | 1298 | GGAAGAAGAAUAU | 1918 | GGAAGAAGAATAT | Intron 55 |
| | UAUUCUUCUUCC | | GUGCAGAAUUAC | | GTGCAGAATTAC | |
| 679 | UUUAUCACAACCU | 1299 | CUGUUUUCAUCCA | 1919 | CTGTTTTCATCCA | Exon 56 |
| | GGAUGAAAACAG | | GGUUGUGAUAAA | | GGTTGTGATAAA | |
| 680 | GUGGAUCCAAUUG | 1300 | AGAAUUGUUCAAU | 1920 | AGAATTGTTCAAT | Intron 55 |
| | AACAAUUCU | | UGGAUCCAC | | TGGATCCAC | |
| 681 | UCCAAUUGAACAA | 1301 | AAUGCUGAGAAUU | 1921 | AATGCTGAGAATT | Intron 55 |
| | UUCUCAGCAUU | | GUUCAAUUGGA | | GTTCAATTGGA | |
| 682 | CCAGGGACAAAAC | 1302 | AACUAUUUUGUUU | 1922 | AACTATTTTGTTT | Intron 55 |
| | AAAAUAGUU | | UGUCCCUGG | | TGTCCCTGG | |
| 683 | UCCAAAUUCACAU | 1303 | AAGCGAUGAAUGU | 1923 | AAGCGATGAATGT | Intron 55 |
| | UCAUCGCUU | | GAAUUUGGA | | GAATTTGGA | |
| 684 | CCAAAUUCACAUU | 1304 | CAAGCGAUGAAUG | 1924 | CAAGCGATGAATG | Intron 55 |
| | CAUCGCUUG | | UGAAUUUGG | | TGAATTTGG | |
| 685 | UGGUAAUUCUGCA | 1305 | GAAGAAUAUGUGC | 1925 | GAAGAATATGTGC | Intron 55 |
| | CAUAUUCUUC | | AGAAUUACCA | | AGAATTACCA | |
| 686 | GUGGAUCCAAUUG | 1306 | CUGAGAAUUGUUC | 1926 | CTGAGAATTGTTC | Intron 55 |
| | AACAAUUCUCAG | | AAUUGGAUCCAC | | AATTGGATCCAC | |
| 687 | UGGAUCCAAUUGA | 1307 | GCUGAGAAUUGUU | 1927 | GCTGAGAATTGTT | Intron 55 |
| | ACAAUUCUCAGC | | CAAUUGGAUCCA | | CAATTGGATCCA | |
| 688 | GGAUCCAAUUGAA | 1308 | GAGAAUUGUUCAA | 1928 | GAGAATTGTTCAA | Intron 55 |
| | CAAUUCUC | | UUGGAUCC | | TTGGATCC | |
| 689 | CAAGCCAGGGACA | 1309 | CUAUUUUGUUUUG | 1929 | CTATTTTGTTTTG | Intron 55 |
| | AAACAAAAUAG | | UCCCUGGCUUG | | TCCCTGGCTTG | |
| 690 | AAGCCAGGGACAA | 1310 | ACUAUUUUGUUUU | 1930 | ACTATTTTGTTTT | Intron 55 |
| | AACAAAAUAGU | | GUCCCUGGCUU | | GTCCCTGGCTT | |
| 691 | AGCCAGGGACAAA | 1311 | AACUAUUUUGUUU | 1931 | AACTATTTTGTTT | Intron 55 |
| | ACAAAAUAGUU | | UGUCCCUGGCU | | TGTCCCTGGCT | |
| 692 | GCCAGGGACAAAA | 1312 | CAACUAUUUUGUU | 1932 | CAACTATTTTGTT | Intron 55 |
| | CAAAAUAGUUG | | UUGUCCCUGGC | | TTGTCCCTGGC | |
| 693 | UUCUCCAAAUUCA | 1313 | AAGCGAUGAAUGU | 1933 | AAGCGATGAATGT | Intron 55 |
| | CAUUCAUCGCUU | | GAAUUUGGAGAA | | GAATTTGGAGAA | |
| 694 | UCUCCAAAUUCAC | 1314 | CAAGCGAUGAAUG | 1934 | CAAGCGATGAATG | Intron 55 |
| | AUUCAUCGCUUG | | UGAAUUUGGAGA | | TGAATTTGGAGA | |
| 695 | CUCCAAAUUCACA | 1315 | AGCGAUGAAUGUG | 1935 | AGCGATGAATGTG | Intron 55 |
| | UUCAUCGCU | | AAUUUGGAG | | AATTTGGAG | |
| 696 | GUUCCGAUGAUGC | 1316 | CAGGACUGCAUCA | 1936 | CAGGACTGCATCA | Exon 56 |
| | AGUCCUG | | UCGGAAC | | TCGGAAC | |
| 697 | UUCCGAUGAUGCA | 1317 | ACAGGACUGCAUC | 1937 | ACAGGACTGCATC | Exon 56 |
| | GUCCUGU | | AUCGGAA | | ATCGGAA | |
| 698 | UCCGAUGAUGCAG | 1318 | AACAGGACUGCAU | 1938 | AACAGGACTGCAT | Exon 56 |
| | UCCUGUU | | CAUCGGA | | CATCGGA | |
| 699 | UCCGAUGAUGCAG | 1319 | UUGUAACAGGACU | 1939 | TTGTAACAGGACT | Exon 56 |
| | UCCUGUUACAA | | GCAUCAUCGGA | | GCATCATCGGA | |
| 700 | UCCGAUGAUGCAG | 1320 | UUUGUAACAGGAC | 1940 | TTTGTAACAGGAC | Exon 56 |
| | UCCUGUUACAAA | | UGCAUCAUCGGA | | TGCATCATCGGA | |
| 701 | UCCAAUUGAACAA | 1321 | AAAUGCUGAGAAU | 1941 | AAATGCTGAGAAT | Intron 55 |
| | UUCUCAGCAUUU | | UGUUCAAUUGGA | | TGTTCAATTGGA | |
| 702 | CCAGGGACAAAAC | 1322 | CAACUAUUUUGUU | 1942 | CAACTATTTTGTT | Intron 55 |
| | AAAAUAGUUG | | UUGUCCCUGG | | TTGTCCCTGG | |
| 703 | CUCCAAAUUCACA | 1323 | AAGCGAUGAAUGU | 1943 | AAGCGATGAATGT | Intron 55 |
| | UUCAUCGCUU | | GAAUUUGGAG | | GAATTTGGAG | |
| 704 | UCCAAAUUCACAU | 1324 | CAAGCGAUGAAUG | 1944 | CAAGCGATGAATG | Intron 55 |
| | UCAUCGCUUG | | UGAAUUUGGA | | TGAATTTGGA | |
| 705 | UUGGUAAUUCUGC | 1325 | GAAGAAUAUGUGC | 1945 | GAAGAATATGTGC | Intron 55 |
| | ACAUAUUCUUC | | AGAAUUACCAA | | AGAATTACCAA | |
| 706 | UGGUAAUUCUGCA | 1326 | AGAAGAAUAUGUG | 1946 | AGAAGAATATGTG | Intron 55 |
| | CAUAUUCUUCU | | CAGAAUUACCA | | CAGAATTACCA | |
| 707 | UAAUAAUGGGGUG | 1327 | GUUUCACCACCCC | 1947 | GTTTCACCACCCC | Intron 54 |
| | GUGAAAC | | AUUAUUA | | ATTATTA | |
| 708 | UGGAUCCAAUUGA | 1328 | GAGAAUUGUUCAA | 1948 | GAGAATTGTTCAA | Intron 55 |
| | ACAAUUCUC | | UUGGAUCCA | | TTGGATCCA | |
| 709 | CAAGCCAGGGACA | 1329 | ACUAUUUUGUUUU | 1949 | ACTATTTTGTTTT | Intron 55 |
| | AAACAAAAUAGU | | GUCCCUGGCUUG | | GTCCCTGGCTTG | |
| 710 | AGCCAGGGACAAA | 1330 | CAACUAUUUUGUU | 1950 | CAACTATTTTGTT | Intron 55 |
| | ACAAAAUAGUUG | | UUGUCCCUGGCU | | TTGTCCCTGGCT | |
| 711 | UUCCGAUGAUGCA | 1331 | AACAGGACUGCAU | 1951 | AACAGGACTGCAT | Exon 56 |
| | GUCCUGUU | | CAUCGGAA | | CATCGGAA | |
| 712 | UUUGGUAAUUCUG | 1332 | GAAGAAUAUGUGC | 1952 | GAAGAATATGTGC | Intron 55 |
| | CACAUAUUCUUC | | AGAAUUACCAAA | | AGAATTACCAAA | |
| 713 | UUGGUAAUUCUGC | 1333 | AGAAGAAUAUGUG | 1953 | AGAAGAATATGTG | Intron 55 |
| | ACAUAUUCUUCU | | CAGAAUUACCAA | | CAGAATTACCAA | |
| 714 | UGGUAAUUCUGCA | 1334 | AAGAAGAAUAUGU | 1954 | AAGAAGAATATGT | Intron 55 |
| | CAUAUUCUUCUU | | GCAGAAUUACCA | | GCAGAATTACCA | |
| 715 | UGGAUCCAAUUGA | 1335 | UGAGAAUUGUUCA | 1955 | TGAGAATTGTTCA | Intron 55 |
| | ACAAUUCUCA | | AUUGGAUCCA | | ATTGGATCCA | |
| 716 | GUUCCGAUGAUGC | 1336 | AACAGGACUGCAU | 1956 | AACAGGACTGCAT | Exon 56 |
| | AGUCCUGUU | | CAUCGGAAC | | CATCGGAAC | |
| 717 | UUCCGAUGAUGCA | 1337 | UAACAGGACUGCA | 1957 | TAACAGGACTGCA | Exon 56 |
| | GUCCUGUUA | | UCAUCGGAA | | TCATCGGAA | |
| 718 | GGUAAUUCUGCAC | 1338 | GAAGAAUAUGUGC | 1958 | GAAGAATATGTGC | Intron 55 |
| | AUAUUCUUC | | AGAAUUACC | | AGAATTACC | |
| 719 | UCCGAUGAUGCAG | 1339 | UGUAACAGGACUG | 1959 | TGTAACAGGACTG | Exon 56 |
| | UCCUGUUACA | | CAUCAUCGGA | | CATCATCGGA | |
| 720 | GGUAAUUCUGCAC | 1340 | AGAAGAAUAUGUG | 1960 | AGAAGAATATGTG | Intron 55 |
| | AUAUUCUUCU | | CAGAAUUACC | | CAGAATTACC | |
| 721 | UUCCGAUGAUGCA | 1341 | UGUAACAGGACUG | 1961 | TGTAACAGGACTG | Exon 56 |
| | GUCCUGUUACA | | CAUCAUCGGAA | | CATCATCGGAA | |
| 722 | GUAAUUCUGCACA | 1342 | GAAGAAGAAUAUG | 1962 | GAAGAAGAATATG | Intron 55 |
| | UAUUCUUCUUC | | UGCAGAAUUAC | | TGCAGAATTAC | |
| 723 | AUUGAACAAUUCU | 1343 | GUACAAAUGCUGA | 1963 | GTACAAATGCTGA | Intron 55 |
| | CAGCAUUUGUAC | | GAAUUGUUCAAU | | GAATTGTTCAAT | |
| 724 | AUCACAACCUGGA | 1344 | GCUGUUUUCAUCC | 1964 | GCTGTTTTCATCC | Exon 56 |
| | UGAAAACAGC | | AGGUUGUGAU | | AGGTTGTGAT | |
| 725 | UCACAACCUGGAU | 1345 | GCUGUUUUCAUCC | 1965 | GCTGTTTTCATCC | Exon 56 |
| | GAAAACAGC | | AGGUUGUGA | | AGGTTGTGA | |
| 726 | CACAACCUGGAUG | 1346 | GCUGUUUUCAUCC | 196 | GCTGTTTTCATCC | Exon 56 |
| | AAAACAGC | | AGGUUGUG | | AGGTTGTG | |
| 727 | GAAGGUUCCGAUG | 1347 | GACUGCAUCAUCG | 1967 | GACTGCATCATCG | Exon 56 |
| | AUGCAGUC | | GAACCUUC | | GAACCTTC | |
| 728 | GAAGGUUCCGAUG | 1348 | AGGACUGCAUCAU | 1968 | AGGACTGCATCAT | Exon 56 |
| | AUGCAGUCCU | | CGGAACCUUC | | CGGAACCTTC | |
| 729 | AAGGUUCCGAUGA | 1349 | GACUGCAUCAUCG | 1969 | GACTGCATCATCG | Exon 56 |
| | UGCAGUC | | GAACCUU | | GAACCTT | |
| 730 | AAGGUUCCGAUGA | 1350 | AGGACUGCAUCAU | 1970 | AGGACTGCATCAT | Exon 56 |
| | UGCAGUCCU | | CGGAACCUU | | CGGAACCTT | |
| 731 | GGAGCUUGGGAGG | 1351 | CGUCUUGAACCCU | 1971 | CGTCTTGAACCCT | Intron 54 |
| | GUUCAAGACG | | CCCAAGCUCC | | CCCAAGCTCC | |
| 732 | CCUGAGAUCCCUG | 1352 | GGAACCUUCCAGG | 1972 | GGAACCTTCCAGG | Exon 56 |
| | GAAGGUUCC | | GAUCUCAGG | | GATCTCAGG | |
| 733 | UGGCUGUAAUAAU | 1353 | ACCACCCCAUUAU | 1973 | ACCACCCCATTAT | Intron 54 |
| | GGGGUGGU | | UACAGCCA | | TACAGCCA | |
| 734 | GGCUGUAAUAAUG | 1354 | ACCACCCCAUUAU | 1974 | ACCACCCCATTAT | Intron 54 |
| | GGGUGGU | | UACAGCC | | TACAGCC | |
| 735 | GGCUGUAAUAAUG | 1355 | CACCACCCCAUUA | 1975 | CACCACCCCATTA | Intron 54 |
| | GGGUGGUG | | UUACAGCC | | TTACAGCC | |
| 736 | UUGGCUGUAAUAA | 1356 | ACCACCCCAUUAU | 1976 | ACCACCCCATTAT | Intron 54 |
| | UGGGGUGGU | | UACAGCCAA | | TACAGCCAA | |
| 737 | UUGGCUGUAAUAA | 1357 | CACCACCCCAUUA | 1977 | CACCACCCCATTA | Intron 54 |
| | UGGGGUGGUG | | UUACAGCCAA | | TTACAGCCAA | |
| 738 | AUGGCAAGUAAGU | 1358 | AUGCCUGACUUAC | 1978 | ATGCCTGACTTAC | Exon 55/intron 55 |
| | CAGGCAU | | UUGCCAU | | TTGCCAT | junction |
| 739 | UGGCUGUAAUAAU | 1359 | UUCACCACCCCAU | 1979 | TTCACCACCCCAT | Intron 54 |
| | GGGGUGGUGAA | | UAUUACAGCCA | | TATTACAGCCA | |
| 740 | GGCUGUAAUAAUG | 1360 | UUCACCACCCCAU | 1980 | TTCACCACCCCAT | Intron 54 |
| | GGGUGGUGAA | | UAUUACAGCC | | TATTACAGCC | |
| 741 | GCUGUAAUAAUGG | 1361 | CACCACCCCAUUA | 1981 | CACCACCCCATTA | Intron 54 |
| | GGUGGUG | | UUACAGC | | TTACAGC | |
| 742 | GGAGCUUGGGAGG | 1362 | GUCUUGAACCCUC | 1982 | GTCTTGAACCCTC | Intron 54 |
| | GUUCAAGAC | | CCAAGCUCC | | CCAAGCTCC | |
| 743 | AUGGAGUUCACUA | 1363 | GUGCACCUAGUGA | 1983 | GTGCACCTAGTGA | Intron 54 |
| | GGUGCAC | | ACUCCAU | | ACTCCAT | |
| 744 | AAUGGCAAGUAAG | 1364 | AUGCCUGACUUAC | 1984 | ATGCCTGACTTAC | Exon 55/intron 55 |
| | UCAGGCAU | | UUGCCAUU | | TTGCCATT | junction |
| 745 | AUGGCAAGUAAGU | 1365 | AAUGCCUGACUUA | 1985 | AATGCCTGACTTA | Exon 55/intron 55 |
| | CAGGCAUU | | CUUGCCAU | | CTTGCCAT | junction |
| 746 | UUGGCUGUAAUAA | 1366 | UUCACCACCCCAU | 1986 | TTCACCACCCCAT | Intron 54 |
| | UGGGGUGGUGAA | | UAUUACAGCCAA | | TATTACAGCCAA | |
| 747 | UAAUGGGGUGGUG | 1367 | CCAGUUUCACCAC | 1987 | CCAGTTTCACCAC | Intron 54 |
| | AAACUGG | | CCCAUUA | | CCCATTA | |
| 748 | UGGCAAGUAAGUC | 1368 | CGGAAAUGCCUGA | 1988 | CGGAAATGCCTGA | Exon 55/intron 55 |
| | AGGCAUUUCCG | | CUUACUUGCCA | | CTTACTTGCCA | junction |
| 749 | GCUGUAAUAAUGG | 1369 | UUCACCACCCCAU | 1989 | TTCACCACCCCAT | Intron 54 |
| | GGUGGUGAA | | UAUUACAGC | | TATTACAGC | |
| 750 | AAUGGCAAGUAAG | 1370 | AAUGCCUGACUUA | 1990 | AATGCCTGACTTA | Exon 55/intron 55 |
| | UCAGGCAUU | | CUUGCCAUU | | CTTGCCATT | junction |
| 751 | AUGGCAAGUAAGU | 1371 | AAAUGCCUGACUU | 1991 | AAATGCCTGACTT | Exon 55/intron 55 |
| | CAGGCAUUU | | ACUUGCCAU | | ACTTGCCAT | junction |
| 752 | GCAAGUAAGUCAG | 1372 | AGCGGAAAUGCCU | 1992 | AGCGGAAATGCCT | Exon 55/intron 55 |
| | GCAUUUCCGCU | | GACUUACUUGC | | GACTTACTTGC | junction |
| 753 | AUAAUGGGGUGGU | 1373 | CCAGUUUCACCAC | 1993 | CCAGTTTCACCAC | Intron 54 |
| | GAAACUGG | | CCCAUUAU | | CCCATTAT | |
| 754 | AAUGGCAAGUAAG | 1374 | UGCCUGACUUACU | 1994 | TGCCTGACTTACT | Exon 55/intron 55 |
| | UCAGGCA | | UGCCAUU | | TGCCATT | junction |
| 755 | AUGGCAAGUAAGU | 1375 | CGGAAAUGCCUGA | 1995 | CGGAAATGCCTGA | Exon 55/intron 55 |
| | CAGGCAUUUCCG | | CUUACUUGCCAU | | CTTACTTGCCAT | junction |
| 756 | CGAUGAUGCAGUC | 1376 | UCUUUGUAACAGG | 1996 | TCTTTGTAACAGG | Exon 56 |
| | CUGUUACAAAGA | | ACUGCAUCAUCG | | ACTGCATCATCG | |
| 757 | AAUGGCAAGUAAG | 1377 | AAAUGCCUGACUU | 1997 | AAATGCCTGACTT | Exon 55/intron 55 |
| | UCAGGCAUUU | | ACUUGCCAUU | | ACTTGCCATT | junction |
| 758 | GCAAGUAAGUCAG | 1378 | GGAAAUGCCUGAC | 1998 | GGAAATGCCTGAC | Exon 55/intron 55 |
| | GCAUUUCC | | UUACUUGC | | TTACTTGC | junction |
| 759 | GCAAGUAAGUCAG | 1379 | AAGCGGAAAUGCC | 1999 | AAGCGGAAATGCC | Exon 55/intron 55 |
| | GCAUUUCCGCUU | | UGACUUACUUGC | | TGACTTACTTGC | junction |
| 760 | CAAGUAAGUCAGG | 1380 | AGCGGAAAUGCCU | 2000 | AGCGGAAATGCCT | Exon 55/intron 55 |
| | CAUUUCCGCU | | GACUUACUUG | | GACTTACTTG | junction |
| 761 | AAUAAUGGGGUGG | 1381 | CCAGUUUCACCAC | 2001 | CCAGTTTCACCAC | Intron 54 |
| | UGAAACUGG | | CCCAUUAUU | | CCCATTATT | |
| 762 | GGCAAGUAAGUCA | 1382 | GAAAUGCCUGACU | 2002 | GAAATGCCTGACT | Exon 55/intron 55 |
| | GGCAUUUC | | UACUUGCC | | TACTTGCC | junction |
| 763 | CCGAUGAUGCAGU | 1383 | CUUUGUAACAGGA | 2003 | CTTTGTAACAGGA | Exon 56 |
| | CCUGUUACAAAG | | CUGCAUCAUCGG | | CTGCATCATCGG | |
| 764 | GCAAGUAAGUCAG | 1384 | CGGAAAUGCCUGA | 2004 | CGGAAATGCCTGA | Exon 55/intron 55 |
| | GCAUUUCCG | | CUUACUUGC | | CTTACTTGC | junction |
| 765 | CAAGUAAGUCAGG | 1385 | GCGGAAAUGCCUG | 2005 | GCGGAAATGCCTG | Exon 55/intron 55 |
| | CAUUUCCGC | | ACUUACUUG | | ACTTACTTG | junction |
| 766 | GUUUGGUAAUUCU | 1386 | GAAUAUGUGCAGA | 2006 | GAATATGTGCAGA | Intron 55 |
| | GCACAUAUUC | | AUUACCAAAC | | ATTACCAAAC | |
| 767 | UAAUAAUGGGGUG | 1387 | CCAGUUUCACCAC | 2007 | CCAGTTTCACCAC | Intron 54 |
| | GUGAAACUGG | | CCCAUUAUUA | | CCCATTATTA | |
| 768 | UGGCAAGUAAGUC | 1388 | GAAAUGCCUGACU | 2008 | GAAATGCCTGACT | Exon 55/intron 55 |
| | AGGCAUUUC | | UACUUGCCA | | TACTTGCCA | junction |
| 769 | GGCAAGUAAGUCA | 1389 | GGAAAUGCCUGAC | 2009 | GGAAATGCCTGAC | Exon 55/intron 55 |
| | GGCAUUUCC | | UUACUUGCC | | TTACTTGCC | junction |
| 770 | CCGAUGAUGCAGU | 1390 | UAACAGGACUGCA | 2010 | TAACAGGACTGCA | Exon 56 |
| | CCUGUUA | | UCAUCGG | | TCATCGG | |
| 771 | CGAUGAUGCAGUC | 1391 | GUAACAGGACUGC | 2011 | GTAACAGGACTGC | Exon 56 |
| | CUGUUAC | | AUCAUCG | | ATCATCG | |
| 772 | CCAAAUUCACAUU | 1392 | ACAAGCGAUGAAU | 2012 | ACAAGCGATGAAT | Intron 55 |
| | CAUCGCUUGU | | GUGAAUUUGG | | GTGAATTTGG | |
| 773 | GUUUGGUAAUUCU | 1393 | AGAAUAUGUGCAG | 2013 | AGAATATGTGCAG | Intron 55 |
| | GCACAUAUUCU | | AAUUACCAAAC | | AATTACCAAAC | |
| 774 | GUAAUAAUGGGGU | 1394 | UUUCACCACCCCA | 2014 | TTTCACCACCCCA | Intron 54 |
| | GGUGAAA | | UUAUUAC | | TTATTAC | |
| 775 | GUAAUAAUGGGGU | 1395 | CAGUUUCACCACC | 2015 | CAGTTTCACCACC | Intron 54 |
| | GGUGAAACUG | | CCAUUAUUAC | | CCATTATTAC | |
| 776 | GGCAAGUAAGUCA | 1396 | CGGAAAUGCCUGA | 2016 | CGGAAATGCCTGA | Exon 55/intron 55 |
| | GGCAUUUCCG | | CUUACUUGCC | | CTTACTTGCC | junction |
| 777 | CCGAUGAUGCAGU | 1397 | UGUAACAGGACUG | 2017 | TGTAACAGGACTG | Exon 56 |
| | CCUGUUACA | | CAUCAUCGG | | CATCATCGG | |
| 778 | GUAAUAAUGGGGU | 1398 | AGUUUCACCACCC | 2018 | AGTTTCACCACCC | Intron 54 |
| | GGUGAAACU | | CAUUAUUAC | | CATTATTAC | |
| 779 | CUCCAAAUUCACA | 1399 | ACAAGCGAUGAAU | 2019 | ACAAGCGATGAAT | Intron 55 |
| | UUCAUCGCUUGU | | GUGAAUUUGGAG | | GTGAATTTGGAG | |

†Each thymine base (T) in any one of the oligonucleotides and/or target sequences provided in Table 8 may independently and optionally be replaced with a uracil base (U), and/or each U may independently and optionally be replaced with a T. Target sequences listed in Table 8 contain U's, but binding of a DMD-targeting oligonucleotide to RNA and/or DNA is contemplated.

In some embodiments, an oligonucleotide useful for targeting DMD (e.g., for exon skipping) targets a region of a DMD sequence. In some embodiments, an oligonucleotide useful for targeting DMD (e.g., for exon skipping) targets a region of a DMD RNA (e.g., the Dp427m transcript of SEQ ID NO: 130). In some embodiments, an oligonucleotide useful for targeting DMD (e.g., for exon skipping) comprises a region of complementarity to a DMD RNA (e.g., the Dp427m transcript of SEQ ID NO: 130). In some embodiments, an oligonucleotide useful for targeting DMD (e.g., for exon skipping) comprises a region of complementarity to an exon of a DMD RNA (e.g., SEQ ID NO: 2142, 2152, or 2165). In some embodiments, an oligonucleotide useful for targeting DMD (e.g., for exon skipping) comprises a region of complementarity to an intron of a DMD RNA (e.g., SEQ ID NO: 2145 or 2157). In some embodiments, an oligonucleotide useful for targeting DMD (e.g., for exon skipping) comprises a region of complementarity to a portion of a DMD sequence (e.g., a sequence provided by any one of SEQ ID NOs: 2143, 2144, 2146-2151, 2153-2156, 2158-2164, and 2166-2169). Examples of DMD sequences are provided below. Each of the DMD sequences provided below include thymine nucleotides (T's), but it should be understood that each sequence can represent a DNA sequence or an RNA sequence in which any or all of the T's would be replaced with uracil nucleotides (U's).

Homo sapiens dystrophin (DMD), transcript variant Dp427m, mRNA (NCBI
Reference Sequence: NM_004006.2)
(SEQ ID NO: 130)
TCCTGGCATCAGTTACTGTGTTGACTCACTCAGTGTTGGGATCACTCACTTTCCCCCTACAGGACTCAGATCTGGGA
GGCAATTACCTTCGGAGAAAAACGAATAGGAAAAACTGAAGTGTTACTTTTTTTAAAGCTGCTGAAGTTTGTTGGTT
TCTCATTGTTTTTAAGCCTACTGGAGCAATAAAGTTTGAAGAACTTTTACCAGGTTTTTTTTATCGCTGCCTTGATA
TACACTTTTCAAAATGCTTTGGTGGGAAGAAGTAGAGGACTGTTATGAAAGAGAAGATGTTCAAAAGAAAACATTCA
CAAAATGGGTAAATGCACAATTTTCTAAGTTTGGGAAGCAGCATATTGAGAACCTCTTCAGTGACCTACAGGATGGG
AGGCGCCTCCTAGACCTCCTCGAAGGCCTGACAGGGCAAAAACTGCCAAAAGAAAAAGGATCCACAAGAGTTCATGC
CCTGAACAATGTCAACAAGGCACTGCGGGTTTTGCAGAACAATAATGTTGATTTAGTGAATATTGGAAGTACTGACA
TCGTAGATGGAAATCATAAACTGACTCTTGGTTTGATTTGGAATATAATCCTCCACTGGCAGGTCAAAAATGTAATG
AAAAATATCATGGCTGGATTGCAACAAACCAACAGTGAAAAGATTCTCCTGAGCTGGGTCCGACAATCAACTCGTAA
TTATCCACAGGTTAATGTAATCAACTTCACCACCAGCTGGTCTGATGGCCTGGCTTTGAATGCTCTCATCCATAGTC
ATAGGCCAGACCTATTTGACTGGAATAGTGTGGTTTGCCAGCAGTCAGCCACACAACGACTGGAACATGCATTCAAC
ATCGCCAGATATCAATTAGGCATAGAGAAACTACTCGATCCTGAAGATGTTGATACCACCTATCCAGATAAGAAGTC
CATCTTAATGTACATCACATCACTCTTCCAAGTTTTGCCTCAACAAGTGAGCATTGAAGCCATCCAGGAAGTGGAAA
TGTTGCCAAGGCCACCTAAAGTGACTAAAGAAGAACATTTTCAGTTACATCATCAAATGCACTATTCTCAACAGATC
ACGGTCAGTCTAGCACAGGGATATGAGAGAACTTCTTCCCCTAAGCCTCGATTCAAGAGCTATGCCTACACACAGGC
TGCTTATGTCACCACCTCTGACCCTACACGGAGCCCATTTCCTTCACAGCATTTGGAAGCTCCTGAAGACAAGTCAT
TTGGCAGTTCATTGATGGAGAGTGAAGTAAACCTGGACCGTTATCAAACAGCTTTAGAAGAAGTATTATCGTGGCTT
CTTTCTGCTGAGGACACATTGCAAGCACAAGGAGAGATTTCTAATGATGTGGAAGTGGTGAAAGACCAGTTTCATAC
TCATGAGGGGTACATGATGGATTTGACAGCCCATCAGGGCCGGGTTGGTAATATTCTACAATTGGGAAGTAAGCTGA
TTGGAACAGGAAAATTATCAGAAGATGAAGAAACTGAAGTACAAGAGCAGATGAATCTCCTAAATTCAAGATGGGAA
TGCCTCAGGGTAGCTAGCATGGAAAAACAAAGCAATTTACATAGAGTTTTAATGGATCTCCAGAATCAGAAACTGAA
AGAGTTGAATGACTGGCTAACAAAAACAGAAGAAAGAACAAGGAAAATGGAGGAAGAGCCTCTTGGACCTGATCTTG
AAGACCTAAAACGCCAAGTACAACAACATAAGGTGCTTCAAGAAGATCTAGAACAAGAACAAGTCAGGGTCAATTCT
CTCACTCACATGGTGGTGGTAGTTGATGAATCTAGTGGAGATCACGCAACTGCTGCTTTGGAAGAACAACTTAAGGT
ATTGGGAGATCGATGGGCAAACATCTGTAGATGGACAGAAGACCGCTGGGTTCTTTTACAAGACATCCTTCTCAAAT
GGCAACGTCTTACTGAAGAACAGTGCCTTTTTAGTGCATGGCTTTCAGAAAAAGAAGATGCAGTGAACAAGATTCAC
ACAACTGGCTTTAAAGATCAAAATGAAATGTTATCAAGTCTTCAAAAACTGGCCGTTTTAAAAGCGGATCTAGAAAA
GAAAAAGCAATCCATGGGCAAACTGTATTCACTCAAACAAGATCTTCTTTCAACACTGAAGAATAAGTCAGTGACCC
AGAAGACGGAAGCATGGCTGGATAACTTTGCCCGGTGTTGGGATAATTTAGTCCAAAAACTTGAAAAGAGTACAGCA
CAGATTTCACAGGCTGTCACCACCACTCAGCCATCACTAACACAGACAACTGTAATGGAAACAGTAACTACGGTGAC
CACAAGGGAACAGATCCTGGTAAAGCATGCTCAAGAGGAACTTCCACCACCACCTCCCCAAAAGAAGAGGCAGATTA
CTGTGGATTCTGAAATTAGGAAAAGGTTGGATGTTGATATAACTGAACTTCACAGCTGGATTACTCGCTCAGAAGCT
GTGTTGCAGAGTCCTGAATTTGCAATCTTTCGGAAGGAAGGCAACTTCTCAGACTTAAAAGAAAAAGTCAATGCCAT
AGAGCGAGAAAAAGCTGAGAAGTTCAGAAAACTGCAAGATGCCAGCAGATCAGCTCAGGCCCTGGTGGAACAGATGG
TGAATGAGGGTGTTAATGCAGATAGCATCAAACAAGCCTCAGAACAACTGAACAGCCGGTGGATCGAATTCTGCCAG
TTGCTAAGTGAGAGACTTAACTGGCTGGAGTATCAGAACAACATCATCGCTTTCTATAATCAGCTACAACAATTGGA
GCAGATGACAACTACTGCTGAAAACTGGTTGAAAATCCAACCCACCACCCCATCAGAGCCAACAGCAATTAAAAGTC
AGTTAAAAATTTGTAAGGATGAAGTCAACCGGCTATCAGGTCTTCAACCTCAAATTGAACGATTAAAAATTCAAAGC
ATAGCCCTGAAAGAGAAAGGACAAGGACCCATGTTCCTGGATGCAGACTTTGTGGCCTTTACAAATCATTTTAAGCA
AGTCTTTTCTGATGTGCAGGCCAGAGAGAAAGAGCTACAGACAATTTTTGACACTTTGCCACCAATGCGCTATCAGG
AGACCATGAGTGCCATCAGGACATGGGTCCAGCAGTCAGAAACCAAACTCTCCATACCTCAACTTAGTGTCACCGAC
TATGAAATCATGGAGCAGAGACTCGGGGAATTGCAGGCTTTACAAAGTTCTCTGCAAGAGCAACAAAGTGGCCTATA
CTATCTCAGCACCACTGTGAAAGAGATGTCGAAGAAAGCGCCCTCTGAAATTAGCCGGAAATATCAATCAGAATTTG
AAGAAATTGAGGGACGCTGGAAGAAGCTCTCCTCCCAGCTGGTTGAGCATTGTCAAAAGCTAGAGGAGCAAATGAAT
AAACTCCGAAAAATTCAGAATCACATACAAACCCTGAAGAAATGGATGGCTGAAGTTGATGTTTTTCTGAAGGAGGA
ATGGCCTGCCCTTGGGGATTCAGAAATTCTAAAAAAGCAGCTGAAACAGTGCAGACTTTTAGTCAGTGATATTCAGA
CAATTCAGCCCAGTCTAAACAGTGTCAATGAAGGTGGGCAGAAGATAAAGAATGAAGCAGAGCCAGAGTTTGCTTCG
AGACTTGAGACAGAACTCAAAGAACTTAACACTCAGTGGGATCACATGTGCCAACAGGTCTATGCCAGAAAGGAGGC
CTTGAAGGGAGGTTTGGAGAAAACTGTAAGCCTCCAGAAAGATCTATCAGAGATGCACGAATGGATGACACAAGCTG
AAGAAGAGTATCTTGAGAGAGATTTTGAATATAAAACTCCAGATGAATTACAGAAAGCAGTTGAAGAGATGAAGAGA
GCTAAAGAAGAGGCCCAACAAAAAGAAGCGAAAGTGAAACTCCTTACTGAGTCTGTAAATAGTGTCATAGCTCAAGC
TCCACCTGTAGCACAAGAGGCCTTAAAAAAGGAACTTGAAACTCTAACCACCAACTACCAGTGGCTCTGCACTAGGC
TGAATGGGAAATGCAAGACTTTGGAAGAAGTTTGGGCATGTTGGCATGAGTTATTGTCATACTTGGAGAAAGCAAAC
AAGTGGCTAAATGAAGTAGAATTTAAACTTAAAACCACTGAAAACATTCCTGGCGGAGCTGAGGAAATCTCTGAGGT
GCTAGATTCACTTGAAAATTTGATGCGACATTCAGAGGATAACCCAAATCAGATTCGCATATTGGCACAGACCCTAA
CAGATGGCGGAGTCATGGATGAGCTAATCAATGAGGAACTTGAGACATTTAATTCTCGTTGGAGGGAACTACATGAA
GAGGCTGTAAGGAGGCAAAAGTTGCTTGAACAGAGCATCCAGTCTGCCCAGGAGACTGAAAAATCCTTACACTTAAT
CCAGGAGTCCCTCACATTCATTGACAAGCAGTTGGCAGCTTATATTGCAGACAAGGTGGACGCAGCTCAAATGCCTC
AGGAAGCCCAGAAAATCCAATCTGATTTGACAAGTCATGAGATCAGTTTAGAAGAAATGAAGAAACATAATCAGGGG
AAGGAGGCTGCCCAAAGAGTCCTGTCTCAGATTGATGTTGCACAGAAAAAATTACAAGATGTCTCCATGAAGTTTCG
ATTATTCCAGAAACCAGCCAATTTTGAGCAGCGTCTACAAGAAAGTAAGATGATTTTAGATGAAGTGAAGATGCACT
TGCCTGCATTGGAAACAAAGAGTGTGGAACAGGAAGTAGTACAGTCACAGCTAAATCATTGTGTGAACTTGTATAAA
AGTCTGAGTGAAGTGAAGTCTGAAGTGGAAATGGTGATAAAGACTGGACGTCAGATTGTACAGAAAAAGCAGACGGA
AAATCCCAAAGAACTTGATGAAAGAGTAACAGCTTTGAAATTGCATTATAATGAGCTGGGAGCAAAGGTAACAGAAA
GAAAGCAACAGTTGGAGAAATGCTTGAAATTGTCCCGTAAGATGCGAAAGGAAATGAATGTCTTGACAGAATGGCTG
GCAGCTACAGATATGGAATTGACAAAGAGATCAGCAGTTGAAGGAATGCCTAGTAATTTGGATTCTGAAGTTGCCTG
GGGAAAGGCTACTCAAAAAGAGATTGAGAAACAGAAGGTGCACCTGAAGAGTATCACAGAGGTAGGAGAGGCCTTGA
AAACAGTTTTGGGCAAGAAGGAGACGTTGGTGGAAGATAAACTCAGTCTTCTGAATAGTAACTGGATAGCTGTCACC
TCCCGAGCAGAAGAGTGGTTAAATCTTTTGTTGGAATACCAGAAACACATGGAAACTTTTGACCAGAATGTGGACCA
CATCACAAAGTGGATCATTCAGGCTGACACACTTTTGGATGAATCAGAGAAAAAGAAACCCCAGCAAAAAGAAGACG
TGCTTAAGCGTTTAAAGGCAGAACTGAATGACATACGCCCAAAGGTGGACTCTACACGTGACCAAGCAGCAAACTTG
ATGGCAAACCGCGGTGACCACTGCAGGAAATTAGTAGAGCCCCAAATCTCAGAGCTCAACCATCGATTTGCAGCCAT
TTCACACAGAATTAAGACTGGAAAGGCCTCCATTCCTTTGAAGGAATTGGAGCAGTTTAACTCAGATATACAAAAAT
TGCTTGAACCACTGGAGGCTGAAATTCAGCAGGGGGTGAATCTGAAAGAGGAAGACTTCAATAAAGATATGAATGAA
GACAATGAGGGTACTGTAAAAGAATTGTTGCAAAGAGGAGACAACTTACAACAAAGAATCACAGATGAGAGAAAGCG
AGAGGAAATAAAGATAAAACAGCAGCTGTTACAGACAAAACATAATGCTCTCAAGGATTTGAGGTCTCAAAGAAGAA
AAAAGGCTCTAGAAATTTCTCATCAGTGGTATCAGTACAAGAGGCAGGCTGATGATCTCCTGAAATGCTTGGATGAC
ATTGAAAAAAAATTAGCCAGCCTACCTGAGCCCAGAGATGAAAGGAAAATAAAGGAAATTGATCGGGAATTGCAGAA
GAAGAAAGAGGAGCTGAATGCAGTGCGTAGGCAAGCTGAGGGCTTGTCTGAGGATGGGGCCGCAATGGCAGTGGAGC
CAACTCAGATCCAGCTCAGCAAGCGCTGGCGGGAAATTGAGAGCAAATTTGCTCAGTTTCGAAGACTCAACTTTGCA
CAAATTCACACTGTCCGTGAAGAAACGATGATGGTGATGACTGAAGACATGCCTTTGGAAATTTCTTATGTGCCTTC
TACTTATTTGACTGAAATCACTCATGTCTCACAAGCCCTATTAGAAGTGGAACAACTTCTCAATGCTCCTGACCTCT
GTGCTAAGGACTTTGAAGATCTCTTTAAGCAAGAGGAGTCTCTGAAGAATATAAAAGATAGTCTACAACAAAGCTCA
GGTCGGATTGACATTATTCATAGCAAGAAGACAGCAGCATTGCAAAGTGCAACGCCTGTGGAAAGGGTGAAGCTACA
GGAAGCTCTCTCCCAGCTTGATTTCCAATGGGAAAAAGTTAACAAAATGTACAAGGACCGACAAGGGCGATTTGACA
GATCTGTTGAGAAATGGCGGCGTTTTCATTATGATATAAAGATATTTAATCAGTGGCTAACAGAAGCTGAACAGTTT
CTCAGAAAGACACAAATTCCTGAGAATTGGGAACATGCTAAATACAAATGGTATCTTAAGGAACTCCAGGATGGCAT
TGGGCAGCGGCAAACTGTTGTCAGAACATTGAATGCAACTGGGGAAGAAATAATTCAGCAATCCTCAAAAACAGATG
CCAGTATTCTACAGGAAAAATTGGGAAGCCTGAATCTGCGGTGGCAGGAGGTCTGCAAACAGCTGTCAGACAGAAAA
AAGAGGCTAGAAGAACAAAAGAATATCTTGTCAGAATTTCAAAGAGATTTAAATGAATTTGTTTTATGGTTGGAGGA
AGCAGATAACATTGCTAGTATCCCACTTGAACCTGGAAAAGAGCAGCAACTAAAAGAAAAGCTTGAGCAAGTCAAGT
TACTGGTGGAAGAGTTGCCCCTGCGCCAGGGAATTCTCAAACAATTAAATGAAACTGGAGGACCCGTGCTTGTAAGT
GCTCCCATAAGCCCAGAAGAGCAAGATAAACTTGAAAATAAGCTCAAGCAGACAAATCTCCAGTGGATAAAGGTTTC
CAGAGCTTTACCTGAGAAACAAGGAGAAATTGAAGCTCAAATAAAAGACCTTGGGCAGCTTGAAAAAAAGCTTGAAG
ACCTTGAAGAGCAGTTAAATCATCTGCTGCTGTGGTTATCTCCTATTAGGAATCAGTTGGAAATTTATAACCAACCA
AACCAAGAAGGACCATTTGACGTTCAGGAAACTGAAATAGCAGTTCAAGCTAAACAACCGGATGTGGAAGAGATTTT
GTCTAAAGGGCAGCATTTGTACAAGGAAAAACCAGCCACTCAGCCAGTGAAGAGGAAGTTAGAAGATCTGAGCTCTG
AGTGGAAGGCGGTAAACCGTTTACTTCAAGAGCTGAGGGCAAAGCAGCCTGACCTAGCTCCTGGACTGACCACTATT
GGAGCCTCTCCTACTCAGACTGTTACTCTGGTGACACAACCTGTGGTTACTAAGGAAACTGCCATCTCCAAACTAGA
AATGCCATCTTCCTTGATGTTGGAGGTACCTGCTCTGGCAGATTTCAACCGGGCTTGGACAGAACTTACCGACTGGC
TTTCTCTGCTTGATCAAGTTATAAAATCACAGAGGGTGATGGTGGGTGACCTTGAGGATATCAACGAGATGATCATC
AAGCAGAAGGCAACAATGCAGGATTTGGAACAGAGGCGTCCCCAGTTGGAAGAACTCATTACCGCTGCCCAAAATTT
GAAAAACAAGACCAGCAATCAAGAGGCTAGAACAATCATTACGGATCGAATTGAAAGAATTCAGAATCAGTGGGATG
AAGTACAAGAACACCTTCAGAACCGGAGGCAACAGTTGAATGAAATGTTAAAGGATTCAACACAATGGCTGGAAGCT
AAGGAAGAAGCTGAGCAGGTCTTAGGACAGGCCAGAGCCAAGCTTGAGTCATGGAAGGAGGGTCCCTATACAGTAGA
TGCAATCCAAAAGAAAATCACAGAAACCAAGCAGTTGGCCAAAGACCTCCGCCAGTGGCAGACAAATGTAGATGTGG
CAAATGACTTGGCCCTGAAACTTCTCCGGGATTATTCTGCAGATGATACCAGAAAAGTCCACATGATAACAGAGAAT
ATCAATGCCTCTTGGAGAAGCATTCATAAAAGGGTGAGTGAGCGAGAGGCTGCTTTGGAAGAAACTCATAGATTACT
GCAACAGTTCCCCCTGGACCTGGAAAAGTTTCTTGCCTGGCTTACAGAAGCTGAAACAACTGCCAATGTCCTACAGG
ATGCTACCCGTAAGGAAAGGCTCCTAGAAGACTCCAAGGGAGTAAAAGAGCTGATGAAACAATGGCAAGACCTCCAA
GGTGAAATTGAAGCTCACACAGATGTTTATCACAACCTGGATGAAAACAGCCAAAAAATCCTGAGATCCCTGGAAGG
TTCCGATGATGCAGTCCTGTTACAAAGACGTTTGGATAACATGAACTTCAAGTGGAGTGAACTTCGGAAAAAGTCTC
TCAACATTAGGTCCCATTTGGAAGCCAGTTCTGACCAGTGGAAGCGTCTGCACCTTTCTCTGCAGGAACTTCTGGTG
TGGCTACAGCTGAAAGATGATGAATTAAGCCGGCAGGCACCTATTGGAGGCGACTTTCCAGCAGTTCAGAAGCAGAA
CGATGTACATAGGGCCTTCAAGAGGGAATTGAAAACTAAAGAACCTGTAATCATGAGTACTCTTGAGACTGTACGAA
TATTTCTGACAGAGCAGCCTTTGGAAGGACTAGAGAAACTCTACCAGGAGCCCAGAGAGCTGCCTCCTGAGGAGAGA
GCCCAGAATGTCACTCGGCTTCTACGAAAGCAGGCTGAGGAGGTCAATACTGAGTGGGAAAAATTGAACCTGCACTC
CGCTGACTGGCAGAGAAAAATAGATGAGACCCTTGAAAGACTCCAGGAACTTCAAGAGGCCACGGATGAGCTGGACC
TCAAGCTGCGCCAAGCTGAGGTGATCAAGGGATCCTGGCAGCCCGTGGGCGATCTCCTCATTGACTCTCTCCAAGAT
CACCTCGAGAAAGTCAAGGCACTTCGAGGAGAAATTGCGCCTCTGAAAGAGAACGTGAGCCACGTCAATGACCTTGC
TCGCCAGCTTACCACTTTGGGCATTCAGCTCTCACCGTATAACCTCAGCACTCTGGAAGACCTGAACACCAGATGGA
AGCTTCTGCAGGTGGCCGTCGAGGACCGAGTCAGGCAGCTGCATGAAGCCCACAGGGACTTTGGTCCAGCATCTCAG
CACTTTCTTTCCACGTCTGTCCAGGGTCCCTGGGAGAGAGCCATCTCGCCAAACAAAGTGCCCTACTATATCAACCA
CGAGACTCAAACAACTTGCTGGGACCATCCCAAAATGACAGAGCTCTACCAGTCTTTAGCTGACCTGAATAATGTCA
GATTCTCAGCTTATAGGACTGCCATGAAACTCCGAAGACTGCAGAAGGCCCTTTGCTTGGATCTCTTGAGCCTGTCA
GCTGCATGTGATGCCTTGGACCAGCACAACCTCAAGCAAAATGACCAGCCCATGGATATCCTGCAGATTATTAATTG
TTTGACCACTATTTATGACCGCCTGGAGCAAGAGCACAACAATTTGGTCAACGTCCCTCTCTGCGTGGATATGTGTC
TGAACTGGCTGCTGAATGTTTATGATACGGGACGAACAGGGAGGATCCGTGTCCTGTCTTTTAAAACTGGCATCATT
TCCCTGTGTAAAGCACATTTGGAAGACAAGTACAGATACCTTTTCAAGCAAGTGGCAAGTTCAACAGGATTTTGTGA
CCAGCGCAGGCTGGGCCTCCTTCTGCATGATTCTATCCAAATTCCAAGACAGTTGGGTGAAGTTGCATCCTTTGGGG
GCAGTAACATTGAGCCAAGTGTCCGGAGCTGCTTCCAATTTGCTAATAATAAGCCAGAGATCGAAGCGGCCCTCTTC
CTAGACTGGATGAGACTGGAACCCCAGTCCATGGTGTGGCTGCCCGTCCTGCACAGAGTGGCTGCTGCAGAAACTGC
CAAGCATCAGGCCAAATGTAACATCTGCAAAGAGTGTCCAATCATTGGATTCAGGTACAGGAGTCTAAAGCACTTTA
ATTATGACATCTGCCAAAGCTGCTTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACTAAAAAACAAATTTCGAACCAAAAG
GTATTTTGCGAAGCATCCCCGAATGGGCTACCTGCCAGTGCAGACTGTCTTAGAGGGGGACAACATGGAAACTCCCG
TTACTCTGATCAACTTCTGGCCAGTAGATTCTGCGCCTGCCTCGTCCCCTCAGCTTTCACACGATGATACTCATTCA
CGCATTGAACATTATGCTAGCAGGCTAGCAGAAATGGAAAACAGCAATGGATCTTATCTAAATGATAGCATCTCTCC
TAATGAGAGCATAGATGATGAACATTTGTTAATCCAGCATTACTGCCAAAGTTTGAACCAGGACTCCCCCCTGAGCC
AGCCTCGTAGTCCTGCCCAGATCTTGATTTCCTTAGAGAGTGAGGAAAGAGGGGAGCTAGAGAGAATCCTAGCAGAT
CTTGAGGAAGAAAACAGGAATCTGCAAGCAGAATATGACCGTCTAAAGCAGCAGCACGAACATAAAGGCCTGTCCCC
ACTGCCGTCCCCTCCTGAAATGATGCCCACCTCTCCCCAGAGTCCCCGGGATGCTGAGCTCATTGCTGAGGCCAAGC
TACTGCGTCAACACAAAGGCCGCCTGGAAGCCAGGATGCAAATCCTGGAAGACCACAATAAACAGCTGGAGTCACAG
TTACACAGGCTAAGGCAGCTGCTGGAGCAACCCCAGGCAGAGGCCAAAGTGAATGGCACAACGGTGTCCTCTCCTTC
TACCTCTCTACAGAGGTCCGACAGCAGTCAGCCTATGCTGCTCCGAGTGGTTGGCAGTCAAACTTCGGACTCCATGG
GTGAGGAAGATCTTCTCAGTCCTCCCCAGGACACAAGCACAGGGTTAGAGGAGGTGATGGAGCAACTCAACAACTCC
TTCCCTAGTTCAAGAGGAAGAAATACCCCTGGAAAGCCAATGAGAGAGGACACAATGTAGGAAGTCTTTTCCACATG
GCAGATGATTTGGGCAGAGCGATGGAGTCCTTAGTATCAGTCATGACAGATGAAGAAGGAGCAGAATAAATGTTTTA
CAACTCCTGATTCCCGCATGGTTTTTATAATATTCATACAACAAAGAGGATTAGACAGTAAGAGTTTACAAGAAATA
AATCTATATTTTTGTGAAGGGTAGTGGTATTATACTGTAGATTTCAGTAGTTTCTAAGTCTGTTATTGTTTTGTTAA
CAATGGCAGGTTTTACACGTCTATGCAATTGTACAAAAAAGTTATAAGAAAACTACATGTAAAATCTTGATAGCTAA
ATAACTTGCCATTTCTTTATATGGAACGCATTTTGGGTTGTTTAAAAATTTATAACAGTTATAAAGAAAGATTGTAA
ACTAAAGTGTGCTTTATAAAAAAAAGTTGTTTATAAAAACCCCTAAAAACAAAACAAACACACACACACACACATAC
ACACACACACACAAAACTTTGAGGCAGCGCATTGTTTTGCATCCTTTTGGCGTGATATCCATATGAAATTCATGGCT
TTTTCTTTTTTTGCATATTAAAGATAAGACTTCCTCTACCACCACACCAAATGACTACTACACACTGCTCATTTGAG
AACTGTCAGCTGAGTGGGGCAGGCTTGAGTTTTCATTTCATATATCTATATGTCTATAAGTATATAAATACTATAGT
TATATAGATAAAGAGATACGAATTTCTATAGACTGACTTTTTCCATTTTTTAAATGTTCATGTCACATCCTAATAGA
AAGAAATTACTTCTAGTCAGTCATCCAGGCTTACCTGCTTGGTCTAGAATGGATTTTTCCCGGAGCCGGAAGCCAGG
AGGAAACTACACCACACTAAAACATTGTCTACAGCTCCAGATGTTTCTCATTTTAAACAACTTTCCACTGACAACGA
AAGTAAAGTAAAGTATTGGATTTTTTTAAAGGGAACATGTGAATGAATACACAGGACTTATTATATCAGAGTGAGTA
ATCGGTTGGTTGGTTGATTGATTGATTGATTGATACATTCAGCTTCCTGCTGCTAGCAATGCCACGATTTAGATTTA
ATGATGCTTCAGTGGAAATCAATCAGAAGGTATTCTGACCTTGTGAACATCAGAAGGTATTTTTTAACTCCCAAGCA
GTAGCAGGACGATGATAGGGCTGGAGGGCTATGGATTCCCAGCCCATCCCTGTGAAGGAGTAGGCCACTCTTTAAGT
GAAGGATTGGATGATTGTTCATAATACATAAAGTTCTCTGTAATTACAACTAAATTATTATGCCCTCTTCTCACAGT
CAAAAGGAACTGGGTGGTTTGGTTTTTGTTGCTTTTTTAGATTTATTGTCCCATGTGGGATGAGTTTTTAAATGCCA
CAAGACATAATTTAAAATAAATAAACTTTGGGAAAAGGTGTAAAACAGTAGCCCCATCACATTTGTGATACTGACAG
GTATCAACCCAGAAGCCCATGAACTGTGTTTCCATCCTTTGCATTTCTCTGCGAGTAGTTCCACACAGGTTTGTAAG
TAAGTAAGAAAGAAGGCAAATTGATTCAAATGTTACAAAAAAACCCTTCTTGGTGGATTAGACAGGTTAAATATATA
AACAAACAAACAAAAATTGCTCAAAAAAGAGGAGAAAAGCTCAAGAGGAAAAGCTAAGGACTGGTAGGAAAAAGCTT
TACTCTTTCATGCCATTTTATTTCTTTTTGATTTTTAAATCATTCATTCAATAGATACCACCGTGTGACCTATAATT
TTGCAAATCTGTTACCTCTGACATCAAGTGTAATTAGCTTTTGGAGAGTGGGCTGACATCAAGTGTAATTAGCTTTT
GGAGAGTGGGTTTTGTCCATTATTAATAATTAATTAATTAACATCAAACACGGCTTCTCATGCTATTTCTACCTCAC
TTTGGTTTTGGGGTGTTCCTGATAATTGTGCACACCTGAGTTCACAGCTTCACCACTTGTCCATTGCGTTATTTTCT
TTTTCCTTTATAATTCTTTCTTTTTCCTTCATAATTTTCAAAAGAAAACCCAAAGCTCTAAGGTAACAAATTACCAA
ATTACATGAAGATTTGGTTTTTGTCTTGCATTTTTTTCCTTTATGTGACGCTGGACCTTTTCTTTACCCAAGGATTT
TTAAAACTCAGATTTAAAACAAGGGGTTACTTTACATCCTACTAAGAAGTTTAAGTAAGTAAGTTTCATTCTAAAAT
CAGAGGTAAATAGAGTGCATAAATAATTTTGTTTTAATCTTTTTGTTTTTCTTTTAGACACATTAGCTCTGGAGTGA
GTCTGTCATAATATTTGAACAAAAATTGAGAGCTTTATTGCTGCATTTTAAGCATAATTAATTTGGACATTATTTCG
TGTTGTGTTCTTTATAACCACCAAGTATTAAACTGTAAATCATAATGTAACTGAAGCATAAACATCACATGGCATGT
TTTGTCATTGTTTTCAGGTACTGAGTTCTTACTTGAGTATCATAATATATTGTGTTTTAACACCAACACTGTAACAT
TTACGAATTATTTTTTTAAACTTCAGTTTTACTGCATTTTCACAACATATCAGACTTCACCAAATATATGCCTTACT
ATTGTATTATAGTACTGCTTTACTGTGTATCTCAATAAAGCACGCAGTTATGTTAC

Homo sapiens dystrophin (DMD), transcript variant Dp427m, exon 54 (nucleotide positions 8117-8271 of NCBI Reference Sequence: NM_004006.2; nucleotide positions 1686466-1686620 of NCBI Reference Sequence: NG_012232.1)

(SEQ ID NO: 2142)
CAGTTGGCCAAAGACCTCCGCCAGTGGCAGACAAATGTAGATGTGGCAA
ATGACTTGGCCCTGAAACTTCTCCGGGATTATTCTGCAGATGATACCAG
AAAAGTCCACATGATAACAGAGAATATCAATGCCTCTTGGAGAAGCATT
CATAAAAG

Homo sapiens dystrophin (DMD), exon 54 target sequence 1 (nucleotide positions 1686541-1686602 of NCBI Reference Sequence: NG_012232.1)

(SEQ ID NO: 2143)
GATTATTCTGCAGATGATACCAGAAAAGTCCACATGATAACAGAGAATA
TCAATGCCTCTTG

Homo sapiens dystrophin (DMD) exon 54/intron 54 junction (nucleotide positions 1686591 to 1686650 of NCBI Reference Sequence: NG_012232.1)

(SEQ ID NO: 2144)
CAATGCCTCTTGGAGAAGCATTCATAAAAGGTATGAATTACATTATTTC
TAAAACTACTG

Homo sapiens dystrophin (DMD), intron 54 (nucleotide positions 1686621-1716747 of NCBI Reference Sequence: NG_012232.1)

(SEQ ID NO: 2145)
GTATGAATTACATTATTTCTAAAACTACTGTTGGCTGTAATAATGGGGTGGTGAAACTGGATGGACCATGAGGATTT
GTTTTTCCAATCCAGCTAAACTGGAGCTTGGGAGGGTTCAAGACGATAAATACCAACTAAACTCACGGACTTGGCTC
AGACTTCTATTTTAAAAACGAGGAACATAAGATCTCATTTGCCCGCTGTCACAAAAGTAGTGACATAACCAAGAGAT
TAAACAAAAAGCAAAATACTGATTTATAGCTAGAAGAGCCATTTATCAGTCTACTTTGATAACTCTATCCAAAGGAA
TATCTTTCTATCTCATCATGGCGCACACTGCCTTACCTGTTATCTGATAAATAAGTCACTTTGGGATTCATGATAGA
GTTATAGCTGTACATGGTCTCATCCTAGTATCTCACTCCACACACCCAATGGGAAAATTTGTGGAGGGCAATATGAC
TCGTCACTTCATTTCCCATTATATATGAATGGAAATTAACAGCGCTTATAGACAGTATCTCCTCAAACTAAGCCTTG
TATCCTTATTATACCTCTCTTGATCTCTAGTGCTTTTTTCACTAGCATTTATTCCAATCATAAATAAAAATATAAAT
TATGTAACTAATTGTTAAATATTTGTCCTTTAAATTAATCTAAATGCCATGAGGGCAGAGATTTTGTCTTTCTCATT
TGATACATCCCCAGGTCCTGAACCACGTGATATAATAGGGAGCTAGTAAATGTTTTTTGAATGATGACTCCCTTTGC
AGAATGTACAATTACCTTGTGCAAGCTGAAAAAATAGCACCTGTACAATATGAGGAAGACCACGGTGAAAAATAATT
GAGTTCCAAAATATGACATCAATTACTGAAAAAATAAGCTCGGTGATTTTTAACAAGAAGTAAAAGTCACCACTGGG
GCCAAAACAGATTTTGAACTAAGAGTAGGAAGTCTTAGGAGAAATGAGATAATGATATATGGAAATTAAGCGGCCAA
CTAAATTTTGAAACTGAGCTAGACATTAGAGAGTAAAAACTCCTGTGAAGCTGAATTTAAGCTGGTCACCCTGGGGA
ATAGAGCAACTCTAATCCTGAATTCCAGACAGTAGGTGTATAGATGGAAAAGACCATGGAAAAGAAGATTCAACCTA
AAGTTGGGAAGTTTTAATTGGAGCCCTATGAAAAAGACCCTGGTGGAGAAAGGGCAAACTTGAATATGGAGCTGATA
TTTGGAAAAATTCTCATAGTAACTACTTTTTCTCAATGGCAAGGCTTGGACTTTCTTCTCAAAATACAGATCTTATA
TGTGTTCAATTAAACAGGGACAGATTAGGTTCAGGAAGAATTATTCACATGGAATCAATTGGTATCAGAGAGTCAAC
CATTAGATCTTAGTGGGAAATATCTGCTTCTCAAAGAGAAGTCTTTTGGGGAAAGCAAATTAAAGTCAGAGATTAAT
TTGATGAGTTTAGGTAATATAAACTAAGGGGCCAAGAAAAAAGCTTGCTCATGGTATGAAACTAGAGCTTGAGGACA
CTGATCTAGTCTATCTATACTACTCTTTCTGACAGACCCCTCTCTTCATTCTCATGCTCCTTGATGGCCCAAGCCAC
TCTCTCAGTTTTTTAAAAAATTGTTTTATCAAGGTCTCTGGATTCTTCATGGGAATGACTTCCAGTTTATATTTTTT
GGCTTGGTTCCAAAAAGCTATCAGCTAAGGAATGCATATACTTACTTCCCCTATGGGTAAAGTAAATGAGAATTTTA
GAAGCCAACTCACATTTTTAGCCTGTACAGAATCTGCAATTCACCAAGCTACTTCTGACTCATGTCTATAAAGTTCT
TCCCTGTTCTTTTCTCACTTCACATGTACTCTTTGCAAGAATTCATCCACTTGTGTAGTTTCAGTCTGTTGATGACT
ACCCATCTATAATTCCAGCTGAGAATGATCTTTTGAGTTTTAGACATGTAGATCCTGCTGCTTTCTTTCGATGTTAA
TGTCCCACAGGAACTTCACATTGAAGAGGTCCAAAGCTAAACTCATCTTTGCCTTCTTCCAATCTCTTTCTCCAAAT
GCAACCTACTTCTGTTGTCCTTGTCTTAGTCCTTTTCGTGCTTCCGTAACAAAATACCACAGACTGGGTAATTTATA
ATGAACAGGGATTTGTTGGCTCATAGTTCTGGAGGCTGCGAAGTCCAAGATCAAGGGGCTGGAATCTGGTAAGGGCC
TTCTTGTTGTGTCATGATTCCATGATGGAAGGTGGAAGACCAAAAGAGAGAAAAAATGGGGCCAAACTTGTCCTTAT
ATGAAACTCACTCCCACAATAATGATGCTAATCCGTTCATGAAGGCAGAGCCTTCATGTCCTAATCACCTCTTCAAG
GTCACATTTACTACTGTTGCAATGGCAATTAAATTTTACCATAAGTTTGGGAAGGGAAAAACATTAAACCATAGCAT
TCTGCCCCCTTTTCCCCAAAATTCTTGTTCTTCTCAAAGACAAAATACATTCATTTCATCCCCAAAGCCCCAAAAAT
CTTATTTCAGCATAAACTCAAAAGTGCAATCTAATATAAATTAGATATGGGTGAGACTCAAGGCACAATTCATCGTG
AGGCAAATTCCCTTCCATCTCTGAGCCTGCAAAATCGAATCAAGTTCATCCCCTCACCCCCTACCCTTCCCAGCATC
AGGTAACCACCAATCACAGAAAGTTTTACTGATAGTCCTGCTCTAGATCATCTTTGTCTATGTTCACTTTAGCTATT
TATCCTAGTGTTCCATTATTGGAATACTAAGCATGTGGGAATTATTTATATTCTACTGTTCAAGGTCCTCACCAAGG
TCTGATTGCAAAAATTCAAAAAATTGCAACCTTAGGCATAAATGGGTTAAGCAGTTTAGGGTACATTTATAATAATT
ATTTACTGTGCTACTTCAAAAATCTTATTGCCTCTATTTATAAATAAAAAGTGTTGTCTCTACACAGTGGCTTGTTG
TAATGCATTTACTTGTTTCTGCCTGATTTTTTCTATTTATACATTTTCTTTTTTATTTTTATTTTTATTTTTTCACT
TTTAAGTTCAGGGGTACATGTGCAGGTTTGTTACATAGGTAAACTTGTGTCATGGGGGTCTGTTGTACAGATTATTT
CATCACCTAGGTATTAATCCTGGTACCCGTTAGTTGACTTTCCTGATCCTCTCGCTCCTCCCACCCTCCACACTCTA
ATAGTCCCTAGCATGTGTTGTTCCCCTCTACGTGTCCATGTGTTCTCATCATTTAGCTCCCACTTATAAATGAGAAC
ATGGGGTATTTGGTTTTTTGTTCCTGTATTAGTTTGATAAGGACAATGGCCTCCAGATCCATCTATGTCCCTGCAAA
GGACATGATCTCATTCTTTTTTTATGGCTACGTAGTATTCCATGGTATTTGTGTTGGTCTCAAAAACTACAACTATG
ACAGGATGGCATTTTCACTTTTGTTGTTATATTAAACTCATCTTAAAAAGGAAAGATTAATAATGTCAATATTTGGG
TTATGGAGAAAAAGTATCTCATATCTTTGAAAAAGTTCTGTAACTATAGCTTTTTAGGTAGGAGGGATTCTGTGGAA
AGTTTTCTGATTACATCATTTCTCACAGTTCAGGTTAGACACCATTTTACTATGAAACACTAATGCATTGCCTGCAC
TGAGACTTTCAGTCACATGGAGAAACCTAGGCAAAATTTTTGTACACTTGGAAGAATATTTAAATTAGTAATAAAAT
CTTTAGTTTTAAACTGTTGAATGTTAAATAAGATATAAAATGTACTTGAAAGAAATTTGCTTTGATATCAGACACTG
CCATGTTGCAGTTTCAAGACATAATAAAAAAGTAAACTAATGTTTATATTTTGCTGTTTAAGTTTATTAATACATCA
GATGAGTCTTCAAATTCTACAGTGGCTTTTGATATGATCATTTTTACTTGCCATTTTATATAGAATAAATATAAATA
GGCATTTATGCTTAAAAGGAACTAATCTATCTATGGAAAAAAGAGAAGGCTGCTTCTCAACTAAATTGTACAGTTTA
GAAACCCAGATCTGAACATAGATTATTGTTGTGACCTATGTAGGAAAATATGTTGTTTTCCTTATCGTAGTCCTTAC
AGAGTCCATGATAACATATAAAGCCAGAAATGTGAGCCTCTGCAAGTTCATTTCTTTGTCTTCAATCTCTGTGAATA
GATATGAGTTTGTGAATAAGATAATATTAGATGTGATATTACAAATTATTGTGAGAAGCCTCTAAGGATTAGATTTC
AAGGACTGCCATCTGGCTGATGACTTTATGATGACACTGTCATGAGATTTCATTTCCTTATTTCTGTTCCAGGATCA
CTCTTTAAACAAGAAATAAGCATTAACTCTGAATTGTCTGCTTGTAGCTGTATGAGGGCTTCCACAACTGCCAACTA
GCCAGGTACAAACTCATCAAGCAGAGGAGATGGTCCTTGCATCAGAGGGTTAAACATGCCTAGAAGTTCCTTAGCTA
AGCTCCCAGATACTAAAAAATCCCTCTAGGTTCTAAGAAAGATTCAGCATGTACATGTGTGTACATGTATGTGTGTA
CATATATACATATACGTGTATATGCATATGCATGCATATACATACAAACACATTTTCTTCCATAACATCTCAGTATT
CTCTGTTCTTTATAATACTGTTTTGTATTTTAATGATCAAAATTAATAGITGATCATCTGAAAACATTTTGACCTGT
TTTCTCCGTCTTTGACAACCTTGAAGGCACTTGTAAGTCACTCTTTGCTTCTCTATTCCTAGGTCCTTTCTCATCTT
CATTGCAACAAGAAAAGAGAAAACAATTGAGCCCTATTTTGTGTGTAGCAAGGAGCTACTCTAGTTAAACACTAGAT
CTCTTTTACATTCTCCAACATGTTGTTTTAGTAATTATTCTACTTTCCTTTTTTTGGGATATTCAATTTCTTCTTTC
TTTTTGCTCCTCCCCTTTAGCAGGCCAACATACTCAAGTCTCCCTCATCCTAAGAGAACTTTTTTAGTATATCATTT
TTTTTCTATCCAGCTGTACTTGCTTCTGCTTACTATATCATTTTTAAGCAGTAGTTGGCATTACTGTTTCCTGTTCT
TTAGCTACTAGTTGTACTTTGACCCACTCCAGTCTCACTTCCCCAGCACCACCACTTTATGAAAACAAGGACTTACT
AAGATCATCAGTGACTTTGTAATAGCTAATTAGTGTATTTTAATTCGTCCATCTTCTTGACTATATTTTAACATTGA
TCCTGTTGGTCAACTCTGCTAATCAAAACTTTATCCTCCTTGGTTCCCAGAACAATATTATCTTGAATATCTCATTT
CTCTAATCATATAATAATTGTGAGGTGCTTGGCACAATGCCTAGTGCGTAGTAAGAACTCAGTAAAATATCATCTGC
CATCGACACCATAAAAATTAATTTACTTACTCAACAAATACTTTTGTATGAAGTTTGTGCTAGGTAGGCCCAGTAAT
TGGTACTTGGTATAGAGCAATGAAAAGCCCTACCCTCATAAAGCTTATATTCTTGGAAGCAGAAGTTGGAAGACAGA
CATTGACAAATAAAAATTAAATACATGATGTGTCAGATGGTCATACACACAGTGTGGAAGAACAAAGAGGAAAACAA
GTGGAGAGAGAGAGGGAGGTGGAAGAGGAGTGCTGCCATGAAAATGTGGTAATCAAAAAAGGTCTTACTGAAAAGGT
GGCATTTAAGCAAATTCTAAAAGACCTGAGGATGTGGGCCATATGTATAATTGGGGGGGAAAAAGTAGTCCAGGAGA
GTCCTAATAAGTTAAAATGCCCCAAAGCAGGAATATTCTTGGCATGTTGAAGGAACCTTAAAAGGGAGATCAGTTAG
GCAGAAAAGGATCAAGCGAGCAGGAAGGTAGTTGACAATAAATTTAGAGGGGTAACTGGCATCTGATTATATTGGCC
TTTTAGGCCTGTGGACTTTAGCTTTTAATCTGAATGAGATGGGAGTTATTGGAGGGTTTTGAATGGAGGAGTGACAT
GTTTTGTCTTATCTGGCTCCTCTGTTACAATAGACTAAACAGAAGTAGTGAGACCATTAGGAAACTGTTGTCATAAT
TCAGTCAAGAGATGACTGTGGCTGGGATCAGAATGGGAGAGGTGAATGTGGTGAGGAGTGGTTGGATTCTACTATAT
TTTGGGTACAGAGCACAACAGATTTTATAATGGAATAAATTTAGGTGTGAGAGAAAGAGTCAAGAAGACTCAAGAAT
TTTTAGCCTGAGCAACGGAAAGATGGGGTCATCATTTACTGAGATGGGGAAGGCTCCAGGAGTAACATATTTTGGGA
GGAAGATGTGGATATGTTACATTTGAAATGCCTATTATACATCTAGGAGATGTGTGGAGTAGATAGCTGGATATATG
AATCTTAAGTTATGGGGAGTAGCTCAAGATACAAAGTTGGGAGTTGTAACAATGATCAGTGCAAGTTCTCTGTCTTC
AATGCAATTTTAAATGTTGATGTTCCATTCTTAATTGTCTCTCTTCTTTCTCTCTGCACATTTTGAGTAGCTTTGTC
TGTTGGCTTCAGTTAACATTAAGACTCCTCAGTGTCAACTTCCATCTTACACTCTTCTCCTGATCTCCAGAACTGTA
CTTTCTGCCACCTAACCTACATTACCACCTGGATATGCTACAGGCTGCAAAATGTGTCAAGTAGAATGCATTATCTT
GCCCCTAAAAGAAAGTTAAATTTTCTGTGTTTTCAGTGTAGTGTAATTGTCTAACTTAATTGTCTCTAAAACTGGAA
ACCTAAGAATTACCTTCTACCTTTCTCTTGATCTCTCTTTCCCAATCTACTGACACATGTATTAAACTGGCTTCCAA
ATTCTGTGAATTCTACTTCAAAAATTGCTCTAGAAACAATTCCCTCTCTTTATCCCTATTGTCACCTCATCCTAAAG
CCTCTTCATCCTTTGTAGATTTCTGGGAGATTGTAACCAACTTTTCTCTATTCTGCCAGTTATCAAGTCTTTACGCT
CATTTGACATTCACAACAGCCTTGGATCTGTCTTCCTTGAAATGAATCTTCTTGCTTCCCTTTGATTCCAGTGCTTT
TTTTTTACCCTCCTGAGACTTGATGCATGATATTTACATGTATGACATGTTTCCAAAAGCATTCTCAAATTTTTCTG
AAAGTAAAAACAAATGAAAAAGTAAAACATTTTCCTGGGAAGAAAAGCAAATAGTGTTATACATTTTTGCTTGTTCA
TTTGTTTGTTTATTTAGGAGAGGGACAAGCATTAGAACTTCATAAGAGTCTTATATGCTGTATCTACAAATACCGTC
CCTTGGCAATATAATTTTAGAGTTCCTTTTCTGGAACTACTTAAGGACTGTTTTATGATCCTCAGCAGACTGTTATA
TTATTTTATAGCCATACCTTTTATTTGCTGAGTAATTGTACTCAATAATTGTTTGTAATTGAATGAAACAATTCATC
AGATGTTGGGCACTGAATGGCTTTGGATTATTTCCAAAAATTTAAAGGATAAAGATTTGCTGCCTTCAAAGCTATGT
ACAAAAATATGATAGAATGCTAGCGGGATATTTGTTTAAAATACAACCTTTATTACATTGGGGCCTGCTCATAATAT
ATATGTGGCACATTTTATTTAAAATATTAAAGTTCCTGGTGGGACATGTCCCCATAATCCCAGCACTTTGGGAGGCC
GAGGTGGGGGTGGGAGGATCACTAGAGGCCAAGAGTTTGAGACCAGCCTGGGCAACATAGTGAGATACCATTTCTAC
AAAACATAAAAAAAAAAAAAAAAAAGCCAAGTTTGTAGTCCCAGCTACTTGGGAAGCTGAGGCAAGAGGATTTCTTG
AACCTAGGAGTTCAGTTCAAGGCTGCAGTGAGCTATGATCATGCCAGTGTACTCCAGCCTGGGTGACGTAGTGAGAC
TCCATCTCTTAAAATTAAATTAAATTTAAAGCTACAAATGACCCCAAAGCCACCAGTTCAACCCTCTCAATTTTGAA
TACCCTATTTTAAATTCCTCTTATGCGAAATGTACCTTGTAGTCCATTTTAAGGACTGAGAGGATTTGGTATGTTAA
AAAATTCAATCCATTATCAACTCCTTTAGGTACACTTAGCAGTATGAAAATGTGTCTTTCGGCTCTTCAGGAGAGAG
TCATATGTATAGTTACAAGACAATCCCATTTTTATATTGCTGAGACCCAAATCTTCCCAACTGATTATGAAGCATAA
GAACTCTTCGGAGGTTTAAGTGAGCTGAGATTGTGCCACTGCACTACAGCCTGGGCGACAGAGCAAGACTTTGTCTC
AAAAAAAAAAAAAAATTCTCTGCATTCTACAGTAGGGTAATATAACATCTATGATGTGAAATCTTGGGGCTCCGGGC
CAGAGAGTGTCATGATCCATATGGATCTAAAAGGTTCATAGTGGTAACAGCCTGCTTCATTTTATGTCATCTCCTTT
CAAGTAATTAGAATGTTTCTAGCTTGCAGGGATTGCACACAAAGGGAGACATTTGGAACCATGTCATTGGTGATTTA
CTGGTGTGGAAAATTACCTGGTGATGTAGCCAAGTAGCCATTTTCATTCTAACCCAGTCCTACAGTCCTGAACTGGG
CTGAACCAACGCACCAAAATATATGCTTAGAAATGCTCCTATGTATCAGTTTTCCCAGGAAAAACAATAGTATTATC
GAAAACTTACCATTGTTTCCTAATAAAAAATTATAGGATACCAACAGACTGTTTTTTGTTCATAAATTTAATATTAC
AGTATCAAATATTAAAGCAAATGGGAGAAAGTTTTTCTTATTTGGTTTAATTGAACCATTAATGTTAGCTACAATAC
CCATCATGTTACTTTTCAATTATATTTATATTTTCATTTTATTTCTATCTGTATCATTCTCAGAAAGACTTCTTTAA
AACATTCAATAAAAATAGAATTTAGGTAGATTTATTTTTAGAAAGTTGAGTTTTTTTAATAAATGAATATAATCATC
ACTTGACTTAATTTTTTTCTGCACAATTCTAGAAATCTTATAGTTTTGGGATCCTTTGGCTTTATTCAGTATGTAAC
AGGGATCTGTTTCCTTTCTCTAAATCATTAATTCAAATGATTTCTTATATTAAAAATGTTTGGACATATAGGTATTA
ATGAGTTTTATGAAATCTAATCTTTCCAATTTCCCCCTAAAAAGGGATGTCATTTAATCAGTTCTAGGTTGTGATCA
ATAGCAGATTTTTTTTTTTTTTTTTTTTTTTTTTTTTTTTTTTTTTTTTTTTTTTGCAGCTCCCTTTCACCCCGTAG
GGAAACCTGATATCATCCTTGACTAATTGCAGCAAAGAGCCTGGCTCAGGTCCTTTGTCTTATACCGAGTGTTTATA
GATTCTTGAGCCCAGCAGAATCTGAACTCCTGGCTACTGCTACCTACTTCCCAGCCCAGGCCCCAAAAGCCCTATGT
CTGCAGCCCCGTGCACCACTGTGTGTTTTTGTGGCATTTCTGAAACACAGAGCTACTTAACTTGTTTCTAAGCCCAG
ATTGTGCCTTTTTGATTTTCTATTTTGGTATTTTATCTACCATTTTTCTGTGTTTGGATGTTTCTTCTATATTTTGA
AATAACTTCTTTCCTTTAGTACAAGTGATTCTTATTGTAGAAACTATCAAAAATTTACAAATAAAGAATCATTCTCA
ACATTCTTAGCAATTCCTTCTATCATATTTTTGCAAATATATTTTTGCCTATTTTTATTTTACTTACTCCCTGTTTA
TTAACAGTTAAAAGCATTTTCAGATAGTTTTATTTTTTCATTTAAAAAAATCTTACCACATTTTTATTAGGAAGGAA
ATGGACAGGTGTTTATCTTTTCAATAAAAAACATGGGGGAAATAATTTCTTGAAGTACATAGTGACATTCTTCCAGC
CAATGTTTTATGCTGTGGTCATTCCGTCTGTCATCAGTATTCATAGAAAGAGATGAAAATTATTTAAATTAACTAGG
AAATCAATTCCCCATTCAAAGCAGTAGTTGTGTGTTTCAAATATCTTCTAATAGTCAGTTTCACACTTAGCTTTATC
AAATTCCTAATTATGATACTCATTACATCACTCTGTGTCCAGTCAGTGTGTTTATGCCACAGAGCAATTAAAGCAAA
TCAGGTGAACCAAATTCAATCACCTTTGTAGATAATAACCTACGTTGCTTAAACTTATGGCCGCTCATACAATTACT
GATGGATTGCCTTTTTCTTTTATATTGCCAGTATTTTAAATGTCCTAGTGAAGTTGGGGTAGCTGTTGAACTTCAAC
TTTATCACAACCTCTTTTTTAAAATGTGTAAACGAAAAAACCCTCCATGAAATGACCAAATACAGTTTTCATGCTGG
GACAAATTAGATGAATAATAATCATAAATTCATAATGATTATTTATGATTTTATGTTTTTATAGTGAGATATGTTTT
GTTGAAATGTGTTATATAAGTGATACTTAAGTTTCCTATTAAAATAGAAATGCTAAAATGGCATTGTTCTCTTTAGC
TGTGAGTCTAGCTTTTGACCTCTGCTTAAACGGAACTGTTGTTCCATCCCAAATCTGCAACTCTGAGGCCTATGCTC
CCTTCACTGCTGTCTAATGGATACCTATCAATTTGGAAGGAGGTTTCAGGCAGCTATTCCCGGTAATCTAATCTCAG
CTCTGTCCTTTTCAATATTTTCATCAGTGGCTTGGATGAAGACATAGATAACATTCTTATCAAATCAATGCCACAAA
GCAGGGAGAAATAGCAAATATAGCAGACAAGAGTATCAGGAGCCAAAAAGTTTTCAACAAGTTGGACTGGTAGGCTG
AATACTGAAAGATGTAATGTAAATGCAAGGTGCTACATGTGGGTTCAAAAGAAACATGAAACAAAAAACCCATCTAA
CTTAGACTGGGCTCCCTGGAAATAGACTAAGATAGAGAGTTGTGTGCATAAGGTTTGTTGAGGAGTGTTCCCATGAG
ATACATGTGTAAGGTTGTAAGATAGGCAAGATTGCACAGACGAAGAAGTGCAGTGAAGCCTGCAGTGCGTTGCGGCC
TCATCAGATTTTCAGGGGAGTTCTGGAAATTGCATGGCCCTTTAGAGACACGCTGAATTGAAGCAAGGGATCTGGAC
CTTTGAACCCAATACTAGAGAGTTAATGGTCCTGGGTCACCCCATGGGAAAGAGCAGACTGGAGTAAGATTGTTACC
TACAGCTGAAGGCAATTTCCAGGGAGGGAGGCAGCTGTGAGCTGTTAGTAGTCAATATTCCAACCAGCTAGGGCATG
AGGTCTTGGCAGAGCAACAGTGTACCCAAGACCGCAGTGTTACCCAAAGTATGGTCCTCTGACTGGCAGCATTGGTA
TCACCTATGAGCTCACTAGAAATTTAAATTTGTAGGTCCTACCCCATCCAACTAAATCAGAATCTCIGGGGATGGGA
CTTGGGGAACTITTAACAAGCTTTCAGGCCTCCAAGTTATTTCTATGCATATTAAAATTTGAGAACCACTGCCTACA
CCAACCAAAAACATTCCAAATATGGAGATAACATAGAGTTTTTAGCAACAATAATCTCCTTCTGTTTCACTTCTCTC
TTTACACACACACACACACACACACACACACAACACACAACACACAATGTGATAGAACAGTGGGAAAGGAAAGCCAA
AGGGGATCTTAGGCCGAATAAATTTAAGCATATAACCTAGTCCTAAGAACGTATATTTCAGCTTAATAGAGAGAGGA
ATATTGTTATAAAGCTGTCCAAAGATGGAACAGGCTGCCTTGTAAAGTTGTAGAAGTATTCAGGAACAGGTTGGTGA
TACCTTGGTGGTTGTATGGTATAACATCCTGATCTTCACATACTCATCATCTAGAGTGGGAGTTTTCTTTTTCCAAA
TGGGGTTTTGGCAGAACTAGTTCCACTGTATCTTAATAAGTAATAACTCAAGAAAGGGTTCTATGGATGAAAAAATG
ATTAGGTAATATCAAGTTAAATCAAAGCGAACAGACTTCTTTCCCATAGGAGTAATCAGACCCTTATTACAGTGCAT
GCTTGGTGAATCAACAAAGTATGTGTATTTATGAAAGTATGGGGGGAAGGGATAATCTATACAGTATGCATCCCTTC
TAAAAGTTTGACCATGAAAACAATTTCTCAAGAATCTTATACAACACTACAGTATCTGGTCCAATACTATGCATAGA
ACATGCACTCAGTAAGTGTTTGTAAGATAGATAGCATAGCATATAGGCCAGGCCACTGAAGGGAAATCATCTCACCG
TGAGTTACCTGAATAGTATTCTCTAGTGCCATTAGCTCAATTCTTCACGTAGGCATAAGCCTATACATTTGCCATGC
TAACCAAGGGAATTTGTGTTACGTGAATTTTGACTCTATTCAGACATTTTTTTCTATGACTCCTCCAAGGCTGTTAT
TCTTACCTCATATTCTGGTAGAAGTTTAAGGACTTTTTTCTGGGAATATTGATTAATTAGCTAGCTAGCTAGAGACA
GAGAGAGGATAGAGATTGATTCTCTGGCAGAGCCTATTTGAATCATATTGAATCTTTTTTTTTCCTGAGACTTCCCA
CAAGGAGGATGGAGGAGAAATTTTTTAGAAATCCACCGAAGTAATCAGGGATATCTTCAGTAAAAGAAGCTATACTT
AATAAAGTCTCTATTTTAGCAGATGGCAATCAACAATAGAGGCAATAGACAATAGAGTCTATTAAAATTGCTGGGAT
CTGCTAATAACGTTTTTCTTTTCCCTGAAACAAATGCCATTAACCCTCCTTGACACTCTGTCTTCATCAACATTCTA
ATAGAATGGAAGTAACTCATAATTTTGAGGATTTTTTTCCCACACAAAACCTATAAACCACACCACGCTAGTGATTA
CTTTTAGCCTAGTTGCTAGGTTGCTGCTGGTAACAGTAAAACTTATCCTGACAGGTAGGCAATTCCAGAAGCCCAGC
CAAGCACTTGGTGTGTGTGAGTAAACCCCCATACACTTCTCATGTAGAGTAACCCTGGCCAACCCATAACTCTTAGC
AACTATTCCTGGTGGACGGACCTGGTCTACTCTAAGAAGAGGCCAAGGTTCTTTAATAGTGCAGTTGCAAGAACCAG
AATTGAAAGTCAAAGTTCTAGCAAGATTTTGCAGACTCCTTGGCAAACCAGTGGCTTGGGACTCATTCTTGACTTCA
AGCCCTTAATTGATAATGGTAGGACAGCTTGCTTGCGCTGGGTTCTGCTCCCTGGGATATGCACTGTTTGCCAAATG
AGTAGCAGGTGGACAGACATCTTTACAATTTGCTGTCCCATATTCTAAATGAACGTGACATTCTATAGGTCTGAGTT
AACCTATGAAGTCACCAATTTCAATATCAAAATATTTATGACAGAGAAAAGGATACTGAGGCACAGAGAGTCTGTGA
CTTTCCTAAGCTCAAAACACCAGTTTGTGTTAATTCTGACACAGAAATTCTTGTATTTGCTATCAGTCTCCTTTTTC
TGTGTGTGTGTGTGTTTTTACATTGCAGCATCACCTATATGATGTTAGGTTCTGTAACTTTTTGAGAATTTTCTCAC
ATACAGTGATGTGTTACTTTTTGATATTTCAAATAGTTCTAGTAAGTCTTTTCTACTTTTATTAGCGTATTAACATA
CTGGCTCTAAGAGGGCATCTCACCACATCTTTGCCATTCTTCCTGGAAAGGCAAGTTTCTCTCCATCTTCTTTTTTG
TATTCCAAAGTTTTGCCAAAGTTTGCTTTTGAAAATGGGTTACCTGGCAGAGCTTTATTATTCTAACTTTGAAAGTA
CAAGTCAGAATCAGACAGTGGCAGTTATATATGCACTACTGTGATTACTATATAATGAAAGTATCTATGGTGAAAAT
ACTGATACTGACATATATTTGCCATTTTCTAATTAAGTGCTTCAGTAAAAATTAAGCACTCACTCTTTGCCAGATAC
TGCAATAGATATTGAGCACATTGAACAAAATTCTCCATATACATATATATGAGTCCACATTCTATGAAAGTATAATG
TTTTTCTGAGAAAAGGCATAATATTCTATTAATATCAGCTTTTGCTTCTTCCACCATATATTGAAAGAATTCTGAAT
ACTGTTATAATTTAATGGGAGAATCTAGAGAATTCTGTATTTGCTTTCACTGCATTGATGAACTAAGATTTTTAAAA
AATGTATTCTTCATAGAACTACTTTTCCATATTTACCTAATATTATTCTTATATCATTTGAGCACATATTTCACTAA
CAAAACAAATGTGCAATGTTATTAGTTCTAACATCAAAATTACACTGATACTTTAATTTTTATCCTATTATTTTTCA
TGCAGATTAAAATAATTATAGCTACATCACATGTTGCAAGTTTTAAGAGCTACTTTAAAAATATATGCTTCAGGAAA
GACATGATTAGATGGGGAAATGGATGATGTTCATATTTTCAAATGAAAAGTTTTAAAAAAGTGCCTATCACAAACAC
TAAATTTTTACATAAATTATCAACTACTAATATATCTACAAGAAATACCATTTTTCCCTACAAAAACTCTTAACAAT
AATTGTTAAACTTAGTCCTGGAACCTGCTAATATAATCGGACAAATGTTGTCAATAAGAAGGTGAAAAAGAAAGCAT
ATATAGTTTATCAAACTATAAAATATAGTTTATCAAAACCAATTTTTCCTATTGACATTTATTCAGGAAGGAAAATG
GATGAGTGAAATGAACAATGGTCTCTAAGAGAGGTGGGAGATAGCAATAAATTCAGACCACGTTTCCTGTCATTACA
GCAGGGAAGTAAAAGAGCTACAGTCAACTCTCGAAAGTACTTGGGGGAACTAATGATTCCCTGTAGACCTGTGATGT
TTTTGAAATTTAATTCAACAATTTGATATACACCGCAAAGCGAACAGATAGTCAGATCAAAATCGGAAGAACGATTG
TCTGAATGGCATCCATTTTTCCTAGATGTGCTGTCCCATCCTGTGTCAATTAAACTTTCAGGTGATCTTCAAACATA
TTTCCAAGTAAAAGGTATTGCAGTTATCCTATAAACTGGCCTCTTCCCCAGCACTGCTTTTGCTGTGGTCAACTTTA
TTTCTTTGGGCTCACAAAACTGATAGAGCAAAATAAGGAAAACGGAACATTGGATTAAAATAAATTAATTCCCATTC
TGTGACTCACTAAAAAAAAAATGATAACTATGCTTCTGTGAGCATTAATAAGGAAATGAATAAGGAAATGACCAAAT
TGTTCAGTGGACAACTTGTATGGGATTTTTAAGTATTGTGTCATCATCAATGTTGTCAATTAGCATATACTTTGAAA
TCAACTAAAGCAAATCAGTTGACTAATCATTAAGGGTCTTTTTAAATGACAACATCTAAACAGCAAATGTTTTATTT
TGGAAAATCATGACAGCACAAGAATGAGCCAGATGTTTTACAACATGATATCCATAATTTAAAGTATGTAGTAGTCA
CTCAAAGGATTTCTATTTCAGTTTCCTTATGATTTGGCTAAGCTAGAATTTGGAAAAACACTTTAAGGTAATGTGAG
AAACAGCAAAATTCAACATGTGGATTTTTTCACTAAAGCTTATTTCTGATTATTTTTTACAAACTTTACTAGGTATA
TGTTAACTTCATGACACTTATAGCAGTGGACCGTAGTTTTAATAAAATGTGAATGTATACTCTTTTCTCAATAATAT
TAAAGAATGTTGACTTTCGTGAGGATATTTTTATTTTTCTCAACATTAAGAACTGTCAAAGATTTAATTCTACAACA
GAAGACGTGAATTTTGTTTTCTAAAGGAGAACAGAATCTATAGAAGAAGTGTTGCTCATAGTACTCAGATTGTTGAC
CAATCTTAAAGGAGAAACCGTCAATTAATTTACCGAGAAGTAATAACATTATCTTTTTCTTCAATTATGCACATCCA
CAAAGATTTGGGGCAAAATCCACTTAAATGATATTATACATAATAGATGAGTATTCATATGTTGTAAGAGTCCTGGC
TTCTTTCCTGCAAAATGATTAAAACTTGGATCAGAAACCAATTAAAAATCCATTCTAATTCCCAAATGTATGTAACT
GTACTATAAGAAAAATAAATATTTCTTCTTGAGGGATATCCATTAGTTAAGGATATTCATAACATGGTGTCTTGTAG
GAAATGTTAATCTTTGGGTGAATAGGGATGTTTGGGAATAACAAGACTCAAAGAGATGTTGCACTTACTCACTTTTC
TCTGAGTTGTTATTTCTGTCATTTCCCCAGTGCGCCTGTCCTCAACTTTGCCTCTCTCCTTATTCCTTTTTTTTTTT
TTTTTTTTTTGAGACGGAGTCTCGCTCTCTTGCCCAGGCTGTAGTGCAGTGGTGCGATCTTGGCTCACTGCAACCTC
TCCCTCCTGGGTTCAAGCAATTCTCTGTCTCAGCCTCCCGAGTGGCTGGGATTACAGGCACCCACCACCACGCCCTG
CTAATTTTTTTTGTATTTTTAGTAGAGACAGGGTTTCACCATCTTGGCCAGGCTGGTCTTGAACTCCTGACCTCGTG
ATCCACCCACCTTGGCCTCCCAAAGTGCTGGGATTACAGGCGTGAGCCACCATGGCCGATCCCTCCTTATTTCTTTT
TATCTCTACCTCTGCCTCAATGGTATTTCTCTATTACTGTTAGCATTTGCTTTCTGTGAGCTCTTGCACACTGTCAG
CTTATATACATGTTCCTGTTCACATGTTTTCCTGTCCCCAGTGGTTACAACATGTCTTCTATCTCAGCCCACTCTAG
AATTGTCTTACTTTTCCAGGTCTCCTGCTCCTCAGTATTTTTCCCACTTTTCTAGATTCATGTTTTCCCATCTGCAT
ATTTCTCTTCCATGTCTGCACTGTCATCCGCTTAGAAGACAGCGCATAAGGACACTGTTATCTGAGCAAATCTTCAG
CACAGCCACCATGAAGCATGGTTACCTTGTCACTTTCCATTTTTCCCATAGTGTGTGCAAACTGCCCTGATCTGCAT
AGAAAGGTATCATAATTGAGGAAACAAAATGCACAAAAATGTCCTTGGTTATTCCACCCCTCAGAAATATAGGAGAG
AAGTAATTTACAGAATTACACAGAATAACGCTATGTCACATGGACATGGAGTTATCGGGTTAGCATATAATTGGAAA
ATATTTCCTAGGACCTTGACATTTACTCACTTTTTGTTTTCAAATTACATGTCCCTATCTATTAGTTGCAAATTATT
TTAATGCACCGTTTACCAAAGAAAGGCTGTTTCTTCTGAAAGCTTTCATTTGACAAGTAACTTGTAAAAATATTCAC
ATTGTGTATCTGTTTTCCCCTTCTAGTCCAAACTCTAGTTATCTTAAACTTTGCGCAGTTATAAAAAATCATAACAA
AAAAAGCTTCCTCGTTGTCATTCTTGTCAAAACAGGTTTACCAGACTTAGGTAAACTTAAAATAGTTAGTGTAAAAG
TTAAAAAGCTGATTTGCTCCTTCCAGCGTGTTTGTTGCCTTTTTGCCACAGCAAAATTGTAAATGTAAACGTATTCC
CTAGGAGATGAGCTGGGCTGCAATTTTCAGCTAATTGGGAGAAGCAGCCCTGAGTTGAGCACTGTCAGGCTGATTTG
AGTCTTAAGATATGATGATGATTATTGTGTCAAATGTAATCAAGAACGTGGGCTCTGAACTGACTCAAGGGCTGGCT
GTTTTTAATTCAGGTTCGTATATGAAGTAGACCTCCGGTTCACCGATAGTCACAGCTGGTTGTAGAAGAGAGCAATT
TTTAAAATGCTATTTCATTCTCTATGGAGCTCTAGGGATCAGAGATTGGATGCACAGGGAGGGGACACATCCTCATT
CTCTCCTGAAAAATTCTATTAATTTTCAGTATAATAAACTTTCTCTTGAGATTCCCCAGTGGCTCTGTATCGGTGGT
TTTCAAACTTCTCAGACCCAATGCCACCCCTCTTTTCTTTTTTAAATAACAAATACTTTGTAATACCTTCTTTACGA
TTATAAGCCAAAATATGTAGACAACATACCCTACTTATACAGGCAATAGTTTAAATGATGCCGTAACTCTATTTTAA
AGAGAAATAAGAGTCATTTATAATAAAATAATATGTGTTGTAGTATGCAGTTATTCAGGCAGGATCACACTGGAACA
CAAGTGAAGTTTTTAGATCACGAGACTATCAATGCAGTATAAACAAATGCAGAATGACACCATTGTGTTGTATGGAG
ACTCAAATACCATGAGGGGCATTGGTCATCCATAGCGTAATTTTCCAAAATGCTGAACAACTCTTGGCAAAATTCCT
AACACCATGAAATAAATTTTTTCTTGGATCGTTATGGCAGTTAGTTGCATGGCTGAAAAATTCAATGTCTTAAAATC
ATGAGGAAAATATCTTATGTTTACGTGTAAAATTGAGTTACGTTCCAGGTTTAGGTGTTTATAAACAGGGTTTCCAC
ATACATGCATGTCCAGTGGGATATTCCAAAGTGCTGTCAGACTTGGGAGAGTTCTTTGTTGTATAAGAAGTCTACCA
TCTTCATTCCCTCTCCACAGAATGCTATTATAGTAACACTCTTCAATCACTGTGATAGTCAAATGTCCTCCCTCAAT
TTCTAGGATGCCTCTTTTTTTTGTGGTCTGTATAATTTGGTTAAATATCTTTCCAGACAAATACTGATTTGTGAATT
AATGAAATAGCAGTATTTTCGGAGCACCTAACCTATTTCTGAGTGATACAGTTGCCATTTTTACAAGACTAAATGAA
ATTACCATTTCAGACCTGCCAGATTGTCTAGCCCAGTCTTTTACAATTCTGTGATTATCACTGCAATTATAATCTAT
TTTCACCACTTGAATGGCATGATCTCTATAAAAGGGTGGTGATAACACTCATCTATTCTCCTTCCCCTCACATAGCT
ATATCAATCGCCCCCTAACCAGTTGTTGATAAATGCAGITGAATTTTATGTAAAAATTATAAGAGATATTATTGTAG
CTGTCCAAGACATTTAAAATGCTAAATGCAACTTACGTGGAGGCTATAAGAGAAATATGAACCCATTTATTGAAGAG
ATTAGCTAATTTAGTAAAACAACACAGATATACCTGCATACAGGGATAAATCCCTATTGTCTAAATTATTGAGATAA
AATAATGTTTTACAATGAAAAACTTTTAGACAAGTAGGTAAGTAAAATGCAGCAGTCTATTTGCATTTCATCTGGGC
ATTTGACAAAGTCTTTCGTTATACTCTTGTGAATAAGTTGGAGAAATACTGGCTAGATGCAAGATAAATTGGATGGC
TTAGAAGCCACTTCATGATTTTACGCAAAGGATGTCGATTAATAGACCAGTGTCAGGTGGTGATGGAAGATCTCTGG
TGCTATGTCACAAGCTTCTGTTCTCAACCCTGACACACTGGATGTTTTTGACAGAACATGAGTAGAACTACAGAGAG
GAGGCCCATCAAACTTATGGGTGATAAAAAGCAGGGAGGGCAGGAGTATTTTGGGTGACAGAAGCCAAATGGGTGTC
TGGACAGGATGCGTTTTAAGGCACTTTTGGTACTTGATGTCTGAAGACCAGGATCAAACTTATAGGCAATCTGAACA
TTTGCCAAAATAACAGGTTAATTTTGACAGAAGTTATTATTTGTATGCTGTCTATTTCTTTAATACACCTAGAAAGT
ATTGAAATAACATTTTTTGCAGACACTCATTTTGAAAATTCAGAAAAAAAATTGTTAACTTTCGTGGAAGAGTAACA
GAAACTCAGTCATTGACAGCTAAATACAATGTGTTGCCCAGTAAAATAGTCCACCCCTTCACTTTCATGGCTAATAT
AAAATTTGATGAAAGATACAAATTCCAAAGATTGAATATCTGTACATTTGCAAAGCAAAACACAATTTTGGGCACAG
AATTGCTCATTCTCATTTTTAAACATCTTGGTTATAACTGAACAATAGTTTTTTATAACAAAGATAATATTTTCAAA
TTATTATGAGGTTCAACTGAAATAATTTATGTGAAAGCAATGTCTAAACTCTAAAATTCTATATAAATATAAATTAT
TATTCAATAAATTCACATCAAGAAAATTTTAAGTTTTTTAAGAACAAGAGCCTATGGCCTTGTTTTTAGAAGCTGTA
TACCTTATCGGTAGTAGGTTTATTGACTTTAATTAAATTTATTGAGTATCTATTAAATTGCCAGGAACTGTGGTGTG
AATCTTTGCCCTCAAATAATTTACAGTAAGTIGTGGTTGATGAATGGTGATGACGATGATGAATATCCAGACTATAG
TAAGTGGTATATTCATAAGTCAGAGGATTCTTAAAACCAGATGCACCCTCAGATTCATTCCTTTCATGTTGTACTTC
TAATTGAAAAAAATAAATCCTAAATTATGACTGTTCTTTATAAATTTTAATTGATCTTATAAAAGGCCATCAATACA
TTTCAAAGTATCTAGGTCTTTTAAATGCAATTTTTCACCCTGGTAATTAAAAGTACGAAAGCAAGAAACTTTAAATC
TTTATTTTGATAAGTTTTAATTAGCTCAAGCTACTTGTAATCCCACATCTTGTCTTGTAAATCATATCTGAGCCATT
AAAATAGGTTTACAATTAGAAGGGCAATTCTTTTAGAATCTACTTAAACTAAGTCACTTCGACAAATTAATTCATCG
TTCAGTTGGTTTTATTAAAATGTATTTATTTCACTGTAAAATGTCTAGTAAAGCAATGTATGAAGTATTTTATTTTC
ATGTTAGAAATTTTATGTAAAAGATATCCCAAAATACATAGACATTCAGATACTCTCTGTATCATTAACCAACATTT
ACTAACTTATCATTTAGAGAAGGCCAAAATTGTATGTACTATAACTTTGTATAATTTCATAAGAATTAAAATATTCG
ATTAATGCCTGTAATGCCTTCTTTCTAAATCAAATCCTCAAGCTTACCTCGAGTTCAAAGTTCAGTATTTATTGTAA
CACATCTCATAGATGACGGATGAAGATGGTAAGCAAAGGAATAATAATTTCTTTTCTCTTTTCACACATATATACAC
ACATACCCCATAATCCTAATTCATATAATAATAACAGAAAACAAAGGGCTTTTGAGAATAGTGACATATTAATATCC
ATTATATTTACTTCACAGGGAGACTGGCAAGTCTACCTTGAGAGGTAATGTCTTATAGTACAGTGGACTAGATTGTT
TCAAGATTTGTCATTTATTTTGGCAACTCACCCAGCTTCCCTGAAAGTTAAGTTCCTCATCTATAAACTGTTCATGA
TAATTACAACCTGCCTCATTAGCCTCATCAAGCTATTTAAAATATGAAAGGAGGTGCTATCTGTGGATCCTGTCAAA
GGAGCTTGAAAACTGCAGAACATTATTTTAGTGTAAAATACTATAACAATACATGTTGAATATAAAATGGCTTTTTC
TTAACTTTTATTTTAAGTTCAGGAGCACGTGTGCAGGTTTGTTATATAGGTAAACTCATGTCATGGGGGTTTGTTGT
ACCGATTATTTTGTTACCCAGGTATTAAGCGTAGTACACATTAGATATTTTTCTTGATCCTCTCCCTCCTCCCACCC
TCCCCACTCCAGTAGGCTTCCACGTCTGTTGTTCCTCTCTGTGTCCATGTGTTCTCATCATTTAGCTCCCACTAATA
AGTGAGAACATGCAGTATTTGGTTTTCTGTTCCTGCATTAGTTTGCTAAGGACAATGGCCTGCAGCTCCATCCATGA
TCTCTGAAGAATCTCCACACTGGTTTTCACAATGACTGAAATAACATACACTATAACCAACAGTTTATAAGCAATGC
TTTTTCTCCAGAACCTGTTATTTTTGACTATTTAGTGATAGCCATTCTGACTGGTATGTGATGGTATCTCCTTGTGG
TTTTGATTTGCATTTCTCCAATGATCAGTGATGTTGAGCTTTTTTTCATATGCTTGTTGGTCGCATGTATGTTTTCT
TTTAAAAAGTGTCTGTTCATGTGCTTTGCTAAAAGGGCCCTTTCAAATGTGTATTATTAACCACAAGAGAGTACTGA
GTAAGAGACTAGGTAATAAAAGTCACAAATATTTCGATATCATAATTCAGAATTTAGATCAGCGGTTATGAAATTGT
TCGTATTTCCAAATTCCACTGACAGGACTCTACTATAAGTTTATTTCATCTGTTGATATGTTTTTAGCCACTTCTTT
CTTTTAAAGTGAATCTGTTGTGTGTTTGCCATTTGATATTAGAAAACTGAACCTGCCTGCTTTGCTGTCTTCTGAAT
ATTATGTATCAACAACTAACAAGCTACAGTTAGTTGTTTTGTTCTGTTTTTCTCTAAGTTATTGTGGATGAGGATAT
ATATAACTGCACAGTCTTATCAGGTTTGTAAGAGATGATCTTAGGCTCATCTTTTAAATTGGTTTTTATACTATTTT
AAACAAATCCTTTTAGGAGAGAAGAAAAGCTGCTTAGTCTATCAACATTAGGAAATATATCTTTAAAGAGTTTATCA
CTGCAAGTAACCAAAGCCAACTTAAAAATTCGCATTATACAAATCATTGAGAATTTATTTAGAACAGAAATGTGTCC
AACTATAGGTCAACACCAATTTTAAGTGTGTAATTATCTGGGAAGTAGTGTTAACTGCATTTTTTTCTAAAGATCCC
TTACAGTTGTATAAATGCCCAAAAGGATATTTTGAGTCTCTGTATATTAACCAAACCAAATGTAATTCATTACTCCC
AACATTATATTTCAACCTCTCCAAATAGTACCTTTTCGTATTGTATCAGCAGAAAAATATAAAATGCAGATCTTAAA
GAGTATCAATCTCTTTAAAAATTCAAGAAAGAAAAAAATATGTGTGTATAGAGACGTGTATTTCATCTGCTCATAAC
ACTGTGTACATTTCTTTATCAACTAATTTTTTTCAGTGATTTATGAGTTGAAATACAAATCAAATGAAACGGGTAAT
GCAAAGTAAAGTAGAAAACACATTTTCTACTGCTGTCTCCTAATGCAGGTCTTTTCAGGAAAGTACTAATGGTTTTA
GGGAAAGTGTATAATTATGGTTGTTTCCCTAATGATAAATTCGCAAATCTCTATTTTAAAAACATTCATAAGGTTAA
AAAAATGAGAGATGAAATGTGTCTTTCAAAATTCCTTACGTGATTGATAATGCCTATACTCTCTTACTATCTAAAGT
CTAGGTGATATGTATATTTTTTTTAAAAAATAAAATGTCTGTATCAGTGAAGGAAGTTTACACAGATAGCTTCAAAG
CTGTGGTTTATCTTTGGAGGATTAATCTATTTCTCATGCCAGTGTGTTGCTACTGCACATGTTAAAAAGTCATCCTG
TGGTGTCTGGGGTGACAAAAGATGGGAATGAGTTTTCTGAGAACTAATCAGCAATACTTTGGGAACATTTAGGTCAT
GGTTTCCAATTAACTCTGGAGAGTTTGAGTAATTTAGTACCAGACCTCAAGAGAGAGGGGATGAAAACCTCGTTAAT
TCATATGTTGGTGAACGGCAAACCAGCAAATTTGCATTAAAAATGGATTTTTATTTTAAAGCAAAGAGCAGCCAGAT
CTTTTCTGCAATAGTTTGGGTAGGAGAATATCTTTGTATGTATGTGTTCCCTTATGTGTAGGTATTTGTATGTTTCA
ACGACCCTGCATATGGCAATAACAGAAAATTAAATTTGTGCTCTAAAATGAAGACCAGGATTCAGTGACATAATCTT
CCTTGTGCCTTTCTTTCTTTTAGTACAATGAATATATCAGAGAGGAGTGTATTCCAATATCTGTCTTCAGAGTTACA
AAAACTTCTTTTCTAGAATGCAAGACTTGGGCTATACCCCCAGCTCTGCCACTTAACTTGTATACAACCTTGGGAAC
ATCATTACAATTCTCTCAGAATCAATCTCTCCAGCCCTAAAATGAAACCAGCAAAAGCCTGTACTGTATATCTAAAA
GGTTTTTTATTTTTATGAAAATTAGTTAGGCAAACTTTTGTTAAGCATCCATCACTCTATTTTGAGATAAAGCCTTG
CTGGATGATCTCCACCTCTTTTGATGGAAAGAGTAAAACATGTTTAAGATACATTTATCACTTGTTTGGCAAATTGA
GATAGAAGTTTATGAAAGCAGATTGATATATGTTACATTTGAGCTACTGGGAAGGACTCCAGATGGTTTATAGCCTT
AATTACATTGTAACTCTAGTTAAATGTTTACCTATCTGTACCCTCTGTTAAACTTGAATATGTTAAATACCAAAGTC
CATGTATTATTGGATTTTCTGTCACCATCATCAGGCACAGATCCTGGTACACAATAGGTACGGAATGGATGCATGGA
TGAATTATTGAATTAGATGTTGGTAGGCATGTGGAAATAAGAATGAGGTTCAGAATTAAAGATAATCTGTATCGAGT
GTAAAGCCATTGGCAGAGAATGAAATATCCAGCTGAGTATACATAGAAAAAGAAGGTAGGTAGAAAAATGGAAAATA
TCTTATGAAGTGATGATAGAATAACTCTGAATATGTTTGAAAACATATAAAGAGTTATGTGGATGTTAGCTTTAAAA
ATTATCTTCCATGCTGTACATTAGATCTGCCATTCTTCATGCTGTGGATGAAAAGCAAGCATCAGAAGTTAAATTAA
AATGATGTCATATATTCCTCGCCTTACAGTTTCATAACAGAGGAGAAAAGAGAAACATTCTCTCATTGCCACCACCC
TTCTCCAGTCATATTTCTAGGTAGATGTTGCCCAAAAACAGATAAAACCACAGAGTTGGTTTTGCTAGGAATGGACT
ACTAATCCAGGCAATGTTGACAGCTTTTGCTTCTCATTAGTGCACGTTACTAATAGAATTGCTAGAGATTAAAAGGA
ATCCTTTCTACAAAGTGCTGTATATCCATAGGTGACAAAATTCTAGCTTCCCCTCACAAGTACAATATAAAGTTATG
TTTTAAAATCAAAATGCAATTTACTAGCAAACTAGTAGGAACTGTTATGGTTACAGGAAATTTGAATTTCAGATTAA
CTCTGGTTCTATGAGTAGCGGTTGATATGGCAAGAATCATTTTGATCTTACATCCAGGTGCTACTAAGGTCTCTCTG
ACCTATATCTCACCAAAAAAAGGAACAAAATAATGATCCTTTAATCTTTCTCCTAAAATATCATAGGAAATGATAGT
GGCTAAATTGCAAATAAACTAGGAAGGAAAGATTCAGAGTATTTTATGTGATTACTCTATAACAATGCCAGGCCATA
GTGAAAGTGTTATTTAGCAGAAGACTGAGTTCTTTGAATGTTCCTAATTTATCACATTTTAAAAATAACCTGGGCAA
AATAACCTTTCATATCAGATTGAGCCTTTTTCTAAAAATACTCAATATGTTTCTGTAATTATACCTACACACTTACA
ATTCCACAGTATAATGCACCGATAAAGTATTTTTCATCCATATATCTAATAGTAGAATGGTGTGTATACAATAATTA
AGCTCTTTAGGCTTACCCCGGAAAGCAACAAGTTTCCCTTCCTTTTTCCTTTTTATGTATTATGTTGGCCATAAGAA
ATTGATGATATTCAACTCAATGCAGTCTTAGAGATTTATTCAGAAATACCATGGTGTGTGTGTGTGGCGGGAGTAGG
GTTCTAATGACAGGTCAGAACTTACTTATTTGATTTCTTCATTGATAATCAGGTCTTAAAAAGAAAATGGGTATGCT
GAAAACATGCCTTCTGTGATTCTTTACCTTCATGTGCAGTTGTCTCTGGATAAACACTTTCTTTGGCACGTATAGGG
TTGCACTAAGCTTTATAGCTCCAACACTCCGCCCCTTCAGTAGATTCTTGCTTGTAACTGATGATAATGCAAACCTG
TATTATCTATAGGTCTCCTTAAAGGGCAACCAAAAGTTCAGTAGCAATTCAGGCACAATTACTGCATGTGAGAATCC
TCCATCTTGTTCCCTTTGGAGACCACATATATTTCTTAGGCAAGTATATTTTTAAAATCCTTGTTCAGCATGACAAT
TCAGGAGGTCAAGTTCTCCCAGAAAGCAGATTCTGAGAAAGTGATTAGCATGAAGGAATTTTATTGGAGAGTGCTCT
CAGGATTAACACCTGTGAGCGGAGGAAAGGAAAGGGAGCAGGATTGGGCAGAAGGAGAAGCTGGGCTACCATACAGT
CACAACTACAACACAATCAACCCTCCGCCTCTCCTTCCTAGCCTTCCCCAGGAGGATCTCTGAAGTCTGAAGGTAGA
ATAGCCCTTCAGAATTGTCCTGAGTTGCAGCAAGGGACCCAGGATTTTATACCCCACAACTCTCCCATCAACCAATA
CGTGCAGCCCGTCTCGGGGACATAGTGGGTAACTTTGGGCTAGGCACCTCTCTTTAGCTGAGTCCAGCTCTCAGACA
GGAATAACAGCTGAGGACTGTCAGCCAGTAGCACTACCAGCAGCTGGGGTCAGAAGTATTTCAGTCCTGAAAAGGGG
TCCGGGCAGCCCAGCTTAGCATCTACTATGCCAGTCGTTCTCAAATCTGGTTCCTGGCAACTGTGATTCTCAAGCTT
TAGCATATATTGGAAGGCTTGTTAAAACACAGCTTGCCGGATTTTACCCACAGAGTCTCTGATTCAGTAGAGCTAGG
CTGAGGCCTGGGAATTTGCATTTCTAATAACTTCTCAGACGTTGCTGGTGCTGCTGGTCCATGGACTATGAGAACAC
TGTTTCATGCTGCCCTTATTTACATACTGAGAATGGTACACAGTGCTCTTATGAATAGAATGAAAACCTTTTGAAAT
CACATTATTCCTTACTCCATCAAATTCTCAGCTATTTTTGTGCACCATAAAGCTGGAATAGCTGATTATAAAACTTT
GTTATGTAAAAAAGTACTTAACCAATACAGTAGATTCTGTTTGCAAAGCATTATTACAGTTTCTAATATCTGGTCAT
TGTTACTTGTAAAATTCAGCCAAATTTTCTCCAGGGCCTGTAGTTTGATAACTTGGACAAAGGAATTTAAAAAAAAA
TCTAATTCAAGACCTTTGGTTTTTTTTCTGAACATATCTTTTTTTTCTTTATGATTCTTATTTTTACATTTTACTTA
TCATATAAGCCACTTAAACCCATATGGTTCCGGAAAATTTAAAACTATATGATACATTTAGAGCATGTTGAATGCAC
AGATATGGAAATTAAGTATTCTTGACTCATTCTAGACTAGACCTGGCACAATTAAAATTTAGGGATTCAACGTACAC
ACACATAGATTCCGAGAGAAATGTTGAAGCCGTAAAACCCCCACACAAGCAGGAAACAACAGTCTTACCTATTATTC
AAGAGGCACGTAAAGGAGCTCATTTGAGGAGATTTTCTGCTGTTATTGCCATCGAATTTTTAACGTATTTTCCAAAT
TAGAAAATATTCAGCCTGATGTTGTCAATATTTCAGACCACAAGGGTATCATTTAGGAAAATGGTTTCTTACTGTCC
TGAAAGAGTTACTGTTCTTCCCTAAGGGCCTAATTTACAAAGCAGCAAACTTGCTGGTAGGATTTGGCTGAAAATCA
CATTGTCTCGGTAGAACTCTTTCATCTGATTTATGTGCATTGCATTTTGCAAATAACTCTTGGAAAGTTATTTACTA
GTTACTTTCTCTGGAAGCAGAGGGTAAGCGGCATTTCTAGTTTAAGGATAGAGGAGCTAAGATGCATCAAGCGCAGC
TCATCATGAAGCTGATGCTGATAAAATGCACAATATTACATTCTCTAAGTTTCACTCTGCCATGGGAGAATTTCATA
TTTTTAAATTTTGTTTGAAATTGGACTACATTAGAAAATATGTCAAATGTCTAACCCTGCATTTATATTCTGGAATG
TGACAGCTTATTTCTGTTCCAAATTTTGCACTGGAGATGGAGTAAGTCTTAATGCAAACTGCATGAAACTGCCACTT
TTATAGGTCACACCCAGTCAATTGTCAGCAGTTACACATGGTTCAAACTGTAAGGTGTATGCCCAATTGTAGCATTG
AGATTCGTGGAGTTGTTGCAGTGGTTCTGAATTTTTCAAGCATGATACATAAAAAGATAAATGACTCTTTTGATATT
TCTCCTTGCATTGATAGTTTGCCTGAAAACTAGATAAGCAGGGAGCCGGCAGTCCACGTTAGCCCTTGAACTACATG
AGGTTTAATTTATTTGCCCAACCAGAACCCTACACTACCTTTCAGCTGTGCAGTATTAAAGTTTATTTAGGAGTTGA
TAAATAGCTTAGTGCAATGCTTCCTTTTTTCCAGTAGCTACATCCTCATAAACCTATTCTACCCTCCACCAGTTAAT
GCAGACAGAAGATTTTTATCCAGTATGAGCACTGAAACTCCACTGTGGAAGACTGTGTGCTCAGCAAAAACCTCACC
CATGATGAATAAACAGCTCTTCCGGGGGCTTTGCTGCCGCTGGCTCGGCAGGAGTTGTTTATTGCCTGGTTTGCACA
TCCCATGATAAAGTTGCTGCTGAAATAAATTGCAGTTTTGCATAATTATTGACAATCACATCTTAACAAGCAATGTG
TATCATATTCAAGTGTTCAATTTTTTAAAATCCATTTTTAGCTTATGTTTAATCCCAGAAAGTGTTTGTGTAGTAAT
AGAAGGCAAATAAGACATTTAAATAGAGTACTAATTTCCTCATTGCAGACAAAGTTTACCTGAATCTTTTTCCATAG
GACTGTTACTGCCTAAGGCAATTTTCCTTTCTAAGCTATTATTATATAGATATTTGCTGAGGGCATATGTGTGTGTA
TCCACAATACATGCATTTTATATATATATATATATATATATATATGATCAAAAATATGAATACATTTTTAGAGTTTT
TGTCATGAAAGAGTTTGTTTCATCTTTTTAAAATATTACAGGAATGGGGAAATGGGATATGGGTAGAAGGAACTAAT
GTTTTTGAGTAACTGTAATGTATAACTGTATAACGTGGGGCACTCAACTTCACAGGAATTTTTTATTTTAATTCTCA
TCACAGCAATAGATATTGCAGATGAGAAACTGAGAATCAGAGAGGGAACTTGCCATATCACGTAAGTGGTAAAGAAC
ACTGGGAATTGAACTCAGATCTGCCTAGTTTTTAAAACTCTACTCTTTTTCATTACACATAACATTTTTATTTTGGA
AAATGTTCTCAGTTGTATGATCAAGTAGTTAAATATGAAACTAACACAATAATTATAACTGATGTCATGCAAAATGA
TAGTTTGCACAAAATGATAGTTTCTATGAAATGTTATTTCTTTACTTGTTAAGTCTTTCTTCCTTTGCCCTCCAATC
CCCTTCTTTTTGTCTTTTCCTCTAGTCTTTTCCTTTTGATTCTAGGTTTGTATTTTCTTGACTTTTCTCCTTGCATA
TCAAATCCTTGTTTTCTGCCTCAGAGCAGCATCAAAGACAAGCATGGTACAGGGATTTTAGGGTTTTAACTATAAAG
GTTTGTCTCAAATTTGGCAGTATATTAAAAATAAGCTTTCAAAATTGACCAACAAAAACTACAAAATTGAAAAAAAG
GTACTTTGAACTTTCACATGTTCAAATATATGTATATATATTTCACATATATATATGAAACCTCCTCTGTGGAGAGG
GGTTTATAGAAATCTGTAATTGTCATTCTTGCATGCCTTCCCCCATACAAACGCCTTTAAGTTAAATAAAAATGAAA
GTAAATAGACTGCACAATATTATAGTTGTTGCTTAAAGGAAGAGCTGTAGCAACAACTCACCCCATTGTTGGTATAT
TACAATTTAGTTCCTCCATCTTTCTCTTTTTATGGAGTTCACTAGGTGCACCATTCTGATATTTAATAATTGCATCT
GAACATTTGGTCCTTTGCAG

Homo sapiens dystrophin (DMD), intron 54 target sequence 1 (nucleotide positions 1686621-1686670 of NCBI Reference Sequence: NG_012232.1)

(SEQ ID NO: 2146)
GTATGAATTACATTATTTCTAAAACTACTGTTGGCTGTAATAATGGGGTG

Homo sapiens dystrophin (DMD), intron 54 target sequence 2 (nucleotide positions 1686641-1686695 of NCBI Reference Sequence: NG_012232.1)

(SEQ ID NO: 2147)
AAAACTACTGTTGGCTGTAATAATGGGGTGGTGAAACTGGATGGACCAT
GAGGAT

Homo sapiens dystrophin (DMD), intron 54 target sequence 3 (nucleotide positions 1686710-1686754 of NCBI Reference Sequence: NG_012232.1)

(SEQ ID NO: 2148)
CAGCTAAACTGGAGCTTGGGAGGGTTCAAGACGATAAATACCAAC

Homo sapiens dystrophin (DMD), intron 54 target sequence 4 (nucleotide positions 1716672-1716711 of NCBI Reference Sequence: NG_012232.1)

(SEQ ID NO: 2149)
TTCTCTTTTTATGGAGTTCACTAGGTGCACCATTCTGATA

Homo sapiens dystrophin (DMD), intron 54 target sequence 5 (nucleotide positions 1716498-1716747 of NCBI Reference Sequence: NG_012232.1)

(SEQ ID NO: 2150)
GTTTATAGAAATCTGTAATTGTCATTCTTGCATGCCTTCCCCCATACAA
ACGCCTTTAAGTTAAATAAAAATGAAAGTAAATAGACTGCACAATATTA
TAGTTGTTGCTTAAAGGAAGAGCTGTAGCAACAACTCACCCCATTGTTG
GTATATTACAATTTAGTTCCTCCATCTTTCTCTTTTTATGGAGTTCACT
AGGTGCACCATTCTGATATTTAATAATTGCATCTGAACATTTGGTCCTT
TGCAG

Homo sapiens dystrophin (DMD) intron 54/exon 55 junction (nucleotide positions 1716718-1716777 of NCBI Reference Sequence: NG_012232.1)

(SEQ ID NO: 2151)
AATTGCATCTGAACATTTGGTCCTTTGCAGGGTGAGTGAGCGAGAGGCT
GCTTTGGAAGA

Homo sapiens dystrophin (DMD), transcript variant Dp427m, exon 55 (nucleotide positions 8272-8461 of NCBI Reference Sequence: NM_004006.2; nucleotide positions 1716748-1716937 of NCBI Reference Sequence: NG_012232.1)

(SEQ ID NO: 2152)
GGTGAGTGAGCGAGAGGCTGCTTTGGAAGAAACTCATAGATTACTGCAA
CAGTTCCCCCTGGACCTGGAAAAGTTTCTTGCCTGGCTTACAGAAGCTG
AAACAACTGCCAATGTCCTACAGGATGCTACCCGTAAGGAAAGGCTCCT
AGAAGACTCCAAGGGAGTAAAAGAGCTGATGAAACAATGGCAA

Homo sapiens dystrophin (DMD), exon 55 target sequence 1 (nucleotide positions 1716757-1716809 of NCBI Reference Sequence: NG_012232.1)

(SEQ ID NO: 2153)
GCGAGAGGCTGCTTTGGAAGAAACTCATAGATTACTGCAACAGTTCCCC
CTGG

Homo sapiens dystrophin (DMD), exon 55 target sequence 2 (nucleotide positions 1716821-1716887 of NCBI Reference Sequence: NG_012232.1)

(SEQ ID NO: 2154)
TTTCTTGCCTGGCTTACAGAAGCTGAAACAACTGCCAATGTCCTACAGG
ATGCTACCCGTAAGGAAA

Homo sapiens dystrophin (DMD), exon 55 target sequence 3 (nucleotide positions 1716891-1716937 of NCBI Reference Sequence: NG_012232.1)

(SEQ ID NO: 2155)
TCCTAGAAGACTCCAAGGGAGTAAAAGAGCTGATGAAACAATGGCAA

Homo sapiens dystrophin (DMD) exon 55/intron 55 junction (nucleotide positions 1716908-1716967 of NCBI Reference Sequence: NG_012232.1)

(SEQ ID NO: 2156)
GGAGTAAAAGAGCTGATGAAACAATGGCAAGTAAGTCAGGCATTTCCGC
TTTAGCACTCT

Homo sapiens dystrophin (DMD), intron 55 (nucleotide positions 1716938-1837156 of NCBI Reference Sequence: NG_012232.1)

(SEQ ID NO: 2157)
GTAAGTCAGGCATTTCCGCTTTAGCACTCTTGTGGATCCAATTGAACAATTCTCAGCATTTGTACTTGTA
ACTGACAAGCCAGGGACAAAACAAAATAGTTGCTTTTATACAGCCTGATGTATTTCGGTATTTGGACAAG
GAGGAGAGAGGCAGAGGGAGAAGGAAACATCATTTATAATTCCACTTAACACCCTCGTCTTAGAAAAAGT
ACATGCTCTGACCAGGAAAACATTTGCATATAAAACCAGAGCTTCGGTCAAGGAGAAACTTTGCTCAGAG
AAATAACTTAGGGATTGGTTTATTAAATTTTAAAAGTTGACATTTTTGAGTGTTTATTTAATATTTTACA
GGGAAAGCATCTGTATGAATTGTCTGTTTTATTTAGCGTTGCTAACTGAATCAGTTTCCCTTCATTACTT
TCAAATATGTTTTGAAATGTTAATCTGGCATTTTGTAGCTTTCTTCCTAACATGATCTGTGAAAATAAGA
ATGAGATGGCTGAATTTGTCGTAGTTAATGATCAAACAATTTTCAGACAATTGTTTTTCCTAGAAACAAA
AATTATTTCCATAAAGTTCCATATGCATAAACAGTGAAAACAGAACGTGGGGTAGTTTTGTTTAAATGAA
GTCTTGGTGAGAATCATATTCTGTAGTACAAGGAGGCTCTTAAAGTTTATTCTCAATACCTGATATAATT
TTCCTGAACTATTATGGAGTTTTGTTATGTATAGTTGGTTTTTCTGACTTGATATAATAACTTTACTAGT
CTCTCAAATACAATTTGGATATAAATCATTATAATAAGATGATTGATTTTTTAGACTAACTTTATTTTTT
GATATTTTTAAACTATTATGAAAAACTATTATGAAACTATTATGATATTTTTAAACTATTATGAAAAGTA
TATTCTAGTTTGAATAATTCCAGAATCAAATCATAATAAGCAGAAGTTCTTCTCCTCTCCCTCCTATCGT
TCTCCTTCTCCTGTTTTTCTTTTTTGATATGATAGTTGATCTACTTTGCTGCTCTGTTGCATAGAGTACG
TAACAGTGGCAATGTATGGCTCCTGAATTTATCGTTCTTGCTTCATCATCCTGCTTTGACCCCACTTTCT
CCTCCAAAATGCGTGTTGAGTTAGTTTGATCATTTGGAGGTAATTTGTTTGGAACAGTATCAGACTTTAT
AGATATCTCCCATGGCTTGTGATAGAATATAAGGGCAATGCAAATGTAGAGTTTTTTGCTCACTCTTCGA
TGTATGGTTAGACAATGTACCACTGTAATATATTTGGCTTAGGCTATTTCATAAATAAAATTTTATTATA
AAATATTATAAATGCTGATAAAGCTACTCCAGAATTTTAATAGATATGTGGGTTTCCCGGCCAGATGCGG
TGGCTCATGCCTGTAACCCCAGCACTTTGGGAGGCCGAGGTGGGTGGATCACCTGAAGTCAGGAGTTCGA
GACCAGCCTGGCCAACATGGCGAAACCCCATCTCTACTAAAAATACAAAAATTAGCTGGGTATGGTGACC
TGCGCCTGTAATCCTAGCTACTTGGGAGGCTGAGGTGGGAGAATCGCTTGAACCCAGGAGGCAGAGGTTG
CAGTGAGCCGAGGTGGCGCCACTGCACTCCAGCCTGGGTGACAAAGTGAGACTTCATCTCAAAACAAATA
AATAAATAAATAAAAATACATGGGTTTACATTTTACCCATCAGCTATGGTAGGTAAATAATAAGCTTTGA
TTAAGTCTATTTTAGTCTATTTTTAGCAGATTACTTTGAAAAATAAAGAATAACCCAATGACTAAAAAAT
TATTTTATGTCAGGGATTTAATAAAACATATCTTTAAATCTAGTTGAGGGCAAAAATACGTCTATTTTCT
ACTATACAATTTGTATTTATATCTGCTGTATTATATAATGAAAATTTATCTCTATTTCTAATCTCAAGAA
ACTGCAAGCTTCTGAATCATTAAAGGGAAGATTCACCATGTGTCCTAACTATATTTACTATGGAAGCATG
GAAAATAAATATTTTATGTTTAGATTTCTGATCTCTCTTTCAAAAGCAGTTGGAAATTATGCTGAGAAAA
TGTCTTAGCTTATCCCATGTTACTCAAGAAAATGTATTTATTCGTTTTTGTCCAGTGGCTTAACCAAACC
ACAGTTTATTTGTTGCTCACATAAAGTCCAGTGTCGATCAGGCTACTCTTTTCCATCTTTGAGCTAAGGC
ACATATTACACATAACTTTCAGTGTACCCGAGGTAGAAAAAGAGAGAGCTTGGGAATAAGGCAGGGGCTT
TTTACTGTCTCAACCCCAAAGTGATAAACTACATTTATTCTCAAAATCCAGATAAAACTCCCATAGAGCC
TCTGAAAACCTCAACATTTGCGTCTTAACTATAATAAGGTTAACTAAGATTCCAAAATTATTTTAAAACA
GAGACAGTTTCCCTCTTCCCTGGCAGCTAATATTGTATTTTCTATAAATCCACTTGCCCAAGGTTTAAAC
TACATTTTATGGATTGAAATGACATTTATAGCCAACTCCTGATTTTTAGTTAGATGGTTGGATAATGATC
TTTTGATGAAAGACTCGGAGATGTCATGGTAAAACGGTGAACTACTGAAACTATTGATTATTGTTAATGG
CACATTTCAGCTGATTGAATTGAGTCAAGAAACTGGTGTTGAAGAGCAACAAATGGAAATGCCGAGCTTG
AAAATAAATAAAGCAGCATACCTTAAGAGATTACATGCAATTTCAGTATTTCAGCTAAATGGAAGTGTTT
GCTTTTTTTCCTCTATGAATTTTTATTTTGAACAAAAGGAATTTTCTATAATATGTAGGTAGGAGAAAAG
TGAAATGGCATGCTTTTTCACTTCATTTGAAGAAGCTGGTAGCATTGTATTCATAGATTCATGCTGTATA
GCAATCATAGTTCTCATATATTAAAAAAAAAGGAAATTTGAAATGCCTAGCCAAAGCAACAGCTCTGCCA
ACAGATTTTGATATATCTGTCTACCCCAAAAGTAGTGATGATTTACTTCATACAAATGCTAGTGAATGAA
GAGAGAGGGTGAAAACCTTCACAAAATGTGTTTTTCTCTAAGACTGTCAATCCGTTTTTCTATATATGGA
GACTCCAGCTCTTGCTAGACTACCTATCACTTTCGTCTATCAGCCACTTCGTAAGATATTTATTCTCTCA
GCAATAATCATAATTCATAGATTCTTTAAACATACATGTAATATAAAGCATATACATTCTGAATGGAATT
AACATGATTAATTCTTCTCTGAAAGACATTAGAATTTCCTCCCGTATTATAAAAAGGTGTAACTCACTTT
CCTTACTAAAATCAAGAACTTTACCGTCGTCCTTGTACTTCAGGATAAGGGGGTGTTTCTTATAAATATT
GTTATTTCTGATATGCTAACTGGAATTTTTAAGCAAATGTATTTTTATAGAACGCCATACAAAGCCTTTA
GGGGTGAAAGTTTCAGGATTTTTAAATTGCAGATTTATCCTTTAAATAAAAAAACTATATTCGTAATTGA
ATCGGATTATTTCTCTATCCAAAACATTTTCTGCTTTGGGCCTAAGAAGAGTTGACAAAGCTGTTCATGG
TTCAAAGTACTACCATAAAACCCTGGGTAACTAACTGAAAATGGAAAGACTCTGTCTTTCTGAATATTTC
ACAAGAGTTTCACAAATATTAAGTGGTTCTCTAAGTACCCCTGAGAGATCATTGTAATATTAGCTTGTAA
AGACAATGTGGGGGTGTGGGTATGTGGTGACCTTTATGATGTTCATAAAGGTGGTGTAATTAACATATTT
TTCTCAGCAAGACAAACTAAGGAGCAATAAATATATGAGATACCTTCATCTGTGATCTGGGTCATGTCTC
AGGCCATATCTTTCAAATCACTCCCTTCCCTAATCTCGTGTTTTACCTACGTCTCCTCTCAATCCCCCCA
TTATAAAAATTGTCTTCTGATGAATAAAACATTTCCAGAGAGACAAGTTTCATAAAGTTTGAATTGTACA
TCTGAGTACACCTATGAATTAAGATATCTTTGATTTCTAATATGTTATTAAAATTGGGTGTGGTGGCTCA
CGCCTGTAATCCCAGCACTTTGGGAGGCAGAGGCGGGCGGATCACGAGGTCAAGAGATCGAGACCATCCT
GGCCACAAGGTGAAACCCCCGTCTCTACTAAAAATACAAAAATTAGCCGGGTGTGGTGGAGTACGCCTGT
AGTCCCAGCTACTCAGGAGGCTGAGGCAGGAGAATTGCTTGAACCCAGGAGGTGGAGGTTGCAGTGAGCC
GAGATGGCGCCACTGCACTCCAGCCTGGTGAAAGAGCAGACTCTGTCTCAAAAAAATAAATTAAAATAAA
ATAAAATAAAATTGGAGAAGTTTCTCACCAAAATTTTGGCGCACGGATTAATTCTGAAGAAAGAAGAAAG
AATGCAATCTTAGTAGCACAATTAGTACCTTGAATAAATTGGAGTATCGTATTTCTTGGACTATCTGAGA
ATGCAGAGGCAATTTAAGGATCCCTAATTCTAAGGAGAAGAAACCTTTAGTGTATTCCTTCCTGTTGCTT
TAGTTTGAATTGAGTTTTATATGTATTTTTTAATCTTTCTATTTTGATTGTTGTCTAAAGAGTGTGAAAG
TGAATTTTGATATTTTTATTTTGCCTGGCGATGAATGCCTTCTGCTCTGGATATTTAAAAATTATATACA
CATATATGTGTGTGTGTGTGTGTGTGTGTGTGTGTATATATATATATATATATATATATATATATAAAAT
TTTTCTGAGAACTTTTATTAATTCAGCGTATCTTTGCTAAACACCTGCCATGTGTCGTGGTGTTAGGTCT
GGTGATACAAACATGTTCAGAGAGATGATTTTCTTTCTTTTTTGGGGGGTGGGTAAGGGAAAGAAGGCTT
ATACAACAGAATCTTATTTCTCACAGTTCTGGAGGCTGGGATTCCAAGATCAGGGCCTGGTGAGGGCCCC
TCTTCCTGGTTTGCAGATGGCTTCCTTCTCTCTGTGTCCTAACATAGCAAAGAGAGACAGAGCTCTGATG
ACACTTCCTCTTGTTATAAGGGAACTAATTCCATCATAAGGGCCCCAAGAAAGGTGCTTTTCAAAAACAG
TTCAGTAAAAGTACTGGGTTGTATAATCACTTTAATGAGTATCAATCCATATTTTTAAGATAGAAATGAA
TGAAATTAGTAAAATAGAATAGAAATAAGGAGTCCATCACTTTTAAGTAAGTTTCAATATTGTTCGTAAA
ACTTTGGTTCGGTGGTTTGTGTGTGTGTGTATTTGTGTGTGTGTGTGTGTGTGTCTGTCGGTGTGGAAAT
ACTGGATCACTTTGTAACATATATTCAAAAGCCTCTGTATTTTAACATTATTTCTGCCTTTGAGAGGTTC
ACATTCCAGAGGTGAAGACATACATCCTAAGACAAAATTATAATAGCATTATGAGAATTACAGTAGAGAG
CTGGACAGGGTCTAGCAAAAACAGAAGACTAGGCTAAACCTTCCAAAGAGGCCAGGAAACTCACCTAGAA
CGGTGGATTTTAACCTTGCTTATGCACTGGGGGAGATTTTAAAAATATCTCTGCCCACAATAGATACCAA
CTGAATTGAGCATAGCATGTCCTACCCATGAATCTATTGTCCAGTGAGAACCTCTGTTTAGAGAAAGTCA
CCTTAGAAGAATTGTTAGGAGTTATTTAGGTTCATGGGGTTGAAAAGAGCATTCGTGATAGAGGAAACAC
CATATCCAAAGGCTTAGTCAGTGTGGTAGTGTGAGAATCTGAAGGAACTTGGCTGGGGTATGGTTGCTAC
AAGAAATGAAATTAGATCAACTGGGGCTAAATTATGTGGAAAGACAGCATGATGTAGCAGCTAGAGTATG
GACCTTGTAAGCAGGAAGACCCCTTATTTAGCACTTACTAGCTTATTGTCTGACCTCTGAGTCCCAATTT
TACTCTTCTATACAATGAGTACATCACAGGATTTTATCAGGTTTAAATGATAAGATATATGTAAAATGCA
TACCAGAGAGGCAGACTATTGGACTCGAAGGGCTCAGTAAGTGTAAGCTGGCTCTCTCTGCCCCTTGCCA
CCTATTTTTCAGACTCTGGACTTTTATCACTTTAAGTCATAGCCTAGTTCTAAGCAAGGAAATGGACTAA
TCAGACATGTTTTTAAAAGATCATTCTGGTAGTGGTTAGGAGAATGAATTGGAAAGATATGAGACCCATG
CAGGGACAACAGTTAGGACATTATTTCTGTAATAAGCCAAGCAAGAATTGATGATCAAAGTGGTGAGGTT
GAACAAACAAAACAGATACGTGAGCTATTTGGAGATAAAATCAACACTGTCATATGTTTTGTGGGAGGTG
GAGGTGAGCAGAAAATGTGAGGTAAAATGAGAAATCAGTGCCTGCTTACCACTTGGCATGATTGACTGAA
GGTAGTGTCTTCACTCAATCATGAGTTGCAGAATTCAAGATGGCAAACAGTTGTGAGGAGCAAAGTCAAG
AACGTGTTTGATTTTGAGGTATCTGTAAGTGAAAAATCAGAGGTGAAAACCTTACCTCTCTTGAAGCAGT
TGTGAATGTAAATCTAAGGTTTGGAAAAAGATCTGGGTTAAAGATTTAAAATTGAAGGACATCAACATGG
AAGCCATAGAAATAAATTATATTACACACAAATTTATGTCGTTATTTGAATTTCTCCATGGTCCACTCAG
AAATATATCTAAATGTCACCAAAATGTTACTTACTGTAGTACAGAATTGGTATTAAGTGATACTATTGTC
CATGTTATTCAAAAAGACAGTTATAGGGACCCTCTTAATAAACTAATTGTGAAAAAGGCAAAGAATTAGC
AAAGCTTTGGCATAAAATTCATATCATGGGCCAGGCGTGGTGGCTCATGCATATAATCCCAGCACTTTGG
GAGGCTGAGGTGGGCAGATCACCTGAGGTCGGGAGTTCGAGACCAGCCTGACCAACATGGCGAAACCCCG
TCTCTACTAAAAATACAAAAATTAGCCAGGTGTGGTGGCACACGCCTGTAATCCCAACTACTCGGGAGGC
AGAGGCAGGAGAATCGCTTGAACGTAGGAGGCAGAGGATGCAGTGAGCTGAGATCGTGCCATTGCACTCC
AGCCTGGGTGACACAGTGAGACTCCATCTCAAAAAAAAAAAAAAAAAATTATGTCATGGAAAAAGTAAAA
GTCTTTGCATAATGTATCCAAGATCATGAAAAACTCTTTTCAATAAGATAATTAGTTCCTTTTCTTATAT
AAACATGGAAATTTTCATTTTTCCTTTTATTCTCATATTGATACTATAAAAACCCCATCCTCATTCACAA
TACTACTGTCTCTACCCTCGATAGATACCAGTTCAATTGAACGTAGCATGTTCTACCCATGAATCTATTG
TTCAGTGAGAACCTCTGACTATAATGCTCAGGAATACTCAAGACTCACATGATTGTCTTCTTGCTATATT
TAGTTACTTTATTATTTTCCATTTTGGGACCCTGAATTCCTGTAGATCTCAGAGAAAATCCGAAATGAAA
TAATGAAAATAATTAAAAGTTTAGAAAAGGGAGTCAATGGGGACAAATGTTCAGGACTGGTCTTTTATCT
CCTGCAGGAAGAAAGACTGAATGCAGAAAATTAGAATCCATTTTTCATCCAGTCACCCCAATTTAATGCA
ATATGAGTTTAGCTATTTGATTTTAAGTGTTGTACCGTTTTGGACCATGTTACCATGGTAACATGAACCA
TGTCTCATTCATACGTAAACATGTTAATTGTATTAAAACCTTTAAAACCTACTTCTGGATGTTGCCATTA
CATTAAACAATTATCTAGAATGATACAAAGTAATGACTAAATTGAATAACTTTGTAAATTAACTATTGGA
TTTTGTAATTTTATATCTATAAACCAAAAGAAAAGCCCACATTGGTAAGAAGACACTGTGCATACTGAAA
AGTCAATTTTGTTAGCCTCCAATAACCATTGTGTTTTATTCCTCGCAGAGCTTTTGTGAGGATCTTATAA
GGGAATAAATATGAAAGCACTTTGAAAAAGCTTTCAAGTGAAAGGTCCTTATTAATTTTATGAATTACCA
TTAAACAAAAGTCAAACTGAAGATGTAAATCTAATAGGATGCTCTTAAAAGTCAATGGATCAAAGTTATA
TTAATTAATAAAGAATAATAACTAAATATTTTATGTTTCATAATTGGCAAAGTATCTTTACTGTCATTTT
CTAATTTGATCCTTAGTGAAAACCTGTGATGTTGGTACTCCTATTATTTCCATTTTCATTTGAGAAGAAT
AAAATTGGAGAGGTTAAGTAATTTATCTATTGCTACTTGTTAAAATAACTACTAAATTTTATTACTCCCA
GTTAGGAGGGCAATTATATAAACTAAAAGCTTGTCACAATAAATGTTTACTTTTCTGGGATTAAAGTCAT
CATGTATTTTTCAATTATTAAGGGGGGTAATAATAATAATAGCTACCTTTTTAAAATAGTTACTATGTGC
CAAGGTGTGTACTAAGTGCTTTGCTTGCATGATGTAATACCATCGTATATTTAGTACAGAGGAAAAACTG
AGAGGCTGGGTAACTTCTACTAAGGTAACACACAAGTACTGGTTGAGTATCCCTTATCCAAAACACTTGG
GACCACAAGTGTTATGGATATCAATTTTTTTCTGATTCTTTTTTTGGATTTCAGATTTTTTCAGATTTTG
GATTACTTGCTTTATAATTATGGGTTAAGCATCCCAAACCCCAAAATTCAAAATTGGAAATACTCCAATG
AGCATTTACTTTGAGAATCATGTCGGCGCTCAAAAATTTTCAGCTTTTAGAGTTTTTTGGATTTTGGATT
TTCAGATTTGGGATGCTCAACCCGAATATATAGAAAAGTCAGCATTTGAACCTAAGTTTGACTTTCTGAT
CTTCTACCAACTCTACTGTCCTACCCATTACTCTACATTGACTCAGCATTACAGGGAAAGACCCAAGATC
ACCAAAAGCAAGCTTCAAATCACTCATCTAATAGAAATTAGTGGAAATATTTCTACTTCCTAAACATCCA
TCTTTCCTTTACATTTTAAAGTCAAGTTTCTACATCTGCCTCCCAACTGAAACACTTCTCTATGAAATCA
CCATAACTACCAAATGCAAATATTTTTATCAAGTCCTCATTGCCCTAGAAATCTACTCATATTTTGTTAT
TACTGCTCACTACAGCCTACTGAAAAATGTCTCACCTTTTGACTTGCCAGGGTGATATATTATACTAATT
GTCTCCTTGTCTCTCTAAGCACTCATTCCTTCCTCTTTCTTTCTTCTTTTTTTTTTTTTCACTTTTATTT
TAAGCTCTAGGGGCACATGTGCAGGTTTGTTACATGGGTAAATTGCATGTCATGGGAGTTTGGTGAACAG
ATTATTTTGTCACCCAGATAATAAGCATGGTACCTGATAGGTAGTTTCTCAGTCTTCACCATCCTCCCAC
CCTCCACCCTAGAGTAGATCCTGGTTTCTGTTGTTCCCTTCTTTGTGTTCATATGTACTCAGTGTTTAGC
TCCACTTATAAGTGAGAATATATGGTATTTGGTTTTCTGTTCCTATGTTATTTCACCTAGGATAATGGCC
TCCAGCTCCATCCATGTTGCTGCAAAGAACATAATCTCATTCTTTTTTCTGGCTGCACAGTATTCCCTGG
TGTATATGTACCACATTTTCTATATCTGATCTACCATTGATGGGCATTTAGGTTGATTCCATGTCTTTGG
TATTGGGAATAGTGCAGCAATGAACATACAGCTGCATGTGTCTTTATGGTAGAATGATTTATATTCCTTT
GGGTATATACCCAGTAATGGCATTGCTGGGTTGAACGGTAGTTCAGTTTTGAGTTCTTAGAGGTATTTCC
AAACTGCTTTCCACAGTGGCTGAACTAATTTACATTCCCACCAACAGGGTATAAGCATTCCCCTTTCTTC
ACAACCTCACCAGCATCTGGTATTTTTTGACTTTTTTTTTTTTTTTTTTTTTTTTTTTGAGACGAAGTCT
CGCTCTTGTCCCCCAGGCTGGAGTGCAATGGCGCAATCTTGGCTCACTGCAACCTCCACCTCCCGGGTTC
AAGTGATTCTCCTGCCTCAGCCTCCCAAGTAGCTGGGATTAGAGGCGCCTTCCACCATGCCTGGCTAATT
TTTTATTTTTAGTACAGACAGGGTTTCACCAGGTTGGCCAGGCTGGTCGCAAACTCCTGACCTCAGGTGA
TGCGCCCGCCCCGGCCTCCCAAAACGCTGAGATTACAGGTGTGAGCCACCACACCAAGCCCACAGTATCA
ATTCTATGCATTCTTTTCTGATTTCATTAATCTCATTATCTTCATTTGATATTTAGTCAATAGTTACTGT
CAGTTATGTGTTAGTTATTATACTAGAAACAGTCTTTTCTCCATCTCCTTTAATCCAATGATTTGAACAT
TTTTATTCCTTTCCAATGTCTGTCCCACATTTCTTACTGTATGTAGGACATTTCTTACTCAAATGTCTCA
CAAATGACATAAATTCAGTATGACCCAAATAGGCCATTTTTTATACCAAGTCTTATTTCCTATCCTGCTG
TTCATCCCGGTACCATCTTTTCAGTCAGAGAGTTCAGATCATATAGTCATTTCTAAATCTCCCACTTACT
TGCCTCACTTTCAAGTTCATTTTTAAGGTCTGTAGATTCTGCCTCCCTAATTCTTTATGACCATTCCTTT
CTCACTAGCCCCTTACCTCCACTCTCATTCACACTCTTACTATTTTTTACCCTCCTCCACTCATTCCTGC
CCACCAGTGGCTCCAATCCAACTTGCAGATTTCCATTTAAATTAAGCTTCCTAAAACATAGCTTAGGTTG
TAACTACAATGCAAATTCCATGAGAGCAAAGATTTCATCTGCTTTATTCACTTGTATATATCCATTGTCC
AAGACTGTGTGTGTCACATGAAAAGTGTTCAATAAGTATTTGTCAGTGAACGAAAATAATATATGACTCC
CCTCTTCAAACACCTTTTTTGACTTCAAAGCCCTTCAGAATATTCTACAGACTCCTTCACCTGGCTCTCC
ACAATTGCCCCTGAGTCTCGTTTCCAATCTTATTTCTTATTTTACCTCTCAATGCACCTTCAACTCCTAC
TAAAATGAACAGCTAGCCAGCTTACTTCTGTGTCTTTCGATGATCTTGTTTTTTGTCTTGAGATTCCTTT
TTTTCATCTAAGCTTACCCAAACATTACCTACTTTTCAAGGAAAGCCATTTTCGAATCTTCCCTTTTTCC
CTGAGCCCCCAAGCTGGAAGACATCTTGTCTCCATCTCAATTCCTATAGGCATTTCTCTGCACTTTAAAT
GACGTTTAGTACTTCTGACATTGCATTAGAGAGAGGCTGGGGTGGATAGTGTTTCATAGTGTGAACTTTG
AAGCCCGACTGCCTGAGTTTAAATCGTGATTCTGGGGCTTACTGACCATAGACGCATTTCTGAATTGCTC
TCAGATTATGGAGCATAAATCAAAAGTAATGACAGCTACCTCTTCAGGTTGTTGTGAGGGTGATGCGAAT
TAATGTACTGAAGTGCATGGAACAGTTTCTGGCACACGGTAAGCACCCAATAAACATAGCTAATATTATG
TTATTACTATTTTCAGGCTTATTTTTATGTATACATATAGTATGTAATTTTATGTCAATATGTATAAATA
GACTTTGGTATTGTTTATTTCACTATCACCTTGAGAGCACAATTCTCATTTGATTTGTGTGAGAAACTAC
TTAGAAAGAAATAGACGTGTGAATGAAACTATGCTTGAAATATTGGTTACTGTGAGTGTTGAAAATCCAT
TTTGTTTAAAGAAAGCTTCAATTGTTAATCTTCCATAAATTTTAGTTCTTAAGCGTTCATATTGACTCGT
TTTGGAAAAGCTCTTTAAAGTCTTGGGATATAAACAAGGCTGAATACCCTCATTCATGATAACAAACATA
TTATACTGAAAATTGTAAGAGAGATATTTTATCTTTCATAATGCCCTCCTTGGGAAAATACATTGACTTG
GCCCTTCTCTTTCAATCAGACACCAAAGTTGAGATTGCCTGAAACACAGTTTGGTAAAAGGAGTTTCTTT
TTCCCAAACATCCTGAGTAACACAGGAAATCACACCAATGACTGATAGATAACGTTAATAAAATTAATAA
AGTTGTTTTAAATGCATACCATGGGGCAGTGGCAATGAAAACATTGAGAAGGCTGGGACTATTTGCCAAC
TTTCTTTGATCTCCATTAGAACCTGGACAAGATCCACATAATTTCAGAACTTCTTCTCCAAACAAGAATT
GAAAAGGTCAGGAAAAGTTTGACCACAGAAAAATGTCAAAGAATTTTGTGTCACTTTCTCCTCCTCCCTT
CCTCTAACCTTGAATAATTTTTTAGGGTTATTGGTCTTTGGGAGCAGACTTTCTAGACCAAAACAAAAAA
AATGATATTCCTCTATGTGATAGGTAACAATCACTACCCATCCTACTGGAAAATTCTCAAAGTGTAAATT
GAGGGGATAAAAAAAGAATCTTAAGTCCTTTAAATTATTTTTAAGATGAACTACATTAGTGCCTCTCTTG
TGCCTTTCATAATTCTGATAATAAAACATTCCAGGTATTAGTCAAAGATTAATGGTATTGAAAATAATTT
AGGTTATCAGCATGTGATTTTCATTCCACATGAGGTCCTTTTGCAGTTTACATGGTTTTCTAAATTATAT
TAAAATAAAATGTCAGAAAGTTCACATTTTTTTCATGTTTAACAGCATCAATCTTTAAAGAAAAGTTATT
GCACAAAGGTCTGTGCATAAATCAGCCATTCTCCGAAGAGGTAAAAGAAGTCATTACGCCTGGTTATGAG
AGAGAGTTTCATGAATGTAAGAGACATAAATCATTTCCCACTGGAGATCATATTAGTCTAGATGGAAGAA
TGTCTGTTTCTTGATAGTGAGAAAGCAACAAATTACTTTTGTTTGCTCCTGAGTCTGTGGTTGTCCTTGA
GAGGTCTGTTAGCATGTTGACTATTGACTATTCAATATTAGCATTATAATAACTTACAATGATCTGAGTC
ACATAAATATAATCTTTCAGTTCTCTAAAGATTTTACTTTTTCCTCTCTAATATCTATTCACCTCCAACA
CCTTTGCAAATATATTATTCTCTGGGAGTTACAAAGAAAGTTATTCTCTGCAGGAAGCAGCATTTCAGTT
GCTCTCAGGAGCCAACCACATTTCACCTCAATTCTTTGCTCCCAATTCAACAATTCAATATTGGATTAAA
TTCAAGGCTGTGACCCCAAATAGAATGAGACCTGGATATTTATGAACCACTTGACCAGGCATTCTTCCCA
TGATTTACTCCATAAATCCTTTTTAGTTTTTGCAGTAGCTTTACAAATATTTGGAAAATGGCTGTGCAAT
GCAGTTTTAAAAAGTGCAATGAGTAGAGGTAGCTTCTTCACCTGGTATGGTAAATTGTTGATTCTCTTTT
GGAGTGGAAAACAAGTGTTCTTATTTGGATGCAACCATTGCATTGATTAGACAACCCTAAATTCATCTTT
CATCCATGACCTGAAAGAAATTTTGAAATTCATGCAATATATACCCGTAGTGGAAAATGTACTTTTTGAA
TGGATTCCTGAATGTGACTTTTAAGAAGAGCTATTAAGAAGTGGGATCTTCTACAGAACAGTAAACAGGC
ATGAAAATATACAAGTTGATAAGATATGGAACTACCCCAAAAGAGGAATTAATAGTGGTGGGGCTTGGGG
CAGGAGGACAGAGAGACCTAGCCAAGGAAGGAAGGGCTATATTATAATAGAGTACAAAGTCCTTTAGTCA
TCCAAGAGAAGGGGCACCTTCTGCATCCCTTATGAGTAAGATCAGAGAAGGTATTCTAGTTAACTTTTGC
TACATAACAAGCCAGCCCAAAACTTCATGGCTTCAGTAAAAATTACTTGTTTTGTTCATGAATCTACAGT
TTGCTCAAGGTTCAATGGGGCTTGCTTATCCCTGTTTCAGTTGATATCAGTTGGGGTAGATTGCCTGATG
CTGGAGGATTCACTTCCAAGAGGGCTCACTCACATGCCTGGAAAATAGGTGCTGACTGTCAGTTTTTCTT
CATGTGGACCTCTCCATGGAGCAGTTTGGGCTTTTTCACAGTGTAAGAGTTGGGTCCCAAGAGCAATTAT
CCTAAGGGACAAGAAATTAAAGCTGCAAGCTTCTCAAGGCCTGCCCTAAAAGCAAGAATGGTTTTGCTTC
TCCCATATTCTATTTGTCAATCAGTGACAGAGCTCTGATTCAAGGGGATGAGAACATAAACTCCACCTTT
CCATGGAGAAGTATCAAAAAGTTTTGATGCCATTTAATTAAAGCTGCCATACAAAGTTTCTTATAAATGA
CACTGAGCTGAATGAATACTAAACAGCAAGTAGTCATTATCCCAGTCAAGAGAAGTTATCTTTGCTCAGA
ATACCCTTTCTCTCCTTGTCTACCTGGAAAATTCAACTCTTGGCCAAAGCCCTACCTCTTCTCGAAAGCA
TTACCAGGCCTTGCCTCTAAGTGTACAATTGGAGATACACCAGTATACTGATGTTTTTAAAACTTTAAAC
TTTTTTCTACAATAAAACATAAATTAAATAACTTCCCTTCTGACTTAAAAGCTGCAAAATGCTCATGACA
GTAACTATATAAATTAAAATTAAATCTTAAGCACGATAAATACCTCTCGAATAGCAACATAGATGCTTAC
TTCTTTATTTCACTTCTTTATTTGCTTTTCTTTGTCTATAGTTTGCCCCAAAGGTATTTTAATAATATCG
GGTTCCATGTATACCAGTGTGTACCAATTAATATTTAGAATATACCTGTTAATAACCTCATTTGCATAGC
CCTACTAATCTGAGCACAGCGCAGCCTTAAGAAAGTCTTAGTTTTTCTCAGTTTAGTTCATCTCTCTTCT
CTTCTCCTCCTGTCTCTCTTATTTCCTATTTCTTTTTCTTTTCAAGTGACTTTCAACTAAGTAGAAAATG
CATTTCACATCACTATGCCGGCCTCCAGGCTCTGTCTATTTCATTCACCCAGGAATGCCCTTTCTGAATG
CTTTCTCTCATTTAGCAGCTATCTATTGAAGTTGGACAAATGATAGAAATTCATTTCTTAAAGAGCCAGA
ACATCATCTTGAACAAGAAGTTAAAAGAATTCAGCAAATCAAAAGATGAGCTAATATGGGTGAATCTTAG
AGGCATTATGCTAAGTGAAATAAACCAGACACAAAATGAAAAATATTGTATGATTCCACTGGTATGAGCT
ACCTACAACAGTCAAATTTATACAGACGTAAAGTTGAAGGATGTTACCAGGAGCTGGAGGAAGAAGAGAA
TGAGGGCTTATTGTTTAATGAGTACCTGAGTTTCAGTTTGGGATGATGAAAACATTCTAGAGATGGATAG
TGGTGATGGTTCAACGATAATAATAATATAATATTAATGTACTTAATAGTACTCAACTGTATACTTAAAA
ATGGTCAAGAAAATGGTACCCCGTTATCCTGATGTGATTATTACACATTGTAGGCCTATATCAAAATATC
TCATGTACCCCGTAAATATATGCACCTACTATGTACCCATAAAAAAAATTTAAAGGCTAAATGGCCAGGC
ATTGTGGGTCACTTCTGTAATCCCAGAACTGTGGGAGGCTAAAGCAGGAGGATCACTTGAGCTCAGGAGT
TCAAGACCAGCCTGGGCAACATGGCAAGGCCCCATCTCTACAAAGAATTCAAAAATTAACTGGGTGTGGG
AGCTCATGCTTGTAGTCCCAGACACACTGGAGGCTGAGGCAGGAGGATTCCTTGAACCCAGGAACTGGAG
GAAGCAGTGAATGACACTGTACCCCAGCATGGTCAAGATCCCAAATCAAAAAGAAATGATTAAAATGATC
AATTTTATGTTGTGTATATTTTGCCACAATACAAAAATGGGGAAAAGCCTATTCGCTTTTAAGTATCCTT
AAAAAGGCACAGCTTCTTCAGCTAACAGACTCTAAAACTTTTTTTAATAGAAGTATTAAGGTATTTAGAG
AGTGCAAAATATCTTATTTTAAGTCAAGAAGTTAGGGTCCTGTTCCTAAACACTAGCCTCTGTAATCCTG
GGGAAGTCAGTGCTGTTGGAGATCTCAGGTTGATCTTCTGAAAAATGATGGATCTAGGTAAAAGATATGT
TTCTCCAGGTTTACATACCACGGACACCATCTTTACTTGGAAACTTTATTAAAAATGCATTGTGTCAGAA
GCTCTCTGGGGATGGGTCGTGGAATCTGCATATGTAAAGAGCCCCTAGGTAGTTCTTGTGCCCACTTAAA
TTTGAGAACCACTAGACCAGATGTTTTGCTTATGGCCCTTTCAGCTCTGAAATTTGAAAAAAAAAAAAAT
GATTCTGCAAGACAGAGTCTCTGTGCTTTTGCAGGATAAAGAAATGAAGAAAATAATACTTCCTGCTTGT
GTTGGAGCATTTTTTTCATTTGGTATCCCCATCTCCAGTGGCTAGCCAATCAAGAATAGTATTGTTTATT
CTTCCCACTGTTTTGAAGATACAAAAGGAAAAGCTAAGCCAGATGACACCTAAAGGCTTCCATTACCATT
TTCATGTTTTTCCCTTTGCATAAAAACTGTCCATGCCTCCATCAGAGCCATGATCACTAGTACAATGTTA
CACTCTAATGACTCATGACATTAAATTATATCTTAGCCTAATATGACCAAATTACAATATCAGAATAAAA
ATTTCTTTTTTCAGGTTGAATCCCATAACTTAATCCAATTATAATACTGGCTGAATTTTTCACAATTATG
TCTCAGTCTTGATTTAGGGAATCTTCTCTTTATCATAAAAATGCATTTTGTTAAACATGTTTCATTATAA
TCAATTTCTCAAAAGTAAAGTTAATCAAGAGAAGGAAAAAAGGTTTTGTTTTGATTTGATTTGGAATGTG
TATGTGTGTTTACTGTATTGAAATAGATTCTGTCTGAAAGACTGTATATAAGATAAAAAGTACAGAAGAG
TAGTCAGAGAGTTATTACCCACCCCTGACTGATGGTGAATAGATTATCTAAGTATCCCGTAAAAGGCACA
ACTCCTTCAGGTATATTTTACAAATTAATTAGTAACTTTCTAGCCAAATTTGTGTCTTAAAGACACCAGC
TAGAACTTGGTTAGTTCTAGCAAAGAAGATTATTTTATTCTGAAACAGGTTTTTGTTGTCGTTTTACTTA
TTTGAACTTTTTTCTTGAATATGTATTTCTTTGCACATAAAATATATTGACTTATGAATGTGATTAAAAT
GGAAAATAATTAGTTGATTTTAGAGAGACAGAGAGAGGAGAAGAGAAGTGTGAAGGAGAGAGGGAGGATA
GAAAGGAGAGAGGGAGAACAGGAAGGACAGAGGGAGAATGGGAAGGAGAGGGAGAGAGAGAGACAGAGAG
AGAGGAATGGAGTGGGTAATAAGCAAGAGAAAAATGCCAATCATATGCTTTGCTAGTGTGTAAAGTCTGA
TAACCCAAGGGAGAGAGGACTACTCTGGCCTAGTGAAACAAAGGAAAGAGAAATATGGTAGAATATTCTC
CTGGTGCTTCACCAAATGTGACACCAGAAGTCTGACAGAAGTCATGTCAGCATTTGAGCTCCATAAAACT
CAGGCTATCGACCTACCATGTGAGAGTCTCAAAATGAGTTTAGGTAGGGGCAGAGGAGTTGAAATCCAGT
AACATATGCAACAGTGATCACACCAGGATTGCACATAGAAAGCAAATTAGTCCTCTAATAGAGACGCCAA
TTTGAAATTCACCCTCTGAGCAGGTTTTTAAGCACACTCTTCTTTTACTTTTCTATTTACAAAAATGGAA
CACCACCAGAAAAACAAGAATTTGAAAGACGAGATGAGAAAAGTAAGTTGTAATTGGAAACAGACAGAAT
GTGTACACAAACACACACACACACGCACACACACGTGCATGCACAGGTGATGAGAGAGTAGTTTGCCTAC
ATGGTGTATCTGACTAAGAAGACTTTTTGCTCTGGTTGTCTTACAGGAAGTGACTAAATCTCATGATGTG
AAATATTTTCTTGCATATTGTATTGGAAAAGAAAATAATTTTCCCAAACTCCTTAGGGGCAGTGTTGTCT
TATAATTCCCATATAGTATATGCTCTTCAAGTAAGTAACTCCAGAGTTGAGTAAGACAAGACTCGTGACT
CAGATGGCATGCTCTGCTCCCTAGACTAGACATTGCATCAGTCTGCCTATACTCACATCCGCTGTTAAAG
GATTGCCTCCAGTAAAATATGTCTTTTAATTCCTTATACAAGAATCTGGAAAAAAAAAGTAAGATTCTCT
ATTTCTTAAATTTAGCAGCAGGTTAATCACTGATAACAATAAAAATACATAACAATCATCTAGCACGGGT
AAATATTGTGGCAAAAATTACACCCTGAAGAATTCAGTCAAAGATATAAGTAAGTACACATCATTGTCAT
GTTCCACAATATATCATCTGCTTTAAAGAAACTGTTATGTAGCTGTAGTAGATTTAATCATTAATCCCAT
TTCTTCTCCACCTTCTGCAATCACAACCTTAACAATGCCTCCTTATGAGTGGAATGTACTTCCCAACCCC
TAGTCTTAGGGGTTGGCCATGTGATTTGCTTTAGCAAATGGTAAATGAGCAGGAGTGAGAGGTGACAGTT
TTCAGCCTAGGCCTTAAGAGATCTATACATTCCTGTTTGTGCTTCTGCTATCATTCTGAGAACACGTCCA
TCTAGGCTGCTGGTCTCAGGAAAACGATAAAAGACATGAACAGCAGGGCTGCACTAGCCATTCACATCCA
GGAAAAGAAATGATTGTTGCATAAAGCCATTGAGCTTTATTCTACATTACTGTGACAATAGCTAATTGAA
ATAGTAAATATACTTTGGTTTTTCCTAAATGCATATTGAAAATTAATAATATTAGCCATCTGTATGATAA
AAATATAAAGCCTATGTTTTATTTTTTAATGGTTCACTGCCCTAAATAAATTTCCAAAAAGTAGATGTTC
CCTTGTCTAGTGATGTCATTATATTTTATTTATACATCATAAACACACTGTTTATTTCTGCTCATTTTTT
TGTAAGTAACATGTGTTACCGCCAATCTTGAGATGATACACACACTTCTGTACTAAATTTTGGAAAACAT
ATTAGCTACCCACTCCTTATATCAAAATATTGCCTAATAATGTGTTTTGTTTTAATCCTTCATGAATTTC
CAGGAGAACTGAACTGATACTTGGGTTTGTGAGATATATGAAAATAGTGAACATGAACTTCTGGTTTAAC
CCTTGTGATGATAATGGAATCATAGCTCTGTTAATTACTCTTGTGGTTTGTCTTCCTAGAGATAATCATG
TACAAAATTCCTTTCCAATTTGTTATATAATATTAGAAATACTTCCAAAATTGGCATGGATTTATTGTTA
TCATTTGTTGGCACAATCATTAAAACGAAACCCATAAAGCTAGATAATTAAATGTTTACAAAGCTATAGT
ACTCAAAACAAAAACACTGTGAAAAGAGATTTTTTAAATAATAGTTTTTGCATGCCTTTTGAATAATTGG
ATTATTCTGAATTTCTTCATGTTTAGTCCCTGAATCTAAGTCATACCGTCTACATAAAAATAGATGTCAG
CTGAAGAAAACCAGGCAATGGATTTGTCTTGACGACAATCTTTTTATATGTTCAGACTTCATTTAACATT
AGACTTGTCTGTATTTGAAATTGGTATTTCTTTACATTTCTGAATTTAGGGAAATGGCACAAGAGAATAA
CATTAATTTCCTCTGCATTTTGGCCTAATCAAATTTGAGCCTTTCAAGAGACACAGCCAAGTCAATTCAA
AGAGACATATGAAAAGACTACTGTTAATGTATCTTTAAAATGAATTAGCGGCATGAACTGTTGCTAGGTG
AGTTAGGTATAGTTGTAGTTTTTAGTAACCCTAAGAGAAGATGCAGTGCATTCTAAAATGTCACAAGGAG
TTTGATTGCTCAAAATTCTGGGAGATTGGCTCTCTGCAAGGCTTCTTGATGTCATTGTTCCTAGAGGAAT
GTTGTTCCAGTACCTATAGCGATTGCAGCCATAACTATTTATGTGTCATTGTAGCCATTGTTATTACTAC
ATGCTTCACATACCTCTACTGAGGTCTAAAGAATTAGTGGACTTCATATTCTGGAGAGAACACTTGAAGA
ACCAAACAGAAGTTTGATGTGAATCTGCATATCCACCATTATTGTTCATAGGTTCTCAGGATTAGTTGAG
TGATGCCTTAAAGAAAGAAAGTCAGATGATAGGTCTTCCTGCTGCCCGCACCACATCATGAGTGTTATTC
CTATAGAGGAGGAGTAAAGAGTGGGAAGAAAATGAAATCTGTCAATACTGTGAATATATAAATAATAAAA
GTAGCAGTAGGACTGATTAATTCTGAATCATCTTTATGAAATGACTGGAGCCGTGAAAATGCTCAGTCTG
CACAGCTGATTGAGAAATGTATGCAATCTGTTGATCGGAATTTATTTGTGAATGCTCTCTTCCAGAGATT
TATATACCAGAGTTCTTAAAACGAATTTTGTCCCCATGAAAAGAAAACTACAGATCTGTAAGACTGCAAT
TTAAAATGGAAGAAAACATGTTCCCACTTGAAGAACAACTTTCAAACAAACAACTGATACAAAAAAGTCA
AAAGCTGTTTTGTTTTATATAATAGTTTCAGAATACTTCCAGTCAATATATACCTTGGTTTGGTGAAAAA
ATAAAAAGCTAAATCCTTAGATCATTAACTAGAAATTTTTGTAAAATAAATAAAAGCCGTGGGTTTTAGT
GCAGTGATCCCATGAAGAGGAATATATTCACCATTGGTCTCTTAATCTCAGATAGAATGTACATGTTACT
TTATTTTATAACGAAAGCAACTGTGTTGTGATATTATGTATAATATTATAACAGGAGAAGTCCTCTTAGC
TAACTCAGTAATCAATAACATTGTACGTTGTGTGTTATTGTAACCAAAAACTATGACAGAACCCCATTTC
ATAAGATCAGTTTATCCACCTATATGATTTATATTTGAATATTCATTTCAGTACTTATGTTGCTTAAACA
AAGCTACTGTATTAGTCCATTTTCATACTGCTATAAAGAACTGCCCGAGACTGGGTAATTTCTAAAGGAA
AGAGGTTTAATTGACTCACAGTTCCACATGGCTGGGTAGGCCTCAGGAAACTTACAATCATGGCAGAAGG
TGAAGGGGAAGCAAGCATCTTCTTCACAAGGCCGCAGGAAGGAGAAGCGCCCAGCGAAGTAGGAAGAGCC
CCTTATAAAACCATCAGATCCCGCTATCATGAGAACAGCATGGGAGAAACTGCCCTTATGATTCCATTAC
CTCCACCTGGTCTCTCCCTTGACACGTGGGGATTATGGAGGTTATGGGGATTACAATTTAAGATGAGATT
GTGGGGTGGGGACACAGCCAAGCCATACCAAAAACTCTGTTTTTTGTTTTTGTTTAATGGAAATGATTTA
GAACTTTATTTTCTGATGTTTCTTTTTCATAAAACCACGACACCAAAATCTACTTTTCACTGCTCCATTC
AACTAGTAGAGAATATCTAATCTCTTCTCAAGTATTTCTTTCTCAATTATGGTGGTTTTAGCTAAGAACA
GCTTATGGCATGCTTTTCTAAATAATATTAGAACACATAAATTATCTGTACCTGGTATTACCACATTCAT
TGCTCATTTTAAGATCTCAATTGATACATTCAATTCATATATATTTAAAATTGATTCATTTAGAGCAAGA
GATACAGGCATTTTAATGTATTACACTGCTACTAAAGCTTAGCAAATTATTCTTTTTTGTGCCCACAAAT
TATCATCCATTCATGTCCTAAAAATAAAATTGAATTTATTATACTTTCCCATTTATCCAAAAAAAAGGTT
TTTTTTAACAATTGATGCAGATACACATTTTCAAGCTAAAAATATGTGTGAAAGTGGCCTCTTTCTCATA
GTATTTATTTTAGGAGTCTAGCAATAATTTTTCTTAGGTTATCAGCACATGTCTTAGCCTGAATTATTTG
AATTCAGTCTGTGTCTTCAAGTTCAGATGGTTATGTGATCTTGTTAAGATCTCAAAGTAGTGGGAATGAT
GGAGTATACAACAACCTCATTGTTTTTTATGGCAACTGTCATTTACTGAAGGACATAAGGCTAGCAGAAC
ATGGTCAGAGAAGGAATCAAAGTTTGGTCAGCCAACTCTGCTCCACAGCTACAAGCTGCTAGACAGGCAT
AAATTTTTCCAAACCTACACAAAGGGACTTAGGGCCCTTGGCTGAGAGCGACATTCTAACCACTTCCTTA
TTTATGGCTGGTGGGGTTTGTACATTTTCTCATTTCTGTATAACATTTCTTGACTGTAATAAGCAATGTA
TTCATTCTGCTTTACCACTTTCACTAACCTTAACCTCAATATATACTCAATTAAGCAATTGAAAACAGCA
GTTTTAATCTTTTGACATAAATGATTTCCTCCGAAGCAAAATGCTGGAAATCCCCTCAAATGCACCTTTT
ATTGATGAATACCTATAAGCACCACCTACAGTCGCTGGAGGCTGACAGGAACCAAACTTGATGATAACCA
CTGAGCTGAGAATTTTCAACTCACTCTTTTTCCCTGTATGGTTCTTCTAGCTGCATTATTTCCCACTATT
TAAAGCTACAGCTGGTGAACTATTCAAATATTTAAACTTTGGAGAAGAAAATATCAACTTATCACAACCC
TCTTTTTATATTCTAAATTCATATACCTGTTTGGTACTTAAAGGAAAAATATGCTGAGGAACAGGCTGGT
CATAAGACTGTATAGAACGTGCATCTTCCATCCTATTGAGGTGACTCCTAGACAATGGGAAAAATGCCTT
CACTCGACTTGCTCATTAAATGTGACCGTAGCTGCTAATCTTTTGGCGCTGTCTCGAACTTTAATTAGAT
GTGCTCTTCTCTTGAAGGTTGGAACTACAGTATCCAGAGACCATAGAATCACAGAGTTGAAAACAAAATC
TTGGAAATCATTGAATCCACTTATCAGATGAGAAAAAAAAAATAAGCCCATGGAGATAGCCATTTTAAAA
CATATCATTCTATTTAGCCTCCAATGTAAAACAATGAGTTACTATGTTTCAATAATGTTGATGTTAAGAA
ATTATTTGATAGCTTCCTCACTTGGTCTCCTATATTCCTCCAAGGTTACTAGTTAGGAAGACTGTCATTC
AAATTTGGAGACTACATAAGAAGCAGAAAAAGCATATAAAGAGGCACATGAAATTGGAACTTTTCTGGTA
AAATCTTCTTTCTTAAACTCTCCTCAAATAAGCTGTTGGTGGCAGGAGGTGAAAGACAGCCTCCACCCTT
TAGCACAGTCCGTACTTGTCAGCATTTCCCAGGAAGGGTGATGTCTGGAAATGATAGAGATTGTGGAAGC
ACATTGCATTATGGGTCAAGAATGCGAAGGTCAAGGAGTGGAGTCTTCCTTTACGAAGTAGTGTTAACTG
CTTGGCGTGGCATTGTTGTAAACAGAAGCCACCAGGAAGGATCATCCTTAGGAGGGAACCTGTAGATATG
ACTGAAAACAAGAGAGATCCAGTTTTACCACTCTGGAAACATAGGTAATAGAAAGCCCAAAAGGTACCTT
ATCACTTGTTTGTTCCTTTCTGTACAAAAGGACTTAAATCCTTTCTGAGCAAGAAAGATATTTGAGAATC
CAATTTTGTTTTAAACTTGAGCTTAGCATTTTGGAACTATTCCAAAGACCACAGAATTCACAGTCATTAG
CATACCACAGCAGACTCTTTTCAAATATTGCAAACCAGAACAGTCTGCTTGAAAACCTGGAAATACGACC
TAGTGGGTTCAACTTGACTTTTTTTATTTCTAACCCTTACCCCTAGGCAATTATTGATAACTCATTCTGG
TACCTGGTATGTATATGGACTTTGTTAGAAGAATTTGACAACTTTCTAATCATCTGTTTTTTTTCTTTTG
CTTGATAGACATACATTTAGTAGAACTTTACTGGATTGTATTGATTATAAACCACATTTCAGTTCATATC
AGTCCATTTTGCTGCACAATAAACAACCAAAAAAATTTAATTCAGTGGCTAATAACAACAATATTGATTT
ATTCATGGAGCTGCAGTTTGGTAGGGTTTGGCCAATCATGGCTGGAAATGGTTTAGCTATGCTTATCTCT
AGGCCGTCGGTTCTGTTCGGGTCTATACCACATATTTTCTTCTGAGACTCAAGCTGAAGGGACATCAGCT
ACTCGGGGTATGACAGAGTAGCACAAGGCAATGACAGAAGCACAAACAACACTTTTCAAAATCTCTCCTC
TTGTCACATTTGTTTATAGCCCATTAGACAAAACATGTCTTGTGGCCAAGCCCAAAGTCAAGGGGTAGGA
AAATACTTTCCACCTATGTGAGGCCATGGCTGGAGCGTGAATGTATGATACTACTAGGGATGTGAAAGGA
TTGAGGCCAATAATTCAATCTTCTATTGGAGACAAGCTCAACGAGTTAGTTAAAATGGAAGGCTAATATT
TACTAACTTTGCAACCCAAGGAAGAGAAAGCAGGATCTCTCTGACGATGACGGAATTTCATACCCTCATC
TTTGAAGTTATACTAAAGCTTAGGAACAACCGTCAGATAGGACTGAATTGCTCCCCCTTCCAGATTCAGC
ATGTGAAGTATGCAGCATCTTATTATAGCAGTAGCCAAAACAGCCGTTTTCTTCAATTTGGGAATACAAT
GTAGGTGTGTTAATTTTCAATTAAGAGTTCTAAACTTATTATCTGCTTGGTAGCTCTTCCATGTGACAGT
CATTCCATCTGACTCTTCATGTTGGCTTTTGAACTAAATTTTAAAGGAACCGCCAAAATTTAAGGGCCAT
GTACTTTTTATAACCTGTTTGTGGTCTGGGTAAGAAAATAAAAATTATACAACTGTTCTTTTTGACCAGC
CACAAGCATGTAATGAAAATGACTGTTTTGGCTAGCAGATGTATTAGAAGCTTTCAAGGTGTTTAAAAAA
AAAAAAAAAAACTGGAGAAAGGAGCCAGTGAATTGACCTCAAACAAAACAAGAACAAATAAACAAAACAC
TTGTCTGCACTTCCAAGGAAGGGTGATATCTAGAAAAGATAGAGATGATGGAAGCACCTTGCATTATGGG
TCACAAACGTGAAGGTCAAGGGGTGGCGTCTTCCTTTATGAAGTAGTATTAACTGCTTGGCAGGGCATTG
TTGTAAAAAGAATCCACCAGAAGTGAAACAAGCAGCACTAAAAGTTAAAAGATTTATGTGTAAACCTCAT
CTAAGGCAACAGAAGCCATTTCTATAAAATAGTATAGGACCTTTTATTATATATGGTCCTAGAGTATATT
AAAATAAGTCTGTTTGGGTCCATTTGCAGCTCATTTGAAGATTTTTATAGGAAAAACATCCTCAAAAATA
TCATACTACAGTGCCTTGATGCTTTTTTCTTTTTATAAGGTACTGCCAGCCCAAATAGTAAGAAACCGAT
ATGATTTTTGTCCATGTGAGGTGTTTAATTGCTTCCCAAAATATGGTTATTGTGTAGATGTCACTAACGA
AATATATAAAGAGCAGTATTTGGGAAAATTTATTTTAATACCACCTTTTTCCTTTTTTACCCTAAAAGTA
TTTATTTTTTTCGTAGCATACACTCTGTGTCTCAGTATCATTGTTTTTCATAAAAACATAAATTCTTAAC
AGAAAATTTCCTGCAAGCTCCCCTAAGCTTGAAGAGACAAAGGAGATTTGTAATGTAGCTCAGCCCCAAT
CAGGGTAAAAGAATGCAGGGCTGACTTTATACTTATAACTCAGAAAAAGGTTATGCTTCCCGTCTCTTCA
CAGAGCTAGTCTCTTAATTGATTCCGAACTAGGAACATGTACAAGTGGCCCACGATCTGGAACAGACTGG
CGGATAATGGAATATTGAGACCTTGTCTATGGTCAGCCATATTAACACTGGATAAGTCTGATAACACTGT
GATTACATATGTATCAATATAGTATGCTGTTAATATATTAAAAACTTATTTACAACATGATTATTGGACA
ACTGTTACAGTACAGCCACATCAATCCTATATCAAGTTAGACCATGTCAACTGGTTTTGTGTTGAGACAC
CTGTGTATGGACATAGTCTGAACTTTTCATAGTTTGTGCTAAATGATAGCAATCAACATCGGTATGGCAC
TTACAGTTTACTGATAACTTTCATGCCCATTAACATAGTACCGCAATAACTCTGTGAAGTGCTGAATTTG
TGTCCTGTTTCATGATTGTATTTGTGTTGATATCTCAGTCAGTCAGAGTCCCAACAAGAAACAGATGGCA
CATTCAGATTAGGGTAAGTTGAGGAGTCTTTATTTACAAGGCACTACATACTCAGGATTGGGCAGGGTGT
AGGGAAATCTCACAAGATAGCACAAGACTCTAGGACTAGCAGCAGCAGAGCTGTCACCTCTCCTAGACCT
GAAGCCGTTGTTGGGGAGAGAGGTTTCTCAGAGCCCAGAAAAAAAGAAAAAAAAAAAAAACATCATGCAG
ATTTTAATGCCTTGGGAGGAGCAGTGGCTTTCTCTTAAGGACAGAATTTGCCTCGAAATGATACTCAGGG
AAAAAGAGATGAAGGGAATCAATACTCTGACCCAAGACTCTCCCTTCTCTGCAGTGGTTTGCTAGTCCTC
TCCTTGGTCAAACCCAAACAGAAAACCATAGGGCATAGGAGTCTAATGATGTAATCCAAGTCAGCCCCCT
GGAAGGTGGAAAAAGAAGGGAAAATGGATCTGGATCTGGAGGGATACCAAAAAAAAAAAAAAAAAAAAAA
AACCATAGTTGGCATGCTTGTTTATTGATATTTTCTTGCATGATATAAGAATCCAGATAAATATAGTAAG
AGGTCTATTTTACTAACAATTTTAGGCACCTAATAATAATACTCCTTCTTTGAATGTATAACCTCTAGAA
TTGGTTCAGAAATGTAACTGTGCCGTTACAATTTCTATTAGTATTCAACAGTAGATTCATATCCATTCAT
CTATGACTGGAGTATCTGCCATTTGCTGGTTAGTTACTGTGTAAGGTACTTTGTAAGGTATAGAAATACA
CTTGGGGTGCGATGGCTCATGCCTGTAATCCCAAGGATTTGGGAAGCTGAGGCAGGCAGATCACTTGAGT
CCAGGAGTTTGAGATCAGCCTGGGCAACATGGTGAAACCCCATCTCTACAAAAAATGCAAAAAGAGTACC
TGCGCATGGTGGCATGTGCCTGTAGTCCCAGCTACTCGGGAGCCTGAGGTAGAAGGATCACGTGAACCCA
GGAAGTCGAGGCTGCAGTGAGCCATAATGGCACAACTGCACTCCAGCCTGGATGACAGAGTGAGACCCTA
TCAAAAAAAAATAAGAAATAAATTTGAGCTCAGTGACCTACATTCTAGTGCAGAAAAAAATGACCATAGT
TGATTATGAGATTTTAAAGCAATAAACCACATGAGACATACTAATGAGCTCATAAGATCATTCAGAAATT
GTTTATTATGAACACATAGTACTTTCAGTGTGGCATTAAACAGAGATCACTGTCCTTAAACAAGTTAAAA
GCAGAATCAAATCATCTGCAAATTAACACACCACTAAACTTTAAGCTTCTTGAGTGATTCTGTAATTTTT
AAAATGTCTTCAGCATTTCAGTGTCAAGATAGTGCAAACTCAGTAAAAGCTTGTGGAATTGCATTAAACA
AAACCAAAATAAATAGATTTTATTAAAACTATATACAATTGTCTTTCTAATCATATCCTCTCCATGAATA
GGGAAGAAATAATTTTAGGAATTTAAATATCTTCTATCTTAATAGTTCCTCTTATTTCCCTCTTAAGCAA
TGTTCACTCCTTCAAAAATATTTATTGAGCATCTAATATGTACTTAACACTGTGCCAGGTGCTGTGAAGA
ATGCCAAGGAAATAGAATGAACTTCTAATTCTTTGGAGTTCCAATTAAATAACCTAAAGTTAAATTGGTT
TCGGAGAGAACATTATGCCTTCGAGACTGTAGGCTTCTCTTGATTAGAAAGTCTTAAACATTTTAAGTAA
CTAAACAGATTAAGGAGAATTCAAGGATGCCTCTCACTAGTAAATTTGGATTAGTCTGGCAAACTTCAGA
CCTTAAATGCAAGATTTTTAATAATTAAAAGAAGAGAGAAAATGATAATTACATTTCTAGAGTCTATGTT
TACCATTCAGCCTTCTTAATCATTTCCTAAGTATATCTGGTGATCAGGATTTTATAACTCCAGAAAATCT
TTCTATACATCGCATAAATCTCTTCTTTTAAAAAGCTCTTCAATTTTGTATTTTGTTAAAACTTAAAAGC
CTCCATGAAAAATGAGACAAAAGTCAGTGAGAGGCTGTAGCAATAAAAATCAGATGTGATTTTCTTTTGA
ATAACATCTGTTTTTACAGTCCTTTCATGTTAAACTTTATAAGAATTTATTATAAACAGCTTTATTGACA
GTTCAATCCTATTTCTAAAAGGATTTATTTTCCCCCAATGGTAAGAGTTTTCTTTTCTTAAACCTAACTA
GTTGCAGATATTTCAGATACTACATTTCTCATTGTGTAAGGTAAAGTTTCTGACCACCTGAATATGACTT
GTAGCTCCTGAGAACAATTTGTTTAGTACCGATATCATGCAGTGACATTGGTACAAAGGAATTTTCTTTA
TTTCACTGTACTGTTTTCAGTTTTATTCTATAGTTGTTAAATAAGACCATTAAATATTTTTATTAGTCTT
ATTTCCTGTTTAACTAGGTGGGTTTTTGATCTCTGTTCAGTAAAGCATTGTGCTCTTCAGAGCAAGCAAT
TGAAAAGCAAATAGTGAGTATTTCTACTGTAAAAGTTTAACATTAAAAGATATACACACAGCCAGGCAAG
GTGGCTCACGACTGTAATCCCAGCAATTTGGGAGGCTAAGGCAGGAGAATCGCTTGAGCCCAGGAGTTCG
AGACCAGTCTGGGAACCATAGCAAGACTCCGTCTCTACCAAAAAAATTTTTTAAAAAATAGTTGGATGTG
GTGGAACACCTCTGTAATCCCAGCTACTCAGGACGCTGAGGCAGGAGGATTGCTTGAGCCTGGGAGGTCA
AGGCTGCAAGGCTGCAGGGAGCTGTGACTATGCTACTGTACTCCAGTCTAGGTGACAGAATGAGACCCTC
TCTCTCTCAATTAAAAAAAAAAAAACAAGATACACACACATATATTTGCGTAGGTAACTCTAATTTCATT
TCAAGTATGTTATGTAACAACCATTTGTGTAGTGCTTGTAACAGTCAATATGTAAATACTGACTCATCTT
CTTTGACAATTCTACCTAGATACTTATTAGAGTCCCCCTTAGTCATTGAAAGGAAGGTTAAAATCAAAAG
ACGTTGTTTGCCAAAGTAATGAAAGAAAACTTATAAACACAATGTATCATGTCTGGGGCTGAACTAAAAC
CCTTCTGATATGTGGTATTAACAGATCATCTTTCATGACAGTACCAGTTATTAGAAATAAAATGATTGGA
GTTATTATTAATACTAACAATAGTGGTATTCTTAAAATGACTTCCTTATTTATCTTCACCTTTATACATT
CTACTACTGCTTCAAGACCCATCTTGAATTCTTCTTCCACAGAACATTCTGCATTAATTTCAGCCAACAT
TGATTTCTCTTTTTAAAATTTGTCTTGCACAGTGAATTAGAAAACCAGGAATTGGAAAACCAGAAAAGCT
TATTAAGTAAGAAGCAGAGAGGAGAGAGTTTCAACAAAGGGCCATTCTAAAGTGGTCTACTGCGGACACC
ATACTGATTATAGTTGGTGATTAAATCTTATCTTTCCAACTGATTATAAACTCCTCCAGGGCATACTCTT
ATATTCCACAAGATGCTTATCTGGGTGCAGAGCATGCATGCAGTTGGTATTTGCTGATTTATCAACTAAC
TAAATCTTAACATATTATTATTAACAATTTAAAATAAAGTTAAATGTATCACTCTCCACCCCTCAAAGCC
ATTTCTGTTCTTTGTTTTCATAGCACCATTATTATTTCCTGCATAGTATTTTTTAAAAACCGTATTTTTA
AAATTTATATATTTGTTTATTTGGGTATACTTCACTAGATTGTAAGCGTCACAAAAGCAGAACTATTATA
ACCCCAGCCACTAACACAATGCCTAACAAATAGTAGGTTCTCAATATTTGTTGAATGAATGACCTACAGA
TATTACTTCATTATGAAAGATTTTGCTAAGTTGTTTTACATCTATTTTATCCAAAACTAAAGTTCTTGAG
GCAAAGCCTAGAATATCTTCTATGTTCTCACAATGCTCTGAATCAGTGCTTCTCTTAATATGCATAGCAA
TTGCCTGGAGAGCTTGTTAAAACATAGATTACTTAGCCCCAACCCCAGAGATGCTGATTCAGTAGGTCCC
AGGTGATGCTGCTGCTGTCAGTCTCTGGCGCACACTTTGAGTAGTAGGGCTCTAGGATGTTATATGTACA
GACACATGCTGAATAGTGGGCTATGTGCTTACTTGCTGGCTAAATAATAAATGTTCTCACTGAGTCATAG
AACTTTGAAATTTGCAAGGACTTTTGCTATTATCTAGTCTATGGATAGCAAATAACCTGATACCGTGCTA
TAGTGCTTGACTGCATTTAACCTGCAGAATCCTCATGAGCAGCCCAGCACCATCACTCCAAGTGAAACTA
CTCTCTTCTTGAGGTTGTCCAATTCTATCAATTAAAGATGAAAACCAGGTTCTGAGAGTTGAAATCTCTG
GACTTCAAAGGTCCAACAGCCCAGGTCTTCTCAATTCTCGTTAGTGTTTCAGCAGCTGAATACAAATTTA
TTAAGCTGTATCAGAGTAGTATCTGTCAAATTGGAGTGTCCATAATATGCTTAAACAGAGAACTCCATTC
CAATAACATGAACTTTCCTTATGCTTTATTCATCATCGCTTGAAATTTTGAATTTTGCCCAAAGAAGTTT
ATACCAGTACATGTTAAATTACATCATAGCCTTCTTTGTATAAATCTTAGAGTAGTTTACTGAAGTACAT
CGCAAAGTTTTGTTGTTTCTTAGGTGATTTTAATTATGTATGTTTACTTTCAGTAATGCATCTTTTCTCC
TTCATCAATATTATGTTATGCTAGCTGTAAGTACAAAATAATTGAGAACAAATTATGACAAATTGAACCA
AGCCACAAAAAAAGGAGAAACCAAATACTTTTGTGATTTGAGCTTTTTTCAGTCCTTGAAACTTTAAGAA
TATCTGTCTTTATTAACTTTTGCTTTTTGCTGATGGTTTCTCTCATTTTATTATAGCTTATAGCATTGTA
AATTAATTTAACATGAAAGGATAAAAACGTTGCTTTTGAAATGTTTCTCATTAAATTATGAAAAAATATT
ACACTAAATAAAAGAAAGGAATGCCTCTGGTACCAGCTTCTGTTTGCTCAATTATTGCAGTACCCAAAGT
GAATTATTACACAGTTAACTCAGAGGCAATATTATTGTCATTATATTATAAAATAGATGAGTTGCAATCT
TCAAAAAAAAAAAACAGCATAGGTCCTTTGAAAGTGAAATACCTTTTTTCCTTGTGCTTCATTTAAATAT
ATACTGACCCCAGTTTTGTTTTTGTTTTTCCTTTTTAGAGTTCTTGCTAATGATGGGCCCAAAGTTATAT
TAAGAACTGCAAAGTAAATTTCAACCAATTACTTTATTCAGGGGAGTCATTAAATTGAGGTACCTCTGAA
ATTTTGGAAGGAATGTACTGCCAATTAGCCGAAAGCACTACTCAATGTCCTTTCTATGGTTATAATCTCT
CTAGTGTATTTTTAATTGAAGACAACCTCTATAGAGGAGGTGAGAAGTTGCTATTTATTGGTACTTGTTA
GGATGGAATCAAGGGTGTGGAAGATATTCATCTATTTCTCTCTCCAGCTCCCCCACACAAAAAGAATGGT
GCTTAATCCATCTGAAGCATTTGGGGAGCGAGGGTAAAGATGTAATATTTACCATGAGCCGAAACAGATC
TTCAGAAGTGGAAAATGGAAGCATATTGAAGTCCCTCAACTAAACAGACTTTCTTCCATATGGAATTCAA
TGCATTAATGTTTTCAAATTCTATAGCTTCAAATTCTTAATATTTTCAAATTATGTGAGCTTATGTCAAA
ACATTTAAGTGAGCTTTTAACAATGAGGCAAATATTTGAATCATTTGTCTACATAACAAATACTACTATA
AAGCATATTAAATGTTATAAAAATCCTAATATACTAATGTAAGCTATTATAAAGTACAAATAATTAAACA
ATATTTATATGATCAATGTTTTATAATACGATAAACACATTAAATAATTAAAAACTCTTCCACCGTGCAA
AAATGACTAAATAAATTGTTAATTTCTAAGGCTTTTTGAGATTACTGTGAAAGGGGGTATAGTTTCAGGA
AAGGTGAAACTTCCCTTCAATGTGTAAACCATTAAAGAACATAATAACCTACTGAGTGTGGGTCTCAATG
ATATGCCCTGGAAAGTATGGGCAACTACTCCACACCCAATTTTGTCTTTATATGATAAGGCACAGCAAAT
AATTATAATGCAATGGATAAATGGTAAATCCCACCAAAGATTAACCAATCAGAGCAGGATGAAAATTCTG
AGTTTGGAAATCTATTGGAAGATTTACAGATTAGATTAAAGTGCCCAGTAACCAAACTATCAAAATTATA
TGGCTTCAGTTAATTAATGATTTCCAAGGTTTTTAGTATACTGTATTACAAAACACATTAAGCATCTTAA
GCATTCAAACAACATTTTTTTGATGATTCAGAAAGCATCACAAATTGTTATATCAGCTGATAATAACTTA
GGTACATATCAATTAAACTTGTATTATAGACACGCAGAATTCTTCAGACCAGAAGTCGAAAGGGCTTCTC
TAGTTTGTTTATGCTAAGTTGTTTAGAGATGACATAACTCTGAGCTAATTTGTCTATTGCAATGGTTCTC
AAAATGGGGGGGGGGGGATATTTTTACCTCCACCAAGTGGACATTTAGCAATATCTGGAGGCATTTTTAA
TTATTATTACTGGATTGGAGATACAACTGAAGTCTAGTGGGTAGAGGCCAGATATGGTATAAAATATCCT
ACAATGCATAGGATAGCCCTCCACAAGGAATTATTTAGGCCAAAATGTCAGTAGTATAAAAATTGAGAAA
TGCTAGTCTAATATAGTGTTTACTCACCTTTCCTGAAACTATGTCCCCTTTCACAGTAATCTCAAAAAGC
TTTAGAAATTAAAAAATCGTTTAGTCATTTTTGCATGGTGGAAGAGCTTTTAATTATTTAGTGTGTTTTA
TCTTATGAAATGTTGATAATATAAATATTGTTTAATTATTTGAACTTTATAAGAGCTTATATTAGTATAT
TAATTAGGATTTTATTTAACATTTAATATGCTTTACAATAATATTCATTATATAGACAAACGTTTTATTT
TTTTCACTTTAACAATGATTTTTAACTCTAATTACATAAGAAAAAGTATGAGTTAACAATTTTTTAAATT
ACATGCTTGGTTTGAGGGCCAAATACACATGAAAATGTGGACTAAAATTTAAAATCAAATAAAATCTATA
AAGTCGAGGAAAAAGCTACTTTTATGACGAGGCATGGGGAATTCTTCATAGTTTTTGGGTTTTATCAGAA
GTTAGCTATTTTTTTTCTTTTTGCTCTGTAAACAATCAGATAAGAGAGGCTCAAATGACATTTTCAAGTA
CATCTTAACAAAATACACTTTGAGCATCAATTGAGTAAAGTTTCATTCTTTTGAAACTTTGGTTTTCACA
AGATTTCCTGAGAGTTTTATTTTATTGGTGTTCTGTGGGACTTGGGCATCATAATTCTTACAAACTACTC
AGCTCAATCTAATGTGCAGCGAAGCTCTGGGAACTTTTGTTTTGTCTAGTATCCAGTTGGAAGATTCTAT
AGCTACAGAGCTTGGGTTTAAACCCCCTCCAAGTCTTTACCAGCTACCTTTATGACCCTGGCAAATTACT
TAAACTGTGTGCCACCATTTTCTCCTCTGTAATACGGAGGCAATAAAAATTTCCACTTTTAGATTTTCTA
TATGCGGTTTACAAATTGACTTACTCTGAAGATCATCTGGAGTAAAATCTGGAGAAATATGATCCCTTAT
AACTTCTTCAACCCTTTATATATTTCAACATGAGTAACCAATGCTCTAAATATGGATATAAATTATAAGA
ATAAAAAATCTAGGACTATTATAATGGTCTAAACTCTCTTCATAGCTAAAAGTGTTGAGTAATTAAACCA
GTTGAGCAGCTAAATCATGTACACACTTCTTTGATCCCTCCCACGATCATGTATTTGGCATTGTAATGAA
AAGATATGTTTATTTTCGAGAATAGACATAACTACCTTTAATAATATGATCACCCAGAAATTTTTACAAA
CCCCTGGAAAATTTCATGAATATCAGGCTGTGCTCATAAAACCTTAGAGATGAGATCACAATAGACTGGG
TCAACATATAGTAATGAGCAGGATTAATAAAACCTCAGATGGGCATTTACAAATGAGTCAAAACCATGAG
TATAATTAAATAATTGTAGCAAAAAAAGAGCCTTGGGTAATCCTTTCAGCAAACGTAATCGAAGTGATTG
CATTTAGAAGACAAATATTTAATTTGGTGACTAGAAGGTCTTTTATTATTCCATTATGTCTTTGTGTGTG
TGTGTGTGTGAGACACTTTTCAAGGTCAATTTTTACTTATAAATTGTCTCTAATTAAAAATTGACTTGGT
TATTAAATCATTGAAAATTGGCCATCATCAAATTCCTCATTAAAATATTTCTATGTGCCATATATATATA
TATATATATATAGAATATATATGTAGAATATATGTATACATTTATTTTTACTTTTTTTTTACTGTGCCTA
CTAGAGAAATTTAAACTACATATATGTAGAATATATGTATGTTTTAACTAGACATACTGTTAAGTACACT
ATACCTAATATTTGGCAATATTAATACCATCTCATTGAGAAACCTGGAATATATGCACATTTTGGATGTC
TATTATATGTTGGGCACTGGACTAGTCATTGATAATACAGAGATTAGTAAGACTCAGGTTGACTTCAGCC
ATGTTGTCAGGAAGCACACACTCTAGTTTGGGACAGCGAGGAGAAATTCAATAAGAGAAATATATATAAG
GCATAATGCTCTAGGAGAATATGCAGTGGATAACTGCCCAATAGGACCAGGCAAGGCTTTTTAGAGGAGG
AGGTGGCATTTGAGTCAAGTGTTAAAGGCTGAATGGAAATTCACTGGTTGAGATAAACTCCTTAGGAGGA
ACTACTTTAATAGAACTTGCCGTTAGTCCTGAAATAAATGGTGTGCAAAATCATTACCATCTGTCAATTC
ACTCAGTCTACTTTGCTCTTAACTTCAGAAAAAAATCAGAAATACAATTAAAACATTTGAGCCTATTTTA
CTGTCTTTTAAAATGAGTTAATTCAAAGAGGAAATTAAATATAATGAGAGAGAATCTCCCCCGAGGATTG
GGGGCTGGGGAAATGCTATTGATTCTTTGCTTGTGTTTATTTTCTCTCAAAAATACATTATGCATAAACT
TGATGATCAAAAATTCAGATTATTACATTTCTAAATTGGCAATGCAATTTATTGCATCATACATCAATCA
CAAAAATGCTCATCTTGCTGACTTTCATAAACTTCTAAATGAACAAAAATGCAAAAATAGTTTATACTAT
ATTACACTATAGTAGATTTGTTAAACTAAACCAGAACAATGGTCCATGAAAAATAGGCCTCTGACTCCAA
ACGCTCACACCACAGGATCTCTCTGAGATTTTTGTGTCATTTCAAGTCAGAGAAAATTGTCTAATAAATT
GTTGGCTTGTAACAATGAAAACTAAGATATCTGTGGGGCTATTCTTGTTCTCTTCATTTTACTACAGCAG
CTCTGCCCAGTAGAAATAAAATGTGAGCCACATATGTAATTTAAATTTCTCTAGTAGGCACACTGAAAAA
ATAAAAATAAAGAAGTGAAATTAATTTCAACAGTATGTTGTATATAACCCAATATACCCAAAACATTGTC
ATTTTAACATGTAATTGTTACAAAAGTTATTAATTAGATTTTTTCCGTTAAGTATTTAAAATCTGGTAAG
TTTTACTCTTACAGCGCAACTCAGTTCAGATCAGCCACATTTCAAGTGCTCAGTAGCCATATGTGTCTAG
TGGTTACCATATTAGAAAGTAGTTTGAGAGATCCACATTAAACCAAAAGGAAAAGAACTTCCGGCCCTTC
ACTGATGAGTCACTCTTCACTGCTAACCTTGGAAGCATTCCCAAATGTAGTCTACAGAGTTTAAATAGTC
TATCTTAACATCTCTCAGGGCTTCAGTCTTAATGCCATAGTATTTTTAAAGAATGGTGGATATTCTTTTT
TACAGAACACTCTGTAAGAGCAATTAGAAGTTTATGATGCACGTAATGCAAAATACAGGTCATTTCCCAA
GCCTATTTTAAAAGCGCAAAAACTGTAGTCATTTATCACCCCTGAGAATGTTGTCTTAAATGTCTTGGTT
TGGATATTGGTGATGTGAGAACTTTGTGATAAGAAAGTAGTCTTTAAGAATAAGATATCAGACTAAAATT
CATATCTAGAATGAAAGTCTTGTTTTTAATGGAAGATTAAGAGCAAGTCTGATTCAGATCATGCATGGGG
TACACTAGTCTAGGAAAACACTAGTCTGAAAATATACTAAAAGTTACTTCGCAACTTAACAAGAAAATGT
CTTGTGGGTGATGTCGTTCTTGATTTTTAGGCAAACCTACCTACCTTTGCAAAGCAGCTGGGACCTTTTT
GCATTGGAAGAATCATTTGGAGCACAAACAAAATTAGATTATCAACACTTTGGAAAACAACTACGAATGA
GCAATCAGAAACCTGACCTTAAGATTACTTGTGAATTGTGAATCAGCAAAATAAACTCGATTGTTCATTG
CTAAGTGTATTTCAATTATCAAGGGCCTTCTAGATTATAAGTAGTCTTTTTTTTTTACTTAGTTTACAAT
TAAGATGTGTGGTATTTGAAATACATTTGCCACAGGGAGAAATATAAATTATAATTAATTTCCTAGGCTA
ATTCAATTTATGACATACCTATATACATTATCTGTCATCTATAATTTTTCCCTTATTGTTTACTTCCCAC
TGGAAGAATGAAAATGGAATATTATTACATGGCACATGGCTTGATACTTTTACAAACTCTGACAATTATG
TATTTATTTTGGGAGGCATTGAGTTTATTTGTTTTATTTATATAAATTTATGAGGTACAAGTATAATTTT
GTTACATGCATAGATTGTGTAATGGTCACGTCAGGCCTTTTAGGGTATCCATCACCTTAATAAGATGCAT
TGTACCCATTAAGTAATTTCTCACTCTCATAAAATTCTAATTATGTGAATTTAATTTAATCTATTTAATG
TGTTTTAGGCAAATATAGCCGGTACTATAAACAGTTGATTTTAAGATATCATTGCTTACATTGAGACTAA
GTAAAACAAAATGGGTCAATAAATGTCAATCTAGATAACAATGTCAACTAAATAAGAGGTCAAACATGGC
AGTATTTTTGAAGGTGATCTGTGAAAGTGATTATAGCGTTTACACTCATGGAAAATGCCTTCAGAGTTTC
AACTAAGAATGCCAACAGCTCATTCCTTTATCCTGATGCATATTGTCTTCCTTCTCACCCCCAGTTCCTT
CTTCCCCTAACCCCTACCCGCTTTCCTTTGCTGATTTTGACAGAAATAGGACCCCCAATAAGTCAGGGAG
ATAGCAGGAAATGGGATAGGATAGAACCCGGAATGATAGAATAGCTGAGCCTGAAGGCATGAAGAAAGGC
TCCTCCTGACATCTAAATGGAGACCTAAGAGATGGGTTGGTCAGGTAGGGGGAAGGAAACATGAGGAGTA
TTCTCTAAGCCAGGCAACATACTGTGCACAAGTCTGAAGTCATGGGAAAGTGATTTTGAGAGGATTGCTG
CTTGGTAAACCTAGAGTTTGAATTGGGAGAGATGAAGCTAGAAAGTTAGTAAGGGTCAGATTTTTTTTTT
TTTTACTTGCATGACAATGGTAAAAACCACTAAAGGTTCTGTGTTAAGCAGAGGAGTGACTTCATTTAAA
AAGGTAAATTGGATTGAAATGAAGGGCATAAACTGAGGCAAAAATATCCTTCGTTAAGTTATTGAAGCCC
AGTTGAACACACTGGTGGCTTAAACTGGAGTATTGGTATAAGTGGGGGAAAGAGGTTAATAGATTCCAAG
TTGAAAAAAAAAAAAAAAAACATAGACTTTGCTATCTAGTAATGGATTAATATACAAAAGGAAAAAGTAA
AGTTTCTACTTTTTGGACAGCTAGAAACCTTCACCGAAGTAGGGAACCCAAGACTTAGATTATGTTGGGA
GGGGCAGGGTATTTTAGTTGCACAGGGATTTGCTTTACAGAAATGACTGAATGACAATATAGAGAGATCA
ATTCCATTAAAAGAAGTTTGATTACTCACAGTTCTCAAGGGAAGAGTACATACTACGCCATGCAAAGCCA
TGCAGGAAAAAAGTTCCAGAGTCGGTCAGCAGGCAGAAAAGGAAAGCACAGCCCAAACCCTTTATTGTGG
TTTCCAAGGAAAAGAAATGAGTGAGGTAGAATAGGCAAGTCTGAGCAAGTTTAGGACTGGATAGTTCAAA
TAATTTCCAAAATTTCCTGGCTGTAAAAGTGGTCTCTGGTTGTCTGGTACCAAGCCCTAGGGTGAGGGGA
AAAAGTTAGGGTGGGGGAAATATTGGTTTGGTGTAACAACAGTTAGATGAAGAAGGTAGTTGGGGATACG
GACTTTGGATTAGTTGGTTTGTATAACGAAAAGCAATCCAACAAATCCACAAGGGAGCAAGTTTACAAGT
TATTTGCTATCTTTAGGAATTAGCTAGCCCTGGGAGGGGCAGTCTCTCCCTGGCCTTCCAAGGACCTCAA
GATGTTCAAGCATCCATAAAATATGGAAATTTTTTAAAAACATTATAAATACACAGAGTAAACGCTGGGC
ATGATATAGGACAGTGGTTCTCAAACTTTAGCTCCACTGGAATCTCCTGGAAAACTTGTTAATATGCAGA
TGACCGTTTTACCCTTAAGCTTCTAATTGGGGAGGTCTGGGGAGGGCACAGATAATTTGCATTTCTACAA
AGTTCTTCCATGATTTTGATGCCGCTGGTGAGGGACCAGGCTTTGAGAACACTGATTTAGGACGTGTCCT
GTTTAGGGAATATCCAAAAGGCGGACAAGTTCAGGGAATATTCTTGGGCAGTTGGCTGTGTGAGTCTGAA
ATTCAGGATAGAATATTAAGCTGAAATAAAGATTTGGGAGCTTATCTACAGTCAAATGATAATTGAAATA
CTGAGAGTACGGGGGGAGAGAGGTCCATGTACCAAGAAAAGTGAAAATGACTAATCCCAAGCCTCGCTGA
CCATTGAGAATGGAGCTAAGTGAGAGGAGTTAACAAAGCTGACCCAGAAAAAGTCATAAGGGCCTTAGGA
GGCCAAGGAAAAAAAATACATTCACTGCCAACGAGAGGCACTTACGAATGGCTTGACTGGCTTTGCCAGC
ATGCATGAACTGCTTCATAATTATTTGTATTGATTACAGTAACAGATACATATTTTAACAAGCAACTTAA
GTAATACAACTGATTTTTAATTATCTTGTTTAAATTGATAAAGGTTGTATATATTCATGGTGTACAACAT
GATGTTTTGATATACCCATACATTGTGGAATGGCTAAATCAAGCCAATTATCGTATGCATTACCTCACAT
ACACTTTATTTGTGGTGAGAACACTTAGAGTATACTCTTAGCAAGTATCAAGTATATAATACATTGCTGT
TAACTATAGTATCCATGTTGTACAATAGGTCTCTTGAACATACTCCTCCTGTCTAATTGAAATTGTGTTT
CCTTCGAACAACTGATTTTTTTAAATAAAAAACTTAATACCTGTAAGTTAGAATTCTTAATGGTCACCTT
AGGAGCCTATACAATTATTCCTACGTTGTTGTTACTATTCTGTGTCTTTTTCTTTTTTAACATCTTTAAA
GGTATCAAATTTTTATATTTTGAAAGTAGAATTTATTTTTTGTCAGTCTAAAATATTTTTATGTTGAACA
AAATGCATGAATGGTAAACCTAGATGCAATCAATTTTTCAAATAAAAAAAGTAGATACCCATGAACATTT
CTTTTGTAATTGCAAACTGTCTTGAAAGGCAGTTTCAAAAAGAGTTTAGTTCCTAAATTGTACCATTACT
CACTGCGTTAAAATGCAACATTCATTTGAGCGTATAACCTTTTGATCAATTTGTTTTTGATGTCTTGTTC
CCTGAGAGTTGTCTCAAATAGATACATATAAATATACACATATCTCAGATTGGCTCTGAGAAATGTCTTG
ATTCAAACGTTCTTGATTCTAAGATTCATGGTACATAGGAACTGTATGGTGACAACCTTGTCAGCCTATC
TTTAGAGTAGCTTTGGATTCTATTCAGAACATTTCCCAAAGCTATTCTGCTATCAAGAATATAAACAGGA
ATAGTCAAGGGAAGCTTTTTAAAGGGCAACATTTTCATGTAGGCATTTTTCTCACATTGAAAACTAGTTT
ACTAAATGCAGTGTATTACCTTCTCATTACAAGAAGTCTTTCACATTAGTATAAATGCATATGGCAGTTG
TGCCAGAAATAAATTGCCTCTCAAACTAGCACATGGAAAGAAGAATTCTGAGATTTAGCACATATGTAGC
TTTTAAATAGTATACTCTGTTTCAAACATTATGTGTTAGTCCACGTTCTCTTCAGCCATTTTCAGTTGCA
TTTTTACTTTATATTCCTTTGTATATTTATCTTTGCTAATCATTGTCCTGAGATTCCTTTAGCTCTTGAA
TTCTACGTTTTTAATTAATAGAAAACTTTCTTTTTATTTTTCCCCCGACATAGTTGTTTTCTAGAAAGAA
ACAGTTATAGGTTATAAATCCAACACTTTAGGGCCGACTTGAACATGCATCAAAGCTACTAGAGGACTTG
TAGAAATACAGATTGAATGGTCCCATGCCTAGAGTTTTACATTCAGTTACAGATAGGGTAGGACCTGAGA
ATTCACATTGCTCACAAATTTCCAGTTGATTTTGATGGCATTGGTCTAGAGACCAAACCCTGAGAACCAT
TAAAAAACAAACAAACAAAAACAAACAAACCAAAAAAAAAACTATATACAGAGATTTTCTTCATTGGCTT
TTGCCACTGAAGACATTTAGATGAAGAGACTCCACAAAGTGTAATCATTTAGTTATGAGAGGGGCCTGAT
AATTTGCATTTCTATCAAATTCCCAGGGGATACTATTGTTGCTGATTGAGAACCACACTTGGTGAAACAC
TAATTAAAATACCATTAAAAAGCAAAAACAATTTAGGCCAGCAAAACCTCCTAAAGAATGAGGCCTAAAG
ACTTATTTTGTTTTATTTTTGCCAGAAGCTTCTTATGGGCAAAATTATCACCAACAGAGCTGAGGTTCAA
ACTTGTGTTCATAGCAAGCAAAAGGGATAATTTGGAAAAAAAGCTGAGGTTAGCTTTGTGGTTGGTTTGG
GAGTGGGAATGAGTAGGGAGGAAGAAATTTAAAAAAAAAAAAAAAAGGAAGAAGCCAAATATTAAATTGT
TCACAGGGCGAAAAAAGAGAAAAGGAGTAACTAGAAATATCTTAGACTGGTTCGGCAAGTCGGGTCCCTC
GCAGCTAACAGTGGTCCCACCCTCTGGAGTTTATATGTTTACATTCTTTTTTTTTTTTTTTTTTTTTTTG
AGACAGGGTCTCACTCTGTTGCCTAGGCTGGAGTGCAATGGTATGATCACAGCTCACTGCAACCTCCACC
TCCTGGGCTCGGGTGATCCCCCCAACCTCAGCCTCCCAAGTAGCAGAGACTACAGGCAAGTGCCACCATG
TCCAGCTAATTTTTTGTATTTTTTTGCAGAGATGGGGTTTCACCAGTTGCCTAGGCTGGTCTCAATCTCC
TAGGCTCAAGTGATCTGCCCACCTCAGCCTCCCAAAGTGCTGGGATTACAGGTGTGAGCCACCGCGCCTC
ATTGGAGTTTGCATTCTAGTTGGGAAAATAGCCAATAAATTTGTGACTTATTTTCCTTTAAAAAAAAAAC
TTATTCTGGCTATTGTGTGACTATAGGATATGGAAGGTGCAAGAGTATGAGGCTAACACCCTGTTCTAAA
TTCCGTCTCCTCTGAGCCTTGTTCTGTCAAGAATCTCCTCCTTCTATACTTTTTAAGTCACCTTCCTACT
GATCCTTTGCTGTCAGCTTACCACTCTGGTACCCTTCATTTTAACAAACAAACAATTGTCCAAGCTTACC
GGTGCTGCTCCTTCACCCCTCCACCTGTACCTAGTGTCAATTCTCTCCCTCTTCTGATGGCCAAACTTTG
TGAAACTGTAGCACAGCTCCATATGTGTTCCTGCAAAGGACATGATCTCATTCCTTTTTATGGCTGCAGA
GTATTCCACAGTGTATATGTACCACATTTTCTTTATCCAGTCTATCACTGATGGGCATTTGGGTTGATTC
CATGTCTTTGCTATTGTGAATAGTGCTGCAGTGAACATACGTGTGCATGTATCTTTAAAATAGAATGGTT
TATATTCCTTTGGGTATATAACCAATAATGGGATTGCTGGGTCAAATGGTATTTCTGGTTCTAGATCTTT
GAGGAGCTGGAAGCCATTATCCTCAGCAAACTAACACAGGAACAGAAAAGCAAATATCACATGTTCTCAC
TTAAAAGTGGGAGCTGAACAATGAGAACACATGGACTCATGGAGGGGAACAACACACACTGAGGCCTGTC
GGGGGGTGGGGCGAGGGGAGGGAGAGCATTGGGAAAAATAGCTAATGCATGCTGGGCTTAATATCTAGGT
GATGGGCAATAGCAAAGACTTGGAACCAACCCAAATGTCCAACAATGATAGACTGGATTGAGAAAATGTG
GCACATATACACCATGGAATACTATGCAGCCATAAAAAAGGATGAGTTCATGTCCTTTGTAGGGACATGG
ATGAAGCTGGAAACCATCATTCTCAGCAAACTATGGCAAGGACAAAAAACCAAACACCACATGTTCTCAC
TCACAGGTGGGAATTGAACAATGAGAACACATGGACACAGGAAGGGGAACATCACACACCAGGGCCTGTT
GTGGGGTCGGGGGAGGGGGGAGGGATAGCATTTGGAGATACACCTAATGTTAACTGACGAGTTACTGGGT
GCAGCACACCAACATGGCACATGTATACATATGTAACTAACCTTCACGTTGTGCATATGGACCCTAAAAC
TTAAAGTATAATAATAAAATATATATATATATATCTCTAGGTGATGGGTTAATAGGTGCAGCAAACCACC
ATGGCACATGTTTACCTATGTAACAAACCTGCACATCCTGCACATGTACCACGGAACTTAAAATAAAAAT
TAATCATAGAACTTTAAAAAAAGAAAAGAAAATGTAGCAGAGCTGCCTAGCTCACCTTCTTTACCCACAG
CTCACTTTTCAGTCCATTTATCTGGCTATTACTCCTACCGTGCCAGGCAAACTGCTCTCACTAAGAAAAT
CAATAGCCTACCCCCTGCCAAATTGCCTGACTCAGCTTCTCCTTGTAATTTTCTCCTTTAGTTCTCTAAC
ACCCTCTTCCCCAGGTTTTCACCTGACCTGTCTCCATAGGACATTTGAGTCTCTTTCCTGATGATTCATC
CTCAGCCTCTTCCATAATAAGTATGGCTGCTCCCCAGATCCTACCCTCAGCACTTCTTCACTTTCCATGC
CACATCTCTTCTGTGATCTCATCTTCATCCACGGCTCTAATTAGTATCTATAAGCAGATGACTCTCAAAG
CATATGCTGCCTATGTACCCCTCTTGGCCATTCTACATTGACATCTGCAACTCCCCAGTGAACTTCTATA
TTTAGACCACAGGACTGGTACCTGCTGATAAGCACAGGTAGGGTGTACTGAGTAGTGTTTTCTCTATTTG
GTTGGGCTTTTGCTAAAGCAGTTGTCAAATATTTTGAGTCTTACTCATGGCCACAGACATTTTAACATTA
GCATGTCCCAAACTGAAATCCCCTACTGCCTCTATTCTCTATTTCAGAAGATGGCACCACCATCTACCCA
ATTATTTAAGCTAGAAACTTCTGATTCTGGTGAGACTTCTCTCTTTTATGCATATGTCTACACTGACACA
AAAGACTGCAAATTTTACCTCCTAAGTCTGTCTTAAAGCAGATTTTTCTCTATGATTCTCTATGGTTTCA
GGCCCTTATCACTGTGAAGTCAAGCATACCTGATTCGAATCTTGTCACCGTTGGCAAATTTTTAAATCTC
TTCTAGCCTCAGTTTCCTCATAAAGTTTTCTGTTTCTTAGGGTGACTAAAGGGCTTAAATGAGATTACCA
TACAGAGAGTAAGGTACATAAAATGCAATTAATAAAGAATAGTCACTATAACTGCTGATGATGATGCTAT
TACTATTCGTATCCTAGAAAACTCCGGTAACTTGTTCACTGGTCTTTCTGCATCTAGCATCACTTCCTCA
GCCAGAGTTATCTTCTGACATGAAGGCTGATGCCGTCACCCCCATACTCATGTTTGAAATTCTTCAATAC
CTTTAAGATAAATTCCCACCTCCTTGGTGTAGCATGCAAGGTCACACATGACATAATCTCTCCAAGGCCC
CATTTCTTCCACTCTCCTTGAGTGATATATGTGGCAGAAAATTTAAGGCTGCCTGGATATTATCCACCTT
ACGTCCCAATACTTCCATCTGCCGCAAAGACCTTCTACCCAACTTCCCATCCTCAACGAATTCTTATTCT
TTCTTTAAAAATAACCTCAAACTTCAGACTAGACCTCTGGTCCATAGGGCATTACAAATCTCTCAGTAAG
TTGTACAGGATGAACACGCCCCCTAAAACTTTGTTTCAGATATTTCAATTTTTATTTTATTTTATTATTA
TTATACTTTAAGTTTTAGGGTACATGTGCACAATGTGCAGGTTAGTTACATATGTATACATGTGCCAGAT
ATTTCAGTGTTAAAGGTTTAATAATCACATTTACAGAAAAGGAATTAGCTACAAAATGGTGGCACTGGTA
TACAAGTATGTAAAGATACAGTGCTTACAATTTAGGATTATTGTTGTCGATGTTTTAATATTAAAATGGC
TAATCATACAGCAAAGTCGAAAGAAATTTACGGTCAACATCTGTATACCCAGCACCTATACTTTGCCATT
GAAATTTTACTATACTTGATTTATTACATATTCATCTATCCATCCCTCTTTCTGTGATCAATTTTTAATA
TTCTAATACTCTTACCCTTAAATAATAAGTTATCTTTTCAAAAAATAATGTGTTTTTACATAGATGAAGC
AAAATAAACTTGCCCTTGATAAAACAATATGCACTGTAGTGCCTTCTAATTCAGTGCATTGAAGTATCCA
TTAACAATATAACCAGAGAATATAAAACATGTTTATTAATATTCCACTGTACCTGATTAGATATAGACCA
TTAGGAAGAGTTATTATAATTAAGAATCTAGGTTTGTCAATATAGAAAAAAACCTGTGTTTTTTATCCCA
CTGGAATGTCTTGTGAGGAATATTGTTCCCCTTTTTCTAAAATTTAACTTTGACCTTTATTTTGTTAATG
CACCATGGGTTAAGCCACACTACGACATGTGCTAAATAGACCTGGAAGTTTTCAAACTAGGTTTTTAAAG
TGTATTTGACATTAAATCTTCATAACACCTTATTGATTAATTTAAATCCATTACCATGGTAAGGAAAATT
CGCAGACAGGCAGGTGAAAATTAAAATAGAAACAAAACAACATGGTAAGCAATCCTTCCCCCCAAGCCAA
TCAGCATGTAGTCAGTGTGTCCTTTTAAATTAGCAAGGCGCAGCTTCCCATAAAGTCCCAGCTTGATTTT
ATATGCTGCAATAGTATTGCTAAAATAAAGGAGAAGGCAACTTTTCTCTATAATTTTTTTCTAGAAGTTT
TCACGCAGCTTAGTATACTGCAATGACCACATTACTCAGTTCCAGAATTAGCAGCATTCCATTGTGAATG
ACTTAATTCACATTGTGATTACTCATTTAACAACATTCTTGAGGGTTTACAATGTGCAAAGCATTACATT
AAGTCGTGTGTGGCAGAGGTTCTCAAATGCAGATGATCTGTGAAGAAGATTTCCTCAATAAGCAGAGAAA
TGAGAACTATAAGGACAGAAAGAGAGAGAGAGAGAAAGAGAAATTATTTAAGCTTGTAGCTTGTCATCCT
CCTTTCTTAGGACAGCCTACCATTTAGGCTGAGACTATGTCTTTCTGATTATTTCTTGTGGTTGAAATAC
CCCTTCCTTAACAATATGATGGTAACAGTGGATGGTAAATCTTGTTTTGTTTTAATAGTTTACCTGGCAA
AAGTATCATTTTATGTCTGTATCAGTTATATATAATAGTATATCAGTCCATTACCAAACCGCCTCAAAAC
TCAGTAGCTTAAAACAGTAGGTACTTCTTGAGTTCACTAATTTGTGGGCTGATGGTTTACATTGGGTAGT
TTATCTGCTCTGACTGGGCTCCCTTGGGAATCTGGAGGGTAGCTGAGAGCTTAAGTGTCTGAAAGTGGCT
GGGTCAACACAGCTCTATCGCTCACATCTTGAACATCCCTCCAGCAGGCTAACCAGCCCGAGCAAGTCCT
TTTTGTGAAGGCTGAGGTGAAGAGTGGAAGTGCAAACATGTAAACAATTTGTTGAGCCCCTGCTTCCATT
AAGCCTGCAATATCCGATTGGCTAAAGCAAGTTTTATTTCCCCCTGTGGTAGGCAGAATCATGGGCCCCT
CGAAGATGTTAATCCCCAGAACCTGTGAATATGCTGTGCTCCCTGGCAGTAGGGAATTAATATTCCAGAC
GGAATTAAGGTTGCTAAGCAGGTGACTTTGAGATGGGGAGATTTTATGGACTATTCAGATGGGCCTAATC
TAACCACAGGGTTCATATAAGTGAAAATGGAAGCAGGAGAGTGGGAGTCAGAGAGATGAAGATAGCCCCT
GCTGGCTTTGAAGATAGAGAAAGGGGCCATTAGCCAAACAATGTTGGTAGCATCTAATGCTGGAAAAGGC
AGGGAAATAGATTGTCCTCTAAGCTTCTTCAGAAGGAGTATAGCCCTGCCAACACTTTCATTTTAATCCA
TGAAACCCATTTCAAACTTCTGACTTCCAAAACTATAAGATAATCAATTTGTGTTGTTTTAGGCCAGTAA
GTTTATGGAAAATTGTCACAGAAGCAATAGGAAACAAATATACGCTCCATTGTTCAATCTTTTGAATAAC
ACATATACTATTATTTACTTAATGTTTTTCTTAAAATCAGCTCATTTTGTTTTCTGCTTTTAGCCTTAAG
TGATAATTCCCACAAAACTGTAGTCTGATGTTGCAGTGTTTTTTTCCTTAATACAGATAAAACTAAATGA
ATATTAAAATTTAAACTATAAGCTGTTTATCTGTGTAACATGGTAAATTGGCTCCCTACCACTACTGTTC
AGCAAACCACATTTTGGGAAACAGCGATTTAGGTGGTTCAAAGGAGCAAGTGATTGTGCAAGAACAAGAA
TTTATTAGAGAAAGAAGCATTTGGCCAATGGGTAGAATTGTTGGCAGACAAAGGTAGAAGAGAAAGACAA
ATTATTCAGTATGGCTCTAGCGAACTCTTTGCACTTTTATCACACAATCTGAAGCTTGCTAATCTTGACA
TGTCTTAATGTTGTTGGATTGCTCATTAAACTGGCTGAAATGTTCACAAAGACTCTCACCTGTCTTCTGG
CTTAAGCTGAGATTTATCACACTTCTTGGAAACATCTTCTGGTCTCCAGATCTCCCTCAGCTAAGCTATA
CAGTCAGTCTGTTCTGTAAGAAAGCCCAAACTTCTCTGCAGTGTTCCTCAGTCTTTTTGATATCATGATG
AACAGATTAAGTTGATGTGTTCATCATGATATCAGGTAAGCTGGCCGAAGACTCTAAGCTGCCTAACCAT
CCCAGGGCTGAGAGGGATCGATATCTGAAGTACCTATAACCCAATCAGGGCATGTGCCTTAGCATACCCA
TTGGAAAGCCCTGTTTTAGAGCCTTTATCAGCTGTGAACTTATTGAAGGCAATGATTTTGTCCTGTTAAT
CATTCTATTCATAATTTTCAACAAGATACGTGGTTGTTGTTAATAATAATTGTTGGTTGAAATGAAGTTA
AATAAATAGCAATTGACTTTTCCAAGGTGACGCATTGCACAGATTTATTTATCTTCCCTTTGCTGCCCTG
GAGTACCAGTTGTATCTACCAATAAGCTTCATTTATAGGCCAGCCTCATCTTAGTTTCTGAATTAGTCTA
AGTGGCTCTGGTAGCGCATCAAAAATCTTGCTTTCTGATGGTCTTTGTAATTTGAATTCTGTGACTTACA
GACTTGGTATTCAATATGTCAGGAATAAACCTGGGGTGTGCCCAAATGGTTTGAAAAATCCCAGCCTTCC
TGATTTCCTCTCTTCTCTTTCTCCCCTGGCCACCCTAATAGTCTGATAGTTTTTGTTATTTGGATACTCC
TAAACTCTTGGCAATTTTTCTTACATCTGTTCTCTACAGGCTGTCACAAGTGAGTGGAGGCAGGATGGCA
TGGGCTGCAGTGGAAAGACCAAGAAAATAAGTTAAAAGCCCTGGGTTCCAGTAAATGCTCTGTAGTGGGA
TTTAGGGCAAGTCTCTTAACTTCTCTCAGCATCAATCTCCGCATCTGTGAAATAAGATTAATGACACCTG
TCTTGCCTATACTTCAAGGTTGTTTTGAGGTTCACATGCATTTTCCACCCCATATAGCCTATAAATCTCT
GATGCCTACAGATAACCTATAATGTTCTCCAGTAAGTTTAATATTTCCAGGATTTTAAAACTCAATGACT
AGCACTGCTCTGATCTAACATAACATATTGTGTCAATATGTGTGGGAGTCTCTCTGGTTGATGTTAATGG
AAGTTTGTATAGTTTACCTAAAATAGAATAAAGCTATAATATTAATATATATCATCGATGTGTTTTAGGT
GATTTTTTTCAATATAAAGGCAATTTTGGTTCAAAATTAGGTAGAACATTTAATTTTTACTAATTTACAA
ATAAAATGATAACATCAAAAGGGCCCCTTCTTTTAAAGATAAGTTGTAACTCTCACATTGATAGTAATCT
GTCATTTAGGACAGGGAATCCATGTAGTTTGAAAATTCATTGGCATCATGGAGCTAAAACAGTGGCTTTT
TAAACATGTCGATTTCAGTTTTCTTTGTTTTACAAGTCAAGTAGTGATATTACTGGGTACATATGAAGCA
TACTGATTGACCAAAAAATAGTAACAAATTTTGTAAACCCTTCACTTAACCATTATTCACCTTTCCCAGC
CACATAAGAATCCTTTCTCTTTGTCCTTAGATTAATTGCCTTTCTTTAACCTTTTCAATTCTAAGTCCAG
ACAAGCTGCTGTGGTTCTTTAAAAGGCCACACAAAATAAGTATTGTCCAGTGCTAACACTCTGAAATGTG
ATATTGTAATTACTACCAAGTGAACATTAATCACTACTAGATTAGAATGGAATTACCTGTTATATTCACA
TTAATAGCAAATGAGCTTTCCCTGATTGATGTTGTTATAATGAATACAAAAGGAATTAATAGTGATCTGG
CACTCACCAAAAGAGGGGTAGTCATTAAGGACATGCCATCAAAAGGCGGGTAATACTTTACAAAAAACAA
GTATTAATTAAAGTAATATCACAACGAATGCCTATTGAATAACTTATATCCACATTACAAAGATATTATA
TGGTTGCGATTAATGTGATTGCAATACATTTTGTAAAAATTAATAATGACTAACCCTTTAAAATATTTAG
GAAGCAGATATTTGTTTATATTTGCTAAATAGCTATGCCAACTCTTTAGCTTTTGTGAGTGACTTCTAGC
ATAGGAACAGTGATGGATAATATGAAGCACTATATATAATAACTCATCGGCCGGGCGCGGTGGCTCACGC
CTGTAATCCCAGCACTTTGGGAGGCCGAGGCGGGCGGATCACGAGGTCAGGAGATCGACACCATCCTGGC
TAACACGGTGAAACCCTGTCTCTACTAAAAACACAAAAAATTAGCCGGGCGTGGTGGTGGGCGCCTGTAG
TCCCACTACTCAGGAGGCAGAGCTTGCAGTGAGCCAAGATTGCACCACTGCACTCCAGCCTGGGTGACAG
AGTGAGACTCTGTCAAAAAAAAACAACCTCATATATTTTTACTTGAAAACATACATTTTGCCTTTAGGAT
TTTTACTTGTTAGAATATCCTAAAGGACCTATAATTGTAAATGTAAAATTGACTAATTTCTGGGTTTTAA
AAAAAAGTATTTGAAAGCTGATCTGCTGTGAACATTGAACCAGATGTTAAGAAAAATGCTAGTAAGAAAT
GAGACTTGGGAGCAAAGAAGCAGAACTAAACTTTTCATATATGGTTTCTATGGAGTAATTGAGAACGTAC
ATATTAACAGGGATACAAAGTCAGGCCCTCTCATTCAAGATGCTTTCTGTCTTTAAAAAAAAAAAAAAGT
AATTTTTGAAATTTTCTGTGGCAACAGTCCCATAGCAGAAAGCAAAGAGTTTTGAATTAAGTGATCAGAA
TATCATTCTTATAATTTTACTACACTGAACATTATTTAGAAAATTTTGAATGATATTAAAACCGCTATAA
AACATACTTGCCTACCATAAGACTTAGGATTTAAGCCAGATTAAAATAAATATTTATTTAGAAGGATGTA
TGTAAGAACTGGTGAAATATAAATGAGGTCTGTATTTGAGTTAATAGTATTGTGCCAATGTCAGTTTCCT
AGCTTTGATGATAATGTACTATGGGTATTTAAAATGCTATCATTGGGAGAAGCTGGGTAAAAGGTGCGTG
AGAAGTCTCTGTACTATATTTGCAAGTTTTGTGGTCTTAAACCATTTCAAAGTAAAGTTATTTTAGAAAA
TATCTAAATATATATTTTAGAAAGTATTATCTTTTTCTCTGTAACTAGTGGCTAATTAGCTCAGTCTGAA
AGAGTATGTAGAGGTGGAACTGCTAAATATATTTCTGATCTAGACTTACTTGATGATGCTTGAATTAGTA
AGTGAATGTTATGTGCCAACATATGCTATGATACATATAAATATATAAGATTAAATGATAGGAGCTAATT
ATTTCTTGGCATGTTGCAGTGGGTCCATTTAAAACTGTTTATGTAGGAAACTACTGTAATTATAAAAATG
AGCACAGCCCAACAGCCCAGTATATTAGTTGAAATATAAAAGGCGTTGTGTCCAAGATTTGAAATGCCTT
ACAATAAGCTTGGCACTTACTTACCTTCACACAAAGCAGACACATTTTATTGTGATTTTAGTGTTCCATA
TTATATGGTACAGTACCAAAGGAAAACTCTAAAATATGTACTCAAAATCCTGATGTGCCCTTCTTTCCAA
ACAGGTGGCACCACAATGAATATAACCTTTAGAGTTAATATCTGAGGACAAACCCAGCAGTTACACCAGC
ATGATTTAGGTCCTGCTGTTACAATTATTATTATTGTATTTATTTCACAATTAAGTTGCAGAGTTGAGCT
CGATATAGTTCCAGCTGTGGCTTTTTTTTCAACTGTCTCAATAGTTCATAGATATGGCCAAATGTTCAAT
AATAGTGAAAGCTTATAGTCCACATATTATTTCTGTAGCACCAATTTTATGTGAAAAAATGATTTATCTA
AATCTCAGAGAATTTCCATAACTAGTTTTGTTATACATCTACAAAACTAAGTTAAAAGAACAGAGCAGAC
TTTTTAAATAGCTAGATTGGCCAAATCCACCTCATTATCATCAAGAAGATACTGAAACACCGTGTTTATA
CAACAAGACCAGGCATTGTAAAAGAGGAGGAAAGGTAGAGACAAAATTTATTTAGCCCTCAGGAATCTTT
CATTCTGATAGTGTAAATTTGACAACTTTAGAGGGACTTATTAACATGTATCTTATATATCTTGATACCG
AATATATATTTTGTGATTGCATTAGAGCCATGAAATATTACACAGGTCATTTGAACATAGCATTTTCATA
GAGAAGGTGACATTTGCAAAAGATTAGGAGAAAAGTAACTACGATTAGAAAATCGTAGTTTTATTTTGTC
TCTTGAGAATGAATTGATGTTAATTTTATGTCTGATTTGGCCAAATACGATGTGGAATTTGCTAAAGACT
GAAAAAAGAAGAGACATCAAATAGAGGGTTGCAAGTTAACAGACCATGTTATAATTAAATGAGGGAAAAA
AAAGTAGAGTTGTTAAACTCCCAGAGAAGTCATTTCCCCTTGGTTTGGTGCATTTCACTTTGGTGGTGAA
GTAAATGACCATATGGGCACTTTTCTAGCTCTGTCCGCAGGTAGCACTGGGTATTTGTGGACAAATTACC
TAGCTTTTCATAGCACTAGTTTCCTTGTTGATAGACTTCAGAATTCTAAATTCCATTTTACATCCTTATT
TCTATGTTTAACTTAAAGATAATCCTTTGCAGCCGGGCACAGTGGCTCACACCTGTAATCCCAGCACTTT
GGGAGGCCGAGGCAGGCGGATCACGAGGTCAGGAGATCGAGACCATCCTGGCTAACATGGTGAAACCCCA
TCTCTACTGAAAATACAAAAAATCAGCCGGGTGTTGTGGTGGGCGCCTGTGGTCCCAGCTACTCAGGAGG
CTGAGGCAGGAGGATGGCATGAATCCGGGAGGTGGAGCTTGCGGTGAGCCGAGATCGAGTCACTGCACTC
CAGCCCGGGCAACAGAGCCAGACTCTGCCTCAAAAAAAAAAAAAAACAAAAAAAAAAACAAACAGATCAT
CCTTTGCACTGGAATTATCCTGCAGTGGAGGATAGTAATGAAAGTGTAGACTCTGTTTCTGAACACTAGC
TATGTCACTTTCAAACTGTGTGATTTTCCTTCAAGTTTCTCAATCACTCCAGGTCTGGTTTCTAAATAGA
GGAATAGGAGTAGAGATTAATATTGTGAAGATTAAATGAGAAAACTTATATAAAGCACTTAGTACGGTGC
CCTGCATATTGTGAAGGCTTGGTATGTTGTTAGTAGATTCATTTTATTATCATTATTAATAATACTGAAC
CCTGGCTGTTGGGGGAATTGGTTCTATCCTCCTGTCTCATAGTCAAAATAGGTTAAAGGGCCTTCTATCT
CTTATTTCTGGTGGTGCATTATAATTACTAATAGTAATGTGCTTCATTTGTATATGATCCTTTATAGTTT
ACATGGCGCTGTTTTATGTAATCTTACTAAAATTTCAAAAATAATTTTAAAAAGCCAGAATTCACAAGAA
TGTGACTCGGAGAAGAAGTAGATGTTTTTCTAAGTAGATCTTTCAGTTTAACTGATTCAAATTTTCTCAT
GTTTCATATACATGATTATCATGTCTTTTGATAAACAGAATGTTAACCAGAGTACAACCTTGTATGAACA
TATTTATTCAGCTTAGAAAAGATCCAGAGGTACAAAATCTAGATCCCAGTGTAGAAGTTAGCATACACAG
TACAATTTCTAGTATGTCCATAAACAATATGTTAAAGTATTAGTTTGAGCCATATAGGATTGCCAATATC
TGAGTGTTATAGAGCTACAAAATTAGTAGGAAATTTTGTTGCTTTAACCTAATCATTAAATTAGAATTGT
GTGACTTAAAGTTACAAATGGTTTCCGAATATTTTGCAGTAAAAAAGTAGTGAGGAAAATAAATATAAAT
ACTAAACTAGACCTGGGAAATTTAAGGCTATAAAGAATTCTAGCTTACAGAGAGAGGAGTCTTTGTTTGC
AACCTCCCACTAGCTAAATTTAAATTATCACAAATTTCATCCTCTCCTTTACTTAACCCTTGACTCATGC
AACTAGTCAAATGTCTTTTTCTTGCTAATTTTTTCTTTCCATAGATCACTTATAGGGAGTTCTGGTTAAA
AATGATGTCTCTTTAACCTTCACTAAAATGAGAATAGGGGAATTAAAATGATATTTACCACAAAGAGAAA
AAAATCTGGGAGGAAAAACAATAAAATAAAAAAGATAAAAAATTTAGGAATATGCAGAGAATGGAGGAGT
TAGCATATCTTGGAAACCTGAATTCCAAGTACTTAGAACTTGGGAAGTCCTAGAAATGTGAAGCACCAGC
TACTGCAGAAGGCAGAGATGAATGTGAGGTAAGATAGTGAGACTGTGAAGAGAAATCATTCAGTAAAAAA
TGCATTATCAAGCCAACTGCCACTGGTCTAGTGGAGTTTAATCCCACTGGGGAAATTCTAAATGGATTGA
AGACATGTGTTTAAGAGTTAGTTATTCTTTCAAAGGGGCAAGGGAGCTGGGGTATTTATACACAAAATCC
TGCTAGTCATTGGTTTAGGACTGCTTCCAACGGGGGAATTATTTTCCTAGCATTTCTGGCATACCACCTT
GGCAAGAAAAATTATTTTGTGTCCAGAGTATGTCTAAAGCCATTAGGGAAAAAAAATGTGGATCCTCATA
GTTGAAAGCCAGGCCAGTCTGCACTAAAGTGGTAAGGATGTTTTCTTTTAGAGATACAGGTCTAAGAAAG
AAATCTGAAGGTGGTTACCTCTTATGCAAGAATTTAATTTGATGGATTCAAGGTGTGTTGGTTAAGAGAA
ATGGGGAAGGGTTGCTTCTCATGACTGCGGCACGATTCTACTATACTAAATTTTTCTTTTATTAAGCAGC
ATTGCCTTATGCAATGATAGGAAACATTTGTTATATGTGAAATCACTTTTATTTTTATTTTTTAATCTAT
TCCTATTCTTTTCATTTTTTTAACTTTTATTTTAGGTTTGTGGGGTACATGTGAAGGTTTATTACATAGG
CAAACCGGTGTCACAGGGGTCCGTTTTACATTTTATTTCACCACCGAGGTATTAAGCCAACTACTCAGTA
GTTATCTTTTCTGCTCCTCTCTCTCCTCCCGGCATCTCTTTTAAAAGAAAATAATTTTTAGCAATTCTTT
AGAATAAGTCTTGGCCACCTAAAGGTTTCCAGGACTCTAGTTCAGGGAGTATTTATCTAAGTCAGTAGTT
CTTAACCTGATATAATTTCATCCCCAGAGAACATTTGACAGTATCTCAAGAAATTCTTGGTTGTCACATT
GGGGGGGGGATACTGCTGTCATCAAGTGGGCAGAGGCCAGGGATGCTGCTCAACATGTTGTAATGCACA
AGACAGCCCCCCACAACAAAGAATTATTTGGTCCAATATGTCAGTAGTGCCAAGTTTCAGACATCCTGCT
CTAAATCAGGACTGTGATGTGAATTCTCTGCGATGATGAAGATATTTTATATCCGTGATGTGCAGTACTG
TAGCATATGGCTACTGAGCAATTGAAATATAGCTAGTGTGACTGTACACCAGGTGTGATGTTCCATACCG
AGGAAAGAAGTAGAAATAAGATATAGTCTTTGAAGTCAGAGCTCACAATCTAGTAGCGGAGACAGATTTT
TAAAAATTACAATATTTTAAAAATATTGCAATAGAACATGGTAATGTTAGAAGATTAATAACATGCTAAA
TTTGAGGCATCAGGACTCAGACAGACAATTAAAAATTCTCTGAGGTGAATTTCCACCCTTAGCTCAGAAT
ACTGTAATGTTTAAAAGCTGTTTTCTATACACACACACACACACACACACACACACACACACACACACCC
CTTTAAATCTTTTATCATGTAACTCATTGCTTCTTATTTTACCCTTTTGTCAGAGAATACATATAAAATA
CTGGAATCTGATGGGACATTCTACTTTATTTAACAATGCTATTGAGTTTCTCAAAATAGTTTCCTAAGAA
AGTCTATTAAAGTATTGATTTTTTCATAAAGGATAATACAAATGGCATGAGTCTGTTTAACATTTTAATC
AAGCTTAAAATTAGTCTTGCATTTGAAACAAACTTGCCCAGAGAAATTGTTGAGAAACTTAAGAGAAAAA
CATCATAAAAAATTGATGGGCCAGCCAGGCTGTGAGAATATTAAAATCCAAATCTAAATTATGGTTAACC
ATTGTCACATCTTTCTTTGAAGCTTAAGTAACTCGATATTCCCTGTAGGATACCCAGTGATTCAAAGTGA
CACATATACTGTCAGCTCATTTTCCTTCCCAGCATGCTGGTACAATTTGTATCCATAGAAATATATGGAA
AAACCTATTAGTCTTGAGTGCCAGAACCTACCAAAAGGAATCTTTGTCATCTACAAATAAATTAATAACA
TAAGATAAACAATCCTATTAAGTTATACTGGCCCGAAAAGGGAAAAAAGACCAGTTTATGAATTGACAAA
AGAAGGTAAATGAGATTAGCCATATAGCAACCACTCAGATAATAATGTGTTTTCTCTGTTTAGTAAAAAA
GCATATTTGAGAGAAAATTTTCCCTTATAGAACAATTCTTAATAATATACATAGATACTCCTTTCCTGGG
ATGTAGAGTTTAATCCTCCCCTAAGCCCCTCCATGAACTTGGTAACTTACTTCCAGATAATAGAATATGG
AAAAGTAGGAATAACAATGGAGAAGAAACCAGGCAGGCACCAAGTTAACTAAGTAGTCAAGTATAACATC
GCCAGTGATAATAATATTGATATCATGTCTCCTGTGATATGATGTCATGAAAAGGACATGTTATCTCTCT
GGTATTCTTCCCCAAAACCTGTAACTTCTTCTAATAGGGAAAATACTTCAGTCAAATCTTAAGAGACTTC
TAGAATATACCTGACTAGTCCTATTCAAAAGTTTCAAGGTCATGAAGAACAAGAAGAAACTGAGAGACTG
TCACAGACTAGAGGAGACCAAAAAGACCCAAGGACCAAATGCAGTAGGAGATTCTGGATTGGATCCTGAA
ACAGAAAAATGACATGAGTGGAAAAACTGGTGAAATCTGAATAAAGTCTGTAGTTTTGTTAATAGTGTTG
TATCAGTGTTTGTTTAAATGTTTAGATAAATCTCTCATGCGTACAGAAGAGTTATCATTAGGGGAAGCTG
TGTGTCAGGCACTTAGAAAACTTTCAGATACATAGGTACCTTTTGTAAGTAAAATAATGAATTAATGGGT
CATTTTATGTCTGTATTTTATATAAGGCTACATTTCTAAAGAGACAAAATTGTGAGTCCCATAAAAATAT
AAAATGAATATGTGTAAAACATTTTATTAGATCATTAACTGATGAAGGAATTAGTAAGATGTTAGTTACA
GTTGGTTCAAAGGAGAGTCTGAAGAATTGGCATATATATATACGTATATATACGTATATATACGTATATA
CATATATATACGTATATACGTATATATACGTATATACATATATGTGTATATATATATTTTATATATATAC
ACATATATATATAAAAAACACTCTAGAATGCTGATAGGAATTTTATAACAGATACAATACTGATCACTAA
CTGTAGGGCAGGAATCTATTGCGTTCCATGAGAAAATTTTACTGGCATCTAGTGAACAAGAATCATTTGT
GTCACCATCAGCCCTCCACAAATTGACTTTTAAACGTACAGAATTGCAAAATAGCATAACCAAAGTCTAA
GGTACAGACTCTTAGATAATCAGATAACTCCTAAGGTTTTCCTAAGGAATTAAAGGGAAAGAGACATTCT
CAGATTAAGGAAAACAAAGAATTTCTTGCTAGCAAATCTGCTCTTAAAGAATGACAAAAAGACATTCTCT
AAACAGAAAGGAAATTATAACGAAGTCTTGACATTTCAGAAAGAAAATAGTAGAATGGGTAAAAATGAGA
GTAAAATAATAGACTATCCTATTTACCATAAGTTTGAAGTGAAAACTTTAACACCACCTGATGTGGTTCT
CAATGTATGTAGAGAAAATACTTAAGAGTTATATTTTAAAAGAAGACATACCTAAGTGGAAGTAAGAGTC
CTTCTACACGTCACCTGAAGTCAATTCCAGTAGATTGCAATGTTAATGCGTATCGTAATGCCTGGAAAGA
CCACTAAAAAACTATACAAAGTGATACGTTAAGAAAATACAACAAATAAATTTTGATGGAATCTTAAGAA
ATGTTCAAATAACCCACAAGAAGGTAAGAAAAAAGAAAGAGAAGAATGAGAAATAAAGAAAACAAACAGA
AACCAAATAAGGTGGCAGATTGAAGCCCTAATATATCCATAATTACCTTAAATGCAAATGGTCTAAATAT
ACCAATTAAAAGAGATTTAGCTGAGTGGATTGATAAAAGCTGAGCACACAATATGCCGTCTAAAAGAAGT
TTATTTCAAATACAACCTAGGTAGGTTAAAATTAAAAGAATCGAAAAAGTTACATTATGCAACAATTAAT
CAAAAGAAAGCAGCAGCAGTAATGTTAATATCAGATAAAGTAGGCTTCATTGCAAAGAAAATTACTAGTG
ACAAACAGGGACATTACATAAAGATTAAGTGTTAATTCACTGGGAAGACATAATAATCCTAAATGTGTTT
GCACCTAACAACAGAGCTTCCAAATACATGAAGCAAAAATGAATAGAGCTGAAAAAAGAAACAGACAAAT
CCATATTTCTAGTTAGGGACTTCAACACTCCTCTCTCTTCAGTTGATAGAACTACTAAATGGAAAATAAG
CAAGGGTAAAGAGAACTGAACAACACCATCAACCAATAGGATCTAATTGAAGCACTCCTCCCAACAGTAG
CAGAATACACATTACTTTAAAGCTCTCATGAAACATTCACTGATATAAGCCATATTCTGGACTCCAAGCA
ACTTCAGCAAATTTAGAGAATTCAACTTATATGTTCCCAGAACATAATGAAACCAAGCTAGAAATCAATA
AGAGAAAGACAAAAGAAAAACCTCAAAACACTTGGAAATGAAGCAGCACACCTTTAAATCATTTTCCCCA
GGTCAAGGAGGAGGTTGCAAAGAAAAATTTTTTAAACACAAAGAACTAAATAAAATGAAAATAAAACATC
AACATGAGTGAGATTCTGAAGCAAAGGGCAAGCATATCTACTGTCTATTTTTAAAGATTAAGCTTCCTTA
AGCTCAGGGTTTCTCTCCTGTGATGCAATCCACTGTGTGTACAGGTGTCTCCTGAACTTCTTTGGGATTA
CTCTGTGGGAACTGGCTCAATAAAATGTTGGTTCTTTGACTACTGCTTTGCTGTGAGTAATCTAGTCTTT
TTCTCTGGCAAAAAAAAATAAAGTGAGATGTCATAAAAGCAGTATTGAGAATAAAATGTATAGCATTAGA
TTATTTAGTTAGAAGACAGGAAAGGTCTAAAATAAATAAATGAGCCTAGAGACAAAAACCATCAACAAAA
TATTAAATAACATGCGTCAAAGTTTAAAAAAAGAGTGTCATACCATAACTAACTGGGATTTAGTATTGAA
GGCTGGCTCAACATTTGAAAGTTAATTAGTGTAATCTACCATATCAACAAACTAAAGAAGAAAAAATCAT
ATGATTATATTGATTGATGCAGAAGCATCTGACAACACCCAGCATCCATTCATGATAAAAACTATGAGAA
AACTGGGAATAGAGGATAACTTCCACATCTTAATAAAGGGTATCTACAGAAAACTACAGTTAATAGCATA
ATTTTAATAATGGAAGGCTTAATGTTTCCACCCATGATTGCTAATTAGGGAAGGATGCCCAATTTCACTA
CTCTTTTTTAACATAGTTCTGGAAGTTCCAGACACTACAATAAAGCAAGGAAAAACAATAAAGCATGCAT
ATTGAAAAGTATAAAATAAAATTATTTCTATTTGTGGATGGCATGACTGTGTACGTAGAAAATATCAAAT
ATTCTACAAAAACAAAAGCAAAAATAACCAAAAATGCTCATGGAGCTGAGAAGAGAGGTTAACAAGATCC
AAAAATACAAGATCAACACACCAAAGCTAGTCACATTTTTATATACAGATGCTCCTCATCTTATGATGGG
CTTACATTTAGATAAACCCATCATAAAGTCAAAAAATCATAAGGCAAGCCATCACAACTTACGGATTATC
TATGTTGGAAATGAAGATGTGAAAAGTGAAATTAAAAACACAACACCATTTATAATTGCTTATCCAAAAA
TGAAATACGTAGGTATAAATCTATCATACATGTACAGGATCGGTATGTAGAAAATTATAAAATGCTGATG
AAAGGCATTAAAAACAACCTAAATAAGTGGATTATATGGCATGTTTATAGACTGGAAGAGTCAGCATAGC
AAATATGTCAGTTCTTCTCAAATCAATCTAAAGGTTTAATTTAGTTTCTATCAAAATCTTATCAAGGATT
TCTGTACACATAGACAAGCATACTCTAAAATCTATAAGAAAAGTCACAGGCCACAGAATAACTAAAACAG
TCTTTTAAAAAGGTAAATAAAGTGGGAGTAACCTCTCTACCCAATATTATGGCTAACAATATAGTAAGGC
TATCAATACAGTATGATGTTGCTGGAGGGATAGACTCATAGACCAAATGAAACAGAATAGAGAACCCAAA
AACAGACCCATGCAAATGTGCCCAACAGATTTTTGATAAAGTTGCAAAAGCAATTCAATAGAGAAAGCTC
ACCTTTTCAACAAATGGTCCTGCAGAAATTGGACATCCCTAGAGTGGGAAAAAAAAAGAACTTCAACCTA
AATCTCACACCTTGTAAAAACTTAATTCAAAATAGATCATGGACTTAAATGTAAAACATAAAACTATCAA
AATTTAGGGAAAAATGAGAAAATCTTCAGGCTCTAGGGCTAGAATTGGCATTGAAAGCATGATCCACACA
CAGAAAAAAATCAGTTGGACTGCATCAAGATTTAAAACCTTTGCACTGCAAAAGACCTGTGAGGGAGGAT
GAAAAGACAAGCTACAGACTGATAGAAAATATTTTCAAGCCATATAGCCAAAAGATGGATGTCTAGAATA
TATAAAGAACTCTCAAAACTGCAAGGTAAAACAAGAAACAAACAATGCAATTAGGAAATGGGCAAGACAC
ATCAAGAAACGTTTCACCAAAAAGGATATACAGATAGCAAATAGGTGCATGAAAAGATTATCAAAAACAT
TAGCCATTAGAGAAATGCAAATTAAAATTATTATATATTCCTACACATCTATCAGAATGGCTAAAACAAA
GTAGTTACAACACCAGATGCTAGCAAGGATGTGGAGAAAATGGATCATTCACATATTGCTGGTGGAAATG
TAAAATGGTACAGCCACTGTAGCAAACTGTTTATCAATTTTCTGTAAAACTAAACATGCAGCTACCATAC
AACCCAGCAATTGCACTCTTGGACATTTATCTTACAACCTGTACAAAAATATTCATACCACCATTATTCA
TTATAGCCAAAAACTGGAAAGAACCCAGACGGTCAACAATGAATGGTTGTACAAACTACGGTACATCCAT
ACATACCAGGCAATACTATTCAGCAATAAAATGGAATGAAATATTTATACATGCAACAACTTTTAGATCA
ATCTCCACAGAATTATGCTGAGTAAAAACAGCTCATCTGAAAAGGTTACATAATGAATGATTCTGTTTAT
ATAGCCGTCTTGAAGTGACAGAATTAAAGAATGAAGAACAGATTGGTGATTGCAAGGAGTCCGGGACAAC
AGGGGAAAGAGAGAGAGAGAGATGGATGTGACTCCAAAAGGGCAACACAGGAGGCATCCTTGTAGTGTTG
GAACTGTTCTTTACCTTGATTGTGTCAATGTCAATATCCTGGTTATGATATTGTACTATATTTTTGCAAG
GTTTTACCTTCAGGGAGACGGGGTCAGGGGTATACTGGTTTTCTCTGTATTATTTCTCACAACTACATGT
GAACATACAATTATCTCAAAACTAAAAGTGTAATTTCAAAAAACAAATAAAACAATTCAGAAATTTTAAG
ACTTCAACAGTCATTTATCTCATTTGTTATTTTACTGTTGAGAAAACAGGCACAAAGAAACTGAAGTGAC
TTACTTTCATGCTTCACCTAAGTCTTTTTTTCTTTTCTCCATCACTCAGTTAAGAGCTTCTGTAATACAG
AAAGTATGTCTTGTATTCTTTTAACTCCCATATTACTTCAAGCAATGTTGAACACATGTTAACATTGTAA
AAGTTGTTGTCTGAGTAAATGGGAAAGATAGAGGTCTATGTCTATATGCAAATACTTTGTATTAACATGT
TTCAGTCTGATATAACTTTCCACACAGAAAGTACAAAAGAAGATCTGTTCAAGTTATCTGATTTAATTAA
GATAGTAAAAAGAAAGCTGATAATTTAGGGGGTCTTATTTGATTGTTTTTAATTTTACTTATTTTCCACT
AGGTGATCATTTTGATGATTCAAAAATGAAAATTTACAAAAAGGTATAAAATAAAAATTATTTCTCCTAC
CTCTATGCACTGTCGAATCAATTCCCCTACCCACCACCAATCAGTATTGTCAGCTGTTTGTATATCCTTC
AGGAGATATGTACGAATTTCAAGCGAATATGCATAAGTTTATGTTATGTATATGTGTGTGTCTGTTTTCT
ATATATATGCATCTTTACATTAATGGTAGCATACGATACACATTTTCTTCTGAATTATGCTTCTCTCTCA
ACAATGTTTCTTGGACATTTTCCTGTATCAGTACATAAAGAATGTATTTGTTTCCTATGACTGCAATAGT
GAAATACCACAAACTGGATGACTTAACCAAAAGAAGTCTGTTGTCTTACAGTTCTGGAGGATAGAAGTCT
GAGATCAAGGTGTCAGGAGGGTTGGTTCCTTCTGAGGGCTCGGAAGGAGAATCTGTTCCATTCCATTCCC
CTAGTTTCTGATGGTTTGTTGGCAATCTTTGGTGCTCCTTGTCCTGTAGATGTCTGCCTTCATTTTCACA
TGGCATGCCCCCTGTGTACGTGTCTGTCTCCAACTTCCCCTTTTAAGGACAGAGTCATATTGGACTCAGG
CCCAACCAAATGACCTCATTTTAAGTTGATTATCTCTGTAATGACCCTATCTCCAAATAGGGTCACATTC
TGAGGTACCAGGGGTTAGGACTTCAACATTTAAATTTGGAGAAAATTTGGACAGAATTCAACCCATAGCA
AAGAACTTAACCGTTAGTTTAATGACTACATATTGTTCCATTTTGTGGATGTATCATAATCTATTTAAGC
AGTGCTCTGAACATTTTATTGGTTTTCACTTTCATTGTTTTGCCTTAATATTGGTGTCTGTTTCATAGAA
TAGATTTATAGTATTTTAGGCTTATCAAGATTTTATTTAAATCTTGGAATTTAAATTCCCTGTAAATTTC
AAGTGCCTTGAAGGCAAGATATATTGAGGAGGGGAGACTTTTAAAGTTCATATGAAATAATAAATAATCG
CAAGTATCTCAGGAATGCATGAAAAATAATAAATGTCTCTGCATCAATAATAAGGGAGGGGGCTTGCCTT
ATCCAATATTAAACTTGCTGTAAAGCTACTGTAATCCAAATAGTATAGTATTAGCACAAAACAAGACAAG
TAGATCACTGAAGCAAAATTGAGAGTCCAGAAGCAGATCAGATTGTTTTTGGAGGCCGGGCATGGTGGCT
TACGCCTGTAATCCCAGCACTTTGGGAGTCTGAGGTGGGTGGATTACCTGAGGAGTTCAAGACCAGCCTA
GCCAACTTGGTGAAACCCCGTCTCTACTAAAAATACAAAAATTAGCTGGGCGTGGTGGTGGGCGCCTGTA
GTCCCAGCTACTTGGGAGGCTGAGGCAGGAGAATCGCTTGAACCCAGGAGGCGGAGGTTGCAGTGAGCCA
AGATCGCACCATTGTACTCCAGCCTGGGCAACAAGAGCGAAACTCCATCTCAAAAAATAAATAAATAAAT
AAATAAATAGATACAAATTGTTTTTGGAAACATTATATGGCAAATGTGTTATTTTAATTCAAGGAATAAA
GGTGTTTTATTCAATAAATGGTGCCAGCACTCTTTGCAATTCCTCTTAGAAAACACAGATTGCCTCCTAG
CTTATGCAATGTAAGAATACATTTCAAATGCATTAAAGTTTTAAATGTAAAAACAAAAATTCTTGGAATG
AGGAAGACGTTTTCTAAACAAGACACAAAACTCAAAAGCTATAAGGAAAAAATATACCTTGTTACTGCTT
AAAATAACAGAAGACAAAGTCAAAAGAAAAACAGCAAATAGAGTAGATGTATTCGCAACATGTGACATAA
AGAGATGCATATACCTAATATACAAATTTCTCCTACAAATTTGTTAATTAAATTAATATTTTTTAAAATT
CAAACAACCCAGTACAAAACTGGCCGAAGTATAGGAATATGCAATTCCCAGAAGAGGATATCCAGATAGC
TGGAAAAATAAAACTATGATAATATGCTTCCTCATAGTAGTAAGGGATAGGAAAATAAAGAAATAAGACA
CCATGTCTATCTAACAAATAGACAGAAATTAAGAATGATAATTTTTAATGGAAGAGAGCACTCTCAGATA
TTGCAGATGAAACGCAAATTGCTGTGGTCTTTGAGGAAAGAAATGTGGTATGATCTACAAAAATTTTAAA
TGCACTTACCTTTTGATCAGTCACTCCATTTCTGAGAATCAATACTACAGAAATAAAAGTACCAGTATGA
AAGGCTGTATGTAGAGGATGTGTATTTTGGCATTGTCTATGATGGTCAAAAAGTAGAAATCAAGCAAATA
CCCTTCAGTGTGGAAATTATTGAATATGTTATGGAATTATTTGGGCATCCCAGAATGAATTATTATTTAG
TCTAGTTAGAGCTGTGTCTACTGTCCTGAAAGAATGGTGATGATATCTTTCAAAAATCAAAACAAGTTTC
AATTAACATATTCCATTTTTAAAATAAAAAAGATAAAATTAATCTTATGGGATTACATAACCATGAAGGA
GGAATGGAGAGATATATACTAGGTTATCAGTATTTGTTACCTTGGATTTTCAAAGGAGAATGAAAGAGGA
ACAAATAATGTATCAAGTTTCACAAAAAGTGAAAAGGTGGAATATAAATATTACTGCAAATATATAACCA
TTGAATATGTATATGGACAAGGACGATAAGATAATATAGAAAACTGAATATGTTGGTTTTATTATGAGGT
GGTTGGATTGAAGATATTTTTGTCTCCAAATACTGTTGTTTTAATATGTTGTGTTTTACAAAGAAACATG
GGCTGAGCAGACAGGGAAGCCCTGATAAGCATAGTACCTGCCATGTGGCCATTCAATAAATGATAGTTAT
TGATTATTATTATTAGAGTTGTAGTACAGTAGTGCCTACCTTAATATATTTAGATTGATGCCCAGCAGCA
TTGAGTTAACCCGCATTTTAAGGACAAGTGTTATAGCTATTATATACTAATGGTAAACTTGAGTCTGTAA
CTAGCACTGTTGAAGGAGGACAACAGAGTAATATGATGTGTATTGGCCTGGGGATGGAAGGGTGGTGCTT
AAGGCACAGCAGATTTTCACTCCAGCCAGGTTTCCTTAGGACCTCTCCAATGAACAGGATACCTCCCTTC
CTGTTCTTTCTACCCTCCCACCCCGTTTTTTGCTTTTTCAGTTTCAGCCCAAAGGGGAAGGAAGTATGAT
GACTGACTCCCCATCAGTCCCTGAGGTGAACTGGGATTTTGGGAGAGTGTGGCAGCTGCAAATTTGGCTT
CCTGGAGATAGGATTTTTGCCCTCAATCTGGAGAAAGTTCCTGAGGCTACAGCTGTTCAAGCTTGTGAAG
TAGGAACTTTGATCCCTTTTTTCAAAAGTTTTGTATAATTAGCATCCAACTTGTTAGACAGTATGTGGCT
CATTACAAGATTGCCACAAATTCATGCTGGGCCGTGTCTAAGAACAGGGCAAAGGGAGCCTTTGGAAAGT
GTTATACAGTTGACCCTCAAACAATGTGAGGGTTAGGGGCGCTGAGCCCAACACATTGAAAAATCTAAGT
AGAACTTTTCACTCCCCCAAAACGTAACTACTAATAGGCTACTGTTGACAGAAGCCATACTGATAACATA
AAGAGTGATTAGCGTATACTTTGCATTGTTATATGTAATATATACTGTATTCTTGCAATAAAGTAAGTTA
GAGAAAATACGATGTTACTAAGAAAATCATAAGGAAGAGAAAAATATATTTACTATTAATTAAGTGGAAG
TGGATCATCATATAGGTCTTCATTCTCATTATCTTCACGTTAAGTAGGCTGAGGAGGTGGAGGGAGAGGA
GGGGTTGGTCTTGCTGCCAATCTAAATGCTGGGCCCAGCCAATGGGTATAAGTTTTAAGTGTGCACATAT
TGGTGAACCCTTACAGATCACGGCACTGTCTGTTCGAGTGTCTATTTTGAAATGTCCCTATCCGTAATAT
AAGTTGCAAAGGAGTTTGTGGGCCCACTGAATTCTACCACCCTGATCATTGTGAAGCCCATTCAGCTTTG
TGAAGAGCTTATCTTGGTACTACCTTAGCCAAGGTATGATAACTCAGACATAATGTCTTTTCTTTCATGG
TTCCTTTTTTAGTGATCATAGATTCAGTTCTGTAATAATTAGAGATTTATGTGTCCTATTAGTAATTGCA
TCATTCTTTAAAGACAGTGTCAACCTTGCTATACAGTGTGATTGAAGCCTTGACATAACTTGGGGGTTTG
TTGGCATTTTGAAATCCCAGGCCCCACTTCAAAACTGTTGAATCAGAATCTGCATTGTAAGAAGATCCCC
AAAAGATCTGCATGCACAAGCCTTGAAAGAGAAGAGAAGCCATCTAACATTCCTCACCTAAGATTTGAAG
AATTTCCCACTTATGCAAGAGTAGGGGTGTGATTTCTCAGGCAGGATATCTAACAGAAAACAACACTTAT
GAAGTGTTTCCTGTAGGAGCTAAGCAGGTGGCCAGAAAATGGCAGGCTACAAAGGAGAAGAATGACTAGG
AACCTGAGCAAGGAGAAAGTCTCAAAGACAAGGAAGTGGCTGGCAGTGTCAGGGACTACCAGGCAGCTGA
AAAAGCTAGGTGTGAACACTATTTCTTGGAGTTTTCAGCAAGTAAGAGGTTCATGATGATGCCTCAAAGA
ATTTACAGTGGAACCAGAGCCAAGAAGTCTTATTGTGGTGAGTTGGGAATTAGGGAAAGTGTTATACAGT
TGACCCTCAAACAGTGTGAGGGTTCGGGGCGCTGAGCCCTAACCTCAAGTAAGAGGTTCATGATGATGCA
ATCAAAGAATTTACACTGGAACCAGAGCCAAGAAGTCTTTTTTTTTTTTTTTTTTTTTTTTTGAGACGGA
GTTTCGCTCTGTCGCCCAGGCTGGAGTGCAGTGGCGCGATCTCGACTCACTGCAAGCTCCGCCTCCCGGG
TTCACGCCATTCTCCTGCCTCAGCCTCCCGTGTAGCTGGGACTACAGGCGCGCGCCACCATGCCCAGCTA
ATTTTTGTATTTTTAGTAGAGACGGGGTTTCACCGTGTTAGCCAGGATGGTCTCGATCTCCTGACCTCGT
GATCCGCCCGTCTCGGCCTCCCAAAGTGCTGGGATTACAGGCGTGAGCCACCGCGCCCGGCAGCCAAGAA
GTCTTATGGTGGTGAGTTGAGAAGTAGGGAGGTGGAAATAAGGAATGGAGATGAAGACATGAAAGGAATT
TGAGAAATGAGGCAATAGCTAGAGAAGAATATAGGAATACACAGATCCAGCAACCCAGCAAGGGAAGGGC
TAGATTCTGATTTCATGAGCAAATTGCCTACTAATTAAATTCACAATATCAGAGGAAACTGTTGGACTCA
CTTACTGAATAATAGCTGAAAAGTATCTTTGCTTTATAGAGAGATCACTGTAGATGAAAGTCATCTTCCA
GTGGTGAGATATTCTTCAGTGTCATCTCTTCATTTTTATTTCTAATTTTTCTTAATTTGAATTATTTCTT
CTGATAATGGGAATGATGTGGTAATTTTGTCTCTCCTGTAAATTATTTTAAAGATTTGTACGAACTCCTT
TGGCAGGCTTGAGTGTGTTGTGACAGCTTGGCTTAGATATGCATTGAATGTCATTGAATTCAAACTCCTT
ACCAACATAGTTAGATAGCCCATAGGCATTCTACTTGACCCTTTCAAGGAGATCTGGAGATGCAATTGTA
GGGGAAAAAAGAAGAAAAGAATTCAAGAAGCAACAAAGTGAAAGATAATTTGGCTTGCAGAAGAGAGGTC
TTTCTATCACAGTAATAATAATGCCAATTATATGTCCATATATATATATACGCACACATATATATAGTAT
ATATATATACACATATAACTCAGACATAATTCTTTCATAGTTCTTTCTTTTGTGCACAGATTCACTTCTG
TTATAATTACATACTTATGTGACCTATTTGTTAGTAATTGCTTCAGTTTCTTAAAAGAGCATCACCTTGC
TGTGCAAAGTGTGATTGAAGCCTCAACATCTCTTGGGAGTTTTTTTAGAATTTTGAAATCCCAGGCCCCA
CTTCAGAACTGTTGAATCAGAATCCGCCATTGTAAGACAATCCCCAAGGGATCTGCACGCACGTTGCAGC
TTGAGAAGCACTGCAGTATCACACATATACACACATATTCAACACCAAAGAGAGAGAAAGAGGTCATAAG
CTCTCAGGTGGAGACTAGTTCCATGTATATATGCATAGAGAGAAGAACAAACTCTACCTTCCAGCAACGT
AAAATTCTACTCAATCATGTATTCACCAAAAAAGAAAAGGCTTTCTCCATATAATGTGTATTATTCATAT
ATTGGCACTCTTCAGAGCTCTTCATTCCACCCTAATGTTATCTTTCTTAGATAATTCACATGACACTTTG
TTATCTTCCAATAATTTCTGTCATTGTTATAAGCGAAATTATTCAGGCTTTATCTAAGAGAGTAAATCAA
ACAGTATGCCTCTCTCATTCCAATTCTGCAATATTTTCATTCTAGAATGTCTAAAGGAGCCTTGAAAGAG
AGGAGAAGTCACCTAAGGCCAGCTAGAGGGGATATATAGCAGGGAATGGTGGCAACTCCACTCCTCGTAG
CCCAGTGGGGTTTTTTTTTTTTCCAATCTGTATTTGTATGTGAGTATCACGTCTATGCCGATTTTATGTG
TACATATGTAACTCAAATCTGTTCATTGTGCTAGTTAGAATCTTATTTCCCCCTCTTCTACTACACTCTA
CCCTTTCTCTTTCCCCTCCTTTGGCAACCAAGACACTGAGTTATTAATAAGCAGATTGGAGCAAACATTT
TGATGCACTATTGTTTGATAGATTTGTTGGTTCATTCAATAAGCATTAATTGAGCACTTGCTAAGTGTTA
AACAATGTACTAATTGCTAGATTTAAGGATGAAAATGATAAAACCCTTGGCCTCTAGAGCCTAGAGTGTA
GTTGGGGAGACAAATGGGCAAATTAGTCACACGACAACATATTCCTTGTTAAAACAGACAGTTGTGCATA
AGTCTGTATACCCAGACTGGAGATGACTCTGTTTCCAATGTTGCCCTGGGAAACCTCATGATCAGTTTAA
TAATGATGTTTGGGGTGAGAGGATACTGAGACAGTTTGCTTCTAGCATAGTAATTACCCATAGAAGTTTG
GGGTCTTTATTCAAAAGAGTTTACAGGCCACATGAGCCACTGTCTTGCCTTTTATAGGATCACATCTAAG
TTCCGTGTCATATAATGGCCTTGGCCTTCTTGGCTTTCTCTGTGCTCTTTGCCTGCCAATACCCTAATTA
TTGAAGTACTGTCTCCCGCAGCTCCTCAACCATGAGCTGTTCCCGATCCTCCCAGCAGCTATGTTTCTCC
TTTCTTCAAACCTCTTTAGCTTTTTATTTGTACTTTATTTGTACTAAATTGTATTTAGAGCTTTGGAGAA
CATTCTTCATAGTTGTAATTAGACTGTAAAGTCCTGAGAATGTATTTTTCATCTTCGTAGCCCCCTGCAG
TATCTAGCAGAATGCCTTTAAACAAATGGGCAGTAAATAAATCCCAACAAACTTAAATTAAATTTCTCCA
AATTGCATATTTAATTTTATAGTGGCATTTACTGATAACATACATTGAAATAAAGGCCAGAGCATAATCC
TCTCTGTTTCTGAATATTATTTATTTAAATATTAACTTTCTAATCCAATTAGGTCTTTCAATGACACTTT
AGATCTAAATTTATTTTTGCATTGTTTTAAATGTCATCAAATGATTCATCTCTTGTGTTTTTTAATATTT
TTGGAACGAACGTGTGAAAATGAGCAAGTGTCATCAGAATATGATGCTTGGGTTTTTTTAATTCAACATT
TCTTTGATCATATATTTAAAGACTTTTTCTCAATTCCTTTCTGGATGTGGCCTCACAAATCATTTCAGAA
GTCAATCCATTTCAAGATTTTTTTTTTTTTTTTTGCTTTTTTCACTTCACAGGAAGTCAAGTTCATTCTT
TAAAATGTAGCAAATGATTAAGCAAATTCAACGAATGATCTTCATCAACTCCGAGGTGTTTTTCCCCCTT
GAAAAATTTAAGTTACTATTATTTTTTTTTCTTTTTTTTTTTTTTTGATACAGAGTCTCACTCTGTTACC
CAGGCTAGAGTGCAGTGGTGTGATCTCGGCTCACTGCAAGCTCCACCTCCTGGGTTCACGCCATTCTCCT
GCCTCAGCCTCCCGAGCAGCTGGTACCACAGGCGCCTGCCACCATGTCTGGCTAATTTTGTGCATTTTTA
GTAGAGATGGGGTTTCTCCTTGTTAGCCAGGATGGTCTCGATCTCCTGACCTCGTGATCCACCCACCTCG
GCCTCCCAAAGTGCTGGGATTACAGGCGTGAGCCACTGCGCCCGGCCAATTATTATTATTTTTTTTAAAC
TTCACCTATCATAAATCTTTTAAAATTTCACCTATGATAAACTTCCTCTGTCATCTGGGGAATTACTTAA
ATGCAATGATGGCCTTCAAGTATACTACCAGGCAGCCTATCCAAATCATGAAACAGAAAGGCTCATAGAC
CAAATTAAAATACTTGAATCACAGAGTTTATTAAAATCACAGTGAGAAGCAAACGGGAAAGATATGTGCT
AAGTTAACACGCTTAGAATAGAGTGTAAGCAGACTGTGAAGATTAGAGTACTTGAATTCTGCAGTACACA
ACATTATATGTCTTGTCTGTCTCTGTATTGCATCAGCCTTCCCAATTATGGTGTGCTTACAAGGACCAAA
GTTGACTTCCCAACAAGGGAGTCCAAAATGGGTGGTGCCTGATTCAGTGGCATGTGGTTTATCAGAGACA
CAGAGACAAGAATGCATGGCCACAGCTGTAACTTGCCAAAATAGCCTGATGACTAGCCATGTAATTCTCA
GGCAGAAGACTTACGGTGCTGGAATAGGTATCACCTATGGATGCCTGAATTAAGACCTTGTGAACATTAA
GTGCTCATGTGTATTTATTTGATCTTGAATATTTAGTGCCTCTTGTATATTTGGTCTCATGTGTATTTAG
CATATTATGAATATTTAGTACCCAGGTGCCTCATGAATATTGGGTATCTTTTGGTCCTTCTATCCCTCAC
TATCTATGTTTAGTACACACATGCTCTGCTTGCTAACTACTTATCTTCTAATTAACACATTCCAAGAGCC
AATTATGTGTATCTCTTTCCACTGAGTTCTTCATTCAATGACATCAAGTTAGTTGCTTGAAATCAGCTAT
TGTGAGAGCACTTACACCACAGAAATTGGCAAATGCTACAATTAGCGCCACTGCCCTCCCCTAGAGCCAG
TTATTTGACATTTACTAGCATTCCACTACTTACATGGCCCTTCATGCTCTCACTCCGTGTGACCACTCAA
GCCTTATCCCTCTTTACTGAACTCTACAATCAAATAAAATATCTTTGTTTCTTGCTTCTGGCCCTCCATC
AAATGGCTCCCTCTCCTAGGAACAATCTGTCTCTCCTCTATCACCTTTGTTTAGCTAGTTAATAATCCCT
TTTCAGACAGCACCTCATAAATAGATTCCTGAACATCCCAAACCGGATCTCCTGCACCTTCAATGTGCTA
CTTCAGTACATTTGTCTTACCCTTTGCCATATGGTATTTCAATTGCCCATGAATTTGTATTCCTGTTTAG
ATCTTAAGCTTTGTGAGGTCTTTTTGTACTTTTTTGTACTTCCGTATACCTACCACATAATAAATATTTA
ATGAAAGCATTAATGAATAAGTAAGTGAATGGAGTGAGTGAATGAGTATTCAATTATGATTCATTTGTAT
CAAAGTGATAACATATACTTACAGGGAAAAGGCCAGAGGGGGAAAAAATAAAAAATAATAATATATTTTA
TGTATGACCTTGTGTGGGGAAAGGAACATAGGGCCACTGCCTGGCCTGCTTCTTTTATGCAAATCCTAAT
GTAAAATATGATCAACGCCTGGCTGGGCAGAAATACAAAAACCCAGTACTAGTGATTCTCCCAACCAGAT
ACCAGCTAGTACAGATCATAGCCAGATTTAACTACTGTGAAGTGGTTAGGTTAGAGGTGACCTATAAGGA
AATGACGCTAATGATCATTAGCATCATCTTGGAAGCTTAAAAAAATGCCCCAGCTGTACCCCAAGCCAAT
GATATCAGACTTTTGTGGGGAACCCAGATATCATTGTTTTTAATCTTTAATGATTCCAATTTAGCCAAAG
TTGAGTCTCAACAAACCAAGTTCTTCTTACTCTCATATTCTCTTCTTCTCGCATAGATAAGAATTAACAG
CCAGCTCTTCTACATGTTTCTTAGACACATATATTGTTTCAGTGGTAATTCGTTAACAGTGCATATGTCA
GCAAAGCATGACTGAAAAAAATATCTGCTCCCACACATTCTGATCCCATCTTGACACTGCATAGCTGTTG
GCGAAGGCAATTTCAACAATGAAGAAGTGGGAGAAATGACTACATTTTATGTAAATATGTATTCATTGAA
AATCAAAAGGACATATGTAATGATATTGTTTAAGATTCTAAATGAAGGACAATACTTAAGAGTCCTCTGT
AGTCAAATTTCTCAGCAGTAAAAAAACATTGTCTTTTCTTTACAACTATTAACCATATGGCTGTGAAAAT
GTTATTCTACAAGCCTTTAAGATTTGAAATCTGACTTTATGTTAATACACAGAATTTACCACACAATCCT
GTATGATTTCTAAGTAGATTTAAAGAGTAGCTATTGCTCACCTTTTCAACATAATGGTAATGATGGTGCA
ATGTCAATTACATGATACTCTCATGGGCGTGATTATATGATTGTTAACACACTGAAGTGCTTATATAGAC
ATAGATACTGATTTTTATATGTACATATTTAAAACAAACAAGGACTTAAAATGGCCTGTAAAAGTCTTTC
TAGTCAGTCTTTCTGGTTTTGGACAGAGAACAAATAATCCCTTACAGCTGTTAGGTTGGTGCAAAAGTAA
TTGTGGTCTTTGCCATTGCTTTTAATGGTAAAAAAAACGCAATTACTTTTGCACCAACCTAATAGTTATC
TACTTCCATCTTTAACGGGCCCTACCCAAGACTGCATGGTATATAAGTAAGAAATGTAAATGAAAATCTC
AAATGCTAGATCTGCCCAGGAGGGGACCACTAATGAGAGAGGAAATGTTAACGTCCCATATGAACTAAGC
TCAGCTTAGCATTTACCCTTCCTGCTATTCCGCTAGAGCAGTGCTTCTCAAAAGTTGACCTGTAATGGAA
TCTTCTGAATGCCTTTTTAAAACGTAGCTGGCTGGGCCCCATCCCCAGAGTTTCTGTGTCAGTTGGTCTG
GGATGGGGCCTGAGAATTTGCATCTCTAACAAGTTCTCAGGGGATGTTGCCGGCCCTTGAATCACAACTT
AAAAACCTCTGCTCTGAAGAAAGGGAAAGCTCTCTCTGCTGGATTTCCCCAAGCCTTTTTCAGATTTTCA
GGAGACTTCTGTGCGGTAGCTTGCTTCCTTCTTTCCATACTACTACTACTACCACTACTACTACTACAAA
TAGCAACCTCTAGCATATTTTCAGTACTAAATACCCAGCACTATATATACATCACAAAAGTCCCTTGAGG
AAGGTGGTATTATCATCTCCATTCTGCGGATAAGGAAATAGATAAGAAATTTGCTGAAGATCGCAGAGCC
AAATGAGACTCAAACCCATGTAACCCATGTCTGTTTGACTTTAAAGCCCGGAATCTTAATTTGTTCCAGA
CAAGCTCATTATGTGCTCTGATCTTCACCACTGAAATGTTCTGAATATGAGGCTGAGGGCAGCAGTGAGG
TTGGAAGGAGCAGCCCAGAGGAGCAGGCACTGTGCTGGTAGAATAGTAGTATGGTGGGGCCTGCACTCCC
TAATAAAAGAAGGGGACAATGACTATTTCCTCCTTCTCCAAGGTCGTGCTGCCTCCCATTTCTCTGTCTG
CCTGGTAAGAAGCAGCTCTGGGCCATGTGTGGTGGCTCACACTTGTAATCCCAGTGCTTTGGGAGGCTGA
GGCGGGAGGATCTCTTGAGCCCAGGAAATTAAGACCAACCCTAGCAATCTAGTGGGACTTCATCTCTAAT
AAAAATAAAAAACTTAGCTGGGTGTGGTGGCACACACCTATAATCCCAACTACTCAGGAGGCTGAGGTGG
GAGGATTGCTTGAGCTTGGGAAGTCGAAGCTGCAGTGAGCCGTGGTCTCACCACTGCACTCCAGCTTGGG
CAGCAGGGTGAGACCCTGTCTCCAGAAAAACAAAAAGCAGCAGCTCTGAAAAGAGGATCTAGCAGTTTCT
ATATGCAGGAGAGCATTTGCGCAATTGTTCCTGGGGTTGAATCTGAGAAACTCACAGTGCACATTCAGAT
ACTATTTACAATCTTCTAGGAATAGTATAAATATTGTGGCCAGGGCACCTTCATATTGTGAAACACAAAA
AGACTTCAGACCTTAGATTATGTGTCGAAAGTTAGGCACCAATGATTTTTTTTTCCATTTGTTCTTAAGT
GGCAAATCTTTACATTAACATTTTTGGTACTTGTCTTTAGGGAAATTTCTTCTCTGTTCTGAATGTATAT
ATTGTAATTCCTCATTTACAATTTTGCCTGCAAATGCAAGTGAGTACAGATCATCCAGTTATGAAAATGC
TCTGAGATTTGAGTCTAGCTGTTTCAGCTTTAAGAGCCCTGACCTAGACTTTGAAACTGACATGGTTTTA
TATGTATGTGGTTGGAATTAAACCCAAAGCACATCTTTTAAAACTCTGAGGAACTTCTGTGCCACAGCTT
TCGCTCAGTTGGTGAGATTTTACTTTGAAATTTAAGGGATGAGTCTAGTTTATATGCAAAGAAATGTAGG
GAGCTTTGCAAACCCAATCAAATCCTTTGTGAACAGTGTGTGCATCTGTTTATTTTGCTGTCATTTTGAG
TCCATGATCCTGTATACTGTTTTGTGGGCACATATTGAGGGTAATATCAAATACCATGTAGAACAGATGC
TGCAGGTATCCTTTCCATGTCCTCTTAGCTTTGGGGTGGTAGATGGGCACATGGACCAAGCCCAAAGTGA
CAGGGTATTAACAGGAGCAAGACTCAACCAATAAGGGAGAGTAGATGGGTACAAATCTCAGCTTTCTCTC
CCCTCACTGGGATAATTTTGAGATATATTCCAAAGATCCTCAGAGCATCCCCAACAGCATTGAGCCCCAG
TTCCCCAGATTAGTAATCTACTCAATAAATACCTCTTTTTTTTTTTTTTTTCCCGAGATGGAGTCTCACT
CTGCACCCTGGCTGGAGTGCAGTGGCACAATCTCAGCTCACTGCAACCTCCACCTCCCGAGTTCAAGTGA
TTCTCCTGCCTCAGCCTCTTGAGTAGCTGGGACTACAGGCATGCGCCACCACACCCAACTAATTTTTGTA
TTTTTAGTAGAGATGGGGTTTCACCATTTGGCCAGGCTGGTCTAGAACTCCTGATCTCAAGTGATCCGCC
CGCCTTGGCCTCCCAAAGTCCTGGGATTACAGGCATGAGCCACCACGCCCAGCCCAATAAAGAACTCTGG
ATTGTTTCTTCCTTTTCCTCTCCTCCTTTCCTGTTCCCTACAGTGTTTCCTAGGATCACCTCAGTCTGCT
GTATGGGAAACCCAGACTGAGTCACCATAACAGACAGAGGCATTGTTACTTTAGGACTTTAGTGGATATA
GTTACATGGGAGAGAGAGACTGTGTATGTATATATAACCTTTATAATATTAAACCATGCTATACTCAAAT
TATTTACTGGCCAAGATTTCCAATATAAATTGGAAATAAATTGGATATAAATCAAGAACAGTTAAAATTG
GAAATAGTTAAAATACAAATAAAATAGCTAAAATTGGGCAAAATACCTGACCCAATGCTTTAATATCCGA
TTGCATAATTAAACGAGTAAAGAGGAAAGGAAATTATTAGCAACTCTATATTTAAATGCAACTGACATCC
AAGGAAGTCATGAAGAAAACTCTTTGTGTTGATAAACTGAAGGCCTCTTCTAGCAGACTTCTGTGTTTAT
TGTTCTGTTGCTGACTATTTTATTCCAAACAAATGAACTTGCTTGTCATTATACCCCACCCTTCCCTAGT
ACAGGGCCCCCATTCTTTGAAACAGTAACTCATTCAGTTCCAAGGAGAATATGAAAAGGGAGGGTAATAT
ATAAAAGAACTGAAATGAAAAGTGGCCTAAGTGTGGCACATTTCCATTGTGGATTCCATGGCAATGGAGA
ATTGATGGCAGAGCATGGTGAGAGATGTGAAGCATCAATTGGCTGTATCTCCAGGGAATTCCTGAAGTTC
AGTTGCCACCCTGGAGGGTGGCAAATGCTCTCTCTCACCTTCCTTGAGTTATTGCTTAGATGACTCAAAA
CAAAAAACTGATGAGCTATAAATGGGCTGTATTATTTGTTTTTACCTGCTGAGTAGTTCAGATATTTCAA
AATAATCTCAAACTTAACCTATGGTGTGGTTTCTGTGTTAAACAAAATACCGTAACTTTTAGTTGAAAAT
ACTGTGTAAGCCCACACAATCTCTTGTTCACAGATAATCTTGTTGTCAAACATTCATGATGACAAAAACT
CATAAACGATTCTTTTAAATATCAAGAATAACTTATGCTGTAAGTCATAATTTCATAAGCATGAATTTAT
GAATGTGTTTTGTGTTTGCAATTTTCATTTAGGTTGTCTTAAAATCATGCGTTTTAGCTTAACTTAGGAG
AAATATATCTTTTGTGACAACATAGGATATTCAGAGAAACGTGAAAACTAGGTGATGTGTTTTATGAAAG
AAGGCATAAAGTATATCAAGCATAAGAACTTTGAATTCTATTTGTGTTTTTTGTGGCTTTAGAAAAGATT
GTTCTGGGAATAGAGAATTCCATTTGGGAAACCTAGCACATACACAGTAGCAGAGTTAAAATACTGACTT
GGAGGGTTCATTTGAAGAATTCTATAGAATTTTTGCATGTTGGGAATAGGTTTATATTCTTAAACATTGC
ACTCAGGGTTTCTATTCAAAGCAAAAATAACTTTGCATAGACCTTGGCCATTCTTTCACATTCTAAAGTA
ATCCATTTTTTTTTTTCAGGGTAGTTGTTCTCAGTCCTGATTTTCTGATAATTCAGATCATCTTTAATTT
ACACCAAAAACTTTTAGAAGAGTCAGATAATAATTTAACATAAAATGTAAATGACTGAAATATACATTTT
TTAAAGGAGCAGATATGGAGGGGTCCAATGTACTTAACTATTTGCTCTCTTTGTCTCCTTGCATTCACGG
GAATGTTTCTATGTAGTTTTCTAATTTCACACAATTTCAATAATCCATACCCTCCTCATTTTTATGGGCC
TTCATGATACTAAAAATGTTACCAGAAATTATTTTGTGTTAGTCTCTTTGTTTAGCACATTCATACATAA
GTTTTAACATTTAACTGGCATATTTTTAAAGTAATACATGTTTTTTTTTTAAAAAAAATCAGTTATGTTT
GTGTGTGTGCATATTTTCTTTTGTGGCCAAATGTTGCACGCCCTAGTCCTTCTATTTAAACAATGAGTTT
ACATAACAAATGTTACATGATAAACATGAAGACATTTAGTTTGAAAAAAAATGATTTTCTAGTTTACTCA
TTTAAAAAAAGCTGAAGTAACCGGGAAGAGGAGTGGCAGAACATATTAGTCTTTTTCATAATGCCATCAT
TAAACAAAGATACTTAATTTCCAGGCCTGGTGCAGTGGCGCAGCCTGTAATCCCAGTACTTTGGGAGGCT
GAGGAGGGCAGATCACTTGAGGTCAGGAGTTCGAGACCAGCTTTGCCAATATGGTGAAACCCTGTCTCAA
AAAAAAAAAAAAAAGAAAAAGAAAAAGCTACATAATTTCCAAAATGACTTCAGTGGGACCTGAGGTGAGG
GAATAAAGGCTCTGGAGTAATTTCACTCTCTATTCCTCTCCTAATTTTTTTTCTGTTCCTTTATAACAAC
ATTTTCACTACTTTTGAGCTTGGGAGTTGAGGAATCATGACCAGAAGAAAAGGAAAGACGGGAAAGATGT
TCAAGGGTGAGGATGCTTAAGAATGACCTGGCAAGCTTATGAAAATGCAGTTGTCTGGATCCCACCACAG
AGATTCTGATTTAGCAGGTCTGTGGCAAGGCCTGCGATTCTGCATTGCTAACCAGCTCCCAGGTGATGAC
ACTCATGCTGGCAACCTATGAACCATTGAGTGGCACTGTTCCAGGGGGCAGGGCAATGAGAAATTGAAGT
CAAAAGCCCCAAGACCTGGTGCTACGAAAATACTCTGGTTCCTTCCCTCTCAACTGATTTACTTGTCGGT
GTGATTTTGCAAAAATCCCTGAACTTCTTAAATCCCAGTTACCTCACCTGAAAAGTATGAGTGTTGCTCC
AGATCTGGAGGCTTTCAGACCATGCAAATCGAATTCAAACCATGCAAACCATTCAAGTCATTCTAGAAAG
TTCTGCAAGGTGCCTCAGAGGCCAAAGGGAGAGATGGGAAGAGGGATTGAATGGGCTCTTTCCAAGGTTC
CCTAACCCACTTGAATACTTTCATCTTTTATCTCTTTCATATATTCCACTTTTGAGTATGGTTTCATTTA
GAAAATAGGATTTTATACCAACAGATTTAAAGAAAAACTCCAAGTCTGAAAATGACTCATTTATTTAAAA
CTGTATAGAACAAAGACATTTAGTGCACAATTCCAAAAATTCTCTGATCCTTCCACAGCATGCCCAGTAT
GCTGCAAGAGTGCCAGCAAACACATGCTTACTGCTCACAAATGTGAAATTTAACCCCATGCACTAGGAGG
TCCCTAGTGTGGGGTGGTTTTAGCTAACCAGACTAAGAGAGTACAGGGCAACATCGAGCCTTTCTCTGCG
GTCATGTCTGATTCATTAAAAATCCAGCTTTCCCCGAAGATATATTAATTACCTTCTGTTTCAGAATTTG
TTTTTAGAGCCTAATTCTTAATTATATCTCCAGCCATTGTGTGATTTGACCATTTTGGAACTAAAAAGTT
ATCCTATGAAATTCCACCTCCAACTATTGCCACACTGTTAGTTTGTCTATTTCATACACCATGCCAATCT
TAGCGTGGTGCTAGCATTTCATTATAACCAGCTTTCATTTTTAATAAGACCATGTGTATATGAAATTGTA
GACTTCAGTCTTTGTATGAATTGAAAGCTATTAATCTTCCCAGGGTTAGGTTATGTTAAACAGATTGTAA
TGTTCTTCTTTTTATTATGTTATTTAAATCCCCTTCATTTCATACTGCACCAATACATTTCTACTATCTT
GGAATAAATTAATTCCAGTTACGTGATGGAAAATTTTAGTGTAAAAATATAACCTGCAGTATAATTTTTT
CTGTCAGAATACCAACTAGAACTGGTATGTTTCATTCTAATTGGAAATTTGAGTTATCGCTTTGATTTTT
AACAGTGGGAAAGGAAAATGAAGATTGATATCTTTCAATAGCCGTTCATTCATTCTTCATTCCTTCATTC
ATTCACGTATTAAGAATAGTCTATGTGCTAAGAACAGAAAGAGTGTTAGAGATATGAAGATTAATAAGAC
CAGATCCCTGCCTGCAGGCATTTCCTATTCTATGTCATAGATAGGGGGCTATTCTGTTTAGAGGTAAAGC
ATGACCCACATTGCCTCTGACAAGAAGCATAATGTCTGTAGCAGCTAACTGCTGGAGACAGGAGGCTAGA
GGGCTGCCCTGGTAATTGGTATTCAAGTCTTCAAGAAAGGAAACCAGCTATTCCAAAATCAGTGGGCAAG
AGGAAGTTGTAAAGTTAAGTGAAATGACTAAAATATGAATAACTAAAGGTTGGAATCTGGTAGAAGGAGA
GGAGAGCATCGGTCAGCACCTTAGTTTGGGAAGGTGGTGTGGCCATAGTAGGCTTTATTTAGAAAGAAGC
AATTCTTAGGTACCAGCTAGGTTTCAGTTCCTTAAGGGGAGAAAACTGGCAAAATATAGGCAGGTTTCCA
GGGTGCAAAGCCACGTTCTAGCTTCAGCTCAGGCAAGGCCCTGGGGTATGAATCACCACCAGAGTAGCCC
AGCCAAAATGACTAAGGGATCTAAGCTGGTTGCTAATGAAAGAGGTTGCAGCTCAAGGCAGCTCTGCTGA
CGCCCACTGGATACTGGGATTACATTGATTTAACACATGGAAACCACTTAATATGGTATGTGGCACAACA
CAATTAAGTACTCATAAATATTTGCAGATAATGCTGCTGCCATTGCTGTTTTTGTCGTTAGAAGACTCGG
GAAAATCATCTAATACAGGAATCCATCTGTTGGCGGGGCTTGGGCTTCTAATATTTGACTGGTTGATTTT
TGTCGACCCAATCTTAACAATATTATACACAGCCATTACTTCAGGAAAGGCAGTTGTAAAGAATGGTATA
AATTTCCTGTAACTTGACTGCCACATTCTAGCTGAGTCACCTCTATATACCTCAGTTTCTTTGTATCCGC
AGTGAAGATTAATGACCTCATAGGGTTGTTATTAGAATGAAGTGAATTACTACACTGGACTTATTTAGGA
CAGTAACTCGCACATAGTGAGTGCTCAAGGAAATCTCAGACCCTGCCTGCTAGTGGAGGGTCCAGCTCCT
GATACATTTGGGGGCAGGTTTAAGGAGTTCATTGATTTAGAGCTGTAAGGGCTGATCTTTCACCCTGCAT
GTCTTCAGCAACTGTGGCTGGTAAAGTCCAGAGCAGTCAAAGGCTGACAAATCCTTGTTAGAAATCACAA
ATGCCCATTCTCACAACTTCTGTGGTGTTTTCCATCCTTTCCCTAGAATACTTTCTTTTTAAGGCAAAGG
AAAGAATAATCACTGCAGATAGCACACAGTATTTTTTTGCAACATATTTTCAAAAATTATGATGAGAAAA
GTGTATCATTCCTGTGAAGAAACAGCATAAGGAAAATGATTTGAGAAAGAAACATGGTTCTTAAACTGAA
ACAAGTGTCAGAAGGAATCCCAGAAGGCAGAAGGAAATATAGTAATCATGATGAAGTCTAGAGCTCACAC
CGGTTAACAGAATGGCAGCAGCGATATTCATCTCACGCCTCTTCCATGCTGTCCCTGAGTGAGCTTCTGC
TGAATTGCCTGGCTGGTGAGGATTGGTTTCAGCAGCAGAAGGAATGGGCTGCCAGCTGAAGGCTCTGGTT
CTGATCCTGGGTAGGGTCAGAGAAAGCAAGATGTGACCATCACTTTTGACCTTGGTCTTGAATTTGATTC
CATGGAACAACGATATTTTACAAACCCAGTTGAAGGTTTATCCCTTTTTCTATTCAACACAGGGAGAGTC
CTTAGAGCCCCAGGAAGACTTAGCCCTTTTTCATTCTAAGAGTAAACCACATCTAGGTTTCCAGAGATGA
AAAGACCAGGCTCTGATCTTCCTTCTGGAAGCCCTTGCCTATTCAACAAGCATGAGTATTAAATGCTATT
GCCTTGGAATCATAATTCAGTTTTCACAGTTTGGGCTATGTCAGAACCATTCTTGTCAACCCCCTGTTTT
CTGAGAACCCGAAACCTGCTTGTTTAGAATTTTAGAATCTACTTGACTCTTACAGGGGAGAAAAGATCTC
TTTTCTCACCCATCGCTAGGTTCATGGCTGAGGCACCTATAATGAAGGACAAATCAACAACATAAAAGCA
TGCGAATTTATTTAATATAAGTTTCACATGACACAGGAGCCTTCAGAAATGACCCAAAGAATCAGGGAAA
AGTGTGTATTTTTATGCTCTGATTTGAGGAAAAGTAGATGTCCAGTATGACTGGACAAAGGGGAATGGTA
ATAAACTGGGGTGACCACAGCAAGGCCTGTTTCTGCAGAACCTCCTGTGTCCCTGTGTTTTCAGAGGTAA
AAATTTTCCTTTCCTTCCAGTATAGTAAGGGCACCTCTGGTATGATAGTCTCATGACCTGCTTCAGGGGA
GAAGGGGGAAGGGGAAGGTGAGAGTGACCATCCTGCTTCTGCTGTCTTCTCAAATACCAAGCTGCCATAT
TGTGGATTTTGGAGTAGCGTAACTTGAATCCTTTTTTTTTTTTTTTTTTGAGACGGAGTCTCACCCTGTA
ACCCAGGCTGGAGTGCAATGGCACAATCTCGGCTCACTACAACCTCCACCTCCCAAGTTCAAGTGATTCT
CCTGCCTCAGCCTCCCGAGTAACTGGGATTACAGGCACATGCCACCATGCCTGGCAAATTTTTTGTATCT
TTAGTAGAGATGGGGTTTCACCATGTTAGCCGGACTGGTCTTGAACTCCTGACCTCGTGGTCCGCCCACT
TCGGCCTCCCAAAGTGCTGAGCCACCGCACCCAGCCGCATAGCTTGAATCTTATCAATACCTTAACCAAA
TGACTCTGACAGTTTTCCTCTTCTTATCTAAATTCTTGAGGGTCACCCACACTTCCCAATGTCTTTTGAA
ACTTGACCTCTTTTCTGCTGAATTGAGGAAGATACCTGATTTCTTTAACCTCACCAAATTCCTACTTCTT
ACTGTTGTTCATTGCTGGCTGAAAATTTACTTTGGCGAGTTCACCAAGAACATACTTATCGGTTCACTGT
TTATATTTGCACTCAAGATAACACTTGAGGCCCTGCTACTCAAAGAATTTAGTGACAACTTTCTTCATCA
CTCTCATATCTTATCTGTCATCAAGTCTTTTTTTCCTCGTAAAAATGCTTTTAGCTCTTTAAGTATGTTT
CATATCTATAATAGCTAAGATAGGCTAACAGCTATAATATATTAAACATCCACCAAATGGACTATTAAAA
TGACTTAAACAAAATAGAAATGTATTTCTTTCTCATGTAAACAGTCTAAGGTGAATTCATGTTAGTTGGT
GTTGGATGTGTGTGTGGGAAGGGAGGGGTGACACCCACATAATTATTCAAGAATACAGGCCAGGCCAGGT
GCAGTGGCTCACACCTGTAATCCCAGCACTTTGGGAGGCCAAGGTGGGCGGATCACCTGAGGTCAGGAGC
TCGAGACCATCCTGGCCAACATGATGAAACCCCATCTCTACTAAAAATACAAAAAATAGCTAGGAGTGGT
GGTGGGCACCTGTAATCCCAGCTACTTGGGAGGCTGAAGCAGGAGAATCACTTGAAGCCGGGAGGCGGAG
GTTGCAGTGAGACAAGATCATGCCACTGCACTCCAGCCTGGCGACAGAGCAAGACTCTATCTAAAAAAAA
TAAAATAAAATAAAAATAAAAAATAAAAAATAAATTAAAAAAAAAACAGGCCAGCAAGGGTCTTCCATCT
GCAATAGCCAATTGCCGAGGTTGCCCTCCTGAAGATATTCAGCCAGCCCAAAGGGGAATGAGCTAGAGGA
CTGCACACGGAGGCGTCCCATGTCCTTTGACTCAACCTTCTACTGGCTAGAACTCTGCCCTGTGGCCACA
TGTAACAGCAGAGGGGCTGGAAAATGAAGTCTAGCTAGATACCTAAAAAGAAGCAGAGAAAGGTTTCAAG
AGCATTTAGCAACCATATCCACCTTATTCATGCCCTGCCCTCTATTCGCAGTGGCCCCGCAGCACTGCTC
AACTAGCTTGCTGCATTGGCCTCTTATCTCTTATCTATTGCCTTAGATCCATCTAAATGCTCTGCTACTC
TTATGCCTGGAATATGTTTTCAAGATGTGACTAATCCTCTCACAGCTTGAAGGATAAAAGGTCAAACTGC
TCTGGTGAATGCATGATGCCTGGTCACCTCTGTAGCCCCATCTTCCCTGACACTTTCACAGACAGTATTC
CCTTTACTCCAACCTGTGGGAGTATTTTCTAATTCACAATAATAGCAGGAAATACAATGTGGACCAGACA
CAGTTCTGAGCAGTATATTAACTCATGCATTTCTTACGATAACTTTATAAGGTTGACAGTAGTAGTATCC
CTATTTCACAGAGAAGGAAAGAGATACAGATAAGTAATTTACATATGATCTCACAGATAGTAAGTGGTAC
AGCTTGAGTGCATATGACTCAAAGGGTAGAGGTTCTAGATTCTTAATCACTGTATTGTACTACTTCTCCC
AATGTTATCGTACATGCCATTCCGTCTTCCTGGAATACCCTTCGTCTTTCTTCATCTAACTTCCACTCAA
ACTTTAAGGATCAATTTAAGCATGCCTTATTTTAGGTAGCCATGGTTGACATCAGCCTAATTTAATTGCT
ACTCATCTATGTCCCCATAGCGTCCTTTGCATTCCTCTATATCTCTCTGCTATAGCAGTAAATGTACCAC
CATTCCGTAAAATCCTTAAGGGAATGTTTAGTTTTATGTTCCCAATGCCAGCACAATGTCCAGAACAGTG
TTGTCCCATAGAAATGAGAGCCACCTATGTAATCTTAAGTATTCTAGTAGCCACGTTCTTTAAAAGTAGA
AGTGAAACTAATATTTTATTGACCCTGATATATCCAACATATTATTATTTCAATATGTAATCAATAAAAA
GTATTAATAAAATTTGCTTTTTCCATTCTAAGTCTTTGAAATCTGGCATGTGTCTTTCAATTGCATCCCA
TCTCAATTTGGACACCGTATTTTCATTGAAAATATTTGGTCTCACCTGCACACGTATGTTTATTGCGGCA
CTATTTACAGTAGCAAAGACTTGGAACCAACCCAAATGTCCATCAATGATAGACCGGATTAAGAAAATGT
GGCACATATATATCATGGAATACTATGCAGCCATAAAGAGGATGAGTTCAAGTCCTTTGTAGGGACGTGG
ATGAAGCTGGAAACCATCATTCTGAGCAAACTATCACAAGGACAGAAAACCAAACACCACATGTTCTTAC
TCACAGGCGGGAATTGAGCAATGAGAACACTTGGACACAGGGTGGGGAACATCACACACCAGGGCCTGTC
GTGGGGTGGGGGGAGGGGGGAGGGATAGCGTTAGGAGATATACCTAATGTAAATGACGAGTTAATGGGTG
CAGCACACCAACATGGCACATGTATACATATGTAACAAACCCGCACGTTGTGCACATGTGCCCTAGAACT
TAAAGTATAATAAAAAAAAAAGAAAATATTTGGTCTCTATTTACATTTCATAAACTTTATAGTTGAAAAA
AGAAGATTCACATTCCTAAGTTGTTCCAAACATACACAAAAGTTTTTCAATAACTGAACCAAGAGTCAAT
TTTTAAATTTATATTTAAATTTAATAAAATGGAATAAAAATTTGTTAAACTTCAGTCTCTCCGTCTCACT
AGCCTGATTTCAATTGCTCGGTAGCTACCTACAGCCAGTGGCTCCTGTGTTAGACAGAGCAGCACAGCCC
TAGAACACAGTAGATCCTAAATCGATGTTTATTGAAGAAATTAATCAATGACAGTGTAGAAAATTTGCAG
TGATTATGTCAGAATCAATAGTTCTCCACCCATTTTCTCCCACACTCTCAAAAGGGCCAAGTTTTATATC
ACCAAATGATATTCCTCTTACTTCTTTCTGAGCAGAAACAGTTTTGGAAATTAAGATCTTTTTCAAATTT
TCCAGACTCGGCATTTTAGCAGCGTTTCTATTTGTACCAACAATGCCTTTCTACCTATTTTCCTTGCTTC
TTAATAAGTTAACTTTGTGCGAAGGTCATTTTGTAGGTCAGTGTAATATTGTGCATTAAGGGCTTCTAAG
TTTTCTGGTATTATAAGAACTCCTTGGTTTCCTTCTACTTTTCAGAATGGAAAATCCTCAGAGCAATTTT
CATCTAAAAGTGCTGCATTTAGGTTGTTTCACAATTCCCCAACCCTGAGTCAAATATAGGTTGGTGTATG
AGCAGCAGTGTCTCTTGGCTAATCAAGAGCGTCTCCTTTTGCTACGCTCAGTGTTAGAGAAATGGAGAAA
GTCAGCTGGGTTTAGAGATTAGGTGAGAGACTCAGGCATATCCTTTGATAAGTCATAAATCATTTCCTGT
TTAGAAAAGCACATGTTTAGACACCCATAAAATCTCCAAATGAAGGGTGTTTTACTTTTCCTTCAAAATC
TCACTGGGAAAAGGTACTTCTGACTTTCCAAGTGAATAAAAATAATGACTCCTGATTACCATGTATGTTT
AAACTGATTTGCAAAGCAAGTGAAAAAGAGTCTAGTGAGTAGTGATAAGCATCTTTTAGACATCAGAAGA
TGTACTGATTTAAAGGTCCGTATCATTTTATAACTAGTATCTATTGAGATTCAAATGGTTATTACTCTGT
GTGAATCTGTCTTTTCTAATTGTTTTTACTTATTTTAGAATATCGATTTGTGAATATTAAATTCCTAAGT
TTTCCAGCAATCCAGTGTTTGTTTTGGATATCCAGCCTGGATGCAGAATAGCTGCAGAAAGTTATCACAA
ATTGATCTCTATATTCTGTTTCCGAGTGGCAATTGTCAAAAATTTGGGGTCATCGGCTACCCCTCCCACC
CCTAAGAAGTTCCTTGTACTTCCTCTTTCAAAACACTCACATCATTGTTCAGTGCCTCACTTCTCTACTA
AAATGTAACCAACCACAAAGATAGGGACTATGTCTTTCCTGTTTACTGGTGGATTCTCAGTATCTAGCAC
CATGACCAATGTTAATAGACGTTGAATCAATTCCAGTTGTTACCTCTTCACACTGGGACAAAAGTCCTTG
CAAGTATTCTGCTGCCATTTGTATAGATTCAAGCCAAATATGTCTCAAAACGATATTACAGATGATCTCT
TCGTTGTTCCTCTGACAATTTCTTTCCCCCCTGCATTGCTTAACTTGATTGACAATGACCCCTACTACTT
ATAACATGTGCCTTTTAGGTAGTGCACTTGGCACTACATTTTATGTGATAGTTTTATGATGCTAAAGACT
ATTTGCTGTGATGATGCTGTGTTCTCACATGGCATATCCAGATTTATTTATGCTGGTGACCAAAGGCAGG
TAGTTAACCTTGAAAATAGGTTAAAATTTGAAAGGCAGCAAATCTTAGGGCTAGAATTTATAATTTATCT
TTAAGGAATCTTGAAACCAGGTGTGAAGGAAGGGACGTAGGCTAGAAGTACAGAAACCTGGGTTCTGCTC
CAGCACTGATGTTAAGAGCAAGTTGCATTGCTTTTCTGGACCTTAATTTTCTCTTCTGGAAAATGAATAG
ATTAACTGGAACAAGGAAGGAAAATACGTGAATGGCTTCCGTTTCTGTCTCGTTTACCCCTGAAAGACAT
GGCTAGTCAGTCAGCTCTGTATCAGAGCACTTCTCAAGGCAATGCTCCAGGTAGCTACCACTCACTAATG
AGAGTTAGCACATAGGTAAAACCTCTTTGTCATCTCTAGGCTACTTCATGTTTAAGATACTCTCCAGCTT
TAAAATTCTAATAACTCTATTAGACTGAAATTTAAGAATACGAGAATAATCATCCCTCACCATGAAGAGA
GAGTCTGAGGAAAAAATAATGAGAACGAATAACCCTTCTCTTTTACTACAATTCAGGACTGCCATGAAGA
GCCGTCCAGATTGTGAAACATACAACTCATGATGTGAATGGTACTTCTTTGTTTTTCTCGGTGTACAACT
TGCACAGCTGTTCATGGCCCTCTGCTTCCACAAATTCATTTCTAAATAGCTGTACCTCAGTTCTTTGACT
TCTAGTATGTCTAATTTAATACACATTTCTAGATTTACGATATATAAGAAATATCTCCATGAAGGAAAAA
TGTAATAGCCCATGCTTTTCATTATAATAGAATTTTATGAAACAATGTCTTTTAAAAACAGAAACATATG
TACTACTACTTCGCAGGACATTAGCCCTTGTATATAAATCAATAATACAAAAAATTCAAATTACCAAGGA
TTAGAAAAGACTGCTGTGGGATATCTTCTGGTGCAAGCATACAGTTATTTATCCATTTCTTTCATGAATA
TTTATTGATGTTCCAAACATTAGGCTAGACACTAGAGACACATCAATAAATAAAGGAAATAGGTTTGATC
TCTATCTTCTTTGATCTGTAGTTTAGTGGGGGAGGAAGGAAATTAAACAAGTAACTACTACAGGTTGAAC
ATCCCTGATCCAAAAACCTGAAATCCAAATGTTCCAAATTCCAAAACTGTTTGAATGCTGACATGACATC
ACAAATGGAAAACTCCACTTCTGACCTCATGTGACAAGTCACAGTGAAAATGCAGGCACACCACATAGAG
TTTATTCAGCATCCCCAAGGGAAGAAAGATCCTCTCAGCCCCCGTTAGCTGTGATATATCTTTTCCACCC
ACACCCAGATTCCATCATACAAGCAAACCCACAAAAGGTACGAAAAATGGCACATGTGCGGGCTAGACGC
GACAACGGCAGGTACCCTACAATGTCCAGCATGGGGCCAAAACCTACGTGCATTAATCACTGTGTTTGCT
GGTATATTCTCTGGTGGTGTCAAGATATTGTTGAAAATGCCCTAAAGGCCTGCATGATATCCATAGGGTA
ATGCAAATATTCCAAAACCTGAAATTTGAAATACTTTCAGTCGCAAGTATTTTGGATATGAGATATTCAA
CCTATAGATGGTAAGGGTATTACTATGATAGTGCTACGGGTGTACATCAAGGTAATTGACCCTGGCTTGG
CAGGATGCAGAAGGCTTTCCCAAGGAAGCCTTACCTCAGCTGAGACCTGAAGAGAAGCAGGAGTTAGACA
GGTCAAGTTGGGGATTTGGAGGAGGTGGAGTCCCAGCAGATGGAATACTATGCATAAAGGCCTGGAAGTG
AGAAAGTCATGTCATGTTATTTCAAGGGACTAGAGGAAGCTTAGCAAACTGGAGGCAAGAAGATAGCTTC
AGCACACTATTGAAATTGTCCAGGTGAGTAATGATGATAGTGTAACTAAGTTGTGATACCTAGGTATGTG
AGCTGAACCTATGGAGAAATGTTCTAGGCTAGAGAATCTTTAATTGGATATTTAATTATCAGTATATACG
TAATTAAAACCTTGCAAGGGTTTGAAATGGTTCAGAGTAAAGTTCGTAGATGAGAAGAGGGCCTAGGAGT
GAACCCAGGAAAATGGCAAAGTTTCAGGGGTAAATAAAGAAAAATAAGCTTTCAGTGGAGACAGGGAAAT
TTGCAGTTCAGTAGATAGGAGACAGACTAGGTTCGTGTGATGTCACAGAATCCAAGGGAAGAGAGGTTTT
CAAGAAAAAGTAACATTTAGAGGTGTCAAATACTACAAAAGCATCATGAAAGATAAGACCAAAATATATC
CTATTAATTTAGCAACAAGGAAGGTATTGACAACCTTTATGCAAGTGATTTCAGTGTTGATTATGGAGAA
CTCAGTAATTACTTGGTGGTAACTAAAGAACCAAGATTGCAGTACGTTCAGGCATGATTGAGAATTGAGA
AAGTGGGGGAGCAAGTGTAAAACAATTATTTTAAGACTTTTGGCTGCGATGGGAAGAGAGAAAGGGCCAT
AGTAGCAGAAGATGGATGTAGGGGCAGGAAGAACACACTCTTAAAAGGGTAGTGACTTACACATGTTTAA
ATGGCAATGAGAAGAAGATGGTAGAGAGGGAGAGGTTGAGGATGCAGGAGAAATTAGAGATAATCAATAG
CACAGGTACTTGAGAAGGCAGAAAGATGAAATTTAGAAATTAGCTTCAGATAGGAAGGAAAGTACAGCTT
CTATTACAACATCAGGGGAGAAGGGAAGGAGGATGGGCATAGCTACTGGTAGTTTTGTAAGTTTGGTGAA
AGGTTAAGTAGGATTGTTGTATTGGATTTATTTTTTATTGAAGTGGAAGCTGCAGCTAAATGCCCAGTGA
TGAGGAAGGTGTTGGAGTCTGAGATTTAAGGTGAGTGGCAATTTGAAATAGCTGCTCTAGGATCCTATTT
AACAGAGAAAATGTTGAGTACACAATCAGTGAGCAGTTTTAAGTCCACTCTATTCTGTTTGCAGTTTCAA
GTACCTTTCATTGCTTCTAAGTTTATGAAAACTGGTTCACAATCTTCTTGTGCTTCTTATTTCTACCTCT
TTTCCTTCTGTTTTCTCACCTCCCCAGTTTAAACAGTCCCGAATTTTTTACAATTAAATATACAGCAACT
GCCATGAAATCTACTGATAAAAGATACTGCAAAATCAGTTTGGGATTGGGTTCATTAGCTTACTTATTAT
TATCAATCCTAGGCCACTAAGCAACCTTGCATAAAATGCATAAAATGAGGAGATTCTAGTGGAGGATAGT
TTTCAATTATCTCATTAATTTCAGGCCATGTGACTAGTCCAAATAGATATTATAGGCCAAGAAGAGCCTA
TCTTGAGATTTTAACTCCCAGGATAGGTTTTCTACCTGATCAAAAGAATCTAATAACTATTCAATCTCTT
CTTAAATGGTTTGGTTTTCTGTGCAAACAGTTTTACCCTTTTAGCTGATTTTCTAGGTGTTAAATTAAGA
AAATTCTCTCAGATACTTGTTCATCATGTACTAGGATCCCTGATGTGTTCAGAGTTGTCCAACTTTCAAA
GGGCTTTGCATTCAGAGTACCTAATCTAAACCCTGATATCATTCTTTTATAACAGAAAACCCCGGATTAG
ACTGGGACAGTGTCTGTCATGTTCATCACTGCATCTCCCTCAGTATTTGTAGAATGAATGAAGGGACAAT
GGCAAACTATAGTCCTACCATCACACTTTTGGTAGTGAGGAGAACTGCTGTAACTTGGAAGATTGGAGGG
GGAAAAGGTGGCTAAAACAATCATACAGTAAACTGGGCTGCTATCAAGAGAAACCATTTGTCAATTTTGG
CTTTTGTTGCCATTGCTTTTGGTGTTTTGGACATGAAGTCCTTGCCCACGCCTATGTCCTGAATGGTAAT
GCCTAGGTTTTCTTCTAGGGTTTTTATGGTTTTAGGTCTAACGTTTAAATCTTTAATCCATCTTGAATTG
ATTTTTGTATAAGGTGTAAGGAAGGGATCCAGTTTCAGCTTTCTACATATGGCTAGCCAGTTTTCCCAGC
ACCATTTATTAAATAGGGAATCCTTTCCCCATTGCTTGTTTTTCTCAGGTTTGTCAAAGATCAGATAGTT
GTAGATATGCGGCATTATTTCTGAGGGCTCTGTTCTGTTCCATTGATCTATATCTCTGTTTTGGTACCAG
TACCATGCTGTTTTGGTTACTGTAGCCTTGTAGTATAGTTTGAAGTCAGGTAGTGTGATGCCTCCAGCTT
TGTTCTTTTGGCTTAGGATTGACTTGGCGATGCGGGCTCTTTTTTGGTTCCATATGAACTTTAAAGTAGT
TTTTTCCAATTCTGTGAAGAAAGTCATTGGTAGCTTGATGGGGATGGCATTGAATCTGTAAATTACCTTG
GGCAGTATGGCCATTTTCACGATATTGATTCTTCCTACCCATGAGCATGGAATATTCTTCCATTTGTTTG
TGTCCTCTTTTATTTCCTTGAGCAGTGGTTTGTAGTTCTCCTTGAAGAGGTCCTTCACATCCCTTGTAAG
TTGGATTCCTAGGTATTTTATTCTCTTTGAAGCAATTGTGAATGGGAGTTCACTCATGATTTGGCTCTCT
GTTTGTCTGTTGTTGGTGTATAAGAATGCTTGTGATTTTAGTACATTGATTTTGTATCCTGAGACTTTGC
TGAAGTTGCTTATCAGCTTAAGGAGATTTTGGGCTGAGACGATGGGGTTTTCTAGATAAACAATCATGTC
GTCTGCAAACAGGGACAATTTGACTTCCTCTTTTCCTAATTGAATACCCTTTATTTCCTTCTCCTGCCTG
ATTGCCCTGGCCAGAACTTCCAACACTATGTTGAATAGGAGTGGTGAGAGAGGGCATCCCTGTCTTGTGC
CAGTTTTTAAAGGGAATGCTTCCAGTTTTTGCCCATTCAGTATGATATTGGCTGTGGGTTTGTCATAGAT
AGCTCTTATTATTTTGAAATACGTCCCATCAATACCTAATTTATTGAGAGTTTTTAGCATGAAGGGTTGT
TGAATTTTGTCAAAGGCTTTTTCTGCATCTATTGAGATAATCATGTGGTTTTTGTCTTTGGCTCTGTTTA
TATGCTGGATTACATTTATTGATTTGCGTATATTGAACCAGCCTTGCATCCCAGGGATGAAGCCCACTTG
ATCATGGTGGATAAGCTTTTTGATGTGCTGCTGGATTCGGTTTGCCAGTATTTTATTGAGGAGTTTTGCA
TCAATGTTCATCAAGGATATTGGTCTAAAATTCTCTTTTTTGGTTGTGTCTCTGCCCGGCTTTGGTATCA
GAATGATGCTGGCCTCATAAAATGAGTTAGGGAGGATTCCCTCTTTTTCTATTGATTGGAATAGTTTCAG
AAGGAATGGTACCAGTTCCTCCTTGTACCTCTGGTAGAATTCGGCTGTGAATCCATCTGGTCCTGGACTC
TTTTTGGTTGGTAAAATATTGATTATTGCCACAATTTCAGAGCCTGTTATTGGTCTATTCAGAGATTCAA
CTTCTTCCTGGTTTAGTCTTGGGAGAGTGTATGTGTCGAGGAATGTATCCATTTCTTCTAGATTTTCTAG
TTTATTTGCATAGAGGTGTTTGTAGTATTCTCTGATGGTAGTTTGTATTTCTGTGGGATCGGTGGTGATA
TCCCCTTTATCATTTTTTATTGTGTCTATTTGATTCTTCTCTCTTTTTTTCTTTATTAGTCTTGCTAGCG
GTCTATCAATTTTGTTGATCCTTTCAAAAAACCAGCTCCTGGATTCATTGATTTTTTGAAGGGTTTTTTG
TGTCTCTATTTCCTTCAGTTCTGCTCTGATTTTAGTTATTTCTTGCCTTCTGCTAGCTTTTGAATGTGTT
TGCTCTTGCTTTTCTAGTTCTTTTAATTGTGATGTTAGGGTGTCAATTTTGGATCTTTCCTGCTTTCTCT
TGTAGGCATTTAGTGCTATAAATTTCCCTCTACACACTGCTTTGAATGCGTCCCAGAGATTCTGGTATGT
GGTGTCTTTGTTCTCGTTGGTTTCAAAGAACATCTTTATTTCTGCCTTCATTTCGTTATGTACCCAGTAG
TCATTCAGGAGCAGGTTGTTCAGTTTCCATGTAGTTGAGCGGCTTTGAGTGAGATTCTTAATCCTGAGTT
CTAGTTTGATTGCACTGTGGTCTGAGAGATAGTTTGTTATAATTTCTGTTCTTTTACATTTGCTGAGGAG
AGCTTTACTTCCAAGTATGTGGTCAATTTTGGAATAGGTGTGGTGTGGTGCTGAAAAAAATGTATATTCT
GTTGATTTGGGGTGGAGAGTTCTGTAGATGTCTATTAGGTCTCCTTGGTGCAGAGCTGAGTTCAATTCCT
GGGTATCCTTGTTGACTTTCTGTCTCGTTGATCTGTCTAATGTTGACAGTGGGGTGTTAAAGTCTCCCAT
TATTAATGTGTGGGAGTCTAAGTCTCTTTGTAGGTCACTCAGGACTTGCTTTATGAATCTGGGTGCTCCT
GTATTGGGTGCATAAATATTTAGGATAGTTAGCTCCTCTTGTTGAATTGATCCCTTTACCATTATGTAAT
GGCCTTCTTTGTCTCTTTTGATCTTTGTTGGTTTAAAGTCTGTTTTATCAGAGACTAGGATTGCAACCCC
TGCCTTTTTTTGTTTTCCATTTGCTTGGTAGATCTTCCTCCATCCTTTTATTTTGAGCCTATGTGTGTCT
CTGCACGTGAGATGGGTTTCCTGAATACAGCACACTGATGGGTCTTGACTCTTTATCCAACTTGCCAGTC
TGTGTCTTTTAATTGCAGAATTTAGTCCATTTATATTTAAAGTTAATATTGTTATGTGTGAATTTGATCC
TGTCATTATGATGTTAGCTGGTGATTTTGCTCATTAGTTGATGCAGTTTCTTCCTAGTCTCGATGGTCTT
TACATTTTGGCATGATTTTGCAGCGGCTGGTACCGGTTGTTCCTTTCCATGTTTAGCGCTTCCTTCAGGA
GCTCTTTTAGGGCAGGCCTGGTGGTGACAAAATCTCTCAGCATTTGCTTGTCTATAAAGTATTTTATTTC
TCCTTCACTTATGAAGCTTAGTTTGGCTGGATATGAAATTCTGGGTTGAAAATTCTTTTCTTTAAGAATG
TTGAATATTGGCCCCCACTCTCTTCTGGCTTGTAGGGTTTCTGCCGAGAGATCCGCTGTTAGTCTGATGG
GCTTTCCTTTGAGGGTAACTCGACCTTTCTCTCTGGCTGCCCTTAACATTTTTTCCTTCATTTCAACTTG
GTGAATCTGACAATTATGTGTCTTGGAGTTGCTCTTCTCGAGGAGTATCTTTGTGGCGTTCTCTGTATTT
CCTGAATCTGAACGTTGGCCTGCCTTACTAGATTGGGGAAGTTCTCCTGGATAATATCCTGCAGAGTGTT
TTCCAACTTGGTTCCATTCTCCACATCACTTTCAGGTACACCAATCAGACGTAGATTTGGTCTTTTCACA
TAGTCCCATATTTCTTGGAGGCTTTGCTCATTTCTTTTTATTCTTTTTTCTCTAAACTTCCCTTCTCGCT
TCATTTCATTCATTTCATCTTCCATTGCTGATACCCTTTCTTCCAGTTGATCGCATCGGCTCCTGAGGCT
TCTGCATTCTTCACGTAGTTCTCGAGCCTTGGTTTTCAGCTCCATCAGCTCCTTTAAGCACTTCTCTGTA
TTCGTTATTCTAGTTATACATTCTTCTAAATTTTTTTCAAAGTTTTTCAAAAGCAATGGCAACAAAAGCC
AAAATTGACAAATGGGATCTAATTAAACTCAAGAGCTTCTGCACAGCAAAAGAAACTACCATCAGAGTGA
ACAGGCAACCTACAACATGGGAGAAAATTTCCGCAACCTACTCATCTGACAAAGGGCTAATATCCAGAAT
CTACAATGAACTCAAACAAATTTACAAGAAAAAAACAAACAACCCCATCAAAAAGTGGGCGAAGGACATG
AACAGACACTTCTCAAAAGAAGACATTTATGCAGCCAAAAAACACATGAAGAAATGCTCATCATCACTGG
CCATCAGAGAAATGCAAATCAAAACCACTATGAGATATCATCTCACACCAGTTAGAATGGCAATCATTAA
AAAGTCAGGAAACAACAGGTGCTGGAGAGGATGTGGAGAAATAGGAACACTCTTACACTGTTGGTGGGAC
TGTAAACTAGTTCAACCATTGTGGAAGTCAGTGTGGCGATTCCTCAGGGATCTAGAACTAGAAATACCAT
TTGACCCAGCCATCCCATTACTGGGTATATACCCAAAGGACTATAAATCATGCTGCTATAAAGACACATG
CACACGTATGTTTATTGCGGCACTATTCACAATAGCAAAGACTTGGAACCAACCCAAATGTCCAACAATG
ATAGACTGGATTAAGAAAATGTGGCACATATCCACCATGGAATACTATGCAGCCATAAAAAATGATGAGT
TCATGTCCGTTGTAGGGACATGGATGAAATTGGAAACCATCATTCTCAGTAAACTATCGCAAGAACAAAA
AACCAAACACCGCATATTCTCACTCATAGGTGGGAATTGAACAATGAGATCACATGGACACAGGAAGGGG
AATATCACACTCTGGGGACTGTGGTGGGGTCGGGGGAGGGGGGAGGGATAGCATTGGGAGATATACCTAA
TGCTAGATGACACGTTAGTGGGTGCAGCGCACCAGCATGGCACATGTATACATATGTAACTAACCTGCAC
AATGTGCACATGTACCCTAAAACTTAGAGTATAATAAAAAAAAAAAAAAAATTAAAAAAAAAAAAAAAAA
AAAAAGAGAAACCAGTGCTCTATTATCTAGGTATATACCAAGGTTACCCACTGCTTGACTCTCATTATTA
GCCTTCTTTGATGTTCTCTGGTACTTGATGTCTTTCATAACTAATCAATGTATTAATGTATCCAATCATT
TACTCGATAACTTTATTGAAAGCAAAAGCAGTTGCATACCAGCTATCAAGCTGGAAGTGGGAGATACAGC
CGCAGACAAGGCAGATATGGTCCCAGCCCTTAGGAGCTCCCAGAGTAGCAGGAGGTTTCCCCTTCCAGTG
TCTTCTCTCTGCTTTTCTTCAAAAGGAAAAGGCTGATGTGTATAATATACCATATCTCTTTGAAGTTCTC
TGATTATGGATTTTAGGTTTAAACCAGTTCTTCATCCATGACTTTATAAATTGAAAATCCAGGATTTTGC
TGTGTTGTTGTGTTCTTGTTTTGTTTTGATGTCCCTGTTTTCTCTAGATACAGTTAGAAATGTCTAGGAA
GAAATTTTTGGTTAGTATGGGAGCCCCACAAAGCCATTTTTTTAAACATAAAATCTGTATTACATATCAG
GTATGAAATACAGGGGGAATGAATCATTTCTCCGTAAAGGAAAATTTAAAGTAAATTTCAGGAAAGTGAA
TTCTTTCCCGTTTGCATTACCGACAGATGCAGAAACTTTAATCGTCATTTGCTAAGAGGGATATGGCAGA
TAATACACAATAGATGTCGTAGCAACATTCACTCGCATTCTTTTTTTTTTTTTTTAAAGAAATCTTTCTT
TCAAGAAGCTATTCTAGGATCTTTCTCATGACAGTGTCCTAGTTCTTATCTTTGCTACACACAGGCTCAC
AAAGTGTTTTCTTTGAAGGGCATTTTGTTATTGGCCCTCTTTTCATTTTTCTTTTCCGTAGCAAACAGAA
CCGAAGGTGTTTACTCCCCACGGTGAGAGGGCACCTGGGTGCACAAACAGTGGTGTGAACCACTGGCCTT
TCTCTGCTTTCCGTTCCCTGAATGTAAGAAACAGGTGCAGTGATCAATTCACTGCGTGCAGTGAACCCCA
GGCAGAAAGAGAACGTCGTGTCACAGACCTTTTGTTACTTGGAGAGAATGAGCGGGAAGAAAGGCTGCCT
CTGCTGCTACTGAGACCCTTTTGCCCATTTTATTGACTGCTATAGGTTCATCTATCCTAATTTGTCTCCG
GCTGTCCCAGTTTATCCCTGTTATTCTTGTGTTACTTTACTTTACTATATTTTATTTTATTTTATTTTAT
TTATTTTAGAGACAGAGTCTTGCTCTGTCACCCAGGCTGGAGTGGAGTGGCATGATCATAGCTCACTGCA
GCCTCAAACTCCTGAGCTCAAGCAATCCTCCTCCTTCAGCCTACTGAGTAGCCAGGATTATAGCTGTGCA
CCACTATGCCCACCTAATTTTTTTTTTTTTTGAAATGGAGTCTCGCTCTGTCACCCAAGCTGCAGTGCAG
TGGTGCGATCTCGGCTCACTGCAACCTCCACCTCCCGGGTTCAAGCGATTCTCCTGCCTCAGCCTCCTGA
GTAGCTGGGATTACAGGTGCCCACCACCATGCCCTGCTAATTTTTGTATTTTTAGTAGAGACAGAGTTTC
GCCATGTTGGCCAGGCTGTTCTCAAACTCCTTTAACTGTTTTTTTATTTTTATTTTTAATTTTTAAAACA
TATTGTAGAGATAAGAGTCATGCTACATTGCCCAGGCTGATCTCAAACTCCTGGCTTCAAGCAATACTCC
TACCTCGGCCTCCCAAAGCACCTGGATTACAGGCATGAGCCAGTGTGCCTGACCCTGTGTGATTATTATT
AGCATCCTGGACACTCTCAAAAGTGTTCAGGTTTGGACAATGAACTATAGGATCACCCTAATTACATGAG
ATTAAGAGTAGAGACCTTGACCACCAGAAATGGTCAATACTCACCATATATTTTCTTCCTGATGTTAGAA
CCTGGTACTTTTGGGAAATGAAATTGTACATGAGATATATGCAGAATGGGCGAAGGGAGCGAAAAGATTT
AAAAAATTAAGCTCGATTTATTGAGCGCCTCGAGTGCGCTCAGTGCTGTTCCAAGTGCTGACAGCAGAGA
GGTAAGTTCTGTTCTCCAGTGTTCACCTCACACGTGCAAGCCAGGTTTGAAAACACACTGTCTTTCCTTA
GTATCCCTCCACCCCTCCATGTGACTATACGTATGTATCAAGTTTGTGATATTTCACTTCTGGGCTTCTT
TTCATTTGGAAATTTAATGTCAGTGTATCATGTTTTAATTAATAGGACATCATGTTATGAAACTGTTGAA
TCGAATATTTTCCCTAGGCATCAAATTACTTGTCAGTGGAAATTTGACATCTAGATATGAGGGACAAAAG
AGATGAGAAAAATAATAGTAAAGTGTTCCTAAAGGATGCTGGTATACTGTTTAGGTATTTTAATGCACTG
TTACAACCTAAAGTGTCTTGTAAAGTATGTTCTTTAGAAATAAAATAAATAAAACAAGACATCTCTCCAT
AGGTACAAATCCACTTGCCTTCCTCAATTCCTATCCTTCTGTGATGGGAAATCTCTGCTGTGACAAAGAA
CCATGTTAAGAAAACCATAAAGTTGTATTGTTTGTAGATTTTTTTAATGACTAAAGGAAGATATTGCAAG
TAGTAGAAACAAATAGAGGAGGTGGCCCTGAAGGTCAATATAACGGAGTTCACTGCAGAAAAGAGAAACT
ACTCTAGGTACGTTAGACACATATCAAAGTTTTGGAAAGGCTAAAGTAGCAGGTTTTAGACTTGGCTTCG
AGGACAGATTTCTAAAACTATATAGAACTGATCCAATAAGAAACTACCATCTCCGGGGTACCACTGAAGC
AATGATTTCAAGAACATACTTTGTAAATAGGAACTAGGAACCAGGAGGTTGAAATCTAGACGCTACCACT
TTTGAAGCTGCTGTTAGCAACTGCCCTTCTCCAGCCAGGAAGCTGGAGAAAGAACTTTGGAACTCTGATG
TAGGAAATCTCATGTTTCTTTGACTAAGCTCGCCAACAGAAATAGCCAAAAGGGGCAGAAAGGTGACCTA
TGCCTCACTTCCACTTTCCAGATCTCTCACAAGTATACACATTTGGCAAAACGTTGCCAGATTTAGCAAA
TAAAAATAATGCATGCAACATACTTAACACTAAATAAAAAAAGATTGTGTAGAAAATTTAAATTTAACTG
GGTGCCTTGTATTTTATCTGACAACCCTAACTTATTATATCCTAATTCATAATCAGAACACTAGCTGCAT
GGAAGTCTGGCAAATACAGTTTTTAATTTCCAACCTGTCCAACTGGAAGGATGGTAAGTAGATTTAGGTG
AGCCAGTTCACAGTATTAACCAAAGTAGATTGCCTACCAAGAATAGCTAAAGCCTTTCTGCCCCCAGACG
CTTATGCTACCATCTGAATATTTTTACTTTGCATTCTTATATTCTTGGAAATCCTATCAATCTGTGATTC
AGATTGGTTTGGTTTAACTCAGCTTCCCCTTTTTTTTGGAGACAGGGTCATACCCTGTCACCCAGGCTGG
AGTGCAGTGGCACAATCATGGCTCACTGCAACTTCGACATCCCTGGGCTCAGGTGATCCTCCCTCCCACC
TCAGCCTCCCAAGTGGCTGGGACTACAGGCACGTGCCACCACACCCCGCTACTTTTTGTATTTTCTGTAG
AAACAGAGTTTCGCCACATTGCCTAAGCTGGTCTCAGATTCCTGGGCTGAAGTGATCCACCCACCTTGGC
CTGACAACGTGCTGGAATTACAGATGTGAGCCACCATGCCCAGCCCCTCTTTTTAAAATATAAAAATCTC
CCAGAATGTGAAAGTTGTCAGTCTATACTTTGGGAATAAGATTTTCAACAGATAGAAGAGAATGAGGATT
AAAACATAAGGAAGTTTGGGAGTAGAAAATATGGGCACCAGAAGGTGGAAGGAGAGAGCAGATGCCCATT
TATATATCTCCTTTGTTGGGTGTTGGACAAATCCAGGTCTTAAAATAGGAAGTATTTCTTTTCCGTACTT
CTTGAATCTTTCATATCCCAAAAGATGCATATTTCCCAAATCATATAACCCAAAGTCATGCTATCAAAAT
GATATAAATCATCCATGGTATAGGTTAAATAGCTATATATGTATATGCTGGTCTGAGACATGTATATGAC
TATTGTGTCCATGGAAATTTGAGTTTGGGGTTCTGGACCATTTATTTGCAAGTGATTTTTGGTTAGAGAA
CTCTTTGTAAGTTGGGGATTGCTTTTACTTATTTTATGAGTAAAGATGTCAAAAGGATGACTGCTAAATT
TGCACTGTGTTAATTCACTATTTAGTGAGAAGAAATATTAGACTAGCTATGAAAAGTAAAACTGCCTCTC
CAAAAAGTCAAAGCTGATGAAAAACAGTCATACAAGCACAATGCCGCTCTTCGGAAACATGGAAACACTT
TTTCCTTCCCAATTTTCCCTCAGATTTTCTCTTCCGCATTTAAAACACTTGGGTGGTTCAAGTTTCTAGG
CTACCACTGATTGTAACAGCAAACAGTAGCAACTGGAAGCAGTGGGATGTTGGGAGAAGTAATAGAGGTA
GCTGCTACCCAAGTTATCCTGGAGGATTTTCCATGGCAATGAAATCAGGTAGTAGAAGCTTGGCTAACTG
AGTGTAAGCAAACAGTTCTACTGAGAATGGTGTTGTCTTTTCAATCCGTTTATCTGTGATGGTGATAGTG
TGAAACAGGGGAATTTTATCCAAGGTTTAAGGAAGGTTATTTGGTTAAAAGAGGATATTGTTACAGTGAA
GTCAAACTTTCCATTAACTTTTTGCTGTAACAACAGATTGAACGTAGCATTTCACCGTCAACGAGTAAAG
TGAAATTTACAGATTAACTTATGTGCCTCTTTTAAAATATATCAGATTTCTAAATTGCTTTTATTTCAGA
GGTATGGGAGGTTCACTTTCTCTTTGAAAGTGTACATTATTTTTCTAGTGTCTTACATCTGCCTACAAAG
ATGTTATTTTACTTGAAAGCACAGTAACTATTTGATGAGAATTTGTCAGCATCAGTAAATTAAAGACCCT
CAAATGATTTCTACTAATTATAGTTTAATTCCGTACATTTAATGATATTTTAAAACACATGAGTTATTTC
ATAACTCCCAACATCACAAGGATAAATTTTATTCTACAAACAAAATATTGTGCTAAATGAAATAGTTCAT
TTAGGCAAAGAAAGGAGCACAGAAAATTAGTGGAACTCTCTGCTGTAAGTAACGTAGACATTACATGGCA
TATTGAGTCTCCATGAATATTGTCATGTTATGTTTTAAAAAGGTGATCGAACATATGGCATTTAAAAGTT
CCAAGTCCTCTTTTAAATGCTTCAGAATCTATTATTTAATGATCATCTTGGATCTCAAAACTGATCTTTT
GAAAGATTTTATTCGCCCCATGTGTTAATATGATTTCCCTGTCATATGATATGATTATCTATCAATACTT
AAAACCAGCAGCCAAGTAAAAAATCAGTTCATATCATTTAATGAATACTATGAGTCAGGATCTGGGTAGG
CAAGCTATTTTCGGGTTTGAGTAGTTCCAAAGCTTAAAAATCTTATATTGATTTTACAGTGAAGAAGAAA
TAGTCTTAGCTACTTTGGAGGTTTCAAACATTGACTACTCAAGGAGTATTTCCTTGCTTTCTCAGGCACC
AGGCAGTTTTTCAGGAGCAAGCATTCATCCATTCAGGGAATTGTAACCTGTAGTTTCCACTTTTCTAGCA
ATCACACTTAAAACCATGAGAGTAGGCCATAGGACATAAGGAGCTCAGCTTCTCAGGGCAAGCACATCCT
TTCAGCTTTCACCTGTCCGTTTGTTAGTGTTCACTTCCGTGCTCAAGGAGTTTCTTGTTGCCTCTGAGTT
CTAAGAGACAGAACGAAGGGAGAAGGGTGCAGAAGTCTAACGCATGTTCATGGACTTATCTCTCCAATAA
AGAGCTTGTTTTATCTTCTTTTATTTATTTATTTTTTCTAATGAAGCCATTAGCCTCAAACAAAGCCATG
GAATCTTATCAGAGTGAAACCGGGGTCATTCCATAGGCTGGCTGAGTGAGAGCTCCATGGCACGATGATG
TATGGTCACTGCACAACAACGCCTTTGCCACAACACATGTGCCTTTTAATTACACTTTAAATCTCATTTG
AAGAGATGTTATCATTATGGAAATTGCTCTGTAAATGTGCCCAGGATGAGACCCAATAAAAGTTTGCTGA
GAAGAATTGAAGACAGAGGAGATGAATCAGCAGCTAAAACATTACCATCAGACAGAATTTTCTTGGCTGT
AGGCAAAACAGCCCATGCAATAACAGAAAATCTTCATTGACTCAGAGGCGTATTTTCCCTAGATTATTAT
GGGGCACTCCTGCCTGTAGCACTATCACTTCTTTGATAAGCTGAAGGAAGCGTTCTGCTCTCCAGCTCAG
CGGGCCTTTTTCTCCCCAACCTCAGAGCCATCATTTGAATTTATAGTTGCCAAAATGAATAATACAGTAT
TGCCCTTGTGTTCCTGACTTCATGCATGCATGCAGAGCGGGGTTAAGGTTCTTTAAATGAAAGATTGCCT
TCTATTCATGCAATAAAGAACACCTCTGCTTCCTTTCCAGGGTCATTTAAAAATAACTATACCGCTGGGC
TATGGAAAGCACATAAGAAAGATTCTTAGGGTAAAGTCAAAATGCTCTTTTCTCTACAACAGGCATTGTC
TCATATCTTTGTGTAGCACAGCTGATTTGAAGTTTTCTTTTAAGCACATTCTTAATTATCTTTTCCTTTG
ATCTTGAACTGTTTCCCTGGGCTACCAGACAGAGAGCCTAGAGCCCTACCTCCGCTTTCCCCGAGGTGCA
AACTGCTCCGTCCTTCCACAGGCAGGCCCCTGGCTGAATGCACCCTTTTCTCCATGGTTACCCACCCACC
TCTCTGTTATTTGTTACTTCCCAAGTGAATGGCAGGTTAAAATGGGAAAAGGTCAGGTTATCTGAATGTG
GTTAGAGTGAAATGAATTTCCTCATTGCACCCAAGAACTGTCCTTTGACAGGTCTCCTTCCCCAAATCGG
GTCATTTTGTACGTAGGCTCACTGGGAGTAATTCTAAGACAACTAAATAAGTAAAATCACATTTTGGTGC
CATTTTCCAATGTATTCTTCTTCTTGGGGGTTCTCCTTTAAAATGGTACTGGAAGGATACGTTGTCTTCA
TTAATCCATTGTATGTCCCGGGGGTGGAGGTGGAGGTGGCAGTAGCAGAAGCCCGTGAAGTAATAGGTCG
TATTTTGTGTTTATAAATATTTCTGCAGGTTTTTGAGGAGAAGATCCATCATTCTTATAAAGGCATTCAT
GACCTCCAGAAGATTAAGGGCTGTTATGCTAGAACAGTGTTTCATTCTTAAAATGGGGTCTCTGGACTAG
CAGTATCGGCATCACTTGGGAACTTCTAAGAAATGCAAATTCTTGAGTTCTACCCCAGACATACAGAATC
ATGATCTCTCAGAGTGGAGCCCAGCAGCCTGTGTTTTAATGAGCCCTCTGGGTGATTCTCAAGCCCACTC
AAGTTTGAGAACCACTGTACTAAGGGAACTACTGATGCATGATGCAAGTTCACGCTCACAGGCACGTGTG
AATGACACAAAGAACACAGACGCCTGAGAGAGCAAGAAAGACAACATAGACTGTCTGACTCCCTGCTGGG
CCCTTCTTTACCGCCCCTATTTCAGGCTACCATGCCCATGAGTGGATGACACGTACCCCCCGACAAAGGT
CAACACCACTCTCCCTTCCACACCCTATCACTAAGTGACAGGCTAAGCCTATGTTAAACTGCTCACATCT
CCTTGGAAATTCAACACTTTAATAATAGGTAGCATTATCACCCCCATCTTCTTCTCTAAGCCAGAAACCC
AACTTGCCTCCCTATATGTTATCCTTGCATTCAGTCAGTCTCTAAGTTGTATTCATGATCTCTCAAAAAT
ATCTCCCTTTTTCTCATCCTGTGTCTATTACCTCAGTTTAGATCTCCATATTCTCTTGCCTCTAATGTTC
TTGCCTCTAATGTACCTTTTCACTGCCACCAAGATGATGTTACCAAAAAATCTTAAACAGATTAGACATC
TTCACAGGATAAAGTCCAAACCCTTAGCTTGATACACAAGCCCCTTCACAATCCAGGCCCTCCTTCCTGT
GCAGCTATATATATATATATATATATATAGAGAGAGAGAGAGAGAGAGAGAGAGAGAGAGAGAGAGAAAT
TTTGATCTTACCACTCTGTCTCTCTTCCCGCCATGGGTCTTCCCCTCTTCCCTCCGGAGTTACTTAGCTC
TGATGTGTATTCTTATCTCGTCTTATCTTCAACTTCATTCATGTTTTTCCCACTGCTTCAAATTTCACTC
TCCCACTTCTCCCCTGGCCAGCTGCTACTCATCCCTCAAGACCCTGATCAAATATCATCACTTGTATGAT
GGCATCTGCAAATCTTGGGGGGCAAGGCTAATTGTTCTTTGTTCCCACAGGGCTGTGTTCCAGTTTAACA
TGATCACATGTTATTTTGGTTCATTTATTTGCTTAAGACTTTTTCAGAAGGCTATGGGCTCTTTAAAATA
GAGAACTTACATCTTGTAGTTTTAAGAGTATTTTTAGTAAAAGTTTAGAGTGACCCCCATCTTTCTGCCA
GCCCACAAAAGGAAAACATCAAAAAGTGAATGTGTAAAAGGAAGAGAACTCTGACAAAACCAGGCAGAAA
GGTTTTTCAGCAAGTCTTTTTATTTTCTGTTCAGGATAACATTAATAATTATCCACGTTGGTTTCTCATT
CTCCTGTTGGTGAATATTTTTCTGCTAAATTTAAAACCGTATCACAAACTCAAGCAGAGATTTACAACAT
TTCAACAGCTTTTCTACCCCTGCCTTAGAAGGGTGGATCAAAAACATTTGTCCATGGTAAAGCACTATGG
ACATGACTTAGTTAACAATTCTCTGTTTGGGTCACCATGAGGCTTCTTCGTTTATACTCAGGGTCAGCGA
CAATGCTGATATGCAGCTACAATTTCTCATTTCTTACTCAGGGTGTTATGAAGCAGATTTCCACTGTTCT
TTAATCGTTATTAAAATGTAGTCCAGGTGCAGTGGCTCACGCCTATAATCCCAGCACTTTGGGAAGCTGA
GGCAGGTGGGTCACATAAGGTTAGGAGTTCGACACCAGCCTGGCCAACATGGTGAAACCCTGTCTCTACT
AAAAATAAAAAAACTGGCCGGGCATGGTGGCAGGTGCCTGTAATCCCAGCTACTCAGGAGGCTGAGGCAG
GAGAATCGCTTGAACCCAGGAGGGGGACAGAGGTTGCAGTGAGCCGAGATCACACCATTGCACTCCAGCC
TGGGCGACAAGAGCAAAACTCTGTCTCAAAAAAAAAAAAAAGTCATTCTCATGTAAAAATTCTTGTAAAA
TAATCTGTAAAGTCATCCTCTTATCTGTTCTAGTTCTTCATAAGACTTATATAACATGTCATATGGGCAT
GGAAAGGCCTAAGCCTTCCCAAACCTTGCTCTTTTGGGGATGATTTTCCAAATGTACTTGTTCTCAGTTG
AAAAGAGCATTGCGGCCGGGCGCGGTGGCTCAACGCCTGTAATCCCAGCACTTTGGGAGGCTGAGGTGGG
CAGATCACGAGGTCAGGAGATCGAGACCATCCTGGCTAACATGGTGACACCCCGTATCTACTTAAAATAC
AAAAAATTAGCCGGGCGTGGTGGCGGGCGCCTGTAGTCCCAGCTACTTGGGAGGCTGAGGCAGGAGAATG
GCGTGAACCCGGGAGGTGGAGCTTGCAGTGAGCCGAGATCGCGCCACTGCACTCCAGCCTGGGCAACAGA
GCAAGACTCCGTCTCAAAAAAAAAGAAGAAGAAAAGAACATTGCATCATGGCACAAGGACACAAAAAATA
CCCTGGACCTGCTTCAGTGAGATGGTCTAAGGGTCTCTAGCATCTTCTGAACTGAACTGAATGCTTTGGG
AAGAATTAATAGATACACGATGTATATTAGTTCGTTTCACACTGCTATAAAGAACTTCCCTGAGACTGGG
GTAATTTATTTAAAGAAAAAGAGGTTTAGTTGACTCACAGTTCTGTGTGGCTGGGGAGGCCTCAGGAAAC
TTATAATCATGGTGGAAAGCAAAGGGGAAGGAAGCACCTTCACAAGGCAGCAGGAGAGAGAGAGAAAGAG
TGAATGGGAAGAGCCCCTTATAAAACCATCAAATCTCATGAGAACTCACTCACTATCACATGGGAAAACA
GCATGGGGGAAGCCACCCCCATGATCCAATCACCTCCCACCAGGTTCCCCCGGATTACAGTTCTAGATGA
GATTTGGGTGAGGACACAAAGCCAAACCATATCACAATGGAAAGCTCATGAATGGGTTCTAAGAATGAGG
AAATGTACCTTAGCATTTTGCCTACTTTTCCTTTATGACATTTTTTTCCCGGCAAATATGCCAAATATTA
CCTACCTTTACATCAGTGTCCACATGCATATCCCCTGTCTTCCTCCTTTTCCTCATACATTAACAAAAGA
GTAACTTTGTTTTCTCCCCATCACTGTTCACCCTATTGTATAAGAGAAGAAAAGCAAAATAGGATGAAAG
AACTATCTAGGCACACACACAAAAGTCACACTCTCCAGAAGAAAGAATTTGCTCTACTTGGTAGTAGACA
GAAATTAACTCACTGAAGATCACCAGAGAATCAGATCCAATTATATCAGCAGGACTTTAGTTTACATCAT
GGTACTAGAACCTTCTTTAACATTCAAAACTTATGAATACCTAGAAATAGTTTTAAGGTTAATATCTCTA
TGCTGTGGGCTAAAGAGTACCCACAAATGAATACAGTTGTGTCTGATGAGTGTCTGTGATTATTTTGGAA
ATTGTCCTGCTATTTAAAATGAAAAAAATAGAAATGTCTTAGATTTTCCTATCATTAACCTATTGTAAAC
AATTACATCAGTGTAGGGTTGTTTTGTGGTTGCGTGGGAGTATTTTGAGGTTTTTAGGGGGTAAAGTGGG
GGATAGAATGAAGTTGTTGTTTGCATTTACAACCCTAATAATTAAAACAAGCCAGAGGGAATTACCTACA
TGGCTGTTGTGATTTCTAGTGTATGATCAAAAATAATTATGGCACTTTGCCATATGTTCTTGCTTTCTTC
TTAGATATGTGTTATTGGGAAAAGATGAGACTTGACATCAACTAATTGCTTTTTTCTAATATACAACCTT
GAACCACAGTGATTCTCTGGAGGACAAAAAATAGCTTAGTGACAAAGAGATTCCAGAAATAAGAGCTTTT
CGAGCTTTTAACTCTCTATGTAATATAGACAAATTGCACAGATTAATATAACCAAATATGTATTGGTCCA
TGGGAAGAGAGTTACCTATTTGAAGAATAGGAGTGTATTGTGTTCATTTAGAACCATTCAGAAACATCAA
TGATATTAGTTCTGAGTTGACTAAGGATAAATTTTTAAAAGCAATACCTAATTGGAAAATTATTCAGTTG
TTGACCATTCCTATCAGTGCTCTGAAACTAAATATCTCACAGATGCCTTAATGAGTTATTATAATTATGT
TGCTGTGATACATGTAGCCCAAGTCAGAAGTCACTTGCTTTGTATTTAATGGATGGGGAAGACACTGGAG
CTTGGAGGGAAGAGAATAAAAATAACCTAGTTTCAGGAAGATCTATGCTCTAACCCTGCTTCTGCCACAT
AACAACAACTCTATGATTTGTATAAGTTACTTTACCTCTCAAACTCGTGGTTTCCTCGATAGGGGATAAA
GAAGGCCTATTTCATAGAGTTGGTGTAAAGGATTTATAAGGGCTGTAAGTATTAGTTCCTGCCCTGTTTC
ATCCCCCTACCCTACCCCCACCCCTCATCATGGCTCTGCAAAAACAAATATGTCTCAGAGTTGGAAAGAC
CCATCTGGTCTTTCTCAGATAAGGGTAGTTTTCTTCAAACAGACTGATTCCTGCACATAAAATATAATAT
AAAAAACCAAAAGTACCTTCAACATTGTAGACTTTTCATATGTGGTTGTCTCTGTGACTTAAATGTACAA
TCTAGGGTTTGCATGTTAAGGTCTTTCAAGATTACTGTTGGCACTGATCTGAAAGATGTCTCATGCAGGA
AATGCTCACCCAATTATGAGCACTGAGGCTGTATAGCAATATCAGAAATAATATTGCAGCACAGTATTTT
CTATGGATTTTAGATGCAGTATTAAAAAAAGAAAACTCAGCCTGTCTTTAAGACTCGTTTTCTCTTTCAA
CACCAGAATTAAAAGGCATGCCATTCTTTTTTAAAGTTTATGTGCAGAGCCAAATGAATATCAACATTAG
TTCTCCACTGGGTCCACGGCTTCTTTTTAAAAATATCTGAAGCAGTGTTACTCTACCCACTTTTTCTCAG
GAAATTGTGTCCTTTAGAACTGGCACCCATATAGTTTAGGAATGCTTGATAGGGTATATTTTAGGTGGGA
GTCACCTGTGCCTATATGGAGCTTTGATGTCAATGCCCTTGTCATTTGGTGTCAATGGTTTTGTCATTCT
AATTTATTTGGCCCCAAGGTGTGTGTGTGTGTGTGTGTGTGTGTGTGTGTGTGTGTGTGTGTGTTTGAAA
CACAGCATATGTATTTTTTTCTTACTCCTTGAGAAATAGAACTTTAAATAACATTTCTATCTTTAAAATG
TACTTGTGAATAGTTATCACTTTCCTGAATTCTACCTTCCAAAGGTAGGACAAGAGAAGGATGGAATTAA
ATCACACATGCAGCTATTTTTATAACTTATGAGCACTCAATGATGGTAGCATCCCTCTTCTTTACCTTCT
CTCTGATTCTGAAGCCACTGCTGTTTTGGTTCCTGTATGGCTGGGACTGCCTGTCCACTAGACTTCCTGC
CTTCACTCTGACCCTGCTACATTTACCCTGAACACCATCCTCCCAATAATCATTACCAGATGCTATTTTC
ACAATGTTACACCTTTACACAAGAAGCTACAGTGGAATATTATCCTTACAGAATCAGATAAAAATCTCCA
GGCGGCATTCTAAGCTCTCATTGGCTACACTGTACCCTTTGAACCTGAACTTCCTGAGAACTACCTCTTA
GCTCCTGCCTGTAGCTCTGGCTCCCATCTTCATGCAGTAGGTATTCGTTTTCTTTTGAACGGCCTCATAT
CTTCTCTGCTAGCAGGATTGGAGTCATGGCAGAAAGTGAGAGGGGGACTAGAAGGATGCAACATTAATGA
AACCTCACAATAGGTTAGGCATTGGGTTAGGTACTTGGTTGATCTACTCACATCATCTCATCTAATCTTT
GCCAAAACCTTAAAAGGTAGGTATTATCGGCCCTACTTATAGACATACAATAGGTACTTAATGCACATTT
TTGTGATGAATAAATATTCGGGAATTTTTGCATTGTGACTGGATGAGAAGAAACTAGGAAAAACAAGACG
TAGATGAGAAAGATACCTCTCATCTTACTTCCAACCTAGGAGATCTTAAATGAACTCATCAGTTTTAAAA
GGTAATCTTAAGAATGGAGTCAGAGGGTCACACAAGGAGAGAAAGCATCACGTATTACCAGAGTTCGGGA
TTGCTTAAGGCGGTTTGACAGTTTCCTCCGGGGGTAATCCACCCTAGGCCAGGCATTTTTAAAGATTAGA
TTTTGAAATGAAGCTTTGCACTTGGGAATATACTGAGGCAAGAAAGCATATCCCTTCTCCCTGCTGAGAG
AGCATATCCCTTCTCTCTGCTGTTGCAGTGCTTAAGTGTGAGAATTTTCGAATGAGATATGAAAGCAAAG
ACAACAAACCCTGCAGGATAATAGTCTCTGAGGACTTTAATAACAGCCGTTTTTAAAGCAAAGCCTGTGG
ACTCCTAAATCCATAGCTGCTCGTTTAAGATGCAACGCAATGCAGTGGACCTGAAAACATACTCCTTATC
TACCTAGAGCAACCAGGCTCCAAGCCAAACAGCAGTCCTCAAATTAACTCTTGTTTCTCTTGGGACGACA
ACTCTGCTGCTTTTAAAGGTGTTGCGTGGCCACATATAGAATAAGAAGGGAAAAAACAGTCACACCCTCT
TGTGAGTCTGTATCCAACTGCATTTTCTGATCTGGTTAGGAACCTCCGGTGGTTAGATTAGAATCCTGAT
AAGGCCAAGACTGTGGGTCCAATTTCTTCCTATGGATTCTTCTCGAAAAACTTGTTAGGAAAGATCCACT
TCGGGTTTTTTTTTTTTTTTTTTTGCGCTTGTTTTAATTCCCAATCCATAATAGACTGATACTTTTGTAA
CATGCAATAAAAATGAATTATTTTAAAAATTACAAATACCAGACATACAAAATTTAAGCCGATGCTTTTT
ATTTTTAGATTGCTGTACACGTTTCTAAAGATTTGCTTTCAATTTCTGTTCTTATGTCTTCATAATAGAA
TATCTGTTTCTGTGTGTGTATACGTATGAGTGTATGCAAGTGTGCTCCAAAAGCTCTACTATTTAGAGCT
CATGTTTAATGAGTCATGTTGGATAACCAGTCTAAGGGAGTTCTACTTTCATATAATTGTTTTTGTTTTA
TTTTTATTTAAATCATTAACACCTTTTCAAAACAACTGTATCAAATAACAGTCATTTGGTCATTTGAAGC
ATTTACATACACTGCTTTCTTCTTAAACAGATTTTACTGAATGTAAATCTGCTTTCCCTGGCTAATTCAG
CATCATCATCCTGAGCATTAACTATTTTGCTTCGCTATAAAACGAGGTGATGCCTTCAAGGGCTACTGAT
GCATGGAGAGCTGTTAGTTTCCACACTGTGTGACCCTGGTCAATTATGCATGCCCATGGCTCCTTTTACA
CCTTTCTGATTCTCTGTAAGGTGTCACTTCTTCCTACTCTCAATTTAGCCACTTAGGATAATTTCTTTAC
TATTTTGAATTGTATGTTCCTGACCTTCTAAGTTCTTAGAAATCAGACCACTTTTTTTCCCATCAAGCTA
ATTTAAATTAGATAAAAATTATCAGTAAGGAGGAACTAATGGCCTTATAATTATTCATATACTAACTGCT
TTCAGAAAAGCTTAGAGATAATCTGTCTATAATAAAATTCTTAAGGAGATTTGGTCACTTATTGTTATTC
TTTCTACACCATTGTGTTTGTTTCCTTACTTCTCAGCTATATTAAAATGGGGAAGTTTTCATTTGCTGAG
TCCTATTTTAGAGACCAATAATTCCATTTACATAGGAAAGGAAATATGTGGATACGATTATTCATGATGT
TCTAGAATAGTATCACAACCATCTGCTTAATGGTTAATAAAATGGTTAATAATAAAAAGAAGGGTACAGC
ACTAATTCTTGACATCTCCCTTCTTTATTTTTCTTCTAGTAAAACATCCATAACTTTTCCTATTCTTCCC
CAGTTGTATTATTACTTATGAACACCATGGATCATTCTACTTTTTGAATGAAATAGTACAAATTTAATAT
TCGATAATCTTGTCTTTGTACTTTTCTTTCTAAACTTTTATTCTCGGTGGCTTCCTAAAGGAAGACTTTA
TTCTACTTGGTTTAAGCAGTTGGTCTGCATTCTACCTATTTCTCTCAACTCTCATTGTCCATTCCAATCA
GGTTGTGCAATTCATTGTCCCTGCTCATAAACCAAATTCATTTCACCTTTACTCCATTCCCATGCTATTC
TTTTTACCTGGAATATTTTCTTTTGTTCCAATTTCCCAATCGTATACATCCCTCAAGGCTCAAGCCAAGT
CCTGTAACTTCATGAAATCTATTTCTAACATCTGATTTCCACATGGATTATCTTGTAATAGTAAGCCCTG
CATTTGCTATTATTTATTAAAGATCTCTTGTGTTCTAGACGCTATTCAAAGTATTTTACAAAAATTCCAT
GTAATATTCAAAAAATTCAGCAGGTATGTATTATTATTTCATGTTTAGTGAAACCAAAGTAATGAAAGAG
TTTTCATCATTATGTGGCTAATAAGGGAAAGAGAAGAGAAGGGTCAAACCTATGTCTATTTAACTGCACT
TTGCCATGAGTTTTCTCTGGAAGCTGAGAAAGGAGATTCACAACAAGAGTAGATGAGACTCAGATAAGAA
GGAAGTGTGGAATTTGAAAAAGCCCTTCAGAAAACAGGATCTCCCCTACTCCTAAAACTGCTATACTGTG
AACATATTGCTTGTCTCATAACTGAACTTTTTTGGTACTCTCAGCTGAAATTTGCTCTATAATTGTACAG
AACTTTAGGCCAAATGTTTCTTTTATGGGGACACACATTATTTGTTTCGTTTGCACCCATTTTCTCAATT
ACTTTGTGTTTTCTCTTGTTGTATTGGATGCATCTCTATCCACTGCTACAAATCTGTTTAATAGTCTTTA
TATTAATAAGACCATGAAATTGCTCTTTGTGTGCTGACATAGTTATCCTTTATTTTCTAATGGCAGTGCT
AGATTTGCTAAAATTTAGGTAGTAGCATTATTAATAGGAAAAACTACCACCAACAACTAAACTTGAAAGG
TAATATAGCCTAGTGGCTAAGAGCACAATCCCTGAAGTCTGACTGACGAGGTTCTAAATCTTGCTGCATT
TATTAGCTGTGTGAACCTGGGCATTTTTAGTTAACCTTACAGTAGTTTCATTATCTTATATGGAAAATGA
AGATAATAGTAGCCCCTACCCTAGAGGGTTGTTGAGAGGAGAAAATGAGCTCATGTATATACAGTGCTTT
GGACAGCACCTGATGTACAGTAAGGTTCATGTATGTTGTTGTTCTTGCTGCTGCTGCTGCTGCTATGGTT
TTTGTTATGTAACAACTACCTTTTCCCCTTTGTTCATTCGTTATTGCTTTTCCTAAAGACTACAATCACA
AAAAAGAAGAAAAAAATTAGAGAGCATACAGTGAATGCAGTAATGAAGGCTTGAATGATCTTTTCTAGTT
AAGTCAGAAGTGAAATAAAACTATCCAAAAATTTCTATGAAAATTATCCTTTGTCCAGATTGGCTACCCA
CTGAGAACTCCACTTGATTCTCCATATCAATCTTTTGCTCTTTTGTGCTACCTGAGTCTGAGGTGTAGTC
TTTAAATGATGAGTTTATTGGCACAAGACAGGGATGCCCTCTCTCACCACTCCTATTCAACATAGTGTTG
GAAGTTCTGGCCAGGGCAATCAGGCAGGAGAAGGAAATAAAGGGTATTCAATTAGGAAAAGAGGAAGTCA
AATTGTCCCTGTTTGCAGACGACATGATTGTATATCTAGAAAACCCCATCGTCTCAGCCCAAAATCTCCT
TAAGCTGATAAGCAACTTCAGCAAAGTCTCAGGATACAAAATCAATGTACAAAAATCACAAGCATTCTTA
TACACCAACAACAGACAAACAGAGAGCCAAATCATGAGTGAACTCCCATTCACAATTGCTTCAAAGAGAA
TAAAATACCTAGGAATGCAACTTACAAGGGATGTGAAGGACCTCTTCAAGGAGAACTACAAACCACTGCT
CAAGGAAATAAAAGAGGACACAAACAAATGGAAGAACATTCCATGCTCATGGGTAGGAAGAATCAATATT
GTGAAAATGGCCATACTGCCCAAGGTGATTTACAGATTCAATGCCATCCCCATCAAGCTACCAATGCCTT
TCTTCACAGAATTGGAAAAAACTACTTTAAAGTTCATATGGAACCAAAAAAGAGCCCACATCGCCAAGTC
AATCCTGAGCCAAAAGAACAAAGCTGGAGGCATCACACTAGCTGACTTCAAACTATACTACAAGGCTACA
GTAACCAAAACAACATGGTACTGGTACCAAAACAGAGATATAGATCAGTGGAACAGAACAGAGCCCTCAG
AAATAATGCCGCATATCTACAACTATCTGATCTTTAACAAACCTGAGAAAAACAAGCAATGGGGAAAGGA
TTCCCTATTTAATAAATGGTGCTGGGAAAACTGGCTAGCCATATGTAGAAAGCTGAAACTGGATCCCTTC
CTTACACCTTATACAAAAATCAATTCAAGATGGATTAAAGACTTAAACGTTAGACCTAAAACCATAAAAA
CCCTAGAAGAAAACCTAGGCATTACCATTCAGGACATAGGCATGGGCAAGGACTTCATGTCTAAAACACC
AAAAGCAATGGCAACAAAAGCCAGAATTGACAAATGGGATCTAATTAAACTAAAGAGCTTCTGCACAGCA
AAAGAAACTACCATCAGAGTGAACAGGCAACCTACAACATGGGAGAAAATTTTCGCAACCTACTCATCTG
ACAAAGGGCTAATATCCAGAATCTACAATGAACTCAAACAAATGTACAAGAAAAAAACAAACAACCCCAT
CAAAAAGTGGGTGAAGGACATGAACAGACACTTCTCAAAAGAAGACATTTATGCAGCCAAAAGACACATG
AAAAAATGCTCACCATCACTGGCCATCAGAGAAATGCAAATCAAAACCACAATGAGATACCATCTCACAC
CAGTTAGAATGGCAATCATTTAAAAGTCAGGAAACAACAGGTGCTGGAGAGGATGTGGAGAAATAGGAAC
ACTTCTACACTGTTGGTGGGACTGTAAACTAGTTCAACCATTGTGGAAGTCAGTGTGGCGATTCCTCAGG
GATCTAGAACTAGAAATACCATTTGACCCAGCCATCCCATTACTGGGTATATACCCAAAGGACTATAAAT
CATGCTGCTATAAAGACACATGCACACGTATGTTTATTGCGGCACTATTCACAATAGCAAAGACTTGGAA
CCAACCCAAATGTCCAACAGTGATAGACTGGATTAAGAAAATGTGGCACATATACACCATGGAATACTAT
GCAGCCATAAAAAAAGGATGAGTTCACATCCTTTGTAGGGACATGGATGAAATTGGAAATCATTATTCTC
AGTAAACTATCACAAGAACAGAACACCAAACACCGCATATTCTCACTCATAGGTGGGAATTGAACAATGA
GAACACATGGACACAGGAAGGGGAACATCACACTCTGGGGACTGTTGTGGGGGGGGGGAGGGGGGAGGG
ATAGCATTGGGAGATATACCTAATGCTAGATGATGAGTTAGTGGGTGCAGCGCACCAGCATGGCACATGT
ATACATATGTAACTAACCTGCACGTTGTGCACATGTACCCTAAAATTTAAAGTATAATAATAATAATAAA
TAAATAAATAAATAAATAAAAAATGATGAGTTTAGACAAATATCATTATGGTAGTATTATATTATGTTAT
GTTATATTATATTATATTATATTATGTATAATGTATATTCCTTGCAGCCTGCCCTGCATTCCCAATCTAT
GACTCATGCTGCCTTATTGATACTGAAAAATCTCCACTACAGCATGCCAGCTTTTGAAAGAGAGCCTTGG
GTTCTTTCCCAATACTTACCTTCCTTTTAGGGCAACCTATCTGAGTCCTGTAGCTTGAAAGATTTCCTAC
CAGCCTGCCATCCCAAAGGAACATGGATGAACTATGTTTATGCTGATGTGTCAAGTCATTTCTTGGTATG
GTTCATAGTAGTCCACATGGCTCTTGTAGACAAAGAGATGAATTACTGATGGCAGAATTTCTGTTCTGGC
AACAGGGAAATTTGCAGAAAGGAGACCTTTTCAGTGTGAACATTTTTTGCTCACAGGTGGTCCAGGATGC
CCAATGCTAAATGAGAAGTGAAAAGAGCAATCAGGGCCAGGTGTGGTGGCTCACGCCTGTAATCCAAGCA
CTTTGGGAGGCCAAGGTGGGCGGATCACGAGGTCAGGAGATCGAGACCATCCTGGCTAACATGGTGAAAC
CCTGTTTCTACTAAAAATACAAAAAAAATTAGCCAGGCGTGGTGGCGGGCGCCTATAGTCCCAGCTACTT
GGGAGGCTGAGGCAGGAGAATGGCGTGAACCTGGGAGGCGGAGCTTGCAGTGAGCCAAGATCGCGCCACT
GTGCTCCAGCCTGGGTGACAGAGCGAGACTCTGTCTCAAAAAAAAAAAAAAAAAAAGAGCAGTCAGAATT
CAATTTTTCATTCAGAACAAATCAATCCACGTGGGTAAACATTTTATCAAACTCAAGAATGCTGTCTTTC
AGGGTGCTTTTCCCTCAACAGTCACTTATTTCCTCTTGCAAGTAGTATCTTCGTTCAGGTTCAGTGACTA
CTGTGTATTATATCCAATGCTTCTGGCAAGTGGGTTGGTGGAGGCAGCCCAAGATCTTCTAGAATCAAGA
GAATTGGATCCATTTCCCAGTTCTAACACTTATCAGCTATATGGCTTTTAGGCAAGTCAGTTAAACATCC
GAGTCTCAGTGCTCTCATCTGCAAAACAGAAAATGTGATGTACTTCACAGAGCCAGGGGGAGGAATAACT
AAGGTGGTACATTTGTACGTGCTTTGTAACCTGTAAAGCCCTTTACTGTACACGTGTCATTTACAGCTCT
GTATCACCATCATGACCTAGAAAAGCAGTACTGACAGAAGACTTATCTTCTTGCCAATGCTAAGATAACT
TTAGCCATTTCTGCATTTCTAAAGGAAGGAGTCTTTATCCCAGTATCTATGAAGACTTGGCAGGAATTGC
CGTCAATATTTAGTTGGTAATATAAACGAATTAAACAAAAATGCACACTAGGTTTTAGGAAAATTAAAGA
CAGAACTATCATTTGTACTCCTCTTACATTTCCCAAAGTGCTAAAACTAGACAATAAATCAGTCCTCAAT
AAATGCTTGTTTATCAATTTTATATTCATTTATTTGTTGATAATACAACAAAGATGTTTATATGCAGTAT
AATATATATGGCAAAGATGAAAAGTAGCAAATTCATGAATCAACATCCTATTTATGCTTGAGAAGACAAA
GAAAGTGTTGGTGACTTCATGGTATACATAACCTTAAGGAGCTCGTGATTGAGCCTGGGTCTCTGCTATC
AATGTAGGATATAAATTTCAAATGTACTATCCTTTATATGTATGTTAATGTAGTAAACATAGAAAACTGA
TGCTACTAGTGAGAATACTTTTACTTGAACAACTAAAAGTTTGTCTTTAATCCCCTAAGTGCATACACAA
AAGGAAAGTACTGTACAAATCAAGTACAACAGAAGAAGTAAAGTAAAAGACAAGTGAAGGAATTATCTGG
AACTTAGATCTGGTTGGCTTTTTCTCCTGAAGTACTTAGATAAATTAACTCACTTTTCTCTTTTGCTGAA
GAAGTGCAAATTAGGCAGGCATGTTATTCCACGTAGGCAAAAGGAAAAAAGAAAGAAAAACATAAAATGG
CTATATATTTGACCAAACTTCGTTCTGCAAGAATCCCAATACTAACCTTCTACCATATAAACTACTTTCA
AAATCAGGCTATATCCTTCCAGTACAAACCTGGTTTGTACTACTCAGAGATACTACTCAGAAATACTTTC
AGTATTTCCCTTCTTTCAACTTCTGATGTGATTCTATCCATATTCCCCTGCCTGTCTCCCAGTCAAAGAG
AATGGGACACAACTCTCTTTAGAGTCCATCAGTGATGCTTTAGCTGCCAAAAATAGTGACAATAGACATT
CATTGTCTGTCTACCTTACTTGTTCAACATTCAGAATTCTGCATCTTAAGAGGCTGTGGCTGAAAACTTG
AGCCAGTTCTTCAGAATTTCTAACATGTTATTTCTGCCACTTTTTCTCCCTTACTTTAGCAGAGTAATTT
AATTCAATTTGAGAGAGAGAGAGAAAAAAAAAACTTTTCTAGTTACACAGATCAATCCAATTGTTTGGAG
CTTCAGAATGAATTTTTAAACTTGTTGAACAGAAGCATACAAATCTCTAAGAGCAAGTCAGATAATATAC
AAAGCCTCCATTCATTGTGTAGGCAGAAAGGAATGCTGGTACCCGGCAGCTCTCTGAGGAATGTTCCCTT
GGCTTTGACTATTCTGCTGGGAACAAGGAAGGAAACACATATATAAAATGAATTTATAATGTCTCTGGCT
TGTAATGGCAGAATGATAAGAAAAGTTGGCTGTTTAATATAAACTGTCAGTTGCATATTCCAGGCCTCCT
CTCTTTGAGGTTCCTCCCACATCCACACGCCTGGCTACTGTTTAGTGCGGAGTACAAAGTGGCCGTTTAT
TATTATTGACTGGTGAGGCCTGTGCTCCAAAATTCATTCTGTCAACAGAATGTAAGCAAAGTTGGCATTT
TAAAGCAGGGCTCTTTCAGTTTCTGGGTTTTCTCAGGATTGCTATGCAACAGGATCAGTGCTGTAGTGCC
CGGTTCAAGCTGAAAATGTTACACAGGAAGACATACCATGTAAAGGTCAGATTCTTCTACTATAATAATT
TTCTTGATCTGTGTGTATACAAGTGAAGTTGAATGCATAACCTCTTATCATAACTCTTACCAAGGTCCTA
TGTACTTTCCACCTGTCAAGCCTAAAAATGTGTATTAAATGGGAAATCAAAACTAATAAATGTATGATGC
TGTACTATATGTATGATGCTATAATACCAAGGTGAACTTAATTTGTGTTGTCAAGAAGATTTTCTCTCCC
ATGACAGACTCCCAGGAATGTGCTGGTGCTGTGGGCCAAGTGCAATCTTGTTTATTAGTCTCTCCACGCT
TTTATGGTCAGAGTTAACTCTACAGATTACTACGTAAATAGAAAATATGACTTGATCCATATAGTAATGA
AATTATTGGCACTGGGGTACACTTTATCATAGAATTTTATTGCCTATCACTTCCATAAAATAATACATTT
TGTCCATAGACTAGAAGATATAACTTGTGAACTTTATAAAGTTATAAATACATTACTTTCCAACTCATAA
TGGCAAGGAATAAATCTATTACAACTAATAAGATGCCCATTTTAAATCTACATAATAACAGGAGAAGGCA
ATACGCCAAGAAAAGGGATTTGAGATGTATCTTCTTGTTAGTTTAGCCTGATTGAAATGTCTTTTGAACT
AATAATTATTTATATTTTGCAATTCTCCAAATTCACATTCATCGCTTGTTTCTTTTGTTTGGTAATTCTG
CACATATTCTTCTTCCTGCTGTCCTGTAG

Homo sapiens dystrophin (DMD), intron 55 target sequence 1 (nucleotide positions 1716938-1716987 of NCBI Reference Sequence: NG_012232.1)

(SEQ ID NO: 2158)
GTAAGTCAGGCATTTCCGCTTTAGCACTCTTGTGGATCCAATTGAACAAT

Homo sapiens dystrophin (DMD), intron 55 target sequence 2 (nucleotide positions 1716950-1717012 of NCBI Reference Sequence: NG_012232.1)

(SEQ ID NO: 2159)
TTTCCGCTTTAGCACTCTTGTGGATCCAATTGAACAATTCTCAGCATTTG
TACTTGTAACTGA

Homo sapiens dystrophin (DMD), intron 55 target sequence 3 (nucleotide positions 1717003-1717050 of NCBI Reference Sequence: NG_012232.1)

(SEQ ID NO: 2160)
TTGTAACTGACAAGCCAGGGACAAAACAAAATAGTTGCTTTTATACAG

Homo sapiens dystrophin (DMD), intron 55 target sequence 4 (nucleotide positions 1837063-1837116 of NCBI Reference Sequence: NG_012232.1)

(SEQ ID NO: 2161)
TTATTTATATTTTGCAATTCTCCAAATTCACATTCATCGCTTGTTTCTTT
TGTT

Homo sapiens dystrophin (DMD), intron 55 target sequence 5 (nucleotide positions 1837104-1837153 of NCBI Reference Sequence: NG_012232.1)

(SEQ ID NO: 2162)
TGTTTCTTTTGTTTGGTAATTCTGCACATATTCTTCTTCCTGCTGTCCTG

Homo sapiens dystrophin (DMD), intron 55 target sequence 6 (nucleotide positions 1836907-1837156 of NCBI Reference Sequence: NG_012232.1)

(SEQ ID NO: 2163)
CCAACTCATAATGGCAAGGAATAAATCTATTACAACTAATAAGATGCCCA
TTTTAAATCTACATAATAACAGGAGAAGGCAATACGCCAAGAAAAGGGAT
TTGAGATGTATCTTCTTGTTAGTTTAGCCTGATTGAAATGTCTTTTGAAC
TAATAATTATTTATATTTTGCAATTCTCCAAATTCACATTCATCGCTTGT
TTCTTTTGTTTGGTAATTCTGCACATATTCTTCTTCCTGCTGTCCTGTAG

Homo sapiens dystrophin (DMD) intron 55/exon 56 junction (nucleotide positions 1837127-1837186 of NCBI Reference Sequence: NG_012232.1)

(SEQ ID NO: 2164)
GCACATATTCTTCTTCCTGCTGTCCTGTAGGACCTCCAAGGTGAAATTGA
AGCTCACACA

Homo sapiens dystrophin (DMD), transcript variant Dp427m, exon 56 (nucleotide positions 8462-8634 of NCBI Reference Sequence: NM_004006.2; nucleotide positions 1837157-1837329 of NCBI Reference Sequence: NG_012232.1)

(SEQ ID NO: 2165)
GACCTCCAAGGTGAAATTGAAGCTCACACAGATGTTTATCACAACCTGGA
TGAAAACAGCCAAAAAATCCTGAGATCCCTGGAAGGTTCCGATGATGCAG
TCCTGTTACAAAGACGTTTGGATAACATGAACTTCAAGTGGAGTGAACTT
CGGAAAAAGTCTCTCAACATTAG

Homo sapiens dystrophin (DMD), exon 56 target sequence 1 (nucleotide positions 1837157-1837281 of NCBI Reference Sequence: NG_012232.1)

(SEQ ID NO: 2166)
GACCTCCAAGGTGAAATTGAAGCTCACACAGATGTTTATCACAACCTGGA
TGAAAACAGCCAAAAAATCCTGAGATCCCTGGAAGGTTCCGATGATGCAG
TCCTGTTACAAAGACGTTTGGATAA

Homo sapiens dystrophin (DMD), exon 56 target sequence 2 (nucleotide positions 1837157-1837201 of NCBI Reference Sequence: NG_012232.1)

(SEQ ID NO: 2167)
GACCTCCAAGGTGAAATTGAAGCTCACACAGATGTTTATCACAAC

Homo sapiens dystrophin (DMD), exon 56 target sequence 3 (nucleotide positions 1837181-1837237 of NCBI Reference Sequence: NG_012232.1)

(SEQ ID NO: 2168)
CACACAGATGTTTATCACAACCTGGATGAAAACAGCCAAAAAATCCTGAG
ATCCCTG

Homo sapiens dystrophin (DMD), exon 56 target sequence 4 (nucleotide positions 1837225-1837281 of NCBI Reference Sequence: NG_012232.1)

(SEQ ID NO: 2169)
CCTGAGATCCCTGGAAGGTTCCGATGATGCAGTCCTGTTACAAAGACGTT
TGGATAA

In some embodiments, an oligonucleotide useful for targeting DMD (e.g., for exon skipping) targets a splicing feature in a DMD sequence (e.g., a DMD pre-mRNA). In some embodiments, a splicing feature in a DMD sequence is an exonic splicing enhancer (ESE), a branch point, a splice donor site, or a splice acceptor site in a DMD sequence. In some embodiments, an ESE is in exon 55 of a DMD sequence (e.g., a DMD pre-mRNA). In some embodiments, a branch point is in intron 54 or intron 55 of a DMD sequence (e.g., a DMD pre-mRNA). In some embodiments, a splice donor site is across the junction of exon 54 and intron 54, in intron 54, across the junction of exon 55 and intron 55, or in intron 55 of a DMD sequence (e.g., a DMD pre-mRNA). In some embodiments, a splice acceptor site is in intron 54, across the junction of intron 54 and exon 55, in intron 55, or across the junction of intron 55 and exon 56 of a DMD sequence (e.g., a DMD pre-mRNA). In some embodiments, the oligonucleotide useful for targeting DMD promotes skipping of exon 55, such as by targeting a splicing feature (e.g., an ESE, a branch point, a splice donor site, or a splice acceptor site) in a DMD sequence (e.g., a DMD pre-mRNA). Examples of ESEs, branch points, splice donor sites, and splice acceptor sites are provided in Table 9.

In some embodiments, an oligonucleotide useful for targeting DMD (e.g., for exon skipping) targets an exonic splicing enhancer (ESE) in a DMD sequence. In some embodiments, an oligonucleotide useful for targeting DMD (e.g., for exon skipping) targets an ESE in DMD exon 55 (e.g., an ESE listed in Table 9).

In some embodiments, an oligonucleotide useful for targeting DMD (e.g., for exon skipping, such as for skipping exon 55) comprises a region of complementarity to a target sequence comprising one or more full or partial ESEs of a DMD transcript (e.g., one or more full or partial ESEs listed in Table 9). In some embodiments, the oligonucleotide comprises a region of complementarity to a target sequence comprising one or more full or partial ESEs of DMD exon 55. In some embodiments, the oligonucleotide comprises a region of complementarity to a target sequence comprising one or more full or partial ESEs as set forth in any one of SEQ ID NOs: 2020-2027, 2031-2061, and 2064-2080. In some embodiments, the oligonucleotide comprises a region of complementarity to a target sequence comprising at least 4 (e.g., 4, 5, 6, 7, or 8) consecutive nucleotides of an ESE as set forth in any one of SEQ ID NOs: 2020-2027, 2031-2061, and 2064-2080. In some embodiments, the oligonucleotide comprises at least 4 (e.g., 4, 5, 6, 7, or 8) consecutive nucleotides of an ESE antisense sequence as set forth in any one of SEQ ID NOs: 2081-2088, 2092-2122, and 2125-2141.

In some embodiments, the oligonucleotide comprises a region of complementarity to a target sequence comprising at least 6 (e.g., 6, 7, 8, 9, 10, 11, 12, 13, 14, 15, 16, 17, 18, 19, 20, or more) nucleotides of one or more ESEs (e.g., 2, 3, 4, or more adjacent ESEs) of DMD exon 55. In some embodiments, the oligonucleotide comprises a region of complementarity to a target sequence comprising at least 6 (e.g., 6, 7, 8, 9, 10, 11, 12, 13, 14, 15, 16, 17, 18, 19, 20, or more) nucleotides of one or more ESEs (e.g., 2, 3, 4, or more adjacent ESEs) as set forth in any one of SEQ ID NOs: 2020-2027, 2031-2061, and 2064-2080. In some embodiments, the oligonucleotide comprises at least 6 (e.g., 6, 7, 8, 9, 10, 11, 12, 13, 14, 15, 16, 17, 18, 19, 20, or more) nucleotides of one or more ESE antisense sequences (e.g., antisense sequences of 2, 3, 4, or more adjacent ESEs) as set forth in any one of SEQ ID NOs: 2081-2088, 2092-2122, and 2125-2141.

In some embodiments, an oligonucleotide useful for targeting DMD (e.g., for exon skipping, such as for skipping exon 55) is 18-35 nucleotides in length, and comprises a region of complementarity to a target sequence comprising at least 4 (e.g., 4, 5, 6, 7, or 8) consecutive nucleotides of an ESE as set forth in any one of SEQ ID NOs: 2020-2027, 2031-2061, and 2064-2080. In some embodiments, an oligonucleotide useful for targeting DMD (e.g., for exon skipping, such as for skipping exon 55) is 20-30 (e.g., 20, 25, 30) nucleotides in length, and comprises a region of complementarity to a target sequence comprising at least 4 (e.g., 4, 5, 6, 7, or 8) consecutive nucleotides of an ESE as set forth in any one of SEQ ID NOs: 2020-2027, 2031-2061, and 2064-2080. In some embodiments, an oligonucleotide useful for targeting DMD (e.g., for exon skipping, such as for skipping exon 55) is 20-25 (i.e., 20, 21, 22, 23, 24, or 25) nucleotides in length, and comprises a region of complementarity to a target sequence comprising at least 4 (e.g., 4, 5, 6, 7, or 8) consecutive nucleotides of an ESE as set forth in any one of SEQ ID NOs: 2020-2027, 2031-2061, and 2064-2080. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, or 8) consecutive nucleotides of an ESE as set forth in any one of SEQ ID NOs: 2020-2027, 2031-2061, and 2064-2080.

In some embodiments, an oligonucleotide useful for targeting DMD (e.g., for exon skipping) targets a branch point in a DMD sequence. In some embodiments, an oligonucleotide useful for targeting DMD (e.g., for exon skipping) targets a branch point in DMD intron 54 or intron 55 (e.g., a branch point listed in Table 9).

In some embodiments, an oligonucleotide useful for targeting DMD (e.g., for exon skipping, such as for skipping exon 55) comprises a region of complementarity to a target sequence comprising a full or partial branch point of a DMD transcript (e.g., a full or partial branch point listed in Table 9). In some embodiments, the oligonucleotide comprises a region of complementarity to a target sequence comprising a full or partial branch point of DMD intron 54 or intron 55. In some embodiments, the oligonucleotide comprises a region of complementarity to a target sequence comprising a full or partial branch point as set forth in SEQ ID NO: 2029. In some embodiments, the oligonucleotide comprises a region of complementarity to a target sequence comprising at least 4 (e.g., 4, 5, 6, or 7) consecutive nucleotides of a branch point as set forth in SEQ ID NO: 2029. In some embodiments, the oligonucleotide comprises at least 4 (e.g., 4, 5, 6, or 7) consecutive nucleotides of a branch point antisense sequence as set forth in SEQ ID NO: 2090.

In some embodiments, an oligonucleotide useful for targeting DMD (e.g., for exon skipping, such as for skipping exon 55) is 18-35 nucleotides in length, and comprises a region of complementarity to a target sequence comprising at least 4 (e.g., 4, 5, 6, or 7) consecutive nucleotides of a branch point as set forth in SEQ ID NO: 2029. In some embodiments, an oligonucleotide useful for targeting DMD (e.g., for exon skipping, such as for skipping exon 55) is 20-30 (e.g., 20, 25, 30) nucleotides in length, and comprises a region of complementarity to a target sequence comprising at least 4 (e.g., 4, 5, 6, or 7) consecutive nucleotides of a branch point as set forth in SEQ ID NO: 2029. In some embodiments, an oligonucleotide useful for targeting DMD (e.g., for exon skipping, such as for skipping exon 55) is 20-25 (i.e., 20, 21, 22, 23, 24, or 25) nucleotides in length, and comprises a region of complementarity to a target sequence comprising at least 4 (e.g., 4, 5, 6, or 7) consecutive nucleotides of a branch point as set forth in SEQ ID NO: 2029. In some embodiments, an oligonucleotide useful for targeting DMD (e.g., for exon skipping) is 30 nucleotides in length, and comprises a region of complementarity to a target sequence comprising at least 4 (e.g., 4, 5, 6, or 7) consecutive nucleotides of a branch point as set forth in SEQ ID NO: 2029.

In some embodiments, an oligonucleotide useful for targeting DMD (e.g., for exon skipping) targets a splice donor site in a DMD sequence. In some embodiments, an oligonucleotide useful for targeting DMD (e.g., for exon skipping) targets a splice donor site across the junction of exon 54 and intron 54, in intron 54, across the junction of exon 55 and intron 55, or in intron 55 (e.g., a splice donor site listed in Table 9).

In some embodiments, an oligonucleotide useful for targeting DMD (e.g., for exon skipping, such as for skipping exon 55) comprises a region of complementarity to a target sequence comprising a full or partial splice donor site of a DMD transcript (e.g., a full or partial splice donor site listed in Table 9). In some embodiments, the oligonucleotide comprises a region of complementarity to a target sequence comprising a full or partial splice donor site across the junction of exon 54 and intron 54, in intron 54, across the junction of exon 55 and intron 55, or in intron 55 of DMD. In some embodiments, the oligonucleotide comprises a region of complementarity to a target sequence comprising a full or partial splice donor site as set forth in SEQ ID NO: 2028 or 2062. In some embodiments, the oligonucleotide comprises a region of complementarity to a target sequence comprising at least 4 (e.g., 4, 5, 6, 7, or 8) consecutive nucleotides of a splice donor site as set forth in SEQ ID NO: 2028 or 2062. In some embodiments, the oligonucleotide comprises at least 4 (e.g., 4, 5, 6, 7, or 8) consecutive nucleotides of a splice donor site antisense sequence as set forth in SEQ ID NO: 2089 or 2123.

In some embodiments, an oligonucleotide useful for targeting DMD (e.g., for exon skipping, such as for skipping exon 55) is 18-35 nucleotides in length, and comprises a region of complementarity to a target sequence comprising at least 4 (e.g., 4, 5, 6, 7, or 8) consecutive nucleotides of a splice donor site as set forth in SEQ ID NO: 2028 or 2062. In some embodiments, an oligonucleotide useful for targeting DMD (e.g., for exon skipping, such as for skipping exon 55) is 20-30 (e.g., 20, 25, 30) nucleotides in length, and comprises a region of complementarity to a target sequence comprising at least 4 (e.g., 4, 5, 6, 7, or 8) consecutive nucleotides of a splice donor site as set forth in SEQ ID NO: 2028 or 2062. In some embodiments, an oligonucleotide useful for targeting DMD (e.g., for exon skipping, such as for skipping exon 55) is 20-25 (i.e., 20, 21, 22, 23, 24, or 25) nucleotides in length, and comprises a region of complementarity to a target sequence comprising at least 4 (e.g., 4, 5, 6, 7, or 8) consecutive nucleotides of a splice donor site as set forth in SEQ ID NO: 2028 or 2062. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, or 8) consecutive nucleotides of a splice donor site as set forth in SEQ ID NO: 2028 or 2062.

In some embodiments, an oligonucleotide useful for targeting DMD (e.g., for exon skipping) targets a splice acceptor site in a DMD sequence. In some embodiments, an oligonucleotide useful for targeting DMD (e.g., for exon skipping) targets a splice acceptor site in intron 54, across the junction of intron 54 and exon 55, in intron 55, or across the junction of intron 55 and exon 56 (e.g., a splice acceptor site listed in Table 9).

In some embodiments, an oligonucleotide useful for targeting DMD (e.g., for exon skipping, such as for skipping exon 55) comprises a region of complementarity to a target sequence comprising a full or partial splice acceptor site of a DMD transcript (e.g., a full or partial splice acceptor site listed in Table 9). In some embodiments, the oligonucleotide comprises a region of complementarity to a target sequence comprising a full or partial splice acceptor site in intron 54, across the junction of intron 54 and exon 55, in intron 55, or across the junction of intron 55 and exon 56 of DMD. In some embodiments, the oligonucleotide comprises a region of complementarity to a target sequence comprising a full or partial splice acceptor site as set forth in SEQ ID NO: 2030 or 2063. In some embodiments, the oligonucleotide comprises a region of complementarity to a target sequence comprising at least 4 (e.g., 4, 5, 6, 7, 8, 9, 10, or 11) consecutive nucleotides of a splice acceptor site as set forth in SEQ ID NO: 2030 or 2063. In some embodiments, the oligonucleotide comprises at least 4 (e.g., 4, 5, 6, 7, 8, 9, 10, or 11) consecutive nucleotides of a splice acceptor site antisense sequence as set forth in SEQ ID NO: 2091 or 2124.

In some embodiments, an oligonucleotide useful for targeting DMD (e.g., for exon skipping, such as for skipping exon 55) is 18-35 nucleotides in length, and comprises a region of complementarity to a target sequence comprising at least 4 (e.g., 4, 5, 6, 7, 8, 9, 10, or 11) consecutive nucleotides of a splice acceptor site as set forth in SEQ ID NO: 2030 or 2063. In some embodiments, an oligonucleotide useful for targeting DMD (e.g., for exon skipping, such as for skipping exon 55) is 20-30 (e.g., 20, 25, 30) nucleotides in length, and comprises a region of complementarity to a target sequence comprising at least 4 (e.g., 4, 5, 6, 7, 8, 9, 10, or 11) consecutive nucleotides of a splice acceptor site as set forth in SEQ ID NO: 2030 or 2063. In some embodiments, an oligonucleotide useful for targeting DMD (e.g., for exon skipping, such as for skipping exon 55) is 20-25 (i.e., 20, 21, 22, 23, 24, or 25) nucleotides in length, and comprises a region of complementarity to a target sequence comprising at least 4 (e.g., 4, 5, 6, 7, 8, 9, 10, or 11) consecutive nucleotides of a splice acceptor site as set forth in SEQ ID NO: 2030 or 2063. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, 8, 9, 10, or 11) consecutive nucleotides of a splice acceptor site as set forth in SEQ ID NO: 2030 or 2063.

In some embodiments, an oligonucleotide useful for targeting DMD (e.g., for exon skipping) comprises a region of complementarity to a junction of an exon and an intron of a DMD RNA (e.g., any one of the exon/intron junctions provided by SEQ ID NOs: 2144, 2151, 2156, and 2164). In some embodiments, an oligonucleotide useful for targeting DMD (e.g., for exon skipping) comprises a region of complementarity to at least 10 (e.g., 10, 11, 12, 13, 14, 15, 16, 17, 18, 19, 20, 21, 22, 23, 24, 25, or more) consecutive nucleosides of ajunction of an exon and an intron of a DMD RNA (e.g., any one of the exon/intronjunctions provided by SEQ ID NOs: 2144, 2151, 2156, and 2164). In some embodiments, an oligonucleotide useful for targeting DMD (e.g., for exon skipping) is complementary to any one of SEQ ID NOs: 2144, 2151, 2156, and 2164.

In some embodiments, an oligonucleotide useful for targeting DMD (e.g., for exon skipping) comprises a region of complementarity to a target sequence of a DMD RNA (e.g., a target sequence provided by any one of SEQ ID NOs: 2143, 2146-2150, 2153-2155, 2158-2163, and 2166-2169). In some embodiments, an oligonucleotide useful for targeting DMD (e.g., for exon skipping) comprises a region of complementarity to at least 10 (e.g., 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 2143, 2146-2150, 2153-2155, 2158-2163, and 2166-2169). In some embodiments, an oligonucleotide useful for targeting DMD (e.g., for exon skipping) is complementary to any one of SEQ ID NOs: 2143, 2146-2150, 2153-2155, 2158-2163, and 2166-2169.

TABLE 9
Example target sequence motifs

| | | SEQ | | SEQ | Motif |
|---|---|---|---|---|---|
| Location in | | ID | Motif | ID | Antisense |
| DMD | Type | NO: | Sequence† | NO: | Sequence† |
| Exon 54 | ESE | 2020 | ATTCTGC | 2081 | GCAGAAT |
| Exon 54 | ESE | 2021 | CTGCAGA | 2082 | TCTGCAG |
| Exon 54 | ESE | 2022 | TGCAGA | 2083 | TCTGCA |
| Exon 54 | ESE | 2023 | CAGATGA | 2084 | TCATCTG |
| Exon 54 | ESE | 2024 | CCACATG | 2085 | CATGTGG |
| Exon 54 | ESE | 2025 | CACATGA | 2086 | TCATGTG |
| Exon 54 | ESE | 2026 | CAGAGAA | 2087 | TTCTCTG |
| Exon 54 | ESE | 2027 | TCAATGC | 2088 | GCATTGA |
| Across exon | Splice | 2028 | AGGTATG | 2089 | CATACCT |
| 54/intron 54 | Donor | | | | |
| junction | | | | | |
| Intron 54 | Branch | 2029 | TTCTGAT | 2090 | ATCAGAA |
| | Point | | | | |
| Across | Splice | 2030 | TCCTTTGC | 2091 | CCTGCAAA |
| intron | Acceptor | | AGG | | GGA |
| 54/exon 55 | | | | | |
| junction | | | | | |
| Exon 55 | ESE | 2031 | CGAGAGG | 2092 | CCTCTCG |
| Exon 55 | ESE | 2032 | GGCTGCTT | 2093 | AAGCAGCC |
| Exon 55 | ESE | 2033 | GATTACTG | 2094 | CAGTAATC |
| Exon 55 | ESE | 2034 | TTACTGC | 2095 | GCAGTAA |
| Exon 55 | ESE | 2035 | TGCAAC | 2096 | GTTGCA |
| Exon 55 | ESE | 2036 | CCCCCTG | 2097 | CAGGGGG |
| Exon 55 | ESE | 2037 | CCCCTGG | 2098 | CCAGGGG |
| Exon 55 | ESE | 2038 | CCCTGGA | 2099 | TCCAGGG |
| Exon 55 | ESE | 2039 | GTTTCTTG | 2100 | CAAGAAAC |
| Exon 55 | ESE | 2040 | TTTCTTG | 2101 | CAAGAAA |
| Exon 55 | ESE | 2041 | TGCCTGG | 2102 | CCAGGCA |
| Exon 55 | ESE | 2042 | GGCTTACA | 2103 | TGTAAGCC |
| Exon 55 | ESE | 2043 | TTACAGA | 2104 | TCTGTAA |
| Exon 55 | ESE | 2044 | TACAGA | 2105 | TCTGTA |
| Exon 55 | ESE | 2045 | ACAGAAG | 2106 | CTTCTGT |
| Exon 55 | ESE | 2046 | CTGCCAA | 2107 | TTGGCAG |
| Exon 55 | ESE | 2047 | TGCCAATG | 2108 | CATTGGCA |
| Exon 55 | ESE | 2048 | GTCCTACA | 2109 | TGTAGGAC |
| Exon 55 | ESE | 2049 | CTACAGG | 2110 | CCTGTAG |
| Exon 55 | ESE | 2050 | TACAGGA | 2111 | TCCTGTA |
| Exon 55 | ESE | 2051 | GGATGCTA | 2112 | TAGCATCC |
| Exon 55 | ESE | 2052 | CTACCCG | 2113 | CGGGTAG |
| Exon 55 | ESE | 2053 | TACCCGTA | 2114 | TACGGGTA |
| Exon 55 | ESE | 2054 | GGCTCCTA | 2115 | TAGGAGCC |
| Exon 55 | ESE | 2055 | CTAGAAG | 2116 | CTTCTAG |
| Exon 55 | ESE | 2056 | AGACTCC | 2117 | GGAGTCT |
| Exon 55 | ESE | 2057 | GACTCCAA | 2118 | TTGGAGTC |
| Exon 55 | ESE | 2058 | CTCCAAG | 2119 | CTTGGAG |
| Exon 55 | ESE | 2059 | CCAAGGG | 2120 | CCCTTGG |
| Exon 55 | ESE | 2060 | CTGATGA | 2121 | TCATCAG |
| Exon 55 | ESE | 2061 | ACAATGG | 2122 | CCATTGT |
| Across exon | Splice | 2062 | AAGTAAG | 2123 | CTTACTT |
| 55/intron 55 | Donor | | | | |
| junction | | | | | |
| Across | Splice | 2063 | TCCTGTAGG | 2124 | CCTACAGGA |
| intron | Acceptor | | | | |
| 55/exon 56 | | | | | |
| junction | | | | | |
| Exon 56 | ESE | 2064 | GACCTCCA | 2125 | TGGAGGTC |
| Exon 56 | ESE | 2058 | CTCCAAG | 2119 | CTTGGAG |
| Exon 56 | ESE | 2065 | CCAAGGT | 2126 | ACCTTGG |
| Exon 56 | ESE | 2066 | TCACACA | 2127 | TGTGTGA |
| Exon 56 | ESE | 2067 | CACACAG | 2128 | CTGTGTG |
| Exon 56 | ESE | 2068 | ACACAGA | 2129 | TCTGTGT |
| Exon 56 | ESE | 2069 | CACAGA | 2130 | TCTGTG |
| Exon 56 | ESE | 2070 | CAGATGT | 2131 | ACATCTG |
| Exon 56 | ESE | 2071 | TCACAAC | 2132 | GTTGTGA |
| Exon 56 | ESE | 2072 | CAGCCAA | 2133 | TTGGCTG |
| Exon 56 | ESE | 2073 | AAATCCTG | 2134 | CAGGATTT |
| Exon 56 | ESE | 2074 | CTGAGAT | 2135 | ATCTCAG |
| Exon 56 | ESE | 2075 | GATCCCTG | 2136 | CAGGGATC |
| Exon 56 | ESE | 2076 | TCCCTGG | 2137 | CCAGGGA |
| Exon 56 | ESE | 2038 | CCCTGGA | 2099 | TCCAGGG |
| Exon 56 | ESE | 2077 | GGTTCCGA | 2138 | TCGGAACC |
| Exon 56 | ESE | 2078 | CCGATGA | 2139 | TCATCGG |
| Exon 56 | ESE | 2079 | TTACAAA | 2140 | TTTGTAA |
| Exon 56 | ESE | 2080 | AAGACGT | 2141 | ACGTCTT |

†Each thymine base (T) in any one of the sequences provided in Table 9 may independently and optionally be replaced with a uracil base (U). Motif sequences and antisense sequences listed in Table 9 contain T's, but binding of a motif sequence in RNA and/or DNA is contemplated.

In some embodiments, any one of the oligonucleotides useful for targeting DMD (e.g., for exon skipping) is a phosphorodiamidate morpholino oligomer (PMO).

In some embodiments, the oligonucleotide may have region of complementarity to a mutant DMD allele, for example, a DMD allele with at least one mutation in any of exons 1-79 of DMD in humans that leads to a frameshift and improper RNA splicing/processing.

In some embodiments, any one of the oligonucleotides can be in salt form, e.g., as sodium, potassium, or magnesium salts.

In some embodiments, the 5′ or 3′ nucleoside (e.g., terminal nucleoside) of any one of the oligonucleotides described herein is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of any one of the oligonucleotides described herein is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)n- and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR1 antibody, e.g., via the amine group.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, 20 to 25 nucleotides in length, etc.

In some embodiments, a nucleic acid sequence of an oligonucleotide for purposes of the present disclosure is “complementary” to a target nucleic acid when it is specifically hybridizable to the target nucleic acid. In some embodiments, an oligonucleotide hybridizing to a target nucleic acid (e.g., an mRNA or pre-mRNA molecule) results in modulation of activity or expression of the target (e.g., decreased mRNA translation, altered pre-mRNA splicing, exon skipping, target mRNA degradation, etc.). In some embodiments, a nucleic acid sequence of an oligonucleotide has a sufficient degree of complementarity to its target nucleic acid such that it does not hybridize non-target sequences under conditions in which avoidance of non-specific binding is desired, e.g., under physiological conditions. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of a target nucleic acid. In some embodiments a complementary nucleotide sequence need not be 100% complementary to that of its target to be specifically hybridizable or specific for a target nucleic acid. In certain embodiments, oligonucleotides comprise one or more mismatched nucleobases relative to the target nucleic acid. In certain embodiments, activity relating to the target is reduced by such mismatch, but activity relating to a non-target is reduced by a greater amount (i.e., selectivity for the target nucleic acid is increased and off-target effects are decreased).

In some embodiments, an oligonucleotide comprises region of complementarity to a target nucleic acid that is in the range of 8 to 15, 8 to 30, 8 to 40, or 10 to 50, or 5 to 50, 15 to 20, 20 to 25, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide is complementary (e.g., at least 85% at least 90%, at least 95%, or 100%) to a target sequence of the any one of the oligonucleotides described herein (e.g., the oligonucleotides listed in Table 8). In some embodiments, the oligonucleotide is complementary (e.g., at least 85% at least 90%, at least 95%, or 100%) to a target sequence of the any one of the oligonucleotides provided by SEQ ID NO: 780-2019. In some embodiments, such target sequence is 100% complementary to an oligonucleotide listed in Table 8. In some embodiments, such target sequence is 100% complementary to an oligonucleotide provided by SEQ ID NO: 780-2019. In some embodiments, the oligonucleotide is complementary (e.g., at least 85% at least 90%, at least 95%, or 100%) to a target sequence provided herein (e.g., a target sequence listed in Table 8). In some embodiments, the oligonucleotide is complementary (e.g., at least 85% at least 90%, at least 95%, or 100%) to any one of SEQ ID NO: 160-779.

In some embodiments, an oligonucleotide useful for targeting DMD (e.g., for exon skipping) comprises a region of complementarity to a target sequence of a DMD RNA (e.g., a target sequence provided by any one of SEQ ID NOs: 160-779). In some embodiments, an oligonucleotide useful for targeting DMD (e.g., for exon skipping) comprises a region of complementarity to at least 8 (e.g., 8, 9, 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 160-779). In some embodiments, an oligonucleotide useful for targeting DMD (e.g., for exon skipping) is complementary to any one of SEQ ID NOs: 160-779.

In some embodiments, an oligonucleotide useful for targeting DMD (e.g., for exon skipping) comprises a sequence comprising at least 8 (e.g., 8, 9, 10, 11, 12, 13, 14, 15, 16, 17, 18, 19, 20, 21, 22, 23, 24, 25, or more) consecutive nucleobases of a DMD-targeting sequence provided herein (e.g., an antisense sequence listed in Table 8). In some embodiments, the oligonucleotide comprises a sequence comprising at least 8 (e.g., 8, 9, 10, 11, 12, 13, 14, 15, 16, 17, 18, 19, 20, 21, 22, 23, 24, 25, or more) consecutive nucleobases of any one of SEQ ID NOs: 780-2019. In some embodiments, the oligonucleotide comprises the sequence of any one of SEQ ID NOs: 780-2019.

In some embodiments, an oligonucleotide useful for targeting DMD (e.g., for exon skipping) comprises a region of complementarity to a target sequence of a DMD RNA (e.g., a target sequence provided by any one of SEQ ID NOs: 160, 162-166, 168, 169, 173, 178-180, 243-251, 253, 255, 256, 262-266, 268, 270-272, 274, 282-284, 289-291, 294, 295, 319, 343, 347, 351, 356-358, 364, 366, 367, 398, 401, 453-455, 462, 463, 526, 573, 748, and 755). In some embodiments, an oligonucleotide useful for targeting DMD (e.g., for exon skipping) comprises a region of complementarity to at least 8 (e.g., 8, 9, 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 160, 162-166, 168, 169, 173, 178-180, 243-251, 253, 255, 256, 262-266, 268, 270-272, 274, 282-284, 289-291, 294, 295, 319, 343, 347, 351, 356-358, 364, 366, 367, 398, 401, 453-455, 462, 463, 526, 573, 748, and 755). In some embodiments, an oligonucleotide useful for targeting DMD (e.g., for exon skipping) is complementary to any one of SEQ ID NOs: 160, 162-166, 168, 169, 173, 178-180, 243-251, 253, 255, 256, 262-266, 268, 270-272, 274, 282-284, 289-291, 294, 295, 319, 343, 347, 351, 356-358, 364, 366, 367, 398, 401, 453-455, 462, 463, 526, 573, 748, and 755.

In some embodiments, an oligonucleotide useful for targeting DMD (e.g., for exon skipping) comprises a sequence comprising at least 8 (e.g., 8, 9, 10, 11, 12, 13, 14, 15, 16, 17, 18, 19, 20, 21, 22, 23, 24, 25, or more) contiguous nucleobases of a DMD-targeting sequence provided herein (e.g., a sequence of any one of SEQ ID NOs: 1400, 1402-1406, 1408, 1409, 1413, 1418-1420, 1483-1491, 1493, 1495, 1496, 1502-1506, 1508, 1510-1512, 1514, 1522-1524, 1529-1531, 1534, 1535, 1559, 1583, 1587, 1591, 1596, 1597, 1598, 1604, 1606, 1607, 1638, 1641, 1693-1695, 1702, 1703, 1766, 1813, 1988, and 1995). In some embodiments, the oligonucleotide comprises at least 8 (e.g., 8, 9, 10, 11, 12, 13, 14, 15, 16, 17, 18, 19, 20, 21, 22, 23, 24, 25, or more) consecutive nucleosides of a DMD-targeting sequence provided herein (e.g., a sequence of any one of SEQ ID NOs: 1400, 1402-1406, 1408, 1409, 1413, 1418-1420, 1483-1491, 1493, 1495, 1496, 1502-1506, 1508, 1510-1512, 1514, 1522-1524, 1529-1531, 1534, 1535, 1559, 1583, 1587, 1591, 1596, 1597, 1598, 1604, 1606, 1607, 1638, 1641, 1693-1695, 1702, 1703, 1766, 1813, 1988, and 1995). In some embodiments, the oligonucleotide comprises the sequence of any one of SEQ ID NOs: 1400, 1402-1406, 1408, 1409, 1413, 1418-1420, 1483-1491, 1493, 1495, 1496, 1502-1506, 1508, 1510-1512, 1514, 1522-1524, 1529-1531, 1534, 1535, 1559, 1583, 1587, 1591, 1596, 1597, 1598, 1604, 1606, 1607, 1638, 1641, 1693-1695, 1702, 1703, 1766, 1813, 1988, and 1995.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotides listed in Table 8) may independently and optionally be uracil bases (U's), and/or any one or more of the U's in the oligonucleotides provided herein may independently and optionally be T's. In some embodiments, any one or more of the thymine bases (T's) in any one of the oligonucleotides provided by SEQ ID NOs: 1400-2019 or in an oligonucleotide complementary to any one of SEQ ID NOs: 160-779 may optionally be uracil bases (U's), and/or any one or more of the U's in the oligonucleotides may optionally be T's. In some embodiments, any one or more of the uracil bases (U's) in any one of the oligonucleotides provided by SEQ ID NOs: 780-1399 or in an oligonucleotide complementary to any one of SEQ ID NOs: 160-779 may optionally be thymine bases (T's), and/or any one or more of the T's in the oligonucleotides may optionally be U's.

b. Oligonucleotide Modifications:

The oligonucleotides described herein may be modified, e.g., comprise a modified sugar moiety, a modified internucleoside linkage, a modified nucleotide or nucleoside and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide or nucleoside modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide or nucleoside modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides or nucleosides of the oligonucleotide are modified nucleotides/nucleosides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides or nucleosides of the oligonucleotide are modified nucleotides/nucleosides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides or nucleosides of the oligonucleotide are modified nucleotides/nucleosides. Optionally, the oligonucleotides may have every nucleotide or nucleoside except 1, 2, 3, 4, 5, 6, 7, 8, 9, or 10 nucleotides/nucleosides modified. Oligonucleotide modifications are described further herein.

c. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′-O atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1° C., 2° C., 3° C., 4° C., or 5° C. compared with an oligonucleotide that does not have the at least one modified nucleoside. The oligonucleotide may have a plurality of modified nucleosides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′-O-Me modified nucleosides. An oligonucleotide may comprise a mix of 2′-fluoro modified nucleosides and 2′-O-Me modified nucleosides. An oligonucleotide may comprise a mix of 2′-4′ bicyclic nucleosides and 2′-MOE, 2′-fluoro, or 2′-O-Me modified nucleosides. An oligonucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′-O-Me modified nucleosides. An oligonucleotide may comprise alternating 2′-fluoro modified nucleosides and 2′-O-Me modified nucleosides. An oligonucleotide may comprise alternating 2′-4′ bicyclic nucleosides and 2′-MOE, 2′-fluoro, or 2′-O-Me modified nucleosides. An oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues.

d. Internucleoside Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleosides. In some embodiments, the oligonucleotide comprises phosphorothioate internucleoside linkages between all nucleosides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides comprise nucleoside units that are joined together by either substantially all Sp or substantially all Rp phosphorothioate intersugar linkages are provided. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 Al, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Mixmers

In some embodiments, an oligonucleotide described herein may be a mixmer or comprise a mixmer sequence pattern. In general, mixmers are oligonucleotides that comprise both naturally and non-naturally occurring nucleosides or comprise two different types of non-naturally occurring nucleosides typically in an alternating pattern. Mixmers generally have higher binding affinity than unmodified oligonucleotides and may be used to specifically bind a target molecule, e.g., to block a binding site on the target molecule. Generally, mixmers do not recruit an RNase to the target molecule and thus do not promote cleavage of the target molecule. Such oligonucleotides that are incapable of recruiting RNase H have been described, for example, see WO2007/112754 or WO2007/112753.

In some embodiments, the mixmer comprises or consists of a repeating pattern of nucleoside analogues and naturally occurring nucleosides, or one type of nucleoside analogue and a second type of nucleoside analogue. However, a mixmer need not comprise a repeating pattern and may instead comprise any arrangement of modified nucleoside s and naturally occurring nucleoside s or any arrangement of one type of modified nucleoside and a second type of modified nucleoside. The repeating pattern, may, for instance be every second or every third nucleoside is a modified nucleoside, such as LNA, and the remaining nucleoside s are naturally occurring nucleosides, such as DNA, or are a 2′ substituted nucleoside analogue such as 2′-MOE or 2′ fluoro analogues, or any other modified nucleoside described herein. It is recognized that the repeating pattern of modified nucleoside, such as LNA units, may be combined with modified nucleoside at fixed positions—e.g. at the 5′ or 3′ termini.

In some embodiments, a mixmer does not comprise a region of more than 5, more than 4, more than 3, or more than 2 consecutive naturally occurring nucleosides, such as DNA nucleosides. In some embodiments, the mixmer comprises at least a region consisting of at least two consecutive modified nucleosides, such as at least two consecutive LNAs. In some embodiments, the mixmer comprises at least a region consisting of at least three consecutive modified nucleoside units, such as at least three consecutive LNAs.

In some embodiments, the mixmer does not comprise a region of more than 7, more than 6, more than 5, more than 4, more than 3, or more than 2 consecutive nucleoside analogues, such as LNAs. In some embodiments, LNA units may be replaced with other nucleoside analogues, such as those referred to herein.

Mixmers may be designed to comprise a mixture of affinity enhancing modified nucleosides, such as in non-limiting example LNA nucleosides and 2′-O-Me nucleosides. In some embodiments, a mixmer comprises modified internucleoside linkages (e.g., phosphorothioate internucleoside linkages or other linkages) between at least two, at least three, at least four, at least five or more nucleosides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleosides. For example, in some embodiments, a mixmer may comprise morpholino nucleosides mixed (e.g., in an alternating manner) with one or more other nucleosides (e.g., DNA, RNA nucleosides) or modified nucleosides (e.g., LNA, 2′-O-Me nucleosides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′-O-Methyl Mixmer Antisense Oligonucleotide, Molecules 2016, 21, 1582, the contents of each which are incorporated herein by reference.

i. Multimers

In some embodiments, molecular payloads may comprise multimers (e.g., concatemers) of 2 or more oligonucleotides connected by a linker. In this way, in some embodiments, the oligonucleotide loading of a complex can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via an oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

### C. Linkers

Complexes described herein generally comprise a linker that covalently links any one of the anti-TfR1 antibodies described herein to a molecular payload. A linker comprises at least one covalent bond. In some embodiments, a linker may be a single bond, e.g., a disulfide bond or disulfide bridge, that covalently links an anti-TfR1 antibody to a molecular payload. However, in some embodiments, a linker may covalently link any one of the anti-TfR1 antibodies described herein to a molecular payload through multiple covalent bonds. In some embodiments, a linker may be a cleavable linker. However, in some embodiments, a linker may be a non-cleavable linker. A linker is typically stable in vitro and in vivo, and may be stable in certain cellular environments. Additionally, typically a linker does not negatively impact the functional properties of either the anti-TfR1 antibody or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A linker typically will contain two different reactive species that allow for attachment to both the anti-TfR1 antibody and a molecular payload. In some embodiments, the two different reactive species may be a nucleophile and/or an electrophile. In some embodiments, a linker contains two different electrophiles or nucleophiles that are specific for two different nucleophiles or electrophiles. In some embodiments, a linker is covalently linked to an anti-TfR1 antibody via conjugation to a lysine residue or a cysteine residue of the anti-TfR1 antibody. In some embodiments, a linker is covalently linked to a cysteine residue of an anti-TfR1 antibody via a maleimide-containing linker, wherein optionally the maleimide-containing linker comprises a maleimidocaproyl or maleimidomethyl cyclohexane-1-carboxylate group. In some embodiments, a linker is covalently linked to a cysteine residue of an anti-TfR1 antibody or thiol functionalized molecular payload via a 3-arylpropionitrile functional group. In some embodiments, a linker is covalently linked to a lysine residue of an anti-TfR1 antibody. In some embodiments, a linker is covalently linked to an anti-TfR1 antibody and/or (e.g., and) a molecular payload, independently, via an amide bond, a carbamate bond, a hydrazide, a triazole, a thioether, and/or a disulfide bond.

i. Cleavable Linkers

A cleavable linker may be a protease-sensitive linker, a pH-sensitive linker, or a glutathione-sensitive linker. These linkers are typically cleavable only intracellularly and are preferably stable in extracellular environments, e.g., extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g., a cysteine residue.

In some embodiments, a linker comprises a valine-citrulline sequence (e.g., as described in U.S. Pat. No. 6,214,345, incorporated herein by reference). In some embodiments, before conjugation, a linker comprises a structure of:

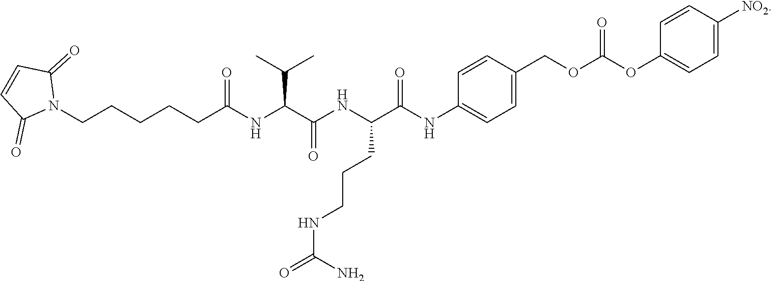 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, a linker comprises a structure of:

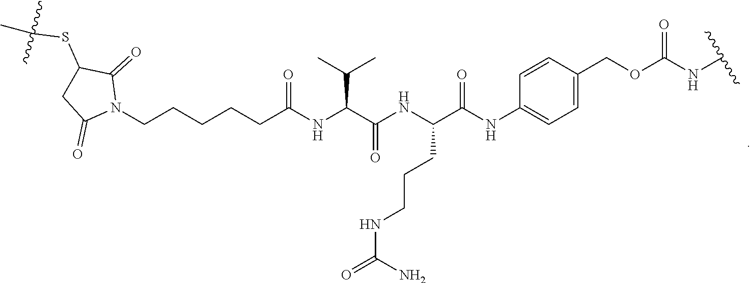 [SMILES: *NC(=O)OCc1ccc(NC(=O)[C@H](CCCNC(N)=O)NC(=O)[C@@H](NC(=O)CCCCCN2C(=O)CC(S*)C2=O)C(C)C)cc1]

In some embodiments, before conjugation, a linker comprises a structure of:

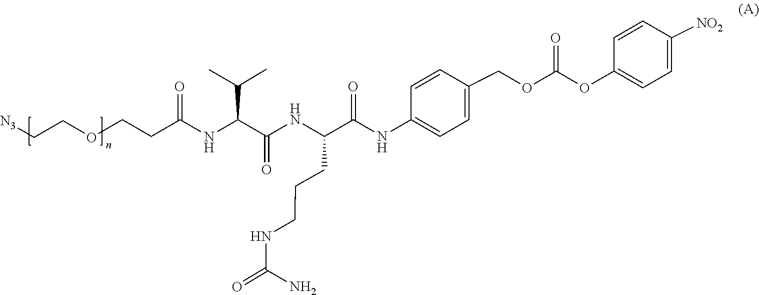

wherein n is any number from 0-10. In some embodiments, n is 3.

In some embodiments, a linker comprises a structure of:

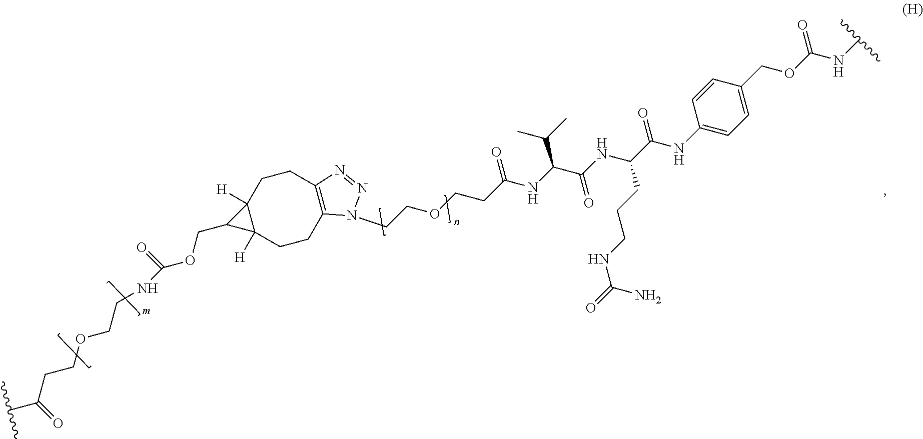

wherein n is any number from 0-10, wherein m is any number from 0-10. In some embodiments, n is 3 and/or (e.g., and) m is 4.

In some embodiments, a linker comprises a structure of:

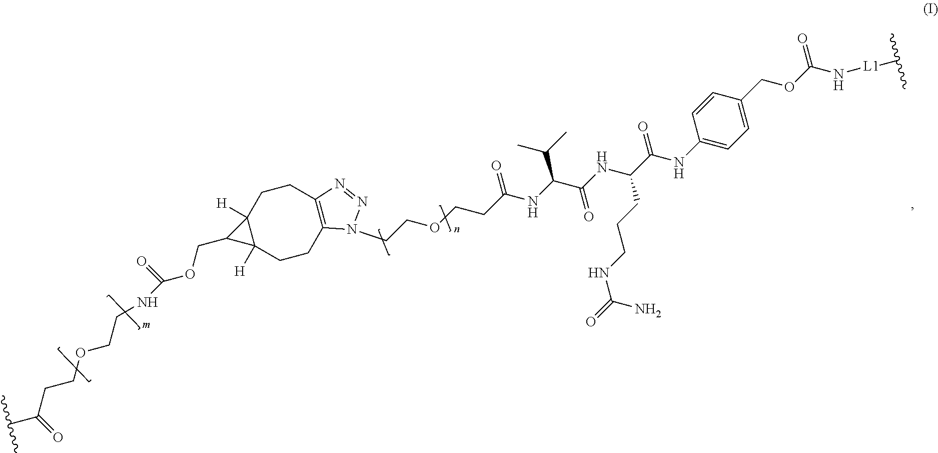

wherein n is any number from 0-10, wherein m is any number from 0-10. In some embodiments, n is 3 and/or (e.g., and) m is 4.
ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation can be utilized to covalently link an anti-TfR1 antibody comprising a LPXT sequence to a molecular payload comprising a (G)ₙ sequence (see, e.g. Proft T. Sortase-mediated protein ligation: an emerging biotechnology tool for protein modification and immobilization. Biotechnol Lett. 2010, 32(1):1-10.).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S, an optionally substituted heterocyclylene further comprising at least one heteroatom selected from N, O, and S, an imino, an optionally substituted nitrogen species, an optionally substituted oxygen species 0, an optionally substituted sulfur species, or a poly(alkylene oxide), e.g. polyethylene oxide or polypropylene oxide. In some embodiments, a linker may be a non-cleavable N-gamma-maleimidobutyryl-oxysuccinimide ester (GMBS) linker.

iii. Linker Conjugation

In some embodiments, a linker is covalently linked to an anti-TfR1 antibody and/or (e.g., and) molecular payload via a phosphate, thioether, ether, carbon-carbon, carbamate, or amide bond. In some embodiments, a linker is covalently linked to an oligonucleotide through a phosphate or phosphorothioate group, e.g. a terminal phosphate of an oligonucleotide backbone. In some embodiments, a linker is covalently linked to an anti-TfR1 antibody, through a lysine or cysteine residue present on the anti-TfR1 antibody.

In some embodiments, a linker, or a portion thereof is covalently linked to an anti-TfR1 antibody and/or (e.g., and) molecular payload by a cycloaddition reaction between an azide and an alkyne to form a triazole, wherein the azide or the alkyne may be located on the anti-TfR1 antibody, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctyne is as described in International Patent Application Publication WO2011136645, published on Nov. 3, 2011, entitled, “Fused Cyclooctyne Compounds And Their Use In Metal-free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A P(1,4)-N-Acetylgalactosaminyltransferase”. In some embodiments, a cycloaddition reaction between an azide and an alkyne to form a triazole, wherein the azide or the alkyne may be located on the anti-TfR1 antibody, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A P(1,4)-N-Acetylgalactosaminyltransferase”.

In some embodiments, a linker comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is covalently linked to an anti-TfR1 antibody and/or (e.g., and) molecular payload by the Diels-Alder reaction between a dienophile and a diene/hetero-diene, wherein the dienophile or the diene/hetero-diene may be located on the anti-TfR1 antibody, molecular payload, or the linker. In some embodiments a linker is covalently linked to an anti-TfR1 antibody and/or (e.g., and) molecular payload by other pericyclic reactions such as an ene reaction. In some embodiments, a linker is covalently linked to an anti-TfR1 antibody and/or (e.g., and) molecular payload by an amide, thioamide, or sulfonamide bond reaction. In some embodiments, a linker is covalently linked to an anti-TfR1 antibody and/or (e.g., and) molecular payload by a condensation reaction to form an oxime, hydrazone, or semicarbazide group existing between the linker and the anti-TfR1 antibody and/or (e.g., and) molecular payload.

In some embodiments, a linker is covalently linked to an anti-TfR1 antibody and/or (e.g., and) molecular payload by a conjugate addition reaction between a nucleophile, e.g. an amine or a hydroxyl group, and an electrophile, e.g. a carboxylic acid, carbonate, or an aldehyde. In some embodiments, a nucleophile may exist on a linker and an electrophile may exist on an anti-TfR1 antibody or molecular payload prior to a reaction between a linker and an anti-TfR1 antibody or molecular payload. In some embodiments, an electrophile may exist on a linker and a nucleophile may exist on an anti-TfR1 antibody or molecular payload prior to a reaction between a linker and an anti-TfR1 antibody or molecular payload. In some embodiments, an electrophile may be an azide, pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, and/or a thiol group.

In some embodiments, a linker comprises a valine-citrulline sequence covalently linked to a reactive chemical moiety (e.g., an azide moiety or a BCN moiety for click chemistry). In some embodiments, a linker comprising a valine-citrulline sequence covalently linked to a reactive chemical moiety (e.g., an azide moiety for click chemistry) comprises a structure of:

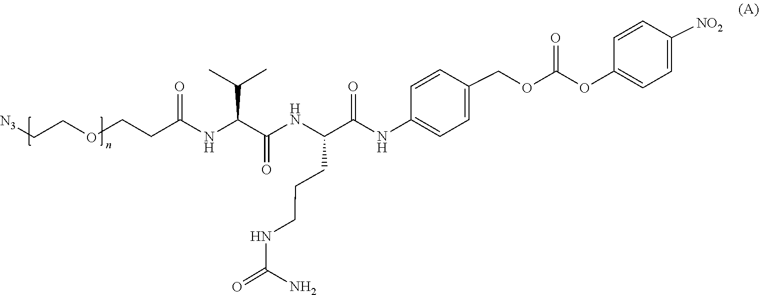

wherein n is any number from 0-10. In some embodiments, n is 3.

In some embodiments, a linker comprising the structure of Formula (A) is covalently linked (e.g., optionally via additional chemical moieties) to a molecular payload (e.g., an oligonucleotide). In some embodiments, a linker comprising the structure of Formula (A) is covalently linked to an oligonucleotide, e.g., through a nucleophilic substitution with amine-L1-oligonucleotides forming a carbamate bond, yielding a compound comprising a structure of:

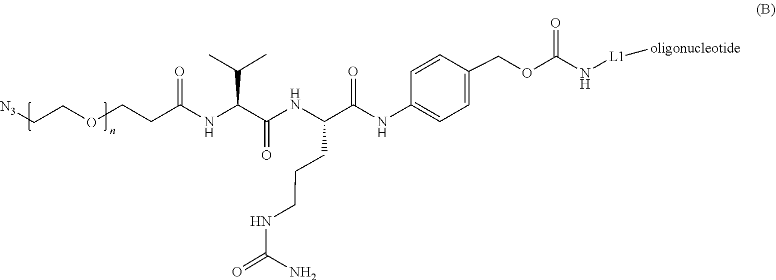

wherein n is any number from 0-10. In some embodiments, n is 3.

In some embodiments, the compound of Formula (B) is further covalently linked via a triazole to additional moieties, wherein the triazole is formed by a click reaction between the azide of Formula (A) or Formula (B) and an alkyne provided on a bicyclononyne. In some embodiments, a compound comprising a bicyclononyne comprises a structure of:

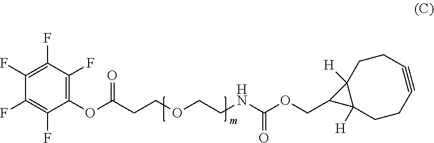

wherein m is any number from 0-10. In some embodiments, m is 4.

In some embodiments, the azide of the compound of structure (B) forms a triazole via a click reaction with the alkyne of the compound of structure (C), forming a compound comprising a structure of:

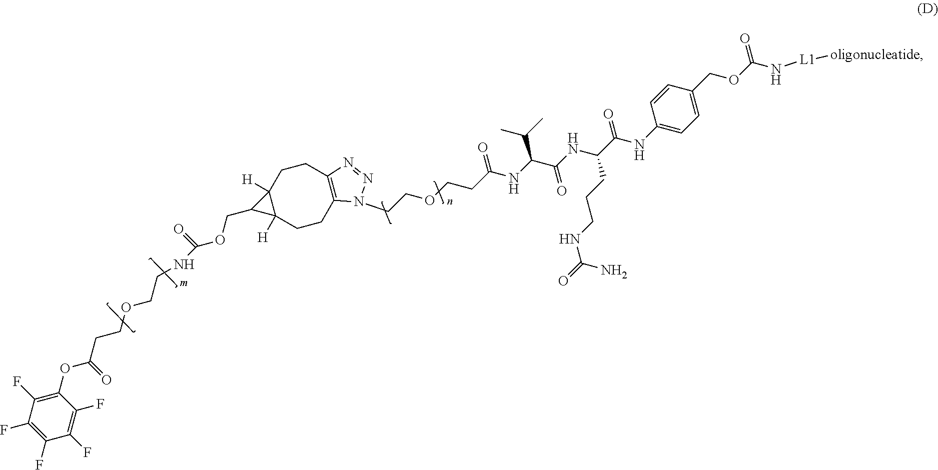

wherein n is any number from 0-10, and wherein m is any number from 0-10. In some embodiments, n is 3 and m is 4.

In some embodiments, the compound of structure (D) is further covalently linked to a lysine of the anti-TfR1 antibody, forming a complex comprising a structure of:

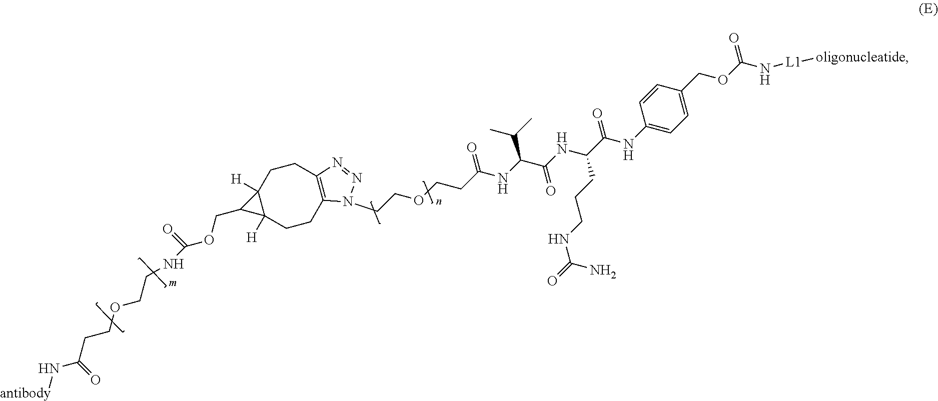

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the compound of Formula (C) is further covalently linked to a lysine of the anti-TfR1 antibody, forming a compound comprising a structure of:

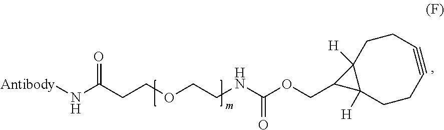

wherein m is 0-15 (e.g., 4). It should be understood that the amide shown adjacent the anti-TfR1 antibody in Formula (F) results from a reaction with an amine of the anti-TfR1 antibody, such as a lysine epsilon amine.

In some embodiments, the azide of the compound of structure (B) forms a triazole via a click reaction with the alkyne of the compound of structure (F), forming a complex comprising a structure of:

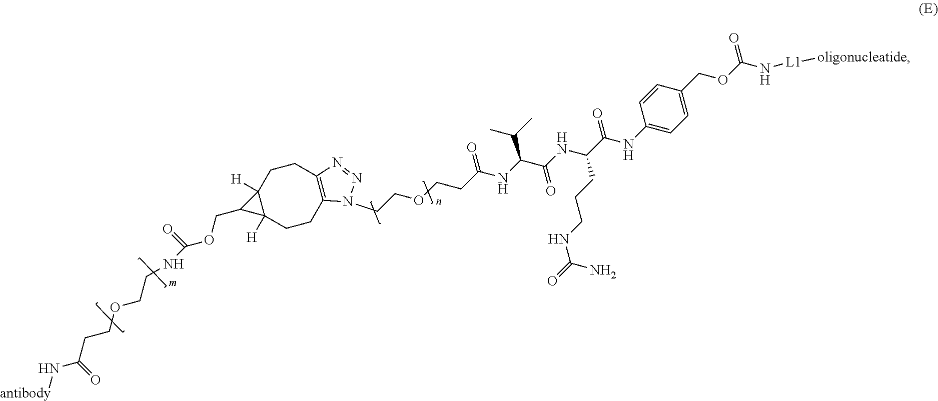

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the azide of the compound of structure (A) forms a triazole via a click reaction with the alkyne of the compound of structure (F), forming a compound comprising a structure of:

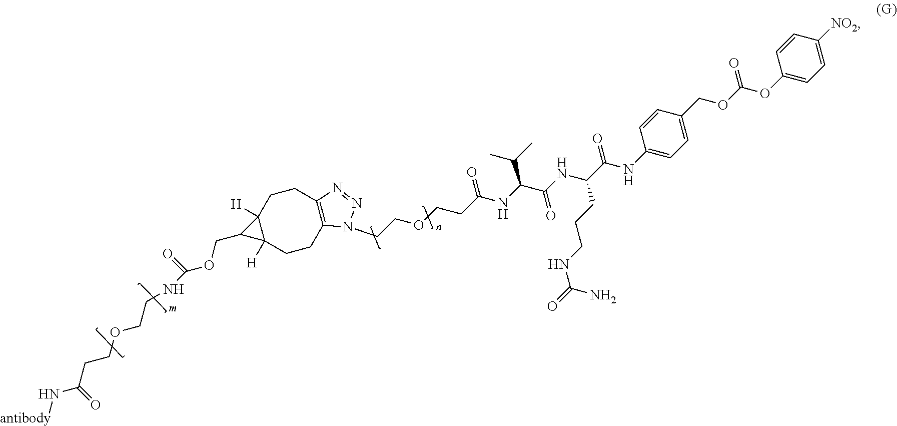

wherein n is any number from 0-10, wherein m is any number from 0-10. In some embodiments, n is 3 and/or (e.g., and) m is 4. In some embodiments, an oligonucleotide is covalently linked to a compound comprising a structure of formula (G), thereby forming a complex comprising a structure of formula (E). It should be understood that the amide shown adjacent the anti-TfR1 antibody in Formula (G) results from a reaction with an amine of the anti-TfR1 antibody, such as a lysine epsilon amine.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

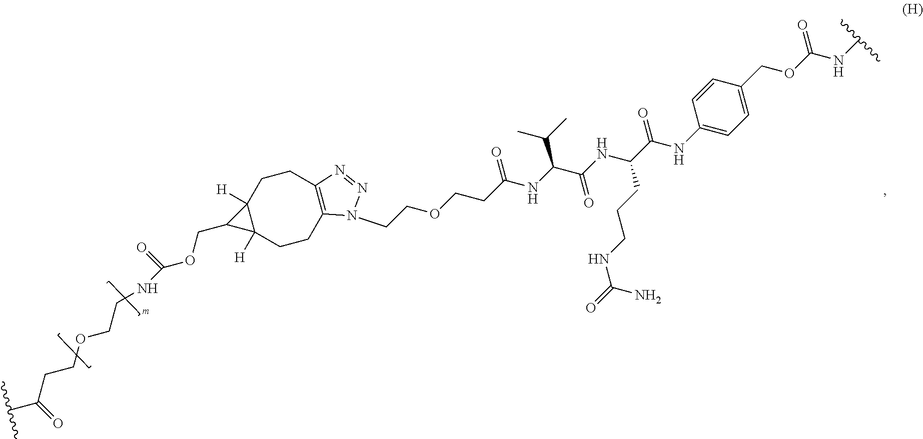

wherein n is any number from 0-10, wherein m is any number from 0-10. In some embodiments, n is 3 and/or (e.g., and) m is 4.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

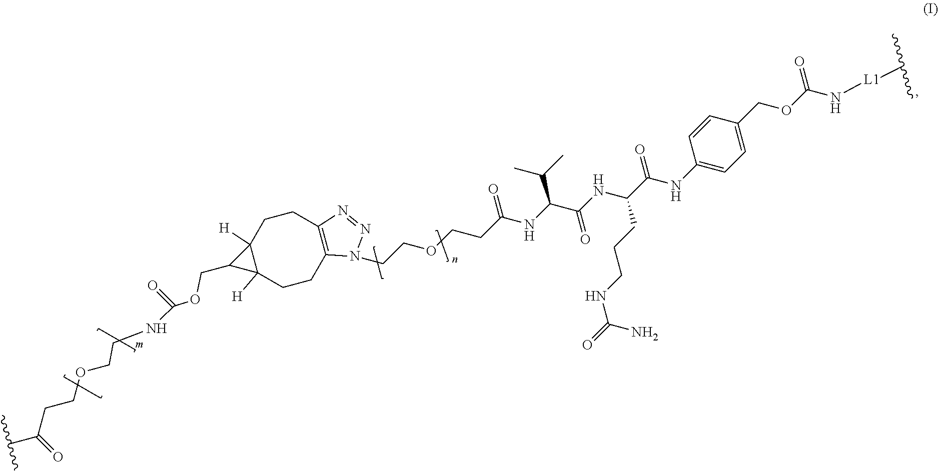

wherein n is any number from 0-10, wherein m is any number from 0-10. In some embodiments, n is 3 and/or (e.g., and) m is 4.

In some embodiments, in formulae (B), (D), (E), and (I), L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof, wherein each R^{A} is independently hydrogen or substituted or unsubstituted alkyl. In some embodiments, L1 is

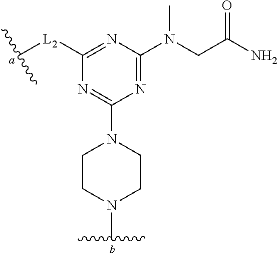

wherein L2 is

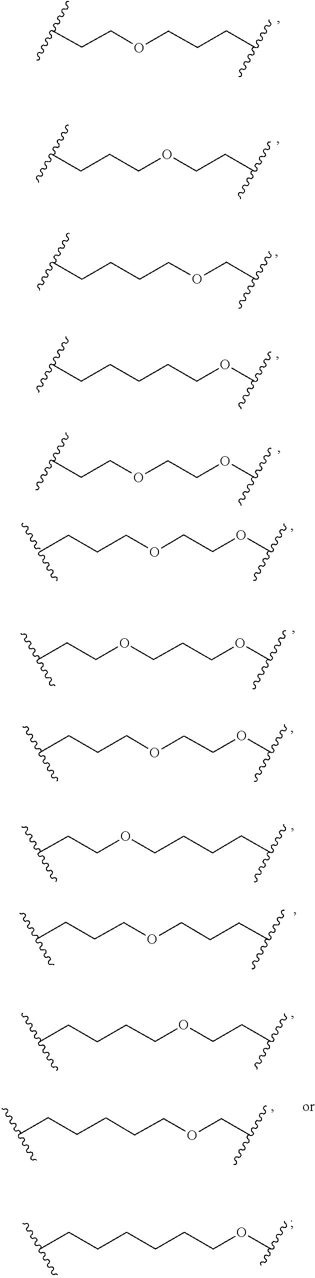

wherein a labels the site directly linked to the carbamate moiety of formulae (B), (D), (E), and (I); and b labels the site covalently linked (directly or via additional chemical moieties) to the oligonucleotide.

In some embodiments, L1 is:

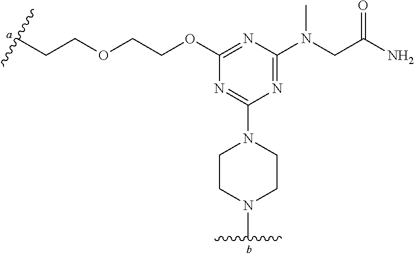 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is

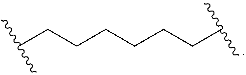 [SMILES: *CCCCCC*]

In some embodiments, L1 is linked to a 5′ phosphate of the oligonucleotide. In some embodiments, the phosphate is a phosphodiester. In some embodiments, L1 is linked to a 5′ phosphorothioate of the oligonucleotide. In some embodiments, L1 is linked to a 5′ phosphonoamidate of the oligonucleotide. In some embodiments, L1 is linked via a phosphorodiamidate linkage to the 5′ end of the oligonucleotide.

In some embodiments, L1 is optional (e.g., need not be present).

In some embodiments, any one of the complexes described herein has a structure of:

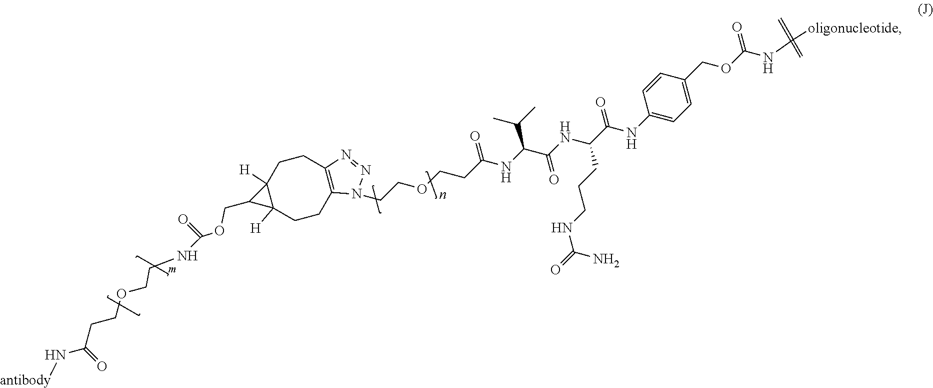

wherein n is 0-15 (e.g., 3) and m is 0-15 (e.g., 4). It should be understood that the amide shown adjacent the anti-TfR1 antibody in Formula (J) results from a reaction with an amine of the anti-TfR1 antibody, such as a lysine epsilon amine.

In some embodiments, any one of the complexes described herein has a structure of:

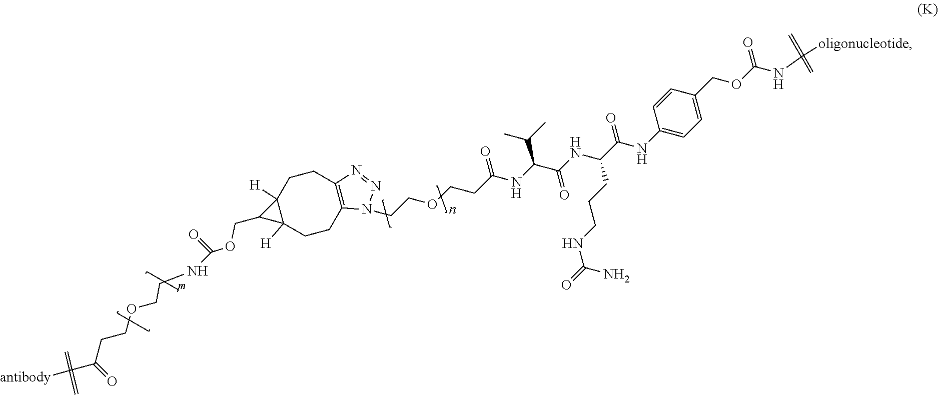

wherein n is 0-15 (e.g., 3) and m is 0-15 (e.g., 4).

In some embodiments, the oligonucleotide is modified to comprise an amine group at the 5′ end, the 3′ end, or internally (e.g., as an amine functionalized nucleobase), prior to linking to a compound, e.g., a compound of formula (A) or formula (G).

Although linker conjugation is described in the context of anti-TfR1 antibodies and oligonucleotide molecular payloads, it should be understood that use of such linker conjugation on other muscle-targeting agents, such as other muscle-targeting antibodies, and/or on other molecular payloads is contemplated.

### D. Examples of Antibody-Molecular Payload Complexes

Further provided herein are non-limiting examples of complexes comprising any one the anti-TfR1 antibodies described herein covalently linked to any of the molecular payloads (e.g., an oligonucleotide) described herein. In some embodiments, the anti-TfR1 antibody (e.g., any one of the anti-TfR1 antibodies provided in Tables 2-7) is covalently linked to a molecular payload (e.g., an oligonucleotide such as the oligonucleotides provided in Table 8) via a linker. Any of the linkers described herein may be used. In some embodiments, if the molecular payload is an oligonucleotide, the linker is linked to the 5′ end of the oligonucleotide, the 3′ end of the oligonucleotide, or to an internal site of the oligonucleotide. In some embodiments, the linker is linked to the anti-TfR1 antibody via a thiol-reactive linkage (e.g., via a cysteine in the anti-TfR1 antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

An example of a structure of a complex comprising an anti-TfR1 antibody covalently linked to a molecular payload via a linker is provided below:

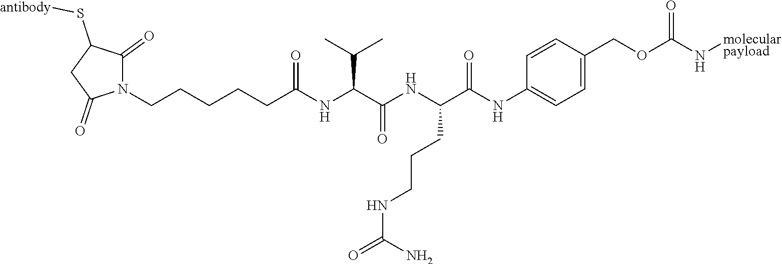

wherein the linker is linked to the antibody via a thiol-reactive linkage (e.g., via a cysteine in the antibody). In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

Another example of a structure of a complex comprising an anti-TfR1 antibody covalently linked to a molecular payload via a linker is provided below:

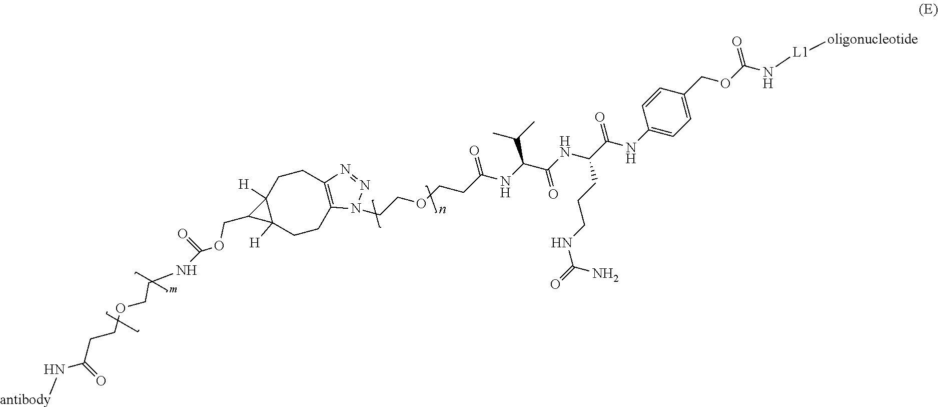

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, the linker is linked to the antibody via a lysine, the linker is linked to the oligonucleotide at the 5′ end, n is 3, and m is 4. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779). It should be understood that the amide shown adjacent the anti-TfR1 antibody in Formula (E) results from a reaction with an amine of the anti-TfR1 antibody, such as a lysine epsilon amine.

It should be appreciated that antibodies can be linked to molecular payloads with different stoichiometries, a property that may be referred to as a drug to antibody ratios (DAR) with the “drug” being the molecular payload. In some embodiments, one molecular payload is linked to an antibody (DAR=1). In some embodiments, two molecular payloads are linked to an antibody (DAR=2). In some embodiments, three molecular payloads are linked to an antibody (DAR=3). In some embodiments, four molecular payloads are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. An average DAR of complexes in a mixture need not be an integer value. DAR may be increased by conjugating molecular payloads to different sites on an antibody and/or (e.g., and) by conjugating multimers to one or more sites on antibody. For example, a DAR of 2 may be achieved by conjugating a single molecular payload to two different sites on an antibody or by conjugating a dimer molecular payload to a single site of an antibody.

In some embodiments, the complex described herein comprises an anti-TfR1 antibody described herein (e.g., the antibodies provided in Tables 2-7) covalently linked to a molecular payload. In some embodiments, the complex described herein comprises an anti-TfR1 antibody described herein (e.g., the antibodies provided in Tables 2-7) covalently linked to molecular payload via a linker (e.g., a linker comprising a valine-citrulline sequence). In some embodiments, the linker (e.g., a linker comprising a valine-citrulline sequence) is linked to the antibody (e.g., an anti-TfR1 antibody described herein) via a thiol-reactive linkage (e.g., via a cysteine in the antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a CDR-H1, a CDR-H2, a CDR-H3, a CDR-L1, a CDR-L2, and a CDR-L3 of any one of the antibodies listed in Table 2. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a VH comprising the amino acid sequence of SEQ ID NO: 69, SEQ ID NO: 71, or SEQ ID NO: 72, and a VL comprising the amino acid sequence of SEQ ID NO: 70. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 74. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 75. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a VH comprising the amino acid sequence of SEQ ID NO: 77, and a VL comprising the amino acid sequence of SEQ ID NO: 78. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a VH comprising the amino acid sequence of SEQ ID NO: 77 or SEQ ID NO: 79, and a VL comprising the amino acid sequence of SEQ ID NO: 80. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a VH comprising the amino acid sequence of SEQ ID NO: 154, and a VL comprising the amino acid sequence of SEQ ID NO: 155. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 84, SEQ ID NO: 86 or SEQ ID NO: 87 and a light chain comprising the amino acid sequence of SEQ ID NO: 85. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 89. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 90. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92 or SEQ ID NO: 94, and a light chain comprising the amino acid sequence of SEQ ID NO: 95. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92, and a light chain comprising the amino acid sequence of SEQ ID NO: 93. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 156, and a light chain comprising the amino acid sequence of SEQ ID NO: 157. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 97, SEQ ID NO: 98, or SEQ ID NO: 99 and a light chain comprising the amino acid sequence of SEQ ID NO: 85. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 89. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 90. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 and a light chain comprising the amino acid sequence of SEQ ID NO: 93. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 or SEQ ID NO: 103 and a light chain comprising the amino acid sequence of SEQ ID NO: 95. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In some embodiments, the complex described herein comprises an anti-TfR1 antibody covalently linked to a molecular payload, wherein the anti-TfR1 antibody comprises a heavy chain comprising the amino acid sequence of SEQ ID NO: 158 or SEQ ID NO: 159 and a light chain comprising the amino acid sequence of SEQ ID NO: 157. In some embodiments, the molecular payload is a DMD-targeting oligonucleotide (e.g., a DMD-targeting oligonucleotide listed in Table 8, provided by any one of SEQ ID NO: 780-2019, or complementary to any one of SEQ ID NO: 160-779).

In any of the example complexes described herein, in some embodiments, the anti-TfR1 antibody is covalently linked to the molecular payload via a linker comprising a structure of:

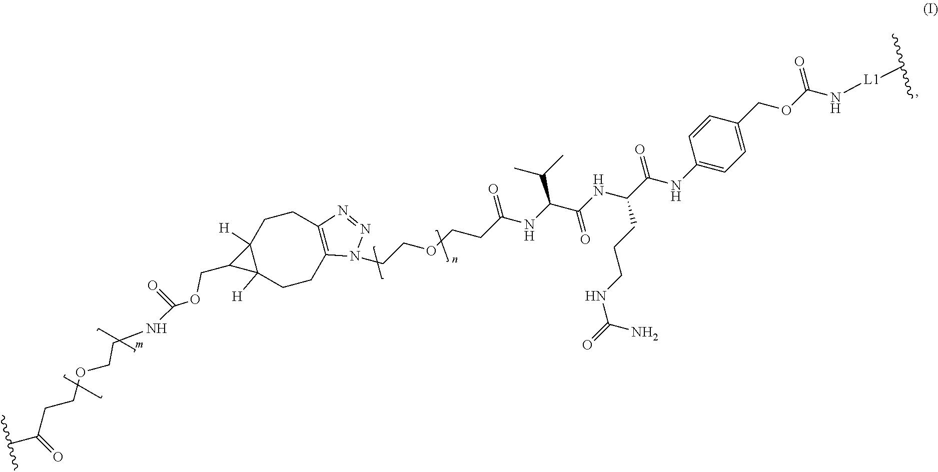

wherein n is 3, m is 4.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 780-2019, or complementary to any one of SEQ ID NO: 160-779) via a lysine in the anti-TfR1 antibody, wherein the anti-TfR1 antibody comprises a CDR-H1, a CDR-H2, a CDR-H3, a CDR-L1, a CDR-L2, and a CDR-L3 of any one of the antibodies listed in Table 2, wherein the complex has a structure of:

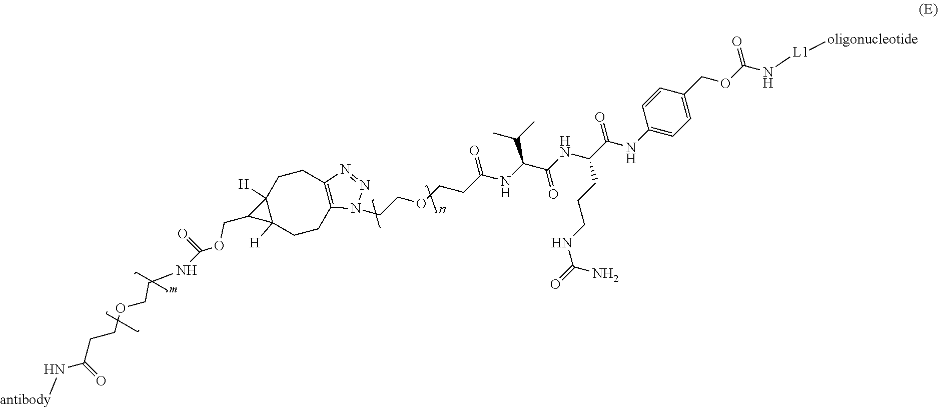

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 780-2019, or complementary to any one of SEQ ID NO: 160-779) via a lysine in the anti-TfR1 antibody, wherein the anti-TfR1 antibody comprises a VH and VL of any one of the antibodies listed in Table 3, wherein the complex has a structure of:

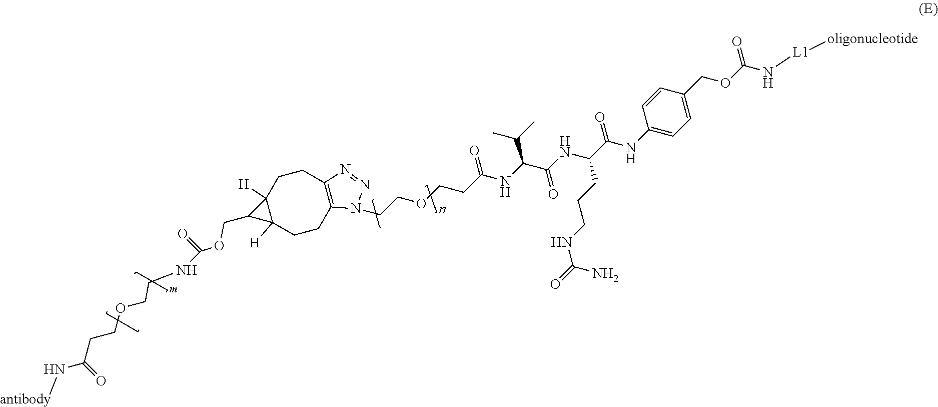

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 780-2019, or complementary to any one of SEQ ID NO: 160-779) via a lysine in the anti-TfR1 antibody, wherein the anti-TfR1 antibody comprises a heavy chain and light chain of any one of the antibodies listed in Table 4, wherein the complex has a structure of:

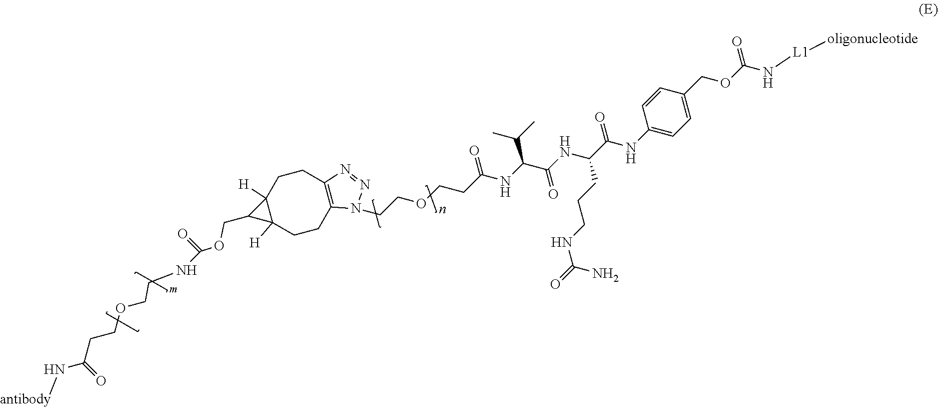

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 Fab covalently linked to the 5′ end of a DMD-targeting oligonucleotide (e.g., a DMD-targeting oligonucleotide listed in Table 8, provided by any one of SEQ ID NO: 780-2019, or complementary to any one of SEQ ID NO: 160-779) via a lysine in the anti-TfR1 antibody, wherein the anti-TfR1 Fab comprises a heavy chain and light chain of any one of the antibodies listed in Table 5, wherein the complex has a structure of:

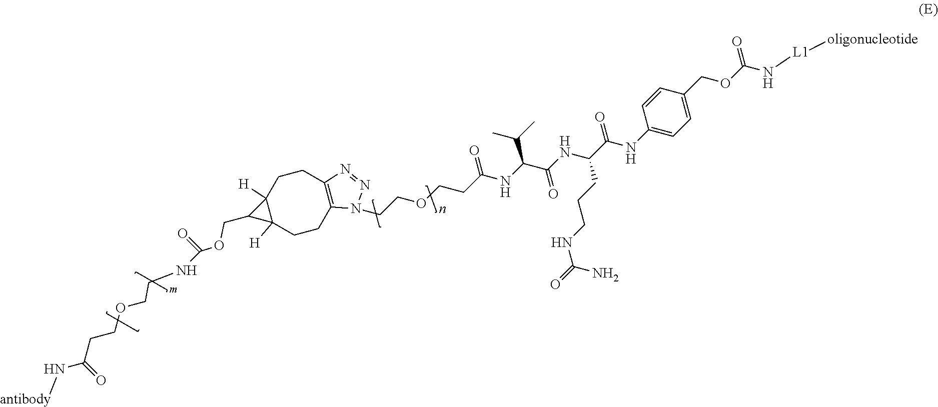

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, in any one of the examples of complexes described herein, L1 is:

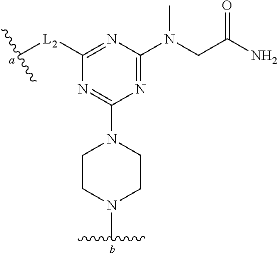

wherein L2 is

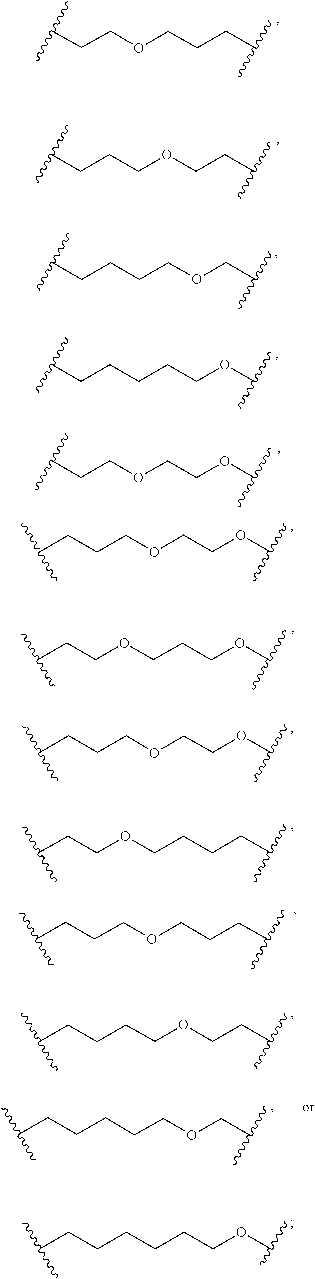

wherein a labels the site directly linked to the carbamate moiety of formulae (B), (D), (E), and (I); and b labels the site covalently linked (directly or via additional chemical moieties) to the oligonucleotide.

In some embodiments, L1 is:

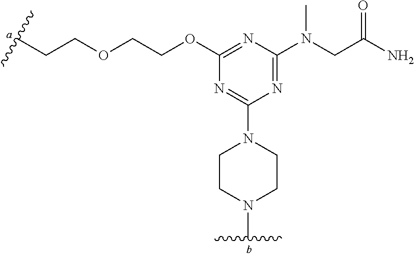 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is linked to a 5′ phosphate of the oligonucleotide. In some embodiments, the phosphate is a phosphodiester. In some embodiments, L1 is linked to a 5′ phosphorothioate of the oligonucleotide. In some embodiments, L1 is linked to a 5′ phosphonoamidate of the oligonucleotide. In some embodiments, L1 is linked via a phosphorodiamidate linkage to the 5′ end of the oligonucleotide.

In some embodiments, L1 is optional (e.g., need not be present).

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or (e.g., and) uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or (e.g., and) therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently linked to a molecular payload as described herein are effective in treating a subject having a dystrophinopathy, e.g., Duchenne muscular dystrophy. In some embodiments, complexes comprise a molecular payload that is an oligonucleotide, e.g., an antisense oligonucleotide that facilitates exon skipping of a pre-mRNA expressed from a mutated DMD allele.

In some embodiments, a subject may be a human subject, a non-human primate subject, a rodent subject, or any suitable mammalian subject. In some embodiments, a subject may have Duchenne muscular dystrophy or other dystrophinopathy. In some embodiments, a subject has a mutated DMD allele, which may optionally comprise at least one mutation in a DMD exon that causes a frameshift mutation and leads to improper RNA splicing/processing. In some embodiments, a subject is suffering from symptoms of a severe dystrophinopathy, e.g. muscle atrophy or muscle loss. In some embodiments, a subject has an asymptomatic increase in serum concentration of creatine phosphokinase (CK) and/or (e.g., and) muscle cramps with myoglobinuria. In some embodiments, a subject has a progressive muscle disease, such as Duchenne or Becker muscular dystrophy or DMD-associated dilated cardiomyopathy (DCM). In some embodiments, a subject is not suffering from symptoms of a dystrophinopathy.

In some embodiments, a subject has a mutation in a DMD gene that is amenable to exon 55 skipping. In some embodiments, a complex comprising a muscle-targeting agent covalently linked to a molecular payload as described herein is effective in treating a subject having a mutation in a DMD gene that is amenable to exon 55 skipping. In some embodiments, a complex comprises a molecular payload that is an oligonucleotide, e.g., an antisense oligonucleotide that facilitates skipping of exon 55 of a pre-mRNA, such as in a pre-mRNA encoded from a mutated DMD gene (e.g., a mutated DMD gene that is amenable to exon 55 skipping).

An aspect of the disclosure includes methods involving administering to a subject an effective amount of a complex as described herein. In some embodiments, an effective amount of a pharmaceutical composition that comprises a complex comprising a muscle-targeting agent covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently linked to a molecular payload is administered at an effective concentration that confers therapeutic effect on a subject. Effective amounts vary, as recognized by those skilled in the art, depending on the severity of the disease, unique characteristics of the subject being treated, e.g., age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g., the half-life of the complex in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of observation of symptoms associated with a dystrophinopathy, e.g., muscle atrophy or muscle weakness, through measures of a subject's self-reported outcomes, e.g., mobility, self-care, usual activities, pain/discomfort, and anxiety/depression, or by quality-of-life indicators, e.g., lifespan.

In some embodiments, a pharmaceutical composition that comprises a complex comprising a muscle-targeting agent covalently linked to a molecular payload described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### ADDITIONAL EMBODIMENTS

1. A complex comprising an anti-transferrin receptor 1 (TfR1) antibody covalently linked to a molecular payload configured for inducing skipping of exon 55 in a DMD pre-mRNA, wherein the anti-TfR1 antibody is an antibody identified in any one of Tables 2-7.
2. The complex of embodiment 1, wherein the anti-TfR1 antibody comprises:
(i) a heavy chain complementarity determining region 1 (CDR-H1) of SEQ ID NO: 33, a heavy chain complementarity determining region 2 (CDR-H2) of SEQ ID NO: 34, a heavy chain complementarity determining region 3 (CDR-H3) of SEQ ID NO: 35, a light chain complementarity determining region 1 (CDR-L1) of SEQ ID NO: 36, a light chain complementarity determining region 2 (CDR-L2) of SEQ ID NO: 37, and a light chain complementarity determining region 3 (CDR-L3) of SEQ ID NO: 32;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6;
(iii) a CDR-H1 of SEQ ID NO: 7, a CDR-H2 of SEQ ID NO: 20, a CDR-H3 of SEQ ID NO: 9, a CDR-L1 of SEQ ID NO: 10, a CDR-L2 of SEQ ID NO: 11, and a CDR-L3 of SEQ ID NO: 6;
(iv) a CDR-H1 of SEQ ID NO: 7, a CDR-H2 of SEQ ID NO: 24, a CDR-H3 of SEQ ID NO: 9, a CDR-L1 of SEQ ID NO: 10, a CDR-L2 of SEQ ID NO: 11, and a CDR-L3 of SEQ ID NO: 6;
(v) a CDR-H1 of SEQ ID NO: 51, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50;
(vi) a CDR-H1 of SEQ ID NO: 64, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50; or
(vii) a CDR-H1 of SEQ ID NO: 67, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50.
   3. The complex of embodiment 1 or embodiment 2, wherein the anti-TfR1 antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a VH comprising an amino acid sequence at least 85% identical to SEQ ID NO: 69; and/or a VL comprising an amino acid sequence at least 85% identical to SEQ ID NO: 70;
(iii) a VH comprising an amino acid sequence at least 85% identical to SEQ ID NO: 71; and/or a VL comprising an amino acid sequence at least 85% identical to SEQ ID NO: 70;
(iv) a VH comprising an amino acid sequence at least 85% identical to SEQ ID NO: 72; and/or a VL comprising an amino acid sequence at least 85% identical to SEQ ID NO: 70;
(v) a VH comprising an amino acid sequence at least 85% identical to SEQ ID NO: 73; and/or a VL comprising an amino acid sequence at least 85% identical to SEQ ID NO: 74;
(vi) a VH comprising an amino acid sequence at least 85% identical to SEQ ID NO: 73; and/or a VL comprising an amino acid sequence at least 85% identical to SEQ ID NO: 75;
(vii) a VH comprising an amino acid sequence at least 85% identical to SEQ ID NO: 76; and/or a VL comprising an amino acid sequence at least 85% identical to SEQ ID NO: 74;
(viii) a VH comprising an amino acid sequence at least 85% identical to SEQ ID NO: 77; and/or a VL comprising an amino acid sequence at least 85% identical to SEQ ID NO: 78;
(ix) a VH comprising an amino acid sequence at least 85% identical to SEQ ID NO: 79; and/or a VL comprising an amino acid sequence at least 85% identical to SEQ ID NO: 80; or
(x) a VH comprising an amino acid sequence at least 85% identical to SEQ ID NO: 77; and/or a VL comprising an amino acid sequence at least 85% identical to SEQ ID NO: 80.
   4. The complex of any one of embodiments 1 to 3, wherein the anti-TfR1 antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 69 and a VL comprising the amino acid sequence of SEQ ID NO: 70;
(iii) a VH comprising the amino acid sequence of SEQ ID NO: 71 and a VL comprising the amino acid sequence of SEQ ID NO: 70;
(iv) a VH comprising the amino acid sequence of SEQ ID NO: 72 and a VL comprising the amino acid sequence of SEQ ID NO: 70;
(v) a VH comprising the amino acid sequence of SEQ ID NO: 73 and a VL comprising the amino acid sequence of SEQ ID NO: 74;
(vi) a VH comprising the amino acid sequence of SEQ ID NO: 73 and a VL comprising the amino acid sequence of SEQ ID NO: 75;
(vii) a VH comprising the amino acid sequence of SEQ ID NO: 76 and a VL comprising the amino acid sequence of SEQ ID NO: 74;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.
   5. The complex of any one of embodiments 1 to 4, wherein the anti-TfR1 antibody is a Fab fragment, a Fab′ fragment, a F(ab′)2 fragment, an scFv, an Fv, or a full-length IgG.
   6. The complex of embodiment 5, wherein the anti-TfR1 antibody is a Fab fragment.
   7. The complex of embodiment 6, wherein the anti-TfR1 antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 97; and/or a light chain comprising an amino acid sequence at least 85% identical to SEQ ID NO: 85;
(iii) a heavy chain comprising an amino acid sequence at least 85% identical to SEQ ID NO: 98; and/or a light chain comprising an amino acid sequence at least 85% identical to SEQ ID NO: 85;
(iv) a heavy chain comprising an amino acid sequence at least 85% identical to SEQ ID NO: 99; and/or a light chain comprising an amino acid sequence at least 85% identical to SEQ ID NO: 85;
(v) a heavy chain comprising an amino acid sequence at least 85% identical to SEQ ID NO: 100; and/or a light chain comprising an amino acid sequence at least 85% identical to SEQ ID NO: 89;
(vi) a heavy chain comprising an amino acid sequence at least 85% identical to SEQ ID NO: 100; and/or a light chain comprising an amino acid sequence at least 85% identical to SEQ ID NO: 90;
(vii) a heavy chain comprising an amino acid sequence at least 85% identical to SEQ ID NO: 101; and/or a light chain comprising an amino acid sequence at least 85% identical to SEQ ID NO: 89;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.
   8. The complex of embodiment 6 or embodiment 7, wherein the anti-TfR1 antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 97; and a light chain comprising the amino acid sequence of SEQ ID NO: 85;
(iii) a heavy chain comprising the amino acid sequence of SEQ ID NO: 98; and a light chain comprising the amino acid sequence of SEQ ID NO: 85;
(iv) a heavy chain comprising the amino acid sequence of SEQ ID NO: 99; and a light chain comprising the amino acid sequence of SEQ ID NO: 85;
(v) a heavy chain comprising the amino acid sequence of SEQ ID NO: 100; and a light chain comprising the amino acid sequence of SEQ ID NO: 89;
(vi) a heavy chain comprising the amino acid sequence of SEQ ID NO: 100; and a light chain comprising the amino acid sequence of SEQ ID NO: 90;
(vii) a heavy chain comprising the amino acid sequence of SEQ ID NO: 101; and a light chain comprising the amino acid sequence of SEQ ID NO: 89;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.
   9. The complex of any one of embodiments 1 to 8, wherein the anti-TfR1 antibody does not specifically bind to the transferrin binding site of the transferrin receptor 1 and/or wherein the anti-TfR1 antibody does not inhibit binding of transferrin to the transferrin receptor 1.
   10. The complex of any one of embodiments 1 to 9, wherein the molecular payload comprises an oligonucleotide.
   11. The complex of embodiment 10, wherein the oligonucleotide promotes antisense-mediated exon skipping in the DMD pre-RNA.
   12. The complex of embodiment 10 or 11, wherein the oligonucleotide comprises a region of complementarity to a splicing feature of the DMD pre-mRNA.
   13. The complex of embodiment 12, wherein the splicing feature is an exonic splicing enhancer (ESE) of the DMD pre-mRNA.
   14. The complex of embodiment 13, wherein the splicing feature is in exon 55 of the DMD pre-mRNA, optionally wherein the ESE comprises a sequence of any one of SEQ ID NOs: 2031-2061.
   15. The complex of embodiment 12, wherein the splicing feature is a branch point, a splice donor site, or a splice acceptor site.
   16. The complex of embodiment 15, wherein the splicing feature is across the junction of exon 54 and intron 54, in intron 54, across the junction of intron 54 and exon 55, across the junction of exon 55 and intron 55, in intron 55, or across the junction of intron 55 and exon 56 of the DMD pre-mRNA, optionally wherein the splicing feature comprises a sequence of any one of SEQ ID NOs: 2028-2030, 2062, and 2063.
   17. The complex of any one of embodiments 12 to 16, wherein the region of complementarity comprises at least 4 consecutive nucleosides complementary to the splicing feature.
   18. The complex of any one of embodiments 1 to 9, wherein the molecular payload comprises an oligonucleotide comprising a sequence complementary to any one of SEQ ID NOs: 160-779 or comprising a sequence of any one of SEQ ID NOs: 780-2019, wherein each thymine base (T) may independently and optionally be replaced with a uracil base (U), and each U may independently and optionally be replaced with a T.
   19. The complex of any one of embodiments 10 to 18, wherein the oligonucleotide comprises at least one modified internucleoside linkage.
   20. The complex of embodiment 19, wherein the at least one modified internucleoside linkage is a phosphorothioate linkage.
   21. The complex of any one of embodiments 10 to 20, wherein the oligonucleotide comprises one or more modified nucleosides.
   22. The complex of embodiment 21, wherein the one or more modified nucleosides are 2′-modified nucleosides.
   23. The complex of any one of embodiments 10 to 18, wherein the oligonucleotide comprises one or more phosphorodiamidate morpholinos, optionally wherein the oligonucleotide is a phosphorodiamidate morpholino oligomer (PMO).
   24. The complex of any one of embodiments 1 to 23, wherein the anti-TfR1 antibody is covalently linked to the molecular payload via a cleavable linker.
   25. The complex of embodiment 24, wherein the cleavable linker comprises a valine-citrulline sequence.
   26. The complex of any one of embodiments 1 to 25, wherein the anti-TfR1 antibody is covalently linked to the molecular payload via conjugation to a lysine residue or a cysteine residue of the antibody.
   27. A complex comprising an anti-TfR1 antibody covalently linked to an oligonucleotide configured for inducing skipping of exon 55 in a DMD pre-mRNA, wherein the oligonucleotide comprises a region of complementarity to any one of SEQ ID NOs: 160-779.
   28. The complex of embodiment 27, wherein the anti-TfR1 antibody is an antibody identified in any one of Tables 2-7.
   29. A complex comprising an anti-TfR1 antibody covalently linked to an oligonucleotide configured for inducing skipping of exon 55 in a DMD pre-mRNA, wherein the oligonucleotide comprises a region of complementarity to a splicing feature of the DMD pre-mRNA.
   30. An oligonucleotide that targets DMD, wherein the oligonucleotide comprises a region of complementarity to any one of SEQ ID NOs: 160-779.
   31. The oligonucleotide of embodiment 30, wherein the region of complementarity comprises at least 15 consecutive nucleosides complementary to any one of SEQ ID NOs: 160-779.
   32. The oligonucleotide of embodiment 30 or 31, wherein the oligonucleotide comprises at least 15 consecutive nucleosides of any one of SEQ ID NOs: 780-2019, optionally wherein the oligonucleotide comprises a sequence of any one of SEQ ID NOs: 780-2019, wherein each thymine base (T) may independently and optionally be replaced with a uracil base (U), and each U may independently and optionally be replaced with a T.
   33. A method of delivering a molecular payload to a cell, the method comprising contacting the cell with the complex of any one of embodiments 1 to 26.
   34. A method of delivering an oligonucleotide to a cell, the method comprising contacting the cell with the complex of any one of embodiments 27 to 29.
   35. A method of promoting the expression or activity of a dystrophin protein in a cell, the method comprising contacting the cell with the complex of any one of embodiments 1 to 26 in an amount effective for promoting internalization of the molecular payload to the cell, optionally wherein the cell is a muscle cell.
   36. A method of promoting the expression or activity of a dystrophin protein in a cell, the method comprising contacting the cell with the complex of any one of embodiments 27 to 29 in an amount effective for promoting internalization of the oligonucleotide to the cell, optionally wherein the cell is a muscle cell.
   37. The method of embodiment 35 or 36, wherein the cell is in vitro.
   38. The method of embodiment 35 or 36, wherein the cell is in a subject.
   39. The method of embodiment 38, wherein the subject is a human.
   40. The method of embodiment 39, wherein the subject has a DMD gene that is amenable to skipping of exon 55.
   41. The method of any one of embodiments 35 to 40, wherein the dystrophin protein is a truncated dystrophin protein.
   42. A method of treating a subject having a mutated DMD allele that is associated with a dystrophinopathy, the method comprising administering to the subject an effective amount of the complex of any one of embodiments 1 to 29.
   43. A method of promoting skipping of exon 55 of a DMD pre-mRNA transcript in a cell, the method comprising contacting the cell with an effective amount of the complex of any one of embodiments 1 to 29.
   44. A method of treating a subject having a mutated DMD allele that is associated with a dystrophinopathy, the method comprising administering to the subject an effective amount of the complex of any one of embodiments 1 to 29.

### EXAMPLES

### Example 1. Exon-Skipping Activity of Anti-TfR1 Antibody Conjugates in Duchenne Muscular Dystrophy Patient Myotubes

In this study, the exon-skipping activities of anti-TfR1 antibody conjugates comprising an anti-TfR1 Fab (3M12 VH4/Vκ3) covalently linked to a DMD exon 51-skipping antisense oligonucleotide (ASO) were evaluated. The DMD exon 51-skipping ASO is a phosphorodiamidate morpholino oligomer (PMO) of 30 nucleotides in length and targets an ESE in DMD exon 51 having the sequence TGGAGGT (SEQ ID NO: 131). Immortalized human myoblasts bearing an exon 52 deletion in the DMD gene were thawed and seeded at a density of 1e6 cell/flask in Promocell Skeletal Cell Growth Media (with 5% FBS and 1× Pen-Strep) and allowed to grow to confluency. Once confluent, cells were trypsinized and pelleted via centrifugation and resuspended in fresh Promocell Skeletal Cell Growth Media. The cell number was counted and cells were seeded into Matrigel-coated 96-well plates at a density of 50,000 cells/well. Cells were allowed to recover for 24 hours. Cells were induced to differentiate into myotubes by aspirating the growth media and replacing with differentiation media with no serum. Cells were then treated with the DMD exon 51-skipping oligonucleotide (not covalently linked to an antibody—“naked”) at 10 pM ASO or the anti-TfR1 Fab (3M12 VH4/Vκ3) covalently linked to the DMD exon 51-skipping oligonucleotide at 10 μM ASO equivalent. Cells were incubated with test articles for ten days then total RNA was harvested from the 96 well plates. cDNA synthesis was performed on 75 ng of total RNA, and mutation specific PCRs were performed to evaluate the degree of exon 51 skipping in the cells. Mutation-specific PCR products were run on a 4% agarose gel and visualized using SYBR gold. Densitometry was used to calculate the relative amounts of the skipped and unskipped amplicon and exon skipping was determined as a ratio of the Exon 51 skipped amplicon divided by the total amount of amplicon present:

$$\% Exon Skipping = \frac{Skipped Amplicon}{( Skipped Amplicon + Unskipped Amplicon )} * 100.$$

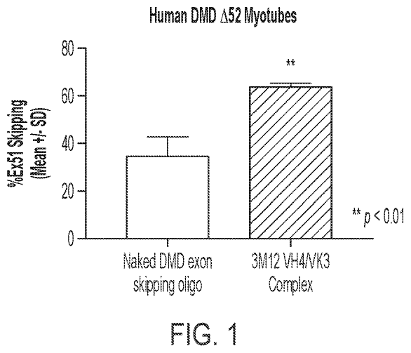

The results demonstrate that the conjugate resulted in enhanced exon skipping compared to the naked DMD exon 51-skipping oligonucleotide in patient myotubes (FIG. 1). This indicates that anti-TfR1 Fab 3M12 VH4/Vκ3 enabled cellular internalization of the conjugate into muscle cells resulting in activity of the exon 51-skipping oligonucleotide in the muscle cells. Similarly, an anti-TfR1 antibody (e.g., anti-TfR1 Fab 3M12 VH4/Vκ3) can enable internalization of a conjugate comprising the anti-TfR1 antibody covalently linked to other exon skipping oligonucleotides (e.g., an exon skipping oligonucleotide provided herein, such as an exon 55 skipping oligonucleotide) into muscle cells and facilitate activity of the exon skipping oligonucleotide in the muscle cells.

### Example 2. Exon Skipping Activity of Anti-TfR1 Fab-ASO Conjugate In Vivo in Cynomolgus Monkeys

Anti-TfR1 Fab 3M12 VH4/Vκ3 was covalently linked to the DMD exon 51-skipping antisense oligonucleotide (ASO) that was used in Example 1. The exon skipping activity of the conjugate was tested in vivo in healthy non-human primates. Naïve male cynomolgus monkeys (n=4-5 per group) were administered two doses of vehicle, 30 mg/kg naked ASO (i.e., not covalently linked to an antibody), or 122 mg/kg anti-TfR1 Fab (3M12 VH4/Vκ3) covalently linked to the DMD exon 51-skipping oligonucleotide (30 mg/kg ASO equivalent) via intravenous infusion on days 1 and 8. Animals were sacrificed and tissues harvested either 2 weeks or 4 weeks after the first dose was administered. Total RNA was collected from tissue samples using a Promega Maxwell® RSC instrument and cDNA synthesis was performed using qScript cDNA SuperMix. Assessment of exon 51 skipping was performed using end-point PCR.

Capillary electrophoresis of the PCR products was used to assess exon skipping, and % exon 51 skipping was calculated using the following formula:

$$\% Exon Skipping = \frac{Molarity of Skipped Band}{Molarity of Skipped Band + Molarity of Unskipped Band} \times 100.$$

Calculated exon 51 skipping results are shown in Table 10.

TABLE 10
Exon 51 skipping of DMD mRNA in cynomolgus monkey

| | Time | | | | |
|---|---|---|---|---|---|
| | 2 weeks | | | 4 weeks | |
| | | Naked | | Naked | |
| Group | Vehicle | ASO^{a} | Conjugate | ASO^{a} | Conjugate |
| Conjugate dose^{b} | 0 | n/a | 122 | n/a | 122 |
| ASO Dose^{c} | 0 | 30 | 30 | 30 | 30 |
| Quadriceps ^{d} | 0.00 | 1.216 | 4.906 | 0.840 | 1.708 |
| | (0.00) | (1.083) | (3.131) | (1.169) | (1.395) |
| Diaphragm ^{d} | 0.00 | 1.891 | 7.315 | 0.717 | 9.225 |
| | (0.00) | (2.911) | (1.532) | (1.315) | (4.696) |
| Heart ^{d} | 0.00 | 0.043 | 3.42 | 0.00 | 4.525 |
| | (0.00) | (0.096) | (1.192) | (0.00) | (1.400) |
| Biceps ^{d} | 0.00 | 0.607 | 3.129 | 1.214 | 4.863 |
| | (0.00) | (0.615) | (0.912) | (1.441) | (3.881) |
| Tibialis | 0.00 | 0.699 | 1.042 | 0.384 | 0.816 |
| anterior ^{d} | (0.00) | (0.997) | (0.685) | (0.615) | (0.915) |
| Gastrocnemius ^{d} | 0.00 | 0.388 | 2.424 | 0.00 | 5.393 |
| | (0.00) | (0.573) | (2.329) | (0.00) | (2.695) |

^{a}ASO = antisense oligonucleotide.
^{b}Conjugate doses are listed as mg/kg of anti-TfR1 Fab 3M12 VH4/Vκ3-ASO conjugate.
^{c}ASO doses are listed as mg/kg ASO or ASO equivalent of the anti-TfR1 Fab 3M12 VH4/Vκ3-ASO dose.
^{d} Exon skipping values are mean % exon 51 skipping with standard deviations (n = 5) in parentheses.

Tissue ASO accumulation was also quantified using a hybridization ELISA with a probe complementary to the ASO sequence. A standard curve was generated and ASO levels (in ng/g) were derived from a linear regression of the standard curve. The ASO was distributed to all tissues evaluated at a higher level following the administration of the anti-TfR1 Fab VH4/Vκ3-ASO conjugate as compared to the administration of naked ASO. Intravenous administration of naked ASO resulted in levels of ASO that were close to background levels in all tissues evaluated at 2 and 4 weeks after the first does was administered. Administration of anti-TfR1 Fab VH4/Vκ3-ASO conjugate resulted in distribution of ASO through the tissues evaluated with a rank order of heart>diaphragm>bicep>quadriceps>gastrocnemius>tibialis anterior 2 weeks after first dosing. The duration of tissue concentration was also assessed. Concentrations of the ASO in quadriceps, bicep and diaphragm decreased by less than 50% over the time period evaluated (2 to 4 weeks), while levels of ASO in the heart, tibialis anterior, and gastrocnemius remained virtually unchanged (Table 11). This indicates that anti-TfR1 Fab 3M12 VH4/Vκ3 enabled cellular internalization of the conjugate into muscle cells in vivo, resulting in activity of the exon skipping oligonucleotide in the muscle cells. Similarly, an anti-TfR1 antibody (e.g., anti-TfR1 Fab 3M12 VH4/Vκ3) in vivo can enable internalization of a conjugate comprising the anti-TfR1 antibody covalently linked to other exon skipping oligonucleotides (e.g., an exon skipping oligonucleotide provided herein, such as an exon 55 skipping oligonucleotide) into muscle cells and facilitate activity of the exon skipping oligonucleotide in the muscle cells.

TABLE 11
Tissue distribution of DMD exon 51
skipping ASO in cynomolgus monkeys

| | Time | | | | |
|---|---|---|---|---|---|
| | 2 weeks | | | 4 weeks | |
| | | Naked | Conju- | Naked | Conju- |
| Group | Vehicle | ASO^{a} | gate | ASO^{a} | gate |
| Conjugate Dose^{b} | 0 | n/a | 122 | n/a | 122 |
| ASO Dose^{c} | 0 | 30 | 30 | 30 | 30 |
| Quadriceps ^{d} | 0 | 696.8 | 2436 | 197 | 682 |
| | (59.05) | (868.15) | (954.0) | (134) | (281) |
| Diaphragm ^{d} | 0± | 580.02 | 6750 | 60 | 3131 |
| | (144.3) | (360.11) | (2256) | (120) | (1618) |
| Heart ^{d} | 0 | 1449 | 27138 | 943 | 30410 |
| | (396.03) | (1337) | (6315) | (1803) | (9247) |
| Biceps ^{d} | 0 | 615.63 | 2840 | 130 | 1326 |
| | (69.58) | (335.17) | (980.31) | (80) | (623) |
| Tibialis | 0 | 564.71 | 1591 | 169 | 1087 |
| anterior ^{d} | (76.31) | (327.88) | (253.50) | (110) | (514) |
| Gastrocnemius ^{d} | 0 | 705.47 | 2096 | 170 | 1265 |
| | (41.15) | (863.75) | (474.04) | (69) | (272) |

^{a}ASO = Antisense oligonucleotide.
^{b}Conjugate doses are listed as mg/kg of anti-TfR1 Fab 3M12 VH4/Vκ3-ASO conjugate.
^{c}ASO doses are listed as mg/kg ASO or ASO equivalent of the anti-TfR1 Fab 3M12 VH4/Vκ3-ASO conjugate dose.
^{d} ASO values are mean concentrations of ASO in tissue as ng/g with standard deviations (n = 5) in parentheses.

### Example 3. Exon-Skipping Activity of Anti-TfR1 Antibody Conjugates in DMD Patient Myotubes

In this study, the exon-skipping activities of anti-TfR1 antibody conjugates comprising an anti-TfR1 Fab (3M12 VH4/Vκ3) covalently linked to a DMD exon 55-skipping antisense oligonucleotide (ASO) are evaluated. The DMD exon 55-skipping ASO is a phosphorodiamidate morpholino oligomer (PMO) and targets a DMD exon 55 splicing feature. Immortalized human myoblasts are thawed and seeded at a density of 1e6 cell/flask in Promocell Skeletal Cell Growth Media (with 5% FBS and 1× Pen-Strep) and allowed to grow to confluency. Once confluent, cells are trypsinized and pelleted via centrifugation and resuspended in fresh Promocell Skeletal Cell Growth Media. The cell number is counted and cells are seeded into Matrigel-coated 96-well plates at a density of 50,000 cells/well. Cells are allowed to recover for 24 hours. Cells are induced to differentiate into myotubes by aspirating the growth media and replacing with differentiation media with no serum. Cells are then treated with the DMD exon 55-skipping oligonucleotide (not covalently linked to an antibody—“naked”) at 10 pM ASO or the anti-TfR1 Fab (3M12 VH4/Vκ3) covalently linked to the DMD exon 55-skipping oligonucleotide at 10 pM ASO equivalent. Cells are incubated with test articles for ten days then total RNA is harvested from the 96 well plates. cDNA synthesis is performed on 75 ng of total RNA, and mutation specific PCRs are performed to evaluate the degree of exon 55 skipping in the cells. PCR products are measured using capillary electrophoresis with UV detection. Molarity is calculated and relative amounts of the skipped and unskipped amplicon are determined. Exon skipping is determined as a ratio of the Exon 55 skipped amplicon divided by the total amount of amplicon present, according to the following formula:

$$\% Exon Skipping = \frac{Skipped Amplicon}{( Skipped Amplicon + Unskipped Amplicon )} * 100$$

The results demonstrate that the conjugates facilitate enhanced exon skipping compared to the naked DMD exon 55-skipping oligonucleotide in patient myotubes. This indicates that anti-TfR1 Fab 3M12 VH4/Vκ3 enables cellular internalization of the conjugate into muscle cells resulting in activity of the exon 55-skipping oligonucleotide in the muscle cells.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides or nucleosides (e.g., an RNA counterpart of a DNA nucleoside or a DNA counterpart of an RNA nucleoside) and/or (e.g., and) one or more modified nucleotides/nucleosides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A complex comprising an anti-transferrin receptor 1 (TfR1) antibody covalently linked to an oligonucleotide configured for inducing skipping of exon 55 in a DMD pre-mRNA, wherein the oligonucleotide comprises a region of complementarity that is complementary with at least 8 consecutive nucleotides of any one of SEQ ID NOs: 160-779.

2.-4. (canceled)

5. The complex of claim 1, wherein the anti-TfR1 antibody is a Fab fragment, a Fab′ fragment, a F(ab′)2 fragment, an scFv, an Fv, or a full-length IgG.

6. The complex of claim 5, wherein the anti-TfR1 antibody is a Fab fragment.

7.-8. (canceled)

9. The complex of claim 1, wherein the anti-TfR1 antibody does not specifically bind to the transferrin binding site of the transferrin receptor 1 and/or wherein the anti-TfR1 antibody does not inhibit binding of transferrin to the transferrin receptor 1.

10. The complex of claim 1, wherein the oligonucleotide comprises a region of complementarity to at least 4 consecutive nucleotides of a splicing feature of the DMD pre-mRNA.

11. The complex of claim 10, wherein the splicing feature is an exonic splicing enhancer (ESE) in exon 55 of the DMD pre-mRNA, optionally wherein the ESE comprises a sequence of any one of SEQ ID NOs: 2031-2061.

12. The complex of claim 10, wherein the splicing feature is a branch point, a splice donor site, or a splice acceptor site, optionally wherein the splicing feature is across the junction of exon 54 and intron 54, in intron 54, across the junction of intron 54 and exon 55, across the junction of exon 55 and intron 55, in intron 55, or across the junction of intron 55 and exon 56 of the DMD pre-mRNA, and further optionally wherein the splicing feature comprises a sequence of any one of SEQ ID NOs: 2028-2030, 2062, and 2063.

13. The complex of claim 1, wherein the oligonucleotide comprises a sequence complementary to any one of SEQ ID NOs: 160-779 or comprises a sequence of any one of SEQ ID NOs: 780-2019, wherein each thymine base (T) may independently and optionally be replaced with a uracil base (U), and each U may independently and optionally be replaced with a T.

14. The complex of claim 1, wherein the oligonucleotide comprises a sequence of any one of SEQ ID NOs: 1400, 1402-1406, 1408, 1409, 1413, 1418-1420, 1483-1491, 1493, 1495, 1496, 1502-1506, 1508, 1510-1512, 1514, 1522-1524, 1529-1531, 1534, 1535, 1559, 1583, 1587, 1591, 1596, 1597, 1598, 1604, 1606, 1607, 1638, 1641, 1693-1695, 1702, 1703, 1766, 1813, 1988, and 1995, wherein each thymine base (T) may independently and optionally be replaced with a uracil base (U), and each U may independently and optionally be replaced with a T.

15. The complex of claim 1, wherein the oligonucleotide comprises one or more phosphorodiamidate morpholinos, optionally wherein the oligonucleotide is a phosphorodiamidate morpholino oligomer (PMO).

16. The complex of claim 1, wherein the anti-TfR1 antibody is covalently linked to the oligonucleotide via a cleavable linker, optionally wherein the cleavable linker comprises a valine-citrulline sequence.

17. The complex of claim 1, wherein the anti-TfR1 antibody is covalently linked to the oligonucleotide via conjugation to a lysine residue or a cysteine residue of the antibody.

18. An oligonucleotide that targets DMD, wherein the oligonucleotide comprises a region of complementarity to any one of SEQ ID NOs: 160-779, optionally wherein the region of complementarity comprises at least 15 consecutive nucleosides complementary to any one of SEQ ID NOs: 160-779.

19. The oligonucleotide of claim 18, wherein the oligonucleotide comprises at least 15 consecutive nucleosides of any one of SEQ ID NOs: 780-2019, optionally wherein the oligonucleotide comprises a sequence of any one of SEQ ID NOs: 780-2019, wherein each thymine base (T) may independently and optionally be replaced with a uracil base (U), and each U may independently and optionally be replaced with a T.

20. A method of delivering an oligonucleotide to a cell, the method comprising contacting the cell with the complex of claim 1.

21. A method of promoting the expression or activity of a dystrophin protein in a cell, the method comprising contacting the cell with the complex of claim 1 in an amount effective for promoting internalization of the oligonucleotide to the cell, optionally wherein the cell is a muscle cell.
